(19) United States
(12) Patent Application Publication
(10) Pub. No.: US 2020/0308301 A1
(43) Pub. Date: Oct. 1, 2020

(54) REVERSAL BINDING AGENTS FOR ANTI-FACTOR XI/XIA ANTIBODIES AND USES THEREOF

(71) Applicant: NOVARTIS AG, Basel (CH)

(72) Inventors: Stefan EWERT, Basel (CH); Alexander Wolfgang KOCH, Cambridge, MA (US)

(21) Appl. No.: 16/765,274

(22) PCT Filed: Nov. 20, 2018
(86) PCT No.: PCT/IB2018/059143

§ 371 Date: May 19, 2020

Related U.S. Application Data
(60) Provisional application No. 62/589,809, filed on Nov. 22, 2017.

(51) Int. Cl. C07K 16/36; C07K 16/00; A61P 7/04

(52) U.S. Cl. CPC C07K 16/36; C07K 16/005; A61P 7/04; A61K 2039/505; C07K 2317/565; C07K 2317/55; C07K 2317/92; C07K 2317/76

(57) ABSTRACT

The present disclosure relates to reversal agents, which specifically bind to anti-Factor XI and/or anti-Factor XIa antibodies, and reverse one or more anticoagulant effects of the anti-Factor XI and/or anti-Factor XIa antibodies, as well as to methods of use thereof, such as methods for reversing anticoagulant effects of such anti-Factor XI and/or anti-Factor XIa antibodies, and to related methods for managing bleeding or bleeding risks.

This application claims the benefit of U.S. Provisional Application No. 62/589,809 filed on Nov. 22, 2017 which is hereby incorporated by reference in its entirety.

### TECHNICAL FIELD

The present disclosure relates to binding agents (e.g., anti-idiotype antibodies), which specifically binds to anti-Factor XI and/or anti-Factor XIa (“anti-FXI/FXIa”) antibodies, and reverses one or more anticoagulant effects of the anti-Factor XI and/or anti-Factor XIa antibodies, as well as to pharmaceutical compositions and to methods of use thereof, such as methods for reversing anticoagulant effects of such anti-Factor XI and/or anti-Factor XIa antibodies.

### BACKGROUND

Thrombosis refers to thrombus formation inside blood vessels, subsequent to a combination of hereditary and acquired risk factors, known as thrombophilia or hypercoagulable states. Vessel wall damage, stasis, increased platelets reactivity and activation of clotting factors are some of the fundamental features of thrombosis. Thrombosis can occur in both venous and arterial circulation and can result in the development of deep vein thrombosis (DVT), pulmonary embolism, and stroke. If a thrombus occurs in the arterial system, down-stream ischemia can occur, leading to acute coronary syndromes (ACS), ischemic stroke, and acute limb ischemia. Thrombus formation in the venous system typically leads to deep venous thrombosis, pulmonary embolism and chronic thromboembolic pulmonary hypertension. Clots may also form in the left atrial appendage in patients with atrial fibrillation (AF), and dislodged thrombi may result in potentially devastating complications, i.e. thromboembolic stroke and systemic embolism. The currently available antithrombotic medications, including low molecular weight heparin (LMWH), thrombin inhibitors, and Factor Xa (FXa) inhibitors, are all associated with a significant risk of bleeding (Weitz J. I. (2010) Thromb. Haemost. 103, 62). The development of an antithrombotic agent that does not affect hemostasis, and therefore does not result in bleeding complications, as well as specific reversal agents, would be highly desirable.

Current anticoagulants are either injected or taken orally. The injectable anticoagulant LMWH is widely used and offers an improved therapeutic profile over formerly applied unfractionated heparin. For the past few decades the most commonly used oral anticoagulant has been warfarin. Warfarin has a narrow therapeutic window that requires frequent monitoring of the coagulation status, and shows a variety of drug-drug interactions. More recently, orally available direct FXa and thrombin inhibitors entered the anticoagulant market and are increasingly applied.

LMWHs, FXa inhibitors, and thrombin inhibitors are all efficacious in the prevention of post-operative venous thromboembolic disease, in the treatment of spontaneous DVT and pulmonary embolism, and in the stroke prevention in atrial fibrillation. However, these anticoagulants are also associated with bleeding complications that were generally comparable to those observed with the older drugs warfarin and unfractionated heparin. In the ADVANCE-2 clinical trial, the FXa inhibitor apixaban (Eliquis) was compared to the LMWH enoxaparin in patients after total knee replacement. While acute apixaban therapy was more effective at preventing venous thromboembolic disease than enoxaparin, both agents were associated with a significant risk of bleeding. Clinically relevant bleeding occurred in 4% of patients receiving apixaban and in 5% of patients treated with enoxaparin (Lassen, M. R., et al. (2009) N. Engl. J. Med. 361, 594).

In the RE-LY trial, the direct thrombin inhibitor dabigatran (Pradaxa) was compared to warfarin in patients with atrial fibrillation and a risk of stroke (Connolly, S. J., et al. (2009) N. Engl. J. Med. 361, 1139). Chronic dabigatran therapy was associated with a significantly lower risk of stroke or systemic embolism. However, major bleeding complications occurred in 3.1% of patients receiving 150 mg per day of dabigatran and in 3.4% of patients receiving warfarin (p=0.31).

Atrial fibrillation (AF) remains the most common cardiac arrhythmia in clinical practice, accounting for approximately one third of hospitalizations for cardiac dysrhythmias. Currently, it is estimated to affect more than 6 million patients in Europe and approximately 2.3 million in the United States, and this number continues to grow rapidly because of the increasing proportion of the aging population. It is estimated that approximately 5% of the population over the age of 65 years, and 10% of people aged over 80 years, will develop AF, however, the prevalence of AF is increasing beyond what is explained by age alone. AF risk factors such as hypertension, congestive heart failure, left ventricular hypertrophy, coronary artery disease and diabetes mellitus, and obstructive sleep apnea are also on the rise. As such, the number of affected individuals with AF is expected to increase two to three times over the next three decades in western populations. (Kannel and Benjamin (2008) Med Clin North Am. 2008; 92:17-40; Bunch, et al. (2012) J Innovations of Card Rhythm Manag 2012; 3: 855-63).

The principal risk of AF is a four- to five fold increase in embolic stroke. The attributable risk for stroke associated with AF increases steeply with age to 23.5% at ages 80 to 89. AF is associated with a doubling of mortality in both genders (Kannel and Benjamin 2008). AF is also independently associated with cognitive decline and all forms of dementia (Marzona, et al. (2012) CMAJ 2012; 184: 329-36; Geita et al 2013; Bunch et al 2012).

Most patients with AF require life-long anticoagulation therapy to prevent cardioembolic stroke and systemic embolism. The CHA2DS2-VASc risk score is a validated and widely used stratification tool to predict thromboembolic risk in atrial fibrillation patients and to identify patients who should benefit from anticoagulation therapy (LIP 2011; Camm, et al. (2012) Eur Heart J 2012; 33: 2719-2747); the accumulated evidence shows that CHA2DS2-VASc is at least as accurate as or possibly better than, scores such as CHADS2 in identifying patients who develop stroke and thromboembolism and definitively better at identifying ‘truly low-risk’ patients with AF. It is estimated that 85 to 90% of AF patients will require anticoagulation therapy.

In a meta-analysis comprising 6 trials which evaluated the effect of vitamin K antagonists (VKA) in reducing stroke and systemic embolism, a highly significant risk reduction in stroke incidence (relative risk reduction of 67% for stoke) was observed. All-cause mortality was significantly reduced (26%) by adjusted-dose VKA vs. control (Hart, Pearce, and Aguilar (2007) Ann Intern Med 2007; 146:857-867). An international normalized ratio (INR) target between 2 and 3 was associated with best benefit-risk ratio (Hylek et al (2003) N Engl J Med; 349:1019-1026) and universally adopted by international and national guidelines.

In recent years, new oral anticoagulants (NOAC) also referred to as direct oral anticoagulants (DOAC) have been approved and introduced to clinical practice. These drugs are at least as effective or even better than warfarin for reducing thrombo-embolic disease (Connolly, et al. (2009) N Engl J Med; 361:1139-51; Connolly, et al. (2011) N Engl J Med; 364:806-17; Patel, et al. (2011) N Engl J Med 2011; 365:883-91). NOAC were also associated with large reductions in the most devastating complications of warfarin namely hemorrhagic stroke and intracranial hemorrhage. Major bleeding events were similar or slightly lower than well conducted warfarin therapy. In addition NOAC are associated with a lower potential for drug-drug interaction than warfarin and could be used without routine monitoring; this is expected to ease their use in everyday medical practice.

Despite recent improvements, bleeding risk continues to be high with the use of anticoagulants. For instance, the annual incidence of major and clinically relevant non major bleeding was 14.9% and the annual incidence of major bleeding events was 3.6% in patients treated with rivaroxaban in the ROCKET study (Patel et al 2011). The annual incidence of major bleeding was >5% in patients at a high risk for bleeding defined as HAS Bled risk score ≥3 (Gallego, et al. (2012) Carc Arrhythm Electrophysiol.; 5:312-318). Major bleeding is a particularly relevant clinical outcome; for instance in the ROCKET study, once major bleeding has occurred, all-cause mortality rate was 20.4% in the rivaroxaban group and 26.1% in the warfarin group. Once major bleeding events have occurred stroke and systemic embolism occurred in 4.7% and 5.4% of patients in rivaroxaban and warfarin groups, respectively (Piccini, et al. (2014) Eur Heart J; 35:1873-80). Hospital stay, transfusion of blood products and resources utilization were also severely impacted by the occurrence of major bleeding. Bleeding risk is also a major reason for not receiving anticoagulants in eligible patients. In the Euro Heart Survey on Atrial Fibrillation comprising data from 182 hospitals in 35 countries and 5333 ambulant and hospitalized AF patients, only 67% of eligible patients received oral anticoagulant at discharge (Nieuwlaat, et al (2005) Eur Heart J; 26, 2422-2434). A high unmet medical need therefore exists for a safer therapy which can reduce AF thromboembolic complications such as stroke, systemic embolism, cognitive decline and mortality with comparable efficacy as existing therapy but with a lower bleeding liability.

Factor XI (FXI) holds important roles in both intrinsic and extrinsic coagulation pathways and in bridging the initiation and amplification phases of plasmatic hemostasis (Gailani and Renne (2007) Arterioscler Thromb Vasc Biol; 27(12):2507-13). Both Factor XII and thrombin can activate FXI, resulting in a sustained thrombin generation and fibrinolysis inhibition. FXI plays a minor role in normal hemostasis in a high tissue factor environment “after vessel injury” whereas it appears to play a key role in thrombosis. Severe FXI deficiency is associated with a lower incidence of ischemic stroke and venous thromboembolic events (Salomon et al (2008) Blood; 111(8):4113-7; Salomon et al (2011) Thromb Haemost; 105(2):269-73). Furthermore, in a population-based study, a survival advantage of severe FXI deficiency was evoked as a result of a lower incidence of thromboembolic events (Duga and Salomon, (2013) Semin Thromb Hemost; 39(6):621-31). Bleeding manifestations in subjects with severe FXI deficiency are infrequent, usually mild, injury-related, and affect preferably tissues with increased fibrinolytic activity such as the oral mucosa, nasal mucosa and urinary tract (Bolton-Maggs, (2000) Haemophilia; 6 Suppl 1:100-9). Bleeding in vital organs is extremely rare or not existing.

Accordingly, as part of efforts to lower bleeding liability, there is also a high unmet medical need for specific, reversal agents for anticoagulant therapies, for example, in circumstances when reversal of the anticoagulant effects of a therapy is needed for emergency surgery/urgent procedures and in life-threatening or uncontrolled bleeding.

### SUMMARY

Lower bleeding risk is associated with anticoagulant therapies involving anti-FXI/FXIa antibodies, compared to NOACs. For example, anti-Factor XI/FXIa antibody NOV1401 is a human antibody binding to the catalytic domain of FXI. NOV1401 inhibits both the zymogen (FXI) and the activated factor XI (FXIa) with high potency. Anti-FXI/FXIa antibody NOV1401 dose-dependently prolonged activated partial thromboplastin time (aPTT) in in vitro and in in vivo studies. After a single subcutaneous (s.c.) administration of NOV1401 at a 3 mg/kg dose, a sustained anticoagulant activity lasting more than one month was observed in cynomolgus monkeys. Moreover, Anti-FXI/FXIa antibody NOV1401 prevented experimental carotid artery thrombosis induced by FeCl3 and induced prolongation in aPTT in FXI−/− mice reconstituted with human FXI. NOV1401 was well tolerated in the 13 week Good Laboratory Practice (GLP)-compliant toxicity study conducted in cynomolgus monkeys.

Despite the lower bleeding risk with anti-FXI/FXIa antibodies, such as antibody NOV1401, compared to NOACs, bleeding events may still happen in certain circumstances due to trauma, surgery, procedures, co-medication and high prevalence of comorbidities that increase bleeding risk such as hypertension, heart failure, renal impairment, hepatic impairment, older age, prior bleeding events, risk of falls, use of antiplatelet agents or non-steroidal anti-inflammatory drugs, etc.

Accordingly, as part of efforts to lower bleeding liability, the present disclosure describes strategies to address the high unmet medical need for specific, reversal agents for anticoagulant therapies that are anti-Factor XI/XIa antibodies (e.g., anti-FXI/FXIa antibodies which specifically bind to the catalytic domain of FXI/FXIa). In specific aspects, managing bleeding or bleeding risk is beneficial in circumstances when reversal of the anticoagulant effects of a therapy is needed, for example, for emergency surgery/urgent procedures and in cases of life-threatening or uncontrolled bleeding. In specific aspects, managing bleeding or bleeding risk is beneficial in patients identified as having high bleeding risk (e.g., previous history of bleeding).

The present disclosure relates to binding agents that are anti-idiotype antibodies, e.g., full length IgGs, and fragments thereof such as Fabs, which specifically binds to antibodies that specifically bind coagulation Factor XI and XIa (activated Factor XI) (hereinafter, sometimes referred to as “FXI”, “FXIa,” and similar terms), and which are capable of reversing one or more anticoagulant effects of such anti-FXI/FXIa antibodies (e.g., capable of reducing aPTT or bleeding time) and/or inhibits binding of the antibodies to FXI/FXIa. In particular, the present disclosure also relates to pharmaceutical compositions comprising such binding agents, and methods of reversing one or more anticoagulant effects of an anti-FXI/FXIa antibody in a patient (e.g., human patient) being treated with the anti-FXI/FXIa antibody, comprising administering the binding agent. Such binding agents capable of reversing one or more anticoagulant effects of anti-FXI/FXIa antibodies achieve an unmet need in circumstances when reversal of the anticoagulant effects of a therapy, such as anti-FXI/XIa antibodies, is needed for emergency surgery/urgent procedures and in life-threatening or uncontrolled bleeding.

In specific aspects, such patients (e.g., human patients) are being treated with an anti-FXI/FXIa antibody for the prevention and/or treatment of thrombosis or thromboembolic disease/disorder (e.g., thrombic stroke, atrial fibrillation, stroke prevention in atrial fibrillation (SPAF), deep vein thrombosis, venous thromboembolism, pulmonary embolism, acute coronary syndromes (ACS), ischemic stroke, acute limb ischemia, chronic thromboembolic pulmonary hypertension, systemic embolism). In specific aspects, binding agents provided herein that reverses one or more anticoagulant effects of anti-FXI/FXIa antibodies are anti-idiotype antibodies, and in further specific aspects, such anti-idiotype antibodies are full length IgGs. In further specific aspects, such anti-idiotype antibodies are monoclonal antibodies, such as human monoclonal antibodies, e.g., recombinant human monoclonal antibodies.

In particular aspects, the present disclosure also relates to isolated polynucleotides and nucleic acids comprising a sequence encoding a binding agent provided herein, to vectors comprising one or more of the polynucleotides or nucleic acids provided herein, to host cells comprising such vectors or polynucleotides or nucleic acids. In specific aspects, the host cells are non-human mammalian cells, such as Chinese hamster ovary (CHO) cells.

Non-limiting embodiments of the present disclosure are described in the following aspects:
1. A pharmaceutical composition comprising a binding agent which specifically binds a target antibody that binds human Factor XI (“FXI”) and/or Factor XIa (“FXIa”) within the catalytic domain, wherein the binding agent inhibits an anticoagulant activity of the target antibody, wherein the binding agent is selected from Table 2, and wherein the binding agent is in a liquid formulation comprising sucrose and/or histidine.
2. The pharmaceutical composition of embodiment 1, wherein the liquid formulation comprises sucrose and histidine.
3. The pharmaceutical composition of embodiment 1 or 2, wherein the liquid formulation comprises at least 200, 210, 220, 230, 240, or 250 mM sucrose.
4. The pharmaceutical composition of embodiment 3, wherein the liquid formulation comprises 220 mM sucrose.
5. The pharmaceutical composition of any one of the preceding embodiments, wherein the liquid formulation comprises at least 10 mM or at least 20 mM histidine.
6. The pharmaceutical composition of any one of the preceding embodiments, wherein the liquid formulation comprises 20 mM histidine.
7. The pharmaceutical composition of any one of the preceding embodiments, wherein the liquid formulation is at a pH of 4.5-7.
8. The pharmaceutical composition of any one of the preceding embodiments, wherein the liquid formulation is at a pH of 5.5.
9. The pharmaceutical composition of any one of the preceding embodiments, wherein the binding agent is formulated at a concentration in the range of 50 mg/mL to 150 mg/mL.
10. The pharmaceutical composition of any one of the preceding embodiments, wherein the binding agent is formulated at a concentration of at least 150 mg/mL.
11. The pharmaceutical composition of any one of the preceding embodiments, wherein the liquid formulation comprises 220 mM sucrose, 20 mM histidine, 0.04% Polysorbate 20, and 150 mg/mL concentration of the binding agent, at pH 5.5.
12. The pharmaceutical composition of any one of the preceding embodiments, which is formulated for subcutaneous or intravenous administration.
13. The pharmaceutical composition of any one of the preceding embodiments, wherein the target antibody comprises (i) a heavy chain variable region (VH) comprising the amino acid sequence of SEQ ID NO: 12 and a light chain variable region (VL) comprising the amino acid sequence of SEQ ID NO: 23; or (ii) a heavy chain comprising the amino acid sequence of SEQ ID NO: 14 and a light chain comprising the amino acid sequence of SEQ ID NO: 25.
14. The pharmaceutical composition of any one of the preceding embodiments, wherein the binding agent is an antibody or fragment thereof comprising a variable heavy chain region (VH) and a light chain variable region (VL), wherein:
   a) the VH comprises the amino acid sequence of SEQ ID NO: 39 and the VL comprises the amino acid sequence of SEQ ID NO: 55;
   b) the VH comprises the amino acid sequence of SEQ ID NO: 71 and the VL comprises the amino acid sequence of SEQ ID NO: 87;
   c) the VH comprises the amino acid sequence of SEQ ID NO: 103 and the VL comprises the amino acid sequence of SEQ ID NO: 119;
   d) the VH comprises the amino acid sequence of SEQ ID NO: 135 and the VL comprises the amino acid sequence of SEQ ID NO: 151;
   e) the VH comprises the amino acid sequence of SEQ ID NO: 167 and the VL comprises the amino acid sequence of SEQ ID NO: 183;
   f) the VH comprises the amino acid sequence of SEQ ID NO: 199 and the VL comprises the amino acid sequence of SEQ ID NO: 215;
   g) the VH comprises the amino acid sequence of SEQ ID NO: 231 and the VL comprises the amino acid sequence of SEQ ID NO: 247;
   h) the VH comprises the amino acid sequence of SEQ ID NO: 263 and the VL comprises the amino acid sequence of SEQ ID NO: 279;
   i) the VH comprises the amino acid sequence of SEQ ID NO: 295 and the VL comprises the amino acid sequence of SEQ ID NO: 311; or
   j) the VH comprises the amino acid sequence of SEQ ID NO: 327 and the VL comprises the amino acid sequence of SEQ ID NO: 343.
15. The pharmaceutical composition of any one of the preceding embodiments, wherein the binding agent is antibody Fab IDT1, IDT2, IDT3, IDT4, IDT5, IDT6, IDT7, IDT8, IDT9, or IDT10 as set forth in Table 2, or is antibody IgG IDT11 or IDT12 as set forth in Table 2.
16. A binding agent which specifically binds a target antibody that binds human FXI and/or FXIa within the catalytic domain, wherein the binding agent inhibits an anticoagulant activity of the target antibody,
   wherein the target antibody comprises (i) a heavy chain variable region (VH) comprising the amino acid sequence of SEQ ID NO: 12 and a light chain variable region (VL) comprising the amino acid sequence of SEQ ID NO: 23; or (ii) a heavy chain comprising the amino acid sequence of SEQ ID NO: 14 and a light chain comprising the amino acid sequence of SEQ ID NO: 25; and
   wherein the binding agent is an antibody or antigen-binding fragment thereof comprising (a) a heavy chain comprising the amino acid sequence of SEQ ID NO: 347 and a light chain comprising the amino acid sequence of SEQ ID NO: 57, or (b) a heavy chain comprising the amino acid sequence of SEQ ID NO: 349 and a light chain comprising the amino acid sequence of SEQ ID NO: 89.
17. A binding agent which specifically binds a target antibody that binds human FXI and/or FXIa within the catalytic domain, wherein the binding agent reverses an anticoagulant activity of the target antibody, and wherein the binding agent is anti-idiotype antibody IDT11 or IDT12 as set forth in Table 2.
18. A polynucleotide comprising nucleotide sequences encoding the binding agent of any one of the preceding embodiments.
19. A vector comprising the polynucleotide of embodiment 18.
20. A host cell comprising the polynucleotide of embodiment 18.
21. A host cell comprising the vector of embodiment 19.
22. A method of producing a binding agent, said method comprises culturing the host cell of embodiment 20 or 21 under suitable conditions for expression of the binding agent or a portion thereof, wherein the method optionally comprises purifying the binding agent.
23. A pharmaceutical composition comprising the binding agent of any one of the preceding embodiments.
24. A pharmaceutical composition for use as a medicament for reversing the anticoagulant effect of an anti-FXI/FXIa antibody in a patient being treated with the anti-Factor XI/Factor XIa antibody, wherein the pharmaceutical composition comprises an effective amount of the binding agent of any one of the preceding embodiments.
25. A method for reversing the anticoagulant effect of an anti-FXI/FXIa antibody in a patient being treated with the anti-FXI/FXIa antibody or antigen-binding fragment thereof, comprising administering an effective amount of the binding agent of any one of the preceding embodiments to a patient in need thereof.
26. The method of embodiment 25, wherein the anti-FXI/FXIa antibody or antigen-binding fragment thereof comprises (i) a VH comprising the amino acid sequence of SEQ ID NO: 12 and a VL comprising the amino acid sequence of SEQ ID NO: 23; or (ii) a heavy chain comprising the amino acid sequence of SEQ ID NO: 14 and a light chain comprising the amino acid sequence of SEQ ID NO: 25.
27. The method of embodiment 25, wherein the anti-FXI/FXIa antibody or antigen-binding fragment thereof comprises (i) a VH comprising complementarity determining regions HCDR1, HCDR2 and HCDR3 of a VH comprising the amino acid sequence of SEQ ID NO: 12 and (ii) a VL comprising complementarity determining regions LCDR1, LCDR2, and LCDR3 of a VL comprising the amino acid sequence of SEQ ID NO: 23.
28. The method of any one of the preceding embodiments, wherein the method further comprises applying one of the following to the patient: (i) fluid replacement using colloids, crystalloids, human plasma or plasma proteins such as albumin; (ii) transfusion with packed red blood or whole blood; or (iii) administration of fresh frozen plasma (FFP), prothrombin complex concentrates (PCC), activated PCC (APCC), such as, factor VIII inhibitor, and/or recombinant, activated factor VII.
29. The method of any one of the preceding embodiments, wherein the patient has or is at risk of developing thrombosis.
30. The method of any one of the preceding embodiments, wherein the patient has
   a. atrial fibrillation;
   b. suspected or confirmed cardiac arrhythmia such as paroxysmal, persistent or permanent atrial fibrillation or atrial flutter;
   c. Chronic Thromboembolic Pulmonary Hypertension (CTEPH);
   d. valvular heart disease with or without atrial fibrillation;
   e. pulmonary hypertension;
   f. congenital or acquired thrombophilia including but not exclusively factor V Leiden, prothrombin mutation, antithrombin III, protein C and protein S deficiencies, factor XIII mutation, familial dysfibrinogenemia, congenital deficiency of plasminogen, increased levels of factor XI, sickle cell disease, antiphospholipid syndrome, autoimmune disease, chronic bowel disease, nephrotic syndrome, hemolytic uremia, myeloproliferative disease, disseminated intra vascular coagulation, paroxysmal nocturnal hemoglobinuria and heparin induced thrombopenia; or
   g. chronic kidney disease.
31. The method of any one of the preceding embodiments, wherein the patient has non-valvular atrial fibrillation.
32. The method of any one of the preceding embodiments, wherein the patient has a demonstrated high risk of bleeding.
33. The method of any one of the preceding embodiments, wherein the patient has chronic kidney disease.
34. The method of embodiment 33, wherein patient has end stage renal disease (ESRD).
35. The method of embodiment 34, wherein the patient has ESRD and is undergoing dialysis.
36. The method of embodiment 35, wherein the patient has non-valvular atrial fibrillation.
37. The method of any one of the preceding embodiments, wherein the patient is being administered the anti-FXI/FXIa antibody or antigen-binding fragment thereof to reduce the risk of stroke and/or systemic embolism.
38. The method of any one of the preceding embodiments, wherein reversal of the anticoagulant effect of the anti-FXI/FXIa antibody or antigen-binding fragment thereof is needed for emergency surgery/urgent procedures and in life-threatening or uncontrolled bleeding.

### BRIEF DESCRIPTION OF THE DRAWINGS

FIG. 1 shows representative binding curves from SET experiments for each of the 12 anti-NOV1401 antibodies (Fabs and IgGs) as described in Examples. K_{D} values were determined from fitting the experimental data to a 1:1 binding model for Fabs and IgGs as described in Examples. Average K_{D} values from two to six individual experiments are shown.

FIG. 2 shows representative SPR response curves for binding of NOV1401 and three NOV1401/anti-NOV1401 mixtures to immobilized FXIa. Increasing concentrations of anti-NOV1401 reduce binding of NOV1401 to FXIa with a 10 fold molar excess completely blocking the binding. These data indicate that anti-NOV1401 is capable to bind to and block NOV1401 from interacting with FXIa. Anti-NOV1401 alone did not show any binding to immobilized FXIa (not shown).

FIG. 3 shows aPTT assay results for two representative anti-NOV1401 Fabs when NOV1401 was preincubated for 10 min with anti-NOV1401 before FXI-containing human plasma was added and the intrinsic pathway of the coagulation cascade was triggered. Both anti-NOV1401 Fabs block the aPTT prolonging effect of NOV1401 in a concentration-dependent manner, i.e. inhibit the effect of NOV1401. 100% inhibition (dotted line) was achieved at 3× molar access of anti-NOV1401.

FIG. 4 shows aPTT assay results for 10 anti-NOV1401 Fabs and two anti-NOV1401 IgGs when NOV1401 was pre-incubated for 5 min with FXI-containing human plasma before anti-NOV1401 Fab or IgG was added and the intrinsic pathway of the coagulation cascade was triggered. All 12 anti-NOV1401 show a concentration-dependent partial reversal of the effects of NOV1401 on aPTT.

FIG. 5 shows TGA results for 10 anti-NOV1401 Fabs and two anti-NOV1401 IgGs when NOV1401 was pre-incubated for 5 min with FXI-containing human plasma before anti-NOV1401 Fab or IgG was added and the thrombin feedback loop was triggered. The TGA was conducted at a constant concentration for NOV1401 of 0.05 M, which corresponds to the IC₅₀ value determined in a separate experiment. All 12 anti-NOV1401 show a concentration-dependent partial reversal of the effects of NOV1401 on thrombin generation.

FIG. 6 shows ex-vivo aPTT assay results from blood/plasma samples of cynomolgus monkeys treated with a single 3 mg/kg subcutaneous dose of NOV1401 on study day one followed by two i.v. doses of IDT3 on study days 4 and 5, respectively.

### DETAILED DESCRIPTION

### Terminology

Unless defined otherwise, all technical and scientific terms used herein have the same meaning as commonly understood by those of ordinary skill in the art to which this present disclosure pertains.

As used in the specification and claims, the singular form “a”, “an” and “the” include plural references unless the context clearly dictates otherwise. For example, the term “a cell” includes a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” It also is to be understood, although not always explicitly stated, that the reagents described herein are merely examples and that equivalents of such are known in the art.

The terms “binding agent,” “reversal agent,” and “antidote” are used interchangeably, and, in the context of an antibody which specifically binds to Factor XI and/or Factor XIa (“anti-FXI/FXIa antibody”), refer to a protein, polypeptide, or a complex thereof, such as an anti-idiotype antibody or a fragment thereof such as a Fab fragment, or an inactive FXI/FXIa-derived polypeptide or protein fragment that specifically binds to an anti-FXI/FXIa antibody, such as, the antigen-binding region(s) or variable region(s) of the anti-FXI/FXIa antibody. In specific aspects provided herein, the binding agent is capable of reversing (e.g., partially reversing by at least 10%, at least 20%, at least 30%, at least 40%, at least 50%, at least 60%, at least 70%) one or more anticoagulant effects of the anti-FXI/FXIa antibody (e.g., antibody NOV1401). In further specific aspects provided herein, the binding agent is capable of blocking binding of an anti-FXI/FXIa antibody to its antigen, e.g., FXI/FXIa. In a specific aspect, as used herein, the terms “anti-NOV1401,” “anti-NOV1401 antibody,” “anti-NOV1401 Fab,” “anti-NOV1401 IgG,” “NOV1401 binding agent,” “NOV1401 antidote,” and the likes, are used interchangeably and refer to a binding agent or reversal agent, such as an anti-idiotype antibody or a fragment thereof, which specifically binds to anti-Factor XI antibody NOV1401 (see Table 1). Non-limiting examples of NOV1401 binding/reversal agents are described herein, for example, Table 2.

The terms “anti-idiotype antibody,” “anti-Id antibody,” and “anti-idiotypic antibody” are used interchangeably, and refer to an antibody and fragments thereof (e.g., Fab fragment) that specifically binds to the antigen-binding region(s) of another antibody. Anti-idiotype antibodies are typically raised against the antigen-binding region(s) or complementarity determining regions (CDRs) (idiotype) of a target antibody. Anti-Idiotype antibodies can be produced by various methods described previously, see, e.g., Pan et al., 1995, FASEB J. 9:43-49.

The terms “FXI protein,” “FXI antigen,” and “FXI” are used interchangeably, and refer to the Factor XI protein in different species. Factor XI is the mammalian plasma coagulation factor XI, a glycoprotein present in human plasma at a concentration of 25-30 nM as a zymogen that when converted by limited proteolysis to an active serine protease, participates in the intrinsic pathway of blood coagulation.

The terms “FXIa protein,” “FXIa antigen,” and “FXIa”, are used interchangeably, and refers to the activated FXI protein in different species. The zymogen Factor XI is converted into its active form, the coagulation factor XIa (FXIa), either via the contact phase of blood coagulation or through thrombin-mediated activation on the platelet surface. During this activation of factor XI, an internal peptide bond is cleaved in each of the two chains, resulting in the activated factor XIa, a serine protease composed of two heavy and two light chains held together by disulfide bonds. This serine protease FXIa converts the coagulation Factor IX into IXa, which subsequently activates coagulation Factor X (Xa). Xa then can mediate coagulation Factor 11/Thrombin activation. For example, human FXI has the sequence as set out in Table 1 (SEQ ID NO: 1), and has been described in previous reports and literature (Mandle R J Jr, et al. (1979) Blood; 54(4):850; NCBI Reference Sequence: AAA51985).

In the context of this disclosure, the terms “FXI” and “FXIa” (and the like) include mutants and variants of the natural FXI and FXIa protein, respectively, which have substantially the same amino acid sequence as that of the native primary structure (amino acid sequence) described in the above-mentioned reports.

The term “catalytic domain,” “serine protease catalytic domain,” and similar terms as used herein in the context of human FXI or FXIa, means amino acids Ile370 to Val607, as counted from the Glu1 at the N-terminus of the mature protein that is in circulation. It can also be described as residues 388-625 at the C-terminus of FXI. As used herein, the term “active site” means the catalytic triad comprised of the amino acids His413, Asp462 and Se557. (See, e.g., Bane and Gailani (2014) Drug Disc. 19(9), which is incorporated by reference herein in its entirety).

The term “antibody” as used herein means a whole antibody and any antigen binding fragment (i.e., “antigen-binding portion”) or single chain thereof and is derived from an immunoglobulin (“Ig”) molecule that specifically binds to an antigen. A whole antibody is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. In some specific aspects, an antibody can be a monoclonal antibody, human antibody, humanized antibody, camelised antibody, or chimeric antibody. Antibodies can be of any isotype (e.g., immunoglobulin G (IgG), immunoglobulin E (IgE), immunoglobulin M (IgM), immunoglobulin D (IgD), immunoglobulin A (IgA) and immunoglobulin Y (IgY)), class (e.g., IgG1, IgG2, IgG3, IgG4, IgA1 and IgA2) or subclass.

The term “IgG” or “IgG antibody” as used herein, and unless specified otherwise, means a type G whole antibody or Ig.

The term “antigen binding portion” or “antigen binding fragment” of an antibody, as used herein, refers to one or more fragments of an intact antibody that retain the ability to specifically bind to a given antigen (e.g., anti-FXI/FXIa antibody, such as NOV1401). Antigen binding functions of an antibody can be performed by fragments of an intact antibody. Examples of binding fragments encompassed within the term antigen binding portion or antigen binding fragment of an antibody include a Fab fragment, a monovalent fragment consisting of the VL, VH, CL and CH1 domains; a F(ab)2 fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody; a single domain antibody (dAb) fragment (Ward et al., 1989 Nature 341:544-546), which consists of a VH domain or a VL domain; and an isolated complementarity determining region (CDR).

Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies include one or more antigen binding portions or fragments of an antibody. These antibody fragments are obtained using conventional techniques known to those of skill in the art, and the fragments are screened for utility in the same manner as are intact antibodies.

Antigen binding fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136). Antigen binding portions of antibodies can be grafted into scaffolds based on polypeptides such as Fibronectin type III (Fn3) (see U.S. Pat. No. 6,703,199, which describes fibronectin polypeptide monobodies).

Antigen binding fragments can be incorporated into single chain molecules comprising a pair of tandem Fv segments (VH-CH1-VH-CH1) which, together with complementary light chain polypeptides, form a pair of antigen binding regions (Zapata et al., 1995 Protein Eng.
8(10):1057-1062; and U.S. Pat. No. 5,641,870).

As used herein, the term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity. As used herein, the term “high affinity” for an antibody or antigen binding fragments thereof (e.g., a Fab fragment) generally refers to an antibody, or antigen binding fragment, having a K_{D} of 10-9 M or less (e.g., a K_{D} of 10-10 M or less, a K_{D} of 10-11 M or less, a K_{D} of 10-12 M or less, a K_{D} of 10-13 M or less, a K_{D} of 10-14 M or less, etc.).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “binding specificity” as used herein refers to the ability of an individual antibody combining site to react with only one antigenic determinant.

As used herein, the terms “immunospecifically binds,” “immunospecifically recognizes,” “specifically binds,” and “specifically recognizes” are analogous terms in the context of antibodies and refer to molecules that bind to an antigen (e.g., epitope or immune complex) as such binding is understood by one skilled in the art. For example, a molecule that specifically binds to an antigen may bind to other peptides or polypeptides, generally with lower affinity as determined by, e.g., immunoassays, Biacore™, KinExA 3000 instrument (Sapidyne Instruments, Boise, Id.), or other assays known in the art. In a specific embodiment, molecules that immunospecifically bind to an antigen bind to the antigen with a Ka that is at least 2 logs, 2.5 logs, 3 logs, 4 logs or greater than the Ka when the molecules bind to another antigen. In another specific embodiment, molecules that immunospecifically bind to an antigen do not cross react with other proteins.

The term “FXI and/or FXIa mediated” refers to the fact that FXI and/or FXIa mediates the intrinsic and/or common coagulation pathways by directly or indirectly activating Factor IX (also known as FIX), Factor X (FX), and/or thrombin, and/or by binding to platelet receptors.

The term “hemostasis” represents the principal mechanisms for arresting the flow of blood at sites of injury and restoring vascular patency during wound healing, respectively. During normal hemostasis and pathological thrombosis, three mechanisms become activated simultaneously: primary hemostasis meaning the interactions of activated platelets with the vessel wall, the formation of fibrin, and a process termed as fibrinolysis.

The terms “coagulation and coagulation cascade,” “cascade model of coagulation,” and the like, refer to the protein based system which serves to stabilize a clot that has formed to seal up a wound. The coagulation pathway is a proteolytic cascade. Each enzyme of the pathway is present in the plasma as a Zymogen (in an inactive form), which on activation undergoes proteolytic cleavage to release the active factor from the precursor molecule. The coagulation cascade functions as a series of positive and negative feedback loops which control the activation process. The ultimate goal of the pathway is to produce thrombin, which can then convert soluble fibrinogen into fibrin that forms a clot.

The process of generation of thrombin can be divided into three phases: the intrinsic and extrinsic pathways, which provide alternative routes for the generation of an active clotting factor: FXa (Activated Factor-X), and the final common pathway, which results in thrombin formation (Hoffman M. M. and Monroe D. M. (2005) Curr Hematol Rep. 4:391-396; Johne J, et al. (2006) Biol Chem. 387:173-178).

As used herein, the terms “manage,” “managing,” and “management” refer to the beneficial effects that a subject derives from a therapy (e.g., a prophylactic or therapeutic agent), which does not result in a cure of a disease, disorder, or condition (e.g., thrombosis or thromboembolic disorder). In certain embodiments, a subject is administered one or more therapies (e.g., binding agent or antibody described herein) to “manage” thrombosis or thromboembolic disorder, one or more symptoms thereof, so as to prevent the progression or worsening of the condition or disorder.

“Platelet aggregation” refers to the process whereby when a break in a blood vessel occurs, substances are exposed that normally are not in direct contact with the blood flow. These substances (primarily collagen and von Willebrand factor) allow the platelets to adhere to the broken surface. Once a platelet adheres to the surface, it releases chemicals that attract additional platelets to the damaged area, referred to as platelet aggregation. These two processes are the first responses to stop bleeding.

A “thromboembolic disorder,” or similar terms as used herein, refer to any number of conditions or diseases in which the intrinsic and/or common coagulation pathways are aberrantly activated or are not naturally deactivated (e.g., without therapeutic means). These conditions include but are not limited to thrombic stroke, atrial fibrillation, stroke prevention in atrial fibrillation (SPAF), deep vein thrombosis, venous thromboembolism, and pulmonary embolism. These can also include catheter-related conditions (e.g., Hickman catheter in oncology patients) in which catheters become thrombosed, and extracorporeal membrane oxygenation (ECMO), in which the tubing develops clots.

A “thromboembolic,” or similar terms as used herein, can also refer to any number of the following, which the anti-FXI and/or FXIa Abs or antigen binding fragments thereof of the present disclosure can be used to prevent or treat or to reduce the risk of:
thromboembolism in subjects with suspected or confirmed cardiac arrhythmia such as paroxysmal, persistent or permanent atrial fibrillation or atrial flutter;
stroke prevention in atrial fibrillation (SPAF), a subpopulation of which is AF patients undergoing percutaneous coronary interventions (PCI);
acute venous thromboembolic events (VTE) treatment and extended secondary VTE prevention in patients at high risk for bleeding;
cerebral and cardiovascular events in secondary prevention after transient ischemic attack (TIA) or non-disabling stroke and prevention of thromboembolic events in heart failure with sinus rhythm;
clot formation in left atrium and thromboembolism in subjects undergoing cardioversion for cardiac arrhythmia;
thrombosis before, during and after ablation procedure for cardiac arrhythmia;
venous thrombosis, this includes but not exclusively, treatment and secondary prevention of deep or superficial veins thrombosis in the lower members or upper member, thrombosis in the abdominal and thoracic veins, sinus thrombosis and thrombosis of jugular veins;
thrombosis on any artificial surface in the veins like catheter or pacemaker wires;
pulmonary embolism in patients with or without venous thrombosis;
Chronic Thromboembolic Pulmonary Hypertension (CTEPH);
arterial thrombosis on ruptured atherosclerotic plaque, thrombosis on intra-arterial prosthesis or catheter and thrombosis in apparently normal arteries, this includes but not limited to acute coronary syndromes, ST elevation myocardial infarction, non ST elevation myocardial infarction, unstable angina, stent thrombosis, thrombosis of any artificial surface in the arterial system and thrombosis of pulmonary arteries in subjects with or without pulmonary hypertension;
thrombosis and thromboembolism in patients undergoing percutaneous coronary interventions (PCI);
cardioembolic and cryptogenic strokes;
thrombosis in patients with invasive and non-invasive cancer malignancies;
thrombosis over an indwelling catheter;
thrombosis and thromboembolism in severely ill patients;
cardiac thrombosis and thromboembolism, this includes but not exclusively cardiac thrombosis after myocardial infarction, cardiac thrombosis related to condition such as cardiac aneurysm, myocardial fibrosis, cardiac enlargement and insufficiency, myocarditis and artificial surface in the heart;
thromboembolism in patients with valvular heart disease with or without atrial fibrillation;
thromboembolism over valvular mechanic or biologic prostheses;
thromboembolism in patients who had native or artificial cardiac patches, arterial or venous conduit tubes after heart repair of simple or complex cardiac malformations;
venous thrombosis and thromboembolism after knee replacement surgery, hip replacement surgery, and orthopedic surgery, thoracic or abdominal surgery;
arterial or venous thrombosis after neurosurgery including intracranial and spinal cord interventions;
congenital or acquired thrombophilia including but not exclusively factor V Leiden, prothrombin mutation, antithrombin III, protein C and protein S deficiencies, factor XIII mutation, familial dysfibrinogenemia, congenital deficiency of plasminogen, increased levels of factor XI, sickle cell disease, antiphospholipid syndrome, autoimmune disease, chronic bowel disease, nephrotic syndrome, hemolytic uremia, myeloproliferative disease, disseminated intra vascular coagulation, paroxysmal nocturnal hemoglobinuria and heparin induced thrombopenia;
thrombosis and thromboembolism in chronic kidney disease; and
thrombosis and thromboembolism in patients undergoing hemodialysis and in patients undergoing extra-corporal membrane oxygenation.

The term “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen while having reduced antigenicity in human as compared to the original mouse antibody.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the present disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In some embodiments, the term “conservative sequence modifications” are used to refer to amino acid modifications that do not significantly affect or alter the binding characteristics of the antibody containing the amino acid sequence.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents. Two antibodies are said to “compete” if one antibody is shown to bind the same epitope as the second antibody in a competitive binding assay, by any of the methods well known to those of skill in the art.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies of the present disclosure may include amino acid residues not encoded by human sequences (e.g., mutations introduced by random or site-specific mutagenesis in vitro or by somatic mutation in vivo).

The term “human monoclonal antibody” refers to antibodies displaying a single binding specificity which have variable regions in which both the framework and CDR regions are derived from human sequences. In one embodiment, the human monoclonal antibodies are prepared using phage display methods for screening libraries of human immunoglobulin genes.

A “humanized” antibody is an antibody that retains the reactivity of a non-human antibody while being less immunogenic in humans. This can be achieved, for instance, by retaining the non-human CDR regions and replacing the remaining parts of the antibody with their human counterparts (i.e., the constant region as well as the framework portions of the variable region). See, e.g., Morrison et al., Proc. Natl. Acad. Sci. USA, 81:6851-6855, 1984; Morrison and Oi, Adv. Immunol., 44:65-92, 1988; Verhoeyen et al., Science, 239:1534-1536, 1988; Padlan, Molec. Immun., 28:489-498, 1991; and Padlan, Molec. Immun., 31:169-217, 1994. Other examples of human engineering technology include, but are not limited to Xoma technology disclosed in U.S. Pat. No. 5,766,886.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch, J. Mol. Biol. 48:443, 1970, by the search for similarity method of Pearson and Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., Current Protocols in Molecular Biology, John Wiley & Sons, Inc. (Ringbou ed., 2003)).

Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol, Biol. 48:444-453, 1970) algorithm which has been incorporated into the GAP program in the GCG software package (available on the world wide web at gcg.com), using either a Blossom 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “isolated antibody” refers to an antibody that is substantially free of other antibodies having different antigenic specificities (e.g., an isolated antibody that specifically binds FXI and/or FXIa is substantially free of antibodies that specifically bind antigens other than FXI and/or FXIa, or an isolated anti-idiotype antibody that specifically binds an anti-FXI/FXIa antibody is substantially free of antibodies that specifically bind antigens other than the anti-FXI/FXIa antibody). An isolated antibody that specifically binds FXI and/or FXIa may, however, have cross-reactivity to other antigens. Moreover, an isolated antibody may be substantially free of other cellular material and/or chemicals.

The term “isotype” refers to the antibody class (e.g., IgM, IgE, IgG such as IgG1 or IgG4) that is provided by the heavy chain constant region genes. Isotype also includes modified versions of one of these classes, where modifications have been made to alter the Fc function, for example, to enhance or reduce effector functions or binding to Fc receptors.

The term “kassoc” or “ka”, as used herein, is intended to refer to the association rate of a particular antibody-antigen interaction, whereas the term “kdis” or “kd,” as used herein, is intended to refer to the dissociation rate of a particular antibody-antigen interaction. The term “KD”, as used herein, is intended to refer to the dissociation constant, which is obtained from the ratio of kd to ka (i.e. kd/ka) and is expressed as a molar concentration (M). KD values for antibodies can be determined using methods well established in the art. Methods for determining the KD of an antibody include measuring surface plasmon resonance using a biosensor system such as a Biacore™ system, or measuring affinity in solution by solution equilibrium titration (SET).

The terms “monoclonal antibody” or “monoclonal antibody composition” as used herein refer to a preparation of antibody molecules of single molecular composition. A monoclonal antibody composition displays a single binding specificity and affinity for a particular epitope.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985; and Rossolini et al., Mol. Cell. Probes 8:91-98, 1994).

The term “operably linked” refers to a functional relationship between two or more polynucleotide (e.g., DNA) segments. Typically, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

As used herein, the term, “optimized” means that a nucleotide sequence has been altered to encode an amino acid sequence using codons that are preferred in the production cell or organism, generally a eukaryotic cell, for example, a cell of Pichia, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence. The optimized sequences herein have been engineered to have codons that are preferred in mammalian cells. However, optimized expression of these sequences in other eukaryotic cells or prokaryotic cells is also envisioned herein. The amino acid sequences encoded by optimized nucleotide sequences are also referred to as optimized.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “subject” includes human and non-human animals. Non-human animals include all vertebrates (e.g.: mammals and non-mammals) such as, non-human primates (e.g.: cynomolgus monkey), sheep, rabbit, dog, cow, chickens, amphibians, and reptiles. Except when noted, the terms “patient” or “subject” are used herein interchangeably. As used herein, the terms “cyno” or “cynomolgus” refer to the cynomolgus monkey (Macaca fascicularis). In some specific aspects provided herein, a patient or a subject is a human.

As used herein, the term “treating” or “treatment” of any disease or disorder (e.g., a thromboembolic disorder) refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

“Prevention” as it relates to indications described herein, including, e.g., a thromboembolic disorder, means any action that prevents or slows a worsening in e.g., a thromboembolic disease parameters, as described below, in a patient at risk for being afflicted with a thromboembolic disorder or at risk for said worsening.

The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, such as an adeno-associated viral vector (AAV, or AAV2), wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the present disclosure is intended to include such other forms of expression vectors, such as viral vectors (e.g., replication defective retroviruses, adenoviruses and adeno-associated viruses), which serve equivalent functions.

### Factor XI/XIa and Anti-Factor XI/FXIa Antibodies

This section describes exemplary anti-FXI/FXIa antibodies (e.g., antibodies described in Table 1) to which reversal binding agents provided herein (e.g., anti-idiotype antibodies and fragments thereof) specifically bind, wherein reversal binding agents are capable of reversing one or more anticoagulant effects of such anti-FXI/FXIa antibodies and/or inhibits binding of such anti-FXI/FXIa antibodies to FXI and/or FXIa.

FXI holds important roles in both intrinsic and extrinsic coagulation pathways and in bridging the initiation and amplification phases of plasmatic hemostasis. Both Factor XIIa and thrombin can activate FXI, resulting in a sustained thrombin generation and fibrinolysis inhibition. FXI plays a minor role in normal hemostasis in a high tissue factor environment “after vessel injury” whereas it appears to play a key role in thrombosis. Severe Factor XI deficiency is associated with a lower incidence of ischemic stroke and venous thromboembolic events (Salomon et al 2008; Salomon, et al. (2011) Thromb Haemost.; 105:269-73). Bleeding manifestations in subjects with severe factor XI deficiency are infrequent, often mild, injury-induced and affect preferably tissues with increased fibrinolytic activity such as the oral mucosa, nasal mucosa and urinary tract (Salomon et al 2011). Bleeding in critical organs is extremely rare or not existing.

Plasma coagulation is a sequential process by which coagulation factors in the blood interact and are activated, ultimately resulting in fibrin generation and clot formation. In the classical cascade model of coagulation, the process of fibrin generation can be initiated by two distinct pathways, i.e., the intrinsic and the extrinsic pathway, respectively (Mackman, 2008).

In the extrinsic pathway, vessel injury allows extravascular tissue factor (TF) to interact with and activate factor VII (FVII), which sequentially leads to the activation of factor X and prothrombin. The active thrombin ultimately converts soluble fibrinogen into fibrin. The extrinsic pathway is central for hemostasis, interfering with coagulation factors in this pathway results in a risk of bleeding.

In the intrinsic pathway, factor XII may in some cases be activated by a process referred to as contact activation. Generation of activated factor XIIa leads to the sequential activations of factor XI and factor IX. As factor IXa activates factor X, the extrinsic and intrinsic pathways converge at this stage (at the common pathway). Thrombin activity is boosted by amplifying its own generation through a feed-forward loop in which thrombin activates factor XI independently of factor XII. This feed-forward loop contributes to sustained thrombus growth but is only minimally involved in hemostasis, as the strong activation by extravascular tissue factor is sufficient to clot formation. The intrinsic pathway therefore is not substantially involved in hemostasis (Gailani and Renne (2007) Arterioscler Thromb Vasc Biol. 2007, 27(12):2507-13, Miller, Gailiani, and Renne 2011).

Preclinical studies using a variety of approaches to inhibit FXI or FXIa across a variety of species have contributed to the validation of this target. FXI−/− mice are resistant to experimental venous (Wang, et al. (2006) J Thromb Haemost; 4:1982-8) and arterial (Wang, et al. (2005) J Thromb Haemost; 3:695-702) thrombosis. Treatment of mice with an antibody (Ab, 14E11) that blocks the activation of FXI by FXIIa resulted in inhibition of experimental thrombosis (Cheng, et al. (2010) Blood, 116:3981-9) and reduced cerebral infarct size in a mouse model of ischemic stroke (Leung, et al. (2012) Transl Stroke Res 2012; 3:381-9). In baboons administered an anti-FXI antibody that blocks binding and activation of FIX by FXIa, reduced growth of platelet-rich thrombi was observed on collagen-coated vascular grafts (Tucker, et al. (2009) Blood 2009; 113:936-44), and similar results were found with 14E11 in this model (Cheng 2010). Excessive bleeding was not noted in any of these studies.

Blocking FXI synthesis with antisense oligonucleotides in mice (Zhang, et al. (2010) Blood 2010; 116:4684-92), cynomolgus monkeys (Younis, et al. (2012) Blood 2012; 119:2401-8), and baboons (Crosby, et al. (2013) Arterioscler Thromb Vasc Biol 2013; 33:1670-8) resulted in antithrombotic and anticoagulant effects without excessive bleeding. Moreover, similar effects have been produced by blocking FXIa with low molecular weight inhibitors in venous and arterial models of thrombosis in rats (Schumacher, et al. (2007) Eur J Pharmacol 2007; 570:167-74) and rabbits (Wong, et a. (2011) J Thromb Thrombolysis 2011; 32:129-37).

Patients with severe FXI deficiency rarely bleed spontaneously and they show only mild trauma-induced bleeding, except in tissues with high fibrinolytic activity. The rarity of severe FXI deficiency necessitates the use of population studies for revealing the thrombotic profile of these patients relative to the general population. Notably, such studies report the incidence of ischemic stroke (Salomon 2008) and deep vein thrombosis (DVT) (Salomon, et al. (2011) Blood 2008; 111: 4113-17) to be reduced in these patients. Thus, the number of ischemic strokes (N=1) observed in 115 patients with severe FXI deficiency was less (p<0.003) than the expected incidence (N=8.6) in the general population of Israel, while the incidence of DVT (N=0) was lower (p<0.019) in patients with severe FXI deficiency than expected in the control population (N=4.7). Conversely, individuals with FXI levels above the 90th percentile had a two-fold risk of developing DVT (Meijers, et al. (2000) N Engl J Med. 2000; 342:696-701).

Recently, patients undergoing total knee replacement, a procedure that predisposes to DVT, were treated with FXI antisense therapy or standard of care (enoxaparin). The antisense group (300 mg) showed a 7-fold decreased incidence in venous thrombosis and fewer (not significant) bleeding events compared to standard of care (Büller et al, (2014) N Engl J Med. 372(3):232-40. doi: 10.1056/NEJMoa1405760. Epub 2014 Dec. 7).

Antibodies that specifically bind to FXI and/or FXIa have been described. See for example, PCT International Publication Nos. WO2017/015619, WO2016/207858, WO 2013/167669, WO2009/067660, WO 2009/154461, and WO 2010/080623, each of which is incorporated by reference herein in its entirety. Non-limiting examples of anti-FXI/FXIa antibodies include: 076D-M007-H04, 076D-M007-H04-CDRL3-N110D, and 076D-M028-H17 as described in WO 2013/167669; 1A6 as described in WO2009/067660; and 14E11 as described in WO 2010/080623. In specific aspects, provided herein are binding agents, such as anti-idiotype antibodies, that specifically bind to anti-FXI/FXIa antibody 076D-M007-H04, 076D-M007-H04-CDRL3-N110D, or 076D-M028-H17, and is capable of inhibiting binding of the anti-FXI/FXIa antibody to FXI/FXIa and/or is capable of reversing an anticoagulant effect of the anti-FXI/FXIa antibody. In specific aspects, provided herein are binding agents, such as anti-idiotype antibodies that specifically bind to an anti-FXI/FXIa antibody which competes (e.g., in a dose-dependent manner) with 076D-M007-H04, 076D-M007-H04-CDRL3-N110D, or 076D-M028-H17 for binding to FXI/FXIa, and is capable of inhibiting binding of the anti-FXI/FXIa antibody to FXI/FXIa and/or is capable of reversing an anticoagulant effect of the anti-FXI/FXIa antibody.

Table 1 provides exemplary amino acid sequences and corresponding encoding nucleotide sequences for human FXI and anti-FXI/FXIa antibodies, for example, antibodies NOV1401 and NOV1090. In particular, Table 1 provides the following amino acid sequences for antibodies NOV1401, NOV1090, AM1, AM2, AM3, and AM4, as well as corresponding encoding nucleotide sequences: heavy chain variable region (VH), light chain variable region (VL), heavy chain, light chain, VH complementarity determining regions HCDR1, HCDR2, and HCDR3, VL complementarity determining regions LCDR1, LCDR2, and LCDR3. In specific aspects, reversal binding agents provided herein specifically bind to an anti-FXI/FXIa antibody described in Table 1 and is capable of inhibiting (e.g., in a dose dependent manner) binding of the anti-FXI/FXIa antibody to human FXI/FXIa, and/or of reversing one or more anticoagulant activities of the anti-FXI/FXIa antibody. In specific aspects, reversal binding agents provided herein (e.g., anti-idiotype antibody or antigen-binding fragment thereof such a Fab) specifically bind to anti-FXI/FXIa antibody NOV1401, NOV1090, AM1, AM2, AM3, and/or AM4, and is capable of inhibiting binding of the anti-FXI/FXIa antibody to human FXI/FXIa and/or is capable of reversing an anticoagulant effect of the anti-FXI/FXIa antibody.

Other anti-FXI/FXIa antibodies described in Table 1 herein include NOV1090, AM1, AM2, AM3, and AM4. Antibodies NOV1401 and NOV1090 share the same CDRs. Antibodies AM1, AM2, AM3, and AM4 are exemplary affinity matured variants of antibody NOV1090.

In particular aspects, an anti-FXI/FXIa antibody has one or more of the following anticoagulant activities, which can be reversed (e.g., partially reversed) by a reversal binding agent (e.g., anti-idiotype antibody or fragment thereof such as Fab) provided herein: (i) aPTT prolongation as determined by aPTT assay, (ii) reduction in the amount of thrombin in a thrombin generation assay (TGA) in human plasma, and (iii) inhibition of Factor XI activity. These activities can be readily measured with assays described in the art and provided herein. For example, TGA and aPTT assays are described in the art and herein (e.g., Examples Section). In further aspects, other biomarkers of the extrinsic coagulation pathway can be measured to determine anticoagulant activity, for example, prothrombin time (PT) assay and thrombin time (TT) assay. Other, non-limiting examples of assays for anticoagulation/coagulation activity include chromogenic assays such as ecarin chromogenic assay (ECA), ecarin clotting time (ECT) assay, and anti-Factor Xa activity assay. In specific aspects, reversal binding agents provided herein (e.g., anti-idiotype antibodies) is capable of reversing (e.g., partially reversing) one or more of these anticoagulant activities. In particular aspects, reversal binding agents provided herein is capable of reducing the bleeding time in patients administered an anti-FXI/FXIa antibody.

TABLE 1
Examples of FXI/FXIa Antibodies, Fabs and FXI/FXIa Proteins

| | Sequence | |
|---|---|---|
| | Identifier | |
| Sequence | (SEQ ID | |
| Description | NO:) | Amino acid or polynucleotide sequence |
| Human FXI full- | 1 | MIFLYQVVHF ILFTSVSGEC VTQLLKDTCF EGGDITTVFT |
| length protein | | PSAKYCQVVC TYHPRCLLFT FTAESPSEDP TRWFTCVLKD |
| sequence (NCBI | | SVTETLPRVN RTAAISGYSF KQCSHQISAC NKDIYVDLDM |
| Reference | | KGINYNSSVA KSAQECQERC TDDVHCHFFT YATRQFPSLE |
| Sequence: | | HRNICLLKHT QTGTPTRITK LDKVVSGFSL KSCALSNLAC |
| AAA51985) | | IRDIFPNTVF ADSNIDSVMA PDAFVSGRIC THHPGCLFFT |
| | | FFSQEWPKES QRNLCLLKTS ESGLPSTRIK KSKALSGFSL |
| | | QSCRHSIPVF CHSSFYHDTD FLGEELDIVA AKSHEACQKL |
| | | CTNAVRCQFF TYTPAQASCN EGKGKCYLKL SSNGSPTKIL |
| | | HGRGGISGYT LRLCKMDNEC TTKIKPRIVG GTASVRGEWP |
| | | WQVTLHTTSP TQRHLCGGSI IGNQWILTAA HCFYGVESPK |
| | | ILRVYSGILN QSEIKEDTSF FGVQEIIIHD QYKMAESGYD |
| | | IALLKLETTV NYTDSQRPIC LPSKGDRNVI YTDCWVTGWG |
| | | YRKLRDKIQN TLQKAKIPLV TNEECQKRYR GHKITHKMIC |
| | | AGYREGGKDA CKGDSGGPLS CKHNEVWHLV GITSWGEGCA |
| | | QRERPGVYTN VVEYVDWILE KTQAV |
| Human FXI full- | 2 | AGGCACACAG GCAAAATCAA GTTCTACATC TGTCCCTGTG |
| length | | TATGTCACTT GTTTGAATAC GAAATAAAAT TAAAAAAATA |
| nucleotide | | AATTCAGTGT ATTGAGAAAG CAAGCAATTC TCTCAAGGTA |
| sequence (NCBI | | TATTTCTGAC ATACTAAGAT TTTAACGACT TTCACAAATA |
| Reference | | TGCTGTACTG AGAGAGAATG TTACATAACA TTGAGAACTA |
| Sequence: | | GTACAAGTAA ATATTAAAGT GAAGTGACCA TTTCCTACAC |
| NM_000128.3) | | AAGCTCATTC AGAGGAGGAT GAAGACCATT TTGGAGGAAG |
| | | AAAAGCACCC TTATTAAGAA TTGCAGCAAG TAAGCCAACA |
| | | AGGTCTTTTC AGGATGATTT TCTTATATCA AGTGGTACAT |
| | | TTCATTTTAT TTACTTCAGT TTCTGGTGAA TGTGTGACTC |
| | | AGTTGTTGAA GGACACCTGC TTTGAAGGAG GGGACATTAC |
| | | TACGGTCTTC ACACCAAGCG CCAAGTACTG CCAGGTAGTC |
| | | TGCACTTACC ACCCAAGATG TTTACTCTTC ACTTTCACGG |
| | | CGGAATCACC ATCTGAGGAT CCCACCCGAT GGTTTACTTG |
| | | TGTCCTGAAA GACAGTGTTA CAGAAACACT GCCAAGAGTG |
| | | AATAGGACAG CAGCGATTTC TGGGTATTCT TTCAAGCAAT |
| | | GCTCACACCA AATAAGCGCT TGCAACAAAG ACATTTATGT |
| | | GGACCTAGAC ATGAAGGGCA TAAACTATAA CAGCTCAGTT |
| | | GCCAAGAGTG CTCAAGAATG CCAAGAAAGA TGCACGGATG |
| | | ACGTCCACTG CCACTTTTTC ACGTACGCCA CAAGGCAGTT |
| | | TCCCAGCCTG GAGCATCGTA ACATTTGTCT ACTGAAGCAC |
| | | ACCCAAACAG GGACACCAAC CAGAATAACG AAGCTCGATA |
| | | AAGTGGTGTC TGGATTTTCA CTGAAATCCT GTGCACTTTC |
| | | TAATCTGGCT TGTATTAGGG ACATTTTCCC TAATACGGTG |
| | | TTTGCAGACA GCAACATCGA CAGTGTCATG GCTCCCGATG |
| | | CTTTTGTCTG TGGCCGAATC TGCACTCATC ATCCCGGTTG |
| | | CTTGTTTTTT ACCTTCTTTT CCCAGGAATG GCCCAAAGAA |
| | | TCTCAAAGAA ATCTTTGTCT CCTTAAAACA TCTGAGAGTG |
| | | GATTGCCCAG TACACGCATT AAAAAGAGCA AAGCTCTTTC |
| | | TGGTTTCAGT CTACAAAGCT GCAGGCACAG CATCCCAGTG |
| | | TTCTGCCATT CTTCATTTTA CCATGACACT GATTTCTTGG |
| | | GAGAAGAACT GGATATTGTT GCTGCAAAAA GTCACGAGGC |
| | | CTGCCAGAAA CTGTGCACCA ATGCCGTCCG CTGCCAGTTT |
| | | TTTACCTATA CCCCAGCCCA AGCATCCTGC AACGAAGGGA |
| | | AGGGCAAGTG TTACTTAAAG CTTTCTTCAA ACGGATCTCC |
| | | AACTAAAATA CTTCACGGGA GAGGAGGCAT CTCTGGATAC |
| | | ACATTAAGGT TGTGTAAAAT GGATAATGAG TGTACCACCA |
| | | AAATCAAGCC CAGGATCGTT GGAGGAACTG CGTCTGTTCG |
| | | TGGTGAGTGG CCGTGGCAGG TGACCCTGCA CACAACCTCA |
| | | CCCACTCAGA GACACCTGTG TGGAGGCTCC ATCATTGGAA |
| | | ACCAGTGGAT ATTAACAGCC GCTCACTGTT TCTATGGGGT |
| | | AGAGTCACCT AAGATTTTGC GTGTCTACAG TGGCATTTTA |
| | | AATCAATCTG AAATAAAAGA GGACACATCT TTCTTTGGGG |
| | | TTCAAGAAAT AATAATCCAT GATCAGTATA AAATGGCAGA |
| | | AAGCGGGTAT GATATTGCCT TGTTGAAACT GGAAACCACA |
| | | GTGAATTACA CAGATTCTCA ACGACCCATA TGCCTGCCTT |
| | | CCAAAGGAGA TAGAAATGTA ATATACACTG ATTGCTGGGT |
| | | GACTGGATGG GGGTACAGAA AACTAAGAGA CAAAATACAA |
| | | AATACTCTCC AGAAAGCCAA GATACCCTTA GTGACCAACG |
| | | AAGAGTGCCA GAAGAGATAC AGAGGACATA AAATAACCCA |
| | | TAAGATGATC TGTGCCGGCT ACAGGGAAGG AGGGAAGGAC |
| | | GCTTGCAAGG GAGATTCGGG AGGCCCTCTG TCCTGCAAAC |
| | | ACAATGAGGT CTGGCATCTG GTAGGCATCA CGAGCTGGGG |
| | | CGAAGGCTGT GCTCAAAGGG AGCGGCCAGG TGTTTACACC |
| | | AACGTGGTCG AGTACGTGGA CTGGATTCTG GAGAAAACTC |
| | | AAGCAGTGTG AATGGGTTCC CAGGGGCCAT TGGAGTCCCT |
| | | GAAGGACCCA GGATTTGCTG GGAGAGGGTG TTGAGTTCAC |
| | | TGTGCCAGCA TGCTTCCTCC ACAGTAACAC GCTGAAGGGG |
| | | CTTGGTGTTT GTAAGAAAAT GCTAGAAGAA AACAAACTGT |
| | | CACAAGTTGT TATGTCCAAA ACTCCCGTTC TATGATCGTT |
| | | GTAGTTTGTT TGAGCATTCA GTCTCTTTGT TTTTGATCAC |
| | | GCTTCTATGG AGTCCAAGAA TTACCATAAG GCAATATTTC |
| | | TGAAGATTAC TATATAGGCA GATATAGCAG AAAATAACCA |
| | | AGTAGTGGCA GTGGGGATCA GGCAGAAGAA CTGGTAAAAG |
| | | AAGCCACCAT AAATAGATTT GTTCGATGAA AGATGAAAAC |
| | | TGGAAGAAAG GAGAACAAAG ACAGTCTTCA CCATTTTGCA |
| | | GGAATCTACA CTCTGCCTAT GTGAACACAT TTCTTTTGTA |
| | | AAGAAAGAAA TTGATTGCAT TTAATGGCAG ATTTTCAGAA |
| | | TAGTCAGGAA TTCTTGTCAT TTCCATTTTA AAATATATAT |
| | | TAAAAAAAAT CAGTTCGAGT AGACACGAGC TAAGAGTGAA |
| | | TGTGAAGATA ACAGAATTTC TGTGTGGAAG AGGATTACAA |
| | | GCAGCAATTT ACCTGGAAGT GATACCTTAG GGGCAATCTT |
| | | GAAGATACAC TTTCCTGAAA AATGATTTGT GATGGATTGT |
| | | ATATTTATTT AAAATATCTT GGGAGGGGAG GCTGATGGAG |
| | | ATAGGGAGCA TGCTCAAACC TCCCTAAGAC AAGCTGCTGC |
| | | TGTGACTATG GGCTCCCAAA GAGCTAGATC GTATATTTAT |
| | | TTGACAAAAA TCACCATAGA CTGCATCCAT ACTACAGAGA |
| | | AAAAACAATT AGGGCGCAAA TGGATAGTTA CAGTAAAGTC |
| | | TTCAGCAAGC AGCTGCCTGT ATTCTAAGCA CTGGGATTTT |
| | | CTGTTTCGTG CAAATATTTA TCTCATTATT GTTGTGATCT |
| | | AGTTCAATAA CCTAGAATTT GAATTGTCAC CACATAGCTT |
| | | TCAATCTGTG CCAACAACTA TACAATTCAT CAAGTGTG |
| NOV1401 | | |
| HCDR1 | 3 | GFTFSTAAMS |
| (Combined) | | |
| HCDR2 | 4 | GISGSGSSTYYADSVKG |
| (Combined) | | |
| HCDR3 | 5 | ELSYLYSGYYFDY |
| (Combined) | | |
| HCDR1 (Kabat) | 6 | TAAMS |
| HCDR2 (Kabat) | 4 | GISGSGSSTYYADSVKG |
| HCDR3 (Kabat) | 5 | ELSYLYSGYYFDY |
| HCDR1 | 7 | GFTFSTA |
| (Chothia) | | |
| HCDR2 | 8 | SGSGSS |
| (Chothia) | | |
| HCDR3 | 5 | ELSYLYSGYYFDY |
| (Chothia) | | |
| HCDR1 (IMGT) | 9 | GFTFSTAA |
| HCDR2 (IMGT) | 10 | ISGSGSST |
| HCDR3 (IMGT) | 11 | ARELSYLYSGYYFDY |
| VH | 12 | QVQLLESGGGLVQPGGSLRLSCAASGFTFSTAAMSWVRQAPGK |
| | | GLEWVSGISGSGSSTYYADSVKGRFTISRDNSKNTLYLQMNSL |
| | | RAEDTAVYYCARELSYLYSGYYFDYWGQGTLVTVSS |
| DNA encoding VH | 13 | CAGGTGCAGCTGCTGGAATCAGGCGGCGGACTGGTGCAGCCTG |
| | | GCGGTAGCCTGAGACTGAGCTGCGCTGCTAGTGGCTTCACCTT |
| | | TAGCACCGCCGCTATGAGCTGGGTTCGACAGGCCCCAGGGAAA |
| | | GGCCTCGAGTGGGTCTCAGGGATTAGCGGTAGCGGCTCTAGCA |
| | | CCTACTACGCCGATAGCGTGAAGGGCCGGTTCACTATCTCTAG |
| | | GGATAACTCTAAGAACACCCTGTACCTGCAGATGAATAGCCTG |
| | | AGAGCCGAGGACACCGCCGTCTACTACTGCGCTAGAGAGCTGA |
| | | GCTACCTGTATAGCGGCTACTACTTCGACTACTGGGGTCAAGG |
| | | CACCCTGGTCACCGTGTCTAGC |
| Heavy Chain | 14 | QVQLLESGGGLVQPGGSLRLSCAASGFTFSTAAMSWVRQAPGK |
| | | GLEWVSGISGSGSSTYYADSVKGRFTISRDNSKNTLYLQMNSL |
| | | RAEDTAVYYCARELSYLYSGYYFDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTK |
| | | VDKRVEPKSCDKTHTCPPCPAPELLGGPSVFLFPPKPKDTLMI |
| | | SRTPEVTCVVVAVSHEDPEVKFNWYVDGVEVHNAKTKPREEQY |
| | | NSTYRVVSVLTVLHQDWLNGKEYKCKVSNKALAAPIEKTISKA |
| | | KGQPREPQVYTLPPSREEMTKNQVSLTCLVKGFYPSDIAVEWE |
| | | SNGQPENNYKTTPPVLDSDGSFFLYSKLTVDKSRWQQGNVFSC |
| | | SVMHEALHNHYTQKSLSLSPGK |
| DNA encoding | 15 | CAGGTGCAGCTGCTGGAATCAGGCGGCGGACTGGTGCAGCCTG |
| Heavy Chain | | GCGGTAGCCTGAGACTGAGCTGCGCTGCTAGTGGCTTCACCTT |
| | | TAGCACCGCCGCTATGAGCTGGGTTCGACAGGCCCCAGGGAAA |
| | | GGCCTCGAGTGGGTCTCAGGGATTAGCGGTAGCGGCTCTAGCA |
| | | CCTACTACGCCGATAGCGTGAAGGGCCGGTTCACTATCTCTAG |
| | | GGATAACTCTAAGAACACCCTGTACCTGCAGATGAATAGCCTG |
| | | AGAGCCGAGGACACCGCCGTCTACTACTGCGCTAGAGAGCTGA |
| | | GCTACCTGTATAGCGGCTACTACTTCGACTACTGGGGTCAAGG |
| | | CACCCTGGTCACCGTGTCTAGCGCTAGCACTAAGGGCCCCTCC |
| | | GTGTTCCCTCTGGCCCCTTCCAGCAAGTCTACCTCCGGCGGCA |
| | | CAGCTGCTCTGGGCTGCCTGGTCAAGGACTACTTCCCTGAGCC |
| | | TGTGACAGTGTCCTGGAACTCTGGCGCCCTGACCTCTGGCGTG |
| | | CACACCTTCCCTGCCGTGCTGCAGTCCTCCGGCCTGTACTCCC |
| | | TGTCCTCCGTGGTCACAGTGCCTTCAAGCAGCCTGGGCACCCA |
| | | GACCTATATCTGCAACGTGAACCACAAGCCTTCCAACACCAAG |
| | | GTGGACAAGCGGGTGGAGCCTAAGTCCTGCGACAAGACCCACA |
| | | CCTGTCCTCCCTGCCCTGCTCCTGAACTGCTGGGCGGCCCTTC |
| | | TGTGTTCCTGTTCCCTCCAAAGCCCAAGGACACCCTGATGATC |
| | | TCCCGGACCCCTGAAGTGACCTGCGTGGTGGTGGCCGTGTCCC |
| | | ACGAGGATCCTGAAGTGAAGTTCAATTGGTACGTGGACGGCGT |
| | | GGAGGTGCACAACGCCAAGACCAAGCCTCGGGAGGAACAGTAC |
| | | AACTCCACCTACCGGGTGGTGTCCGTGCTGACCGTGCTGCACC |
| | | AGGACTGGCTGAACGGCAAAGAGTACAAGTGCAAAGTCTCCAA |
| | | CAAGGCCCTGGCCGCCCCTATCGAAAAGACAATCTCCAAGGCC |
| | | AAGGGCCAGCCTAGGGAACCCCAGGTGTACACCCTGCCACCCA |
| | | GCCGGGAGGAAATGACCAAGAACCAGGTGTCCCTGACCTGTCT |
| | | GGTCAAGGGCTTCTACCCTTCCGATATCGCCGTGGAGTGGGAG |
| | | TCTAACGGCCAGCCTGAGAACAACTACAAGACCACCCCTCCTG |
| | | TGCTGGACTCCGACGGCTCCTTCTTCCTGTACTCCAAACTGAC |
| | | CGTGGACAAGTCCCGGTGGCAGCAGGGCAACGTGTTCTCCTGC |
| | | TCCGTGATGCACGAGGCCCTGCACAACCACTACACCCAGAAGT |
| | | CCCTGTCCCTGTCTCCCGGCAAG |
| LCDR1 | 16 | SGSSSNIGSNDVS |
| (Combined) | | |
| LCDR2 | 17 | KNYNRPS |
| (Combined) | | |
| LCDR3 | 18 | SAWDQRQFDVV |
| (Combined) | | |
| LCDR1 (Kabat) | 16 | SGSSSNIGSNDVS |
| LCDR2 (Kabat) | 17 | KNYNRPS |
| LCDR3 (Kabat) | 18 | SAWDQRQFDVV |
| LCDR1 | 19 | SSSNIGSND |
| (Chothia) | | |
| LCDR2 | 20 | KNY |
| (Chothia) | | |
| LCDR3 | 21 | WDQRQFDV |
| (Chothia) | | |
| LCDR1 (IMGT) | 22 | SSNIGSND |
| LCDR2 (IMGT) | 20 | KNY |
| LCDR3 (IMGT) | 18 | SAWDQRQFDVV |
| VL | 23 | QSVLTQPPSASGTPGQRVTISCSGSSSNIGSNDVSWYQQLPGT |
| | | APKLLIYKNYNRPSGVPDRFSGSKSGTSASLAISGLQSEDEAD |
| | | YYCSAWDQRQFDVVFGGGTKLTVL |
| DNA encoding VL | 24 | CAGTCAGTCCTGACTCAGCCCCCTAGCGCTAGTGGCACCCCTG |
| | | GTCAAAGAGTGACTATTAGCTGTAGCGGCTCTAGCTCTAATAT |
| | | CGGCTCTAACGACGTCAGCTGGTATCAGCAGCTGCCCGGCACC |
| | | GCCCCTAAGCTGCTGATCTATAAGAACTATAATAGGCCTAGCG |
| | | GCGTGCCCGATAGGTTTAGCGGATCTAAATCAGGGACTTCTGC |
| | | TAGTCTGGCTATTAGCGGCCTGCAGTCAGAGGACGAGGCCGAC |
| | | TACTACTGTAGCGCCTGGGATCAGCGTCAGTTCGACGTGGTGT |
| | | TCGGCGGAGGCACTAAGCTGACCGTGCTG |
| Light Chain | 25 | QSVLTQPPSASGTPGQRVTISCSGSSSNIGSNDVSWYQQLPGT |
| | | APKLLIYKNYNRPSGVPDRFSGSKSGTSASLAISGLQSEDEAD |
| | | YYCSAWDQRQFDVVFGGGTKLTVLGQPKAAPSVTLFPPSSEEL |
| | | QANKATLVCLISDFYPGAVTVAWKADSSPVKAGVETTTPSKQS |
| | | NNKYAASSYLSLTPEQWKSHRSYSCQVTHEGSTVEKTVAPTEC |
| | | S |
| DNA encoding | 26 | CAGTCAGTCCTGACTCAGCCCCCTAGCGCTAGTGGCACCCCTG |
| Light Chain | | GTCAAAGAGTGACTATTAGCTGTAGCGGCTCTAGCTCTAATAT |
| | | CGGCTCTAACGACGTCAGCTGGTATCAGCAGCTGCCCGGCACC |
| | | GCCCCTAAGCTGCTGATCTATAAGAACTATAATAGGCCTAGCG |
| | | GCGTGCCCGATAGGTTTAGCGGATCTAAATCAGGGACTTCTGC |
| | | TAGTCTGGCTATTAGCGGCCTGCAGTCAGAGGACGAGGCCGAC |
| | | TACTACTGTAGCGCCTGGGATCAGCGTCAGTTCGACGTGGTGT |
| | | TCGGCGGAGGCACTAAGCTGACCGTGCTGGGTCAACCTAAGGC |
| | | TGCCCCCAGCGTGACCCTGTTCCCCCCCAGCAGCGAGGAGCTG |
| | | CAGGCCAACAAGGCCACCCTGGTGTGCCTGATCAGCGACTTCT |
| | | ACCCAGGCGCCGTGACCGTGGCCTGGAAGGCCGACAGCAGCCC |
| | | CGTGAAGGCCGGCGTGGAGACCACCACCCCCAGCAAGCAGAGC |
| | | AACAACAAGTACGCCGCCAGCAGCTACCTGAGCCTGACCCCCG |
| | | AGCAGTGGAAGAGCCACAGGTCCTACAGCTGCCAGGTGACCCA |
| | | CGAGGGCAGCACCGTGGAAAAGACCGTGGCCCCAACCGAGTGC |
| | | AGC |
| NOV1090 | | |
| HCDR1 | 3 | GFTFSTAAMS |
| (Combined) | | |
| HCDR2 | 4 | GISGSGSSTYYADSVKG |
| (Combined) | | |
| HCDR3 | 5 | ELSYLYSGYYFDY |
| (Combined) | | |
| HCDR1 (Kabat) | 6 | TAAMS |
| HCDR2 (Kabat) | 4 | GISGSGSSTYYADSVKG |
| HCDR3 (Kabat) | 5 | ELSYLYSGYYFDY |
| HCDR1 | 7 | GFTFSTA |
| (Chothia) | | |
| HCDR2 | 8 | SGSGSS |
| (Chothia) | | |
| HCDR3 | 5 | ELSYLYSGYYFDY |
| (Chothia) | | |
| HCDR1 (IMGT) | 9 | GFTFSTAA |
| HCDR2 (IMGT) | 10 | ISGSGSST |
| HCDR3 (IMGT) | 11 | ARELSYLYSGYYFDY |
| VH | 12 | QVQLLESGGGLVQPGGSLRLSCAASGFTFSTAAMSWVRQAPGK |
| | | GLEWVSGISGSGSSTYYADSVKGRFTISRDNSKNTLYLQMNSL |
| | | RAEDTAVYYCARELSYLYSGYYFDYWGQGTLVTVSS |
| DNA encoding VH | 390 | CAGGTGCAATTGCTGGAAAGCGGCGGTGGCCTGGTGCAGCCGG |
| | | GTGGCAGCCTGCGTCTGAGCTGCGCGGCGTCCGGATTCACCTT |
| | | TTCTACTGCTGCTATGTCTTGGGTGCGCCAGGCCCCGGGCAAA |
| | | GGTCTCGAGTGGGTTTCCGGTATCTCTGGTTCTGGTTCTTCTA |
| | | CCTACTATGCGGATAGCGTGAAAGGCCGCTTTACCATCAGCCG |
| | | CGATAATTCGAAAAACACCCTGTATCTGCAAATGAACAGCCTG |
| | | CGTGCGGAAGATACGGCCGTGTATTATTGCGCGCGTGAACTGT |
| | | CTTACCTGTACTCTGGTTACTACTTCGATTACTGGGGCCAAGG |
| | | CACCCTGGTGACTGTTAGCTCA |
| Heavy Chain | 360 | QVQLLESGGGLVQPGGSLRLSCAASGFTFSTAAMSWVRQAPGK |
| | | GLEWVSGISGSGSSTYYADSVKGRFTISRDNSKNTLYLQMNSL |
| | | RAEDTAVYYCARELSYLYSGYYFDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTK |
| | | VDKRVEPKSCDKTHTCPPCPAPEAAGGPSVFLFPPKPKDTLMI |
| | | SRTPEVTCVVVDVSHEDPEVKFNWYVDGVEVHNAKTKPREEQY |
| | | NSTYRVVSVLTVLHQDWLNGKEYKCKVSNKALPAPIEKTISKA |
| | | KGQPREPQVYTLPPSREEMTKNQVSLTCLVKGFYPSDIAVEWE |
| | | SNGQPENNYKTTPPVLDSDGSFFLYSKLTVDKSRWQQGNVFSC |
| | | SVMHEALHNHYTQKSLSLSPGK |
| DNA encoding | 361 | CAGGTGCAATTGCTGGAAAGCGGCGGTGGCCTGGTGCAGCCGG |
| Heavy Chain | | GTGGCAGCCTGCGTCTGAGCTGCGCGGCGTCCGGATTCACCTT |
| | | TTCTACTGCTGCTATGTCTTGGGTGCGCCAGGCCCCGGGCAAA |
| | | GGTCTCGAGTGGGTTTCCGGTATCTCTGGTTCTGGTTCTTCTA |
| | | CCTACTATGCGGATAGCGTGAAAGGCCGCTTTACCATCAGCCG |
| | | CGATAATTCGAAAAACACCCTGTATCTGCAAATGAACAGCCTG |
| | | CGTGCGGAAGATACGGCCGTGTATTATTGCGCGCGTGAACTGT |
| | | CTTACCTGTACTCTGGTTACTACTTCGATTACTGGGGCCAAGG |
| | | CACCCTGGTGACTGTTAGCTCAGCCTCCACCAAGGGTCCATCG |
| | | GTCTTCCCCCTGGCACCCTCCTCCAAGAGCACCTCTGGGGGCA |
| | | CAGCGGCCCTGGGCTGCCTGGTCAAGGACTACTTCCCCGAACC |
| | | GGTGACGGTGTCGTGGAACTCAGGCGCCCTGACCAGCGGCGTG |
| | | CACACCTTCCCGGCTGTCCTACAGTCCTCAGGACTCTACTCCC |
| | | TCAGCAGCGTGGTGACCGTGCCCTCCAGCAGCTTGGGCACCCA |
| | | GACCTACATCTGCAACGTGAATCACAAGCCCAGCAACACCAAG |
| | | GTGGACAAGAGAGTTGAGCCCAAATCTTGTGACAAAACTCACA |
| | | CATGCCCACCGTGCCCAGCACCTGAAGCAGCGGGGGGACCGTC |
| | | AGTCTTCCTCTTCCCCCCAAAACCCAAGGACACCCTCATGATC |
| | | TCCCGGACCCCTGAGGTCACATGCGTGGTGGTGGACGTGAGCC |
| | | ACGAAGACCCTGAGGTCAAGTTCAACTGGTACGTGGACGGCGT |
| | | GGAGGTGCATAATGCCAAGACAAAGCCGCGGGAGGAGCAGTAC |
| | | AACAGCACGTACCGGGTGGTCAGCGTCCTCACCGTCCTGCACC |
| | | AGGACTGGCTGAATGGCAAGGAGTACAAGTGCAAGGTCTCCAA |
| | | CAAAGCCCTCCCAGCCCCCATCGAGAAAACCATCTCCAAAGCC |
| | | AAAGGGCAGCCCCGAGAACCACAGGTGTACACCCTGCCCCCAT |
| | | CCCGGGAGGAGATGACCAAGAACCAGGTCAGCCTGACCTGCCT |
| | | GGTCAAAGGCTTCTATCCCAGCGACATCGCCGTGGAGTGGGAG |
| | | AGCAATGGGCAGCCGGAGAACAACTACAAGACCACGCCTCCCG |
| | | TGCTGGACTCCGACGGCTCCTTCTTCCTCTACAGCAAGCTCAC |
| | | CGTGGACAAGAGCAGGTGGCAGCAGGGGAACGTCTTCTCATGC |
| | | TCCGTGATGCATGAGGCTCTGCACAACCACTACACGCAGAAGA |
| | | GCCTCTCCCTGTCTCCGGGTAAA |
| LCDR1 | 16 | SGSSSNIGSNDVS |
| (Combined) | | |
| LCDR2 | 17 | KNYNRPS |
| (Combined) | | |
| LCDR3 | 18 | SAWDQRQFDVV |
| (Combined) | | |
| LCDR1 (Kabat) | 16 | SGSSSNIGSNDVS |
| LCDR2 (Kabat) | 17 | KNYNRPS |
| LCDR3 (Kabat) | 18 | SAWDQRQFDVV |
| LCDR1 | 19 | SSSNIGSND |
| (Chothia) | | |
| LCDR2 | 20 | KNY |
| (Chothia) | | |
| LCDR3 | 21 | WDQRQFDV |
| (Chothia) | | |
| LCDR1 (IMGT) | 22 | SSNIGSND |
| LCDR2 (IMGT) | 20 | KNY |
| LCDR3 (IMGT) | 18 | SAWDQRQFDVV |
| VL | 362 | DIVLTQPPSVSGAPGQRVTISCSGSSSNIGSNDVSWYQQLPGT |
| | | APKLLIYKNYNRPSGVPDRFSGSKSGTSASLAITGLQAEDEAD |
| | | YYCSAWDQRQFDVVFGGGTKLTVL |
| DNA encoding VL | 363 | GATATCGTGCTGACCCAGCCGCCGAGCGTGAGCGGTGCACCGG |
| | | GCCAGCGCGTGACCATTAGCTGTAGCGGCAGCAGCAGCAACAT |
| | | TGGTTCTAACGACGTGTCTTGGTACCAGCAGCTGCCGGGCACG |
| | | GCGCCGAAACTGCTGATCTACAAAAACTACAACCGCCCGAGCG |
| | | GCGTGCCGGATCGCTTTAGCGGATCCAAAAGCGGCACCAGCGC |
| | | CAGCCTGGCGATTACCGGCCTGCAAGCAGAAGACGAAGCGGAT |
| | | TATTACTGCTCTGCTTGGGACCAGCGTCAGTTCGACGTTGTGT |
| | | TTGGCGGCGGCACGAAGTTAACCGTCCTA |
| Light Chain | 364 | DIVLTQPPSVSGAPGQRVTISCSGSSSNIGSNDVSWYQQLPGT |
| | | APKLLIYKNYNRPSGVPDRFSGSKSGTSASLAITGLQAEDEAD |
| | | YYCSAWDQRQFDVVFGGGTKLTVLGQPKAAPSVTLFPPSSEEL |
| | | QANKATLVCLISDFYPGAVTVAWKADSSPVKAGVETTTPSKQS |
| | | NNKYAASSYLSLTPEQWKSHRSYSCQVTHEGSTVEKTVAPTEC |
| | | S |
| DNA encoding | 365 | GATATCGTGCTGACCCAGCCGCCGAGCGTGAGCGGTGCACCGG |
| Light Chain | | GCCAGCGCGTGACCATTAGCTGTAGCGGCAGCAGCAGCAACAT |
| | | TGGTTCTAACGACGTGTCTTGGTACCAGCAGCTGCCGGGCACG |
| | | GCGCCGAAACTGCTGATCTACAAAAACTACAACCGCCCGAGCG |
| | | GCGTGCCGGATCGCTTTAGCGGATCCAAAAGCGGCACCAGCGC |
| | | CAGCCTGGCGATTACCGGCCTGCAAGCAGAAGACGAAGCGGAT |
| | | TATTACTGCTCTGCTTGGGACCAGCGTCAGTTCGACGTTGTGT |
| | | TTGGCGGCGGCACGAAGTTAACCGTCCTAGGTCAGCCCAAGGC |
| | | TGCCCCCTCGGTCACTCTGTTCCCGCCCTCCTCTGAGGAGCTT |
| | | CAAGCCAACAAGGCCACACTGGTGTGTCTCATAAGTGACTTCT |
| | | ACCCGGGAGCCGTGACAGTGGCCTGGAAGGCAGATAGCAGCCC |
| | | CGTCAAGGCGGGAGTGGAGACCACCACACCCTCCAAACAAAGC |
| | | AACAACAAGTACGCGGCCAGCAGCTATCTGAGCCTGACGCCTG |
| | | AGCAGTGGAAGTCCCACAGAAGCTACAGCTGCCAGGTCACGCA |
| | | TGAAGGGAGCACCGTGGAGAAGACAGTGGCCCCTACAGAATGT |
| | | TCA |
| AM1 | | |
| HCDR1 | 3 | GFTFSTAAMS |
| (Combined) | | |
| HCDR2 | 366 | TIDSWGDDTDYADSVKG |
| (Combined) | | |
| HCDR3 | 5 | ELSYLYSGYYFDY |
| (Combined) | | |
| HCDR1 (Kabat) | 6 | TAAMS |
| HCDR2 (Kabat) | 366 | TIDSWGDDTDYADSVKG |
| HCDR3 (Kabat) | 5 | ELSYLYSGYYFDY |
| HCDR1 | 7 | GFTFSTA |
| (Chothia) | | |
| HCDR2 | 367 | DSWGDD |
| (Chothia) | | |
| HCDR3 | 5 | ELSYLYSGYYFDY |
| (Chothia) | | |
| HCDR1 (IMGT) | 9 | GFTFSTAA |
| HCDR2 (IMGT) | 368 | IDSWGDDT |
| HCDR3 (IMGT) | 11 | ARELSYLYSGYYFDY |
| VH | 369 | QVQLLESGGGLVQPGGSLRLSCAASGFTFSTAAMSWVRQ |
| | | APGKGLEMVSTIDSWGDDIDYADSVKGRFTISRDNSKNT |
| | | LYLQMNSLRAEDTAVYYCARELSYLYSGYYFDYWGQGTL |
| | | VTVSS |
| DNA VH | 370 | CAGGTGCAATTGCTGGAAAGCGGCGGTGGCCTGGTGCAG |
| | | CCGGGTGGCAGCCTGCGTCTGAGCTGCGCGGCGTCCGGA |
| | | TTCACCTTTTCTACTGCTGCTATGTCTTGGGTGCGCCAG |
| | | GCCCCGGGCAAAGGTCTCGAGTGGGTTTCCACTATCGAC |
| | | TCTTGGGGCGACGACACTGACTATGCGGATAGCGTGAAA |
| | | GGCCGCTTTACCATCAGCCGCGATAATTCGAAAAACACC |
| | | CTGTATCTGCAAATGAACAGCCTGCGTGCGGAAGATACG |
| | | GCCGTGTATTATTGCGCGCGTGAACTGTCTTACCTGTAC |
| | | TCTGGTTACTACTTCGATTACTGGGGCCAAGGCACCCTG |
| | | GTGACTGTTAGCTCA |
| Heavy Chain | 371 | QVQLLESGGGLVQPGGSLRLSCAASGFTFSTAAMSWVRQ |
| | | APGKGLEMVSTIDSWGDDIDYADSVKGRFTISRDNSKNT |
| | | LYLQMNSLRAEDTAVYYCARELSYLYSGYYFDYWGQGTL |
| | | VTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKRVEPKSCDKTHTCPPC |
| | | PAPEAAGGPSVFLFPPKPKDTLMISRTPEVTCVVVDVSH |
| | | EDPEVKFNWYVDGVEVHNAKTKPREEQYNSTYRVVSVLT |
| | | VLHQDWLNGKEYKCKVSNKALPAPIEKTISKAKGQPREP |
| | | QVYTLPPSREEMTKNQVSLICLVKGFYPSDIAVEWESNG |
| | | QPENNYKTTPPVLDSDGSFFLYSKLTVDKSRWQQGNVFS |
| | | CSVMHEALHNHYTQKSLSLSPGK |
| DNA Heavy | 391 | CAGGTGCAATTGCTGGAAAGCGGCGGTGGCCTGGTGCAG |
| Chain | | CCGGGTGGCAGCCTGCGTCTGAGCTGCGCGGCGTCCGGA |
| | | TTCACCTTTTCTACTGCTGCTATGTCTTGGGTGCGCCAG |
| | | GCCCCGGGCAAAGGTCTCGAGTGGGTTTCCACTATCGAC |
| | | TCTTGGGGCGACGACACTGACTATGCGGATAGCGTGAAA |
| | | GGCCGCTTTACCATCAGCCGCGATAATTCGAAAAACACC |
| | | CTGTATCTGCAAATGAACAGCCTGCGTGCGGAAGATACG |
| | | GCCGTGTATTATTGCGCGCGTGAACTGTCTTACCTGTAC |
| | | TCTGGTTACTACTTCGATTACTGGGGCCAAGGCACCCTG |
| | | GTGACTGTTAGCTCAGCCTCCACCAAGGGTCCATCGGTC |
| | | TTCCCCCTGGCACCCTCCTCCAAGAGCACCTCTGGGGGC |
| | | ACAGCGGCCCTGGGCTGCCTGGTCAAGGACTACTTCCCC |
| | | GAACCGGTGACGGTGTCGTGGAACTCAGGCGCCCTGACC |
| | | AGCGGCGTGCACACCTTCCCGGCTGTCCTACAGTCCTCA |
| | | GGACTCTACTCCCTCAGCAGCGTGGTGACCGTGCCCTCC |
| | | AGCAGCTTGGGCACCCAGACCTACATCTGCAACGTGAAT |
| | | CACAAGCCCAGCAACACCAAGGTGGACAAGAGAGTTGAG |
| | | CCCAAATCTTGTGACAAAACTCACACATGCCCACCGTGC |
| | | CCAGCACCTGAAGCAGCGGGGGGACCGTCAGTCTTCCTC |
| | | TTCCCCCCAAAACCCAAGGACACCCTCATGATCTCCCGG |
| | | ACCCCTGAGGTCACATGCGTGGTGGTGGACGTGAGCCAC |
| | | GAAGACCCTGAGGTCAAGTTCAACTGGTACGTGGACGGC |
| | | GTGGAGGTGCATAATGCCAAGACAAAGCCGCGGGAGGAG |
| | | CAGTACAACAGCACGTACCGGGTGGTCAGCGTCCTCACC |
| | | GTCCTGCACCAGGACTGGCTGAATGGCAAGGAGTACAAG |
| | | TGCAAGGTCTCCAACAAAGCCCTCCCAGCCCCCATCGAG |
| | | AAAACCATCTCCAAAGCCAAAGGGCAGCCCCGAGAACCA |
| | | CAGGTGTACACCCTGCCCCCATCCCGGGAGGAGATGACC |
| | | AAGAACCAGGTCAGCCTGACCTGCCTGGTCAAAGGCTTC |
| | | TATCCCAGCGACATCGCCGTGGAGTGGGAGAGCAATGGG |
| | | CAGCCGGAGAACAACTACAAGACCACGCCTCCCGTGCTG |
| | | GACTCCGACGGCTCCTTCTTCCTCTACAGCAAGCTCACC |
| | | GTGGACAAGAGCAGGTGGCAGCAGGGGAACGTCTTCTCA |
| | | TGCTCCGTGATGCATGAGGCTCTGCACAACCACTACACG |
| | | CAGAAGAGCCTCTCCCTGTCTCCGGGTAAA |
| LCDR1 | 16 | SGSSSNIGSNDVS |
| (Combined) | | |
| LCDR2 | 17 | KNYNRPS |
| (Combined) | | |
| LCDR3 | 18 | SAWDQRQFDVV |
| (Combined) | | |
| LCDR1 (Kabat) | 16 | SGSSSNIGSNDVS |
| LCDR2 (Kabat) | 17 | KNYNRPS |
| LCDR3 (Kabat) | 18 | SAWDQRQFDVV |
| LCDR1 | 19 | SSSNIGSND |
| (Chothia) | | |
| LCDR2 | 20 | KNY |
| (Chothia) | | |
| LCDR3 | 21 | WDQRQFDV |
| (Chothia) | | |
| LCDR1 (IMGT) | 22 | SSNIGSND |
| LCDR2 (IMGT) | 20 | KNY |
| LCDR3 (IMGT) | 18 | SAWDQRQFDVV |
| VL | 362 | DIVLTQPPSVSGAPGQRVTISCSGSSSNIGSNDVSWYQQ |
| | | LPGTAPKLLIYKNYNRPSGVPDRFSGSKSGTSASLAITG |
| | | LQAEDEADYYCSAWDQRQFDVVFGGGTKLTVL |
| DNA VL | 363 | GATATCGTGCTGACCCAGCCGCCGAGCGTGAGCGGTGCA |
| | | CCGGGCCAGCGCGTGACCATTAGCTGTAGCGGCAGCAGC |
| | | AGCAACATTGGTTCTAACGACGTGTCTTGGTACCAGCAG |
| | | CTGCCGGGCACGGCGCCGAAACTGCTGATCTACAAAAAC |
| | | TACAACCGCCCGAGCGGCGTGCCGGATCGCTTTAGCGGA |
| | | TCCAAAAGCGGCACCAGCGCCAGCCTGGCGATTACCGGC |
| | | CTGCAAGCAGAAGACGAAGCGGATTATTACTGCTCTGCT |
| | | TGGGACCAGCGTCAGTTCGACGTTGTGTTTGGCGGCGGC |
| | | ACGAAGTTAACCGTCCTA |
| Light Chain | 364 | DIVLTQPPSVSGAPGQRVTISCSGSSSNIGSNDVSWYQQ |
| | | LPGTAPKLLIYKNYNRPSGVPDRFSGSKSGTSASLAITG |
| | | LQAEDEADYYCSAWDQRQFDVVFGGGTKLTVLGQPKAAP |
| | | SVTLFPPSSEELQANKATLVCLISDFYPGAVTVAWKADS |
| | | SPVKAGVETTTPSKQSNNKYAASSYLSLTPEQWKSHRSY |
| | | SCQVTHEGSTVEKTVAPTECS |
| DNA Light | 365 | GATATCGTGCTGACCCAGCCGCCGAGCGTGAGCGGTGCA |
| Chain | | CCGGGCCAGCGCGTGACCATTAGCTGTAGCGGCAGCAGC |
| | | AGCAACATTGGTTCTAACGACGTGTCTTGGTACCAGCAG |
| | | CTGCCGGGCACGGCGCCGAAACTGCTGATCTACAAAAAC |
| | | TACAACCGCCCGAGCGGCGTGCCGGATCGCTTTAGCGGA |
| | | TCCAAAAGCGGCACCAGCGCCAGCCTGGCGATTACCGGC |
| | | CTGCAAGCAGAAGACGAAGCGGATTATTACTGCTCTGCT |
| | | TGGGACCAGCGTCAGTTCGACGTTGTGTTTGGCGGCGGC |
| | | ACGAAGTTAACCGTCCTAGGTCAGCCCAAGGCTGCCCCC |
| | | TCGGTCACTCTGTTCCCGCCCTCCTCTGAGGAGCTTCAA |
| | | GCCAACAAGGCCACACTGGTGTGTCTCATAAGTGACTTC |
| | | TACCCGGGAGCCGTGACAGTGGCCTGGAAGGCAGATAGC |
| | | AGCCCCGTCAAGGCGGGAGTGGAGACCACCACACCCTCC |
| | | AAACAAAGCAACAACAAGTACGCGGCCAGCAGCTATCTG |
| | | AGCCTGACGCCTGAGCAGTGGAAGTCCCACAGAAGCTAC |
| | | AGCTGCCAGGTCACGCATGAAGGGAGCACCGTGGAGAAG |
| | | ACAGTGGCCCCTACAGAATGTTCA |
| AM2 | | |
| HCDR1 | 3 | GFTFSTAAMS |
| (Combined) | | |
| HCDR2 | 372 | SIEYYDTDTHYADSVKG |
| (Combined) | | |
| HCDR3 | 5 | ELSYLYSGYYFDY |
| (Combined) | | |
| HCDR1 (Kabat) | 6 | TAAMS |
| HCDR2 (Kabat) | 372 | SIEYYDTDTHYADSVKG |
| HCDR3 (Kabat) | 5 | ELSYLYSGYYFDY |
| HCDR1 | 7 | GFTFSTA |
| (Chothia) | | |
| HCDR2 | 373 | EYYDTD |
| (Chothia) | | |
| HCDR3 | 5 | ELSYLYSGYYFDY |
| (Chothia) | | |
| HCDR1 (IMGT) | 9 | GFTFSTAA |
| HCDR2 (IMGT) | 374 | IEYYDTDT |
| HCDR3 (IMGT) | 11 | ARELSYLYSGYYFDY |
| VH | 375 | QVQLLESGGGLVQPGGSLRLSCAASGFTFSTAAMSWVRQ |
| | | APGKGLEWVSSIEYYDTDTHYADSVKGRFTISRDNSKNT |
| | | LYLQMNSLRAEDTAVYYCARELSYLYSGYYFDYWGQGTL |
| | | VTVSS |
| DNA VH | 376 | CAGGTGCAATTGCTGGAAAGCGGCGGTGGCCTGGTGCAG |
| | | CCGGGTGGCAGCCTGCGTCTGAGCTGCGCGGCGTCCGGA |
| | | TTCACCTTTTCTACTGCTGCTATGTCTTGGGTGCGCCAG |
| | | GCCCCGGGCAAAGGTCTCGAGTGGGTTTCCTCTATCGAA |
| | | TACTACGACACTGACACTCATTATGCGGATAGCGTGAAA |
| | | GGCCGCTTTACCATCAGCCGCGATAATTCGAAAAACACC |
| | | CTGTATCTGCAAATGAACAGCCTGCGTGCGGAAGATACG |
| | | GCCGTGTATTATTGCGCGCGTGAACTGTCTTACCTGTAC |
| | | TCTGGTTACTACTTCGATTACTGGGGCCAAGGCACCCTG |
| | | GTGACTGTTAGCTCA |
| Heavy Chain | 377 | QVQLLESGGGLVQPGGSLRLSCAASGFTFSTAAMSWVRQ |
| | | APGKGLEWVSSIEYYDTDTHYADSVKGRFTISRDNSKNT |
| | | LYLQMNSLRAEDTAVYYCARELSYLYSGYYFDYWGQGTL |
| | | VTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKRVEPKSCDKTHTCPPC |
| | | PAPEAAGGPSVFLFPPKPKDTLMISRTPEVTCVVVDVSH |
| | | EDPEVKFNWYVDGVEVHNAKTKPREEQYNSTYRVVSVLT |
| | | VLHQDWLNGKEYKCKVSNKALPAPIEKTISKAKGQPREP |
| | | QVYTLPPSREEMTKNQVSLTCLVKGFYPSDIAVEWESNG |
| | | QPENNYKTTPPVLDSDGSFFLYSKLTVDKSRWQQGNVFS |
| | | CSVMHEALHNHYTQKSLSLSPGK |
| DNA Heavy | 378 | CAGGTGCAATTGCTGGAAAGCGGCGGTGGCCTGGTGCAG |
| Chain | | CCGGGTGGCAGCCTGCGTCTGAGCTGCGCGGCGTCCGGA |
| | | TTCACCTTTTCTACTGCTGCTATGTCTTGGGTGCGCCAG |
| | | GCCCCGGGCAAAGGTCTCGAGTGGGTTTCCTCTATCGAA |
| | | TACTACGACACTGACACTCATTATGCGGATAGCGTGAAA |
| | | GGCCGCTTTACCATCAGCCGCGATAATTCGAAAAACACC |
| | | CTGTATCTGCAAATGAACAGCCTGCGTGCGGAAGATACG |
| | | GCCGTGTATTATTGCGCGCGTGAACTGTCTTACCTGTAC |
| | | TCTGGTTACTACTTCGATTACTGGGGCCAAGGCACCCTG |
| | | GTGACTGTTAGCTCAGCCTCCACCAAGGGTCCATCGGTC |
| | | TTCCCCCTGGCACCCTCCTCCAAGAGCACCTCTGGGGGC |
| | | ACAGCGGCCCTGGGCTGCCTGGTCAAGGACTACTTCCCC |
| | | GAACCGGTGACGGTGTCGTGGAACTCAGGCGCCCTGACC |
| | | AGCGGCGTGCACACCTTCCCGGCTGTCCTACAGTCCTCA |
| | | GGACTCTACTCCCTCAGCAGCGTGGTGACCGTGCCCTCC |
| | | AGCAGCTTGGGCACCCAGACCTACATCTGCAACGTGAAT |
| | | CACAAGCCCAGCAACACCAAGGTGGACAAGAGAGTTGAG |
| | | CCCAAATCTTGTGACAAAACTCACACATGCCCACCGTGC |
| | | CCAGCACCTGAAGCAGCGGGGGGACCGTCAGTCTTCCTC |
| | | TTCCCCCCAAAACCCAAGGACACCCTCATGATCTCCCGG |
| | | ACCCCTGAGGTCACATGCGTGGTGGTGGACGTGAGCCAC |
| | | GAAGACCCTGAGGTCAAGTTCAACTGGTACGTGGACGGC |
| | | GTGGAGGTGCATAATGCCAAGACAAAGCCGCGGGAGGAG |
| | | CAGTACAACAGCACGTACCGGGTGGTCAGCGTCCTCACC |
| | | GTCCTGCACCAGGACTGGCTGAATGGCAAGGAGTACAAG |
| | | TGCAAGGTCTCCAACAAAGCCCTCCCAGCCCCCATCGAG |
| | | AAAACCATCTCCAAAGCCAAAGGGCAGCCCCGAGAACCA |
| | | CAGGTGTACACCCTGCCCCCATCCCGGGAGGAGATGACC |
| | | AAGAACCAGGTCAGCCTGACCTGCCTGGTCAAAGGCTTC |
| | | TATCCCAGCGACATCGCCGTGGAGTGGGAGAGCAATGGG |
| | | CAGCCGGAGAACAACTACAAGACCACGCCTCCCGTGCTG |
| | | GACTCCGACGGCTCCTTCTTCCTCTACAGCAAGCTCACC |
| | | GTGGACAAGAGCAGGTGGCAGCAGGGGAACGTCTTCTCA |
| | | TGCTCCGTGATGCATGAGGCTCTGCACAACCACTACACG |
| | | CAGAAGAGCCTCTCCCTGTCTCCGGGTAAA |
| LCDR1 | 16 | SGSSSNIGSNDVS |
| (Combined) | | |
| LCDR2 | 17 | KNYNRPS |
| (Combined) | | |
| LCDR3 | 18 | SAWDQRQFDVV |
| (Combined) | | |
| LCDR1 (Kabat) | 16 | SGSSSNIGSNDVS |
| LCDR2 (Kabat) | 17 | KNYNRPS |
| LCDR3 (Kabat) | 18 | SAWDQRQFDVV |
| LCDR1 | 19 | SSSNIGSND |
| (Chothia) | | |
| LCDR2 | 20 | KNY |
| (Chothia) | | |
| LCDR3 | 21 | WDQRQFDV |
| (Chothia) | | |
| LCDR1 (IMGT) | 22 | SSNIGSND |
| LCDR2 (IMGT) | 20 | KNY |
| LCDR3 (IMGT) | 18 | SAWDQRQFDVV |
| VL | 362 | DIVLTQPPSVSGAPGQRVTISCSGSSSNIGSNDVSWYQQ |
| | | LPGTAPKLLIYKNYNRPSGVPDRFSGSKSGTSASLAITG |
| | | LQAEDEADYYCSAWDQRQFDVVFGGGTKLTVL |
| DNA VL | 363 | GATATCGTGCTGACCCAGCCGCCGAGCGTGAGCGGTGCA |
| | | CCGGGCCAGCGCGTGACCATTAGCTGTAGCGGCAGCAGC |
| | | AGCAACATTGGTTCTAACGACGTGTCTTGGTACCAGCAG |
| | | CTGCCGGGCACGGCGCCGAAACTGCTGATCTACAAAAAC |
| | | TACAACCGCCCGAGCGGCGTGCCGGATCGCTTTAGCGGA |
| | | TCCAAAAGCGGCACCAGCGCCAGCCTGGCGATTACCGGC |
| | | CTGCAAGCAGAAGACGAAGCGGATTATTACTGCTCTGCT |
| | | TGGGACCAGCGTCAGTTCGACGTTGTGTTTGGCGGCGGC |
| | | ACGAAGTTAACCGTCCTA |
| Light Chain | 364 | DIVLTQPPSVSGAPGQRVTISCSGSSSNIGSNDVSWYQQ |
| | | LPGTAPKLLIYKNYNRPSGVPDRFSGSKSGTSASLAITG |
| | | LQAEDEADYYCSAWDQRQFDVVFGGGTKLTVLGQPKAAP |
| | | SVTLFPPSSEELQANKATLVCLISDFYPGAVTVAWKADS |
| | | SPVKAGVETTTPSKQSNNKYAASSYLSLTPEQWKSHRSY |
| | | SCQVTHEGSTVEKTVAPTECS |
| DNA Light | 365 | GATATCGTGCTGACCCAGCCGCCGAGCGTGAGCGGTGCA |
| Chain | | CCGGGCCAGCGCGTGACCATTAGCTGTAGCGGCAGCAGC |
| | | AGCAACATTGGTTCTAACGACGTGTCTTGGTACCAGCAG |
| | | CTGCCGGGCACGGCGCCGAAACTGCTGATCTACAAAAAC |
| | | TACAACCGCCCGAGCGGCGTGCCGGATCGCTTTAGCGGA |
| | | TCCAAAAGCGGCACCAGCGCCAGCCTGGCGATTACCGGC |
| | | CTGCAAGCAGAAGACGAAGCGGATTATTACTGCTCTGCT |
| | | TGGGACCAGCGTCAGTTCGACGTTGTGTTTGGCGGCGGC |
| | | ACGAAGTTAACCGTCCTAGGTCAGCCCAAGGCTGCCCCC |
| | | TCGGTCACTCTGTTCCCGCCCTCCTCTGAGGAGCTTCAA |
| | | GCCAACAAGGCCACACTGGTGTGTCTCATAAGTGACTTC |
| | | TACCCGGGAGCCGTGACAGTGGCCTGGAAGGCAGATAGC |
| | | AGCCCCGTCAAGGCGGGAGTGGAGACCACCACACCCTCC |
| | | AAACAAAGCAACAACAAGTACGCGGCCAGCAGCTATCTG |
| | | AGCCTGACGCCTGAGCAGTGGAAGTCCCACAGAAGCTAC |
| | | AGCTGCCAGGTCACGCATGAAGGGAGCACCGTGGAGAAG |
| | | ACAGTGGCCCCTACAGAATGTTCA |
| AM3 | | |
| HCDR1 | 3 | GFTFSTAAMS |
| (Combined) | | |
| HCDR2 | 379 | TIEYSSQETYYADSVKG |
| (Combined) | | |
| HCDR3 | 5 | ELSYLYSGYYFDY |
| (Combined) | | |
| HCDR1 (Kabat) | 6 | TAAMS |
| HCDR2 (Kabat) | 379 | TIEYSSQETYYADSVKG |
| HCDR3 (Kabat) | 5 | ELSYLYSGYYFDY |
| HCDR1 | 7 | GFTFSTA |
| (Chothia) | | |
| HCDR2 | 380 | EYSSQE |
| (Chothia) | | |
| HCDR3 | 5 | ELSYLYSGYYFDY |
| (Chothia) | | |
| HCDR1 (IMGT) | 9 | GFTFSTAA |
| HCDR2 (IMGT) | 381 | IEYSSQET |
| HCDR3 (IMGT) | 11 | ARELSYLYSGYYFDY |
| VH | 382 | QVQLLESGGGLVQPGGSLRLSCAASGFTFSTAAMSWVRQ |
| | | APGKGLEWVSTIEYSSQETYYADSVKGRFTISRDNSKNT |
| | | LYLQMNSLRAEDTAVYYCARELSYLYSGYYFDYWGQGTL |
| | | VTVSS |
| DNA VH | 383 | CAGGTGCAATTGCTGGAAAGCGGCGGTGGCCTGGTGCAG |
| | | CCGGGTGGCAGCCTGCGTCTGAGCTGCGCGGCGTCCGGA |
| | | TTCACCTTTTCTACTGCTGCTATGTCTTGGGTGCGCCAG |
| | | GCCCCGGGCAAAGGTCTCGAGTGGGTTTCCACTATCGAA |
| | | TACTCTAGCCAGGAAACTTACTATGCGGATAGCGTGAAA |
| | | GGCCGCTTTACCATCAGCCGCGATAATTCGAAAAACACC |
| | | CTGTATCTGCAAATGAACAGCCTGCGTGCGGAAGATACG |
| | | GCCGTGTATTATTGCGCGCGTGAACTGTCTTACCTGTAC |
| | | TCTGGTTACTACTTCGATTACTGGGGCCAAGGCACCCTG |
| | | GTGACTGTTAGCTCA |
| Heavy Chain | 384 | QVQLLESGGGLVQPGGSLRLSCAASGFTFSTAAMSWVRQ |
| | | APGKGLEWVSTIEYSSQETYYADSVKGRFTISRDNSKNT |
| | | LYLQMNSLRAEDTAVYYCARELSYLYSGYYFDYWGQGTL |
| | | VTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKRVEPKSCDKTHTCPPC |
| | | PAPEAAGGPSVFLFPPKPKDTLMISRTPEVTCVVVDVSH |
| | | EDPEVKFNWYVDGVEVHNAKTKPREEQYNSTYRVVSVLT |
| | | VLHQDWLNGKEYKCKVSNKALPAPIEKTISKAKGQPREP |
| | | QVYTLPPSREEMTKNQVSLTCLVKGFYPSDIAVEWESNG |
| | | QPENNYKTTPPVLDSDGSFFLYSKLTVDKSRWQQGNVFS |
| | | CSVMHEALHNHYTQKSLSLSPGK |
| DNA Heavy | 385 | CAGGTGCAATTGCTGGAAAGCGGCGGTGGCCTGGTGCAG |
| Chain | | CCGGGTGGCAGCCTGCGTCTGAGCTGCGCGGCGTCCGGA |
| | | TTCACCTTTTCTACTGCTGCTATGTCTTGGGTGCGCCAG |
| | | GCCCCGGGCAAAGGTCTCGAGTGGGTTTCCACTATCGAA |
| | | TACTCTAGCCAGGAAACTTACTATGCGGATAGCGTGAAA |
| | | GGCCGCTTTACCATCAGCCGCGATAATTCGAAAAACACC |
| | | CTGTATCTGCAAATGAACAGCCTGCGTGCGGAAGATACG |
| | | GCCGTGTATTATTGCGCGCGTGAACTGTCTTACCTGTAC |
| | | TCTGGTTACTACTTCGATTACTGGGGCCAAGGCACCCTG |
| | | GTGACTGTTAGCTCAGCCTCCACCAAGGGTCCATCGGTC |
| | | TTCCCCCTGGCACCCTCCTCCAAGAGCACCTCTGGGGGC |
| | | ACAGCGGCCCTGGGCTGCCTGGTCAAGGACTACTTCCCC |
| | | GAACCGGTGACGGTGTCGTGGAACTCAGGCGCCCTGACC |
| | | AGCGGCGTGCACACCTTCCCGGCTGTCCTACAGTCCTCA |
| | | GGACTCTACTCCCTCAGCAGCGTGGTGACCGTGCCCTCC |
| | | AGCAGCTTGGGCACCCAGACCTACATCTGCAACGTGAAT |
| | | CACAAGCCCAGCAACACCAAGGTGGACAAGAGAGTTGAG |
| | | CCCAAATCTTGTGACAAAACTCACACATGCCCACCGTGC |
| | | CCAGCACCTGAAGCAGCGGGGGGACCGTCAGTCTTCCTC |
| | | TTCCCCCCAAAACCCAAGGACACCCTCATGATCTCCCGG |
| | | ACCCCTGAGGTCACATGCGTGGTGGTGGACGTGAGCCAC |
| | | GAAGACCCTGAGGTCAAGTTCAACTGGTACGTGGACGGC |
| | | GTGGAGGTGCATAATGCCAAGACAAAGCCGCGGGAGGAG |
| | | CAGTACAACAGCACGTACCGGGTGGTCAGCGTCCTCACC |
| | | GTCCTGCACCAGGACTGGCTGAATGGCAAGGAGTACAAG |
| | | TGCAAGGTCTCCAACAAAGCCCTCCCAGCCCCCATCGAG |
| | | AAAACCATCTCCAAAGCCAAAGGGCAGCCCCGAGAACCA |
| | | CAGGTGTACACCCTGCCCCCATCCCGGGAGGAGATGACC |
| | | AAGAACCAGGTCAGCCTGACCTGCCTGGTCAAAGGCTTC |
| | | TATCCCAGCGACATCGCCGTGGAGTGGGAGAGCAATGGG |
| | | CAGCCGGAGAACAACTACAAGACCACGCCTCCCGTGCTG |
| | | GACTCCGACGGCTCCTTCTTCCTCTACAGCAAGCTCACC |
| | | GTGGACAAGAGCAGGTGGCAGCAGGGGAACGTCTTCTCA |
| | | TGCTCCGTGATGCATGAGGCTCTGCACAACCACTACACG |
| | | CAGAAGAGCCTCTCCCTGTCTCCGGGTAAA |
| LCDR1 | 16 | SGSSSNIGSNDVS |
| (Combined) | | |
| LCDR2 | 17 | KNYNRPS |
| (Combined) | | |
| LCDR3 | 18 | SAWDQRQFDVV |
| (Combined) | | |
| LCDR1 (Kabat) | 16 | SGSSSNIGSNDVS |
| LCDR2 (Kabat) | 17 | KNYNRPS |
| LCDR3 (Kabat) | 18 | SAWDQRQFDVV |
| LCDR1 | 19 | SSSNIGSND |
| (Chothia) | | |
| LCDR2 | 20 | KNY |
| (Chothia) | | |
| LCDR3 | 21 | WDQRQFDV |
| (Chothia) | | |
| LCDR1 (IMGT) | 22 | SSNIGSND |
| LCDR2 (IMGT) | 20 | KNY |
| LCDR3 (IMGT) | 18 | SAWDQRQFDVV |
| VL | 362 | DIVLTQPPSVSGAPGQRVTISCSGSSSNIGSNDVSWYQQ |
| | | LPGTAPKLLIYKNYNRPSGVPDRFSGSKSGTSASLAITG |
| | | LQAEDEADYYCSAWDQRQFDVVFGGGTKLTVL |
| DNA VL | 363 | GATATCGTGCTGACCCAGCCGCCGAGCGTGAGCGGTGCA |
| | | CCGGGCCAGCGCGTGACCATTAGCTGTAGCGGCAGCAGC |
| | | AGCAACATTGGTTCTAACGACGTGTCTTGGTACCAGCAG |
| | | CTGCCGGGCACGGCGCCGAAACTGCTGATCTACAAAAAC |
| | | TACAACCGCCCGAGCGGCGTGCCGGATCGCTTTAGCGGA |
| | | TCCAAAAGCGGCACCAGCGCCAGCCTGGCGATTACCGGC |
| | | CTGCAAGCAGAAGACGAAGCGGATTATTACTGCTCTGCT |
| | | TGGGACCAGCGTCAGTTCGACGTTGTGTTTGGCGGCGGC |
| | | ACGAAGTTAACCGTCCTA |
| Light Chain | 364 | DIVLTQPPSVSGAPGQRVTISCSGSSSNIGSNDVSWYQQ |
| | | LPGTAPKLLIYKNYNRPSGVPDRFSGSKSGTSASLAITG |
| | | LQAEDEADYYCSAWDQRQFDVVFGGGTKLTVLGQPKAAP |
| | | SVTLFPPSSEELQANKATLVCLISDFYPGAVTVAWKADS |
| | | SPVKAGVETTTPSKQSNNKYAASSYLSLTPEQWKSHRSY |
| | | SCQVTHEGSTVEKTVAPTECS |
| DNA Light | 365 | GATATCGTGCTGACCCAGCCGCCGAGCGTGAGCGGTGCA |
| Chain | | CCGGGCCAGCGCGTGACCATTAGCTGTAGCGGCAGCAGC |
| | | AGCAACATTGGTTCTAACGACGTGTCTTGGTACCAGCAG |
| | | CTGCCGGGCACGGCGCCGAAACTGCTGATCTACAAAAAC |
| | | TACAACCGCCCGAGCGGCGTGCCGGATCGCTTTAGCGGA |
| | | TCCAAAAGCGGCACCAGCGCCAGCCTGGCGATTACCGGC |
| | | CTGCAAGCAGAAGACGAAGCGGATTATTACTGCTCTGCT |
| | | TGGGACCAGCGTCAGTTCGACGTTGTGTTTGGCGGCGGC |
| | | ACGAAGTTAACCGTCCTAGGTCAGCCCAAGGCTGCCCCC |
| | | TCGGTCACTCTGTTCCCGCCCTCCTCTGAGGAGCTTCAA |
| | | GCCAACAAGGCCACACTGGTGTGTCTCATAAGTGACTTC |
| | | TACCCGGGAGCCGTGACAGTGGCCTGGAAGGCAGATAGC |
| | | AGCCCCGTCAAGGCGGGAGTGGAGACCACCACACCCTCC |
| | | AAACAAAGCAACAACAAGTACGCGGCCAGCAGCTATCTG |
| | | AGCCTGACGCCTGAGCAGTGGAAGTCCCACAGAAGCTAC |
| | | AGCTGCCAGGTCACGCATGAAGGGAGCACCGTGGAGAAG |
| | | ACAGTGGCCCCTACAGAATGTTCA |
| AM4 | | |
| HCDR1 | 3 | GFTFSTAAMS |
| (Combined) | | |
| HCDR2 | 379 | TIEYSSQETYYADSVKG |
| (Combined) | | |
| HCDR3 | 5 | ELSYLYSGYYFDY |
| (Combined) | | |
| HCDR1 (Kabat) | 6 | TAAMS |
| HCDR2 (Kabat) | 379 | TIEYSSQETYYADSVKG |
| HCDR3 (Kabat) | 5 | ELSYLYSGYYFDY |
| HCDR1 | 7 | GFTFSTA |
| (Chothia) | | |
| HCDR2 | 380 | EYSSQE |
| (Chothia) | | |
| HCDR3 | 5 | ELSYLYSGYYFDY |
| (Chothia) | | |
| HCDR1 (IMGT) | 9 | GFTFSTAA |
| HCDR2 (IMGT) | 381 | IEYSSQET |
| HCDR3 (IMGT) | 11 | ARELSYLYSGYYFDY |
| VH | 382 | QVQLLESGGGLVQPGGSLRLSCAASGFTFSTAAMSWVRQ |
| | | APGKGLEWVSTIEYSSQETYYADSVKGRFTISRDNSKNT |
| | | LYLQMNSLRAEDTAVYYCARELSYLYSGYYFDYWGQGTL |
| | | VTVSS |
| DNA VH | 392 | CAAGTGCAGCTGCTTGAATCTGGCGGCGGACTGGTGCAG |
| | | CCTGGCGGCTCCCTGAGACTGTCTTGCGCCGCCTCCGGC |
| | | TTCACCTTCTCCACCGCCGCTATGTCCTGGGTCCGACAG |
| | | GCTCCCGGCAAGGGCCTGGAATGGGTGTCCACCATTGAG |
| | | TACTCCAGCCAGGAAACCTACTACGCCGACTCCGTGAAG |
| | | GGCCGGTTCACCATCTCCCGGGACAACTCCAAGAACACC |
| | | CTGTACCTGCAGATGAACTCCCTGCGGGCCGAGGACACC |
| | | GCCGTGTACTACTGCGCCAGAGAGCTGTCCTACCTGTAC |
| | | TCCGGCTACTACTTCGACTACTGGGGCCAGGGCACCCTG |
| | | GTCACCGTGTCCTCT |
| Heavy Chain | 384 | QVQLLESGGGLVQPGGSLRLSCAASGFTFSTAAMSWVRQ |
| | | APGKGLEWVSTIEYSSQETYYADSVKGRFTISRDNSKNT |
| | | LYLQMNSLRAEDTAVYYCARELSYLYSGYYFDYWGQGTL |
| | | VTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKRVEPKSCDKTHTCPPC |
| | | PAPEAAGGPSVFLFPPKPKDTLMISRTPEVTCVVVDVSH |
| | | EDPEVKFNWYVDGVEVHNAKTKPREEQYNSTYRVVSVLT |
| | | VLHQDWLNGKEYKCKVSNKALPAPIEKTISKAKGQPREP |
| | | QVYTLPPSREEMTKNQVSLTCLVKGFYPSDIAVEWESNG |
| | | QPENNYKTTPPVLDSDGSFFLYSKLTVDKSRWQQGNVFS |
| | | CSVMHEALHNHYTQKSLSLSPGK |
| DNA Heavy | 393 | CAAGTGCAGCTGCTTGAATCTGGCGGCGGACTGGTGCAG |
| Chain | | CCTGGCGGCTCCCTGAGACTGTCTTGCGCCGCCTCCGGC |
| | | TTCACCTTCTCCACCGCCGCTATGTCCTGGGTCCGACAG |
| | | GCTCCCGGCAAGGGCCTGGAATGGGTGTCCACCATTGAG |
| | | TACTCCAGCCAGGAAACCTACTACGCCGACTCCGTGAAG |
| | | GGCCGGTTCACCATCTCCCGGGACAACTCCAAGAACACC |
| | | CTGTACCTGCAGATGAACTCCCTGCGGGCCGAGGACACC |
| | | GCCGTGTACTACTGCGCCAGAGAGCTGTCCTACCTGTAC |
| | | TCCGGCTACTACTTCGACTACTGGGGCCAGGGCACCCTG |
| | | GTCACCGTGTCCTCTGCTAGCACCAAGGGCCCCTCCGTG |
| | | TTCCCTCTGGCCCCTTCCAGCAAGTCTACCTCCGGCGGC |
| | | ACAGCTGCTCTGGGCTGCCTGGTCAAGGACTACTTCCCT |
| | | GAGCCTGTGACAGTGTCCTGGAACTCTGGCGCCCTGACC |
| | | TCTGGCGTGCACACCTTCCCTGCCGTGCTGCAGTCCTCC |
| | | GGCCTGTACTCCCTGTCCTCCGTGGTCACAGTGCCTTCA |
| | | AGCAGCCTGGGCACCCAGACCTATATCTGCAACGTGAAC |
| | | CACAAGCCTTCCAACACCAAGGTGGACAAGCGGGTGGAG |
| | | CCTAAGTCCTGCGACAAGACCCACACCTGTCCTCCCTGC |
| | | CCTGCTCCTGAAGCTGCTGGCGGCCCTTCTGTGTTCCTG |
| | | TTCCCTCCAAAGCCCAAGGACACCCTGATGATCTCCCGG |
| | | ACCCCTGAAGTGACCTGCGTGGTGGTGGACGTGTCCCAC |
| | | GAGGATCCTGAAGTGAAGTTCAATTGGTACGTGGACGGC |
| | | GTGGAGGTGCACAACGCCAAGACCAAGCCTCGGGAGGAA |
| | | CAGTACAACTCCACCTACCGGGTGGTGTCCGTGCTGACC |
| | | GTGCTGCACCAGGACTGGCTGAACGGCAAAGAGTACAAG |
| | | TGCAAAGTCTCCAACAAGGCCCTGCCTGCCCCTATCGAA |
| | | AAGACAATCTCCAAGGCCAAGGGCCAGCCTAGGGAACCC |
| | | CAGGTGTACACCCTGCCACCCAGCCGGGAGGAAATGACC |
| | | AAGAACCAGGTGTCCCTGACCTGTCTGGTCAAGGGCTTC |
| | | TACCCTTCCGATATCGCCGTGGAGTGGGAGTCTAACGGC |
| | | CAGCCTGAGAACAACTACAAGACCACCCCTCCTGTGCTG |
| | | GACTCCGACGGCTCCTTCTTCCTGTACTCCAAACTGACC |
| | | GTGGACAAGTCCCGGTGGCAGCAGGGCAACGTGTTCTCC |
| | | TGCTCCGTGATGCACGAGGCCCTGCACAACCACTACACC |
| | | CAGAAGTCCCTGTCCCTGTCTCCCGGCAAG |
| LCDR1 | 16 | SGSSSNIGSNDVS |
| (Combined) | | |
| LCDR2 | 17 | KNYNRPS |
| (Combined) | | |
| LCDR3 | 18 | SAWDQRQFDVV |
| (Combined) | | |
| LCDR1 (Kabat) | 16 | SGSSSNIGSNDVS |
| LCDR2 (Kabat) | 17 | KNYNRPS |
| LCDR3 (Kabat) | 18 | SAWDQRQFDVV |
| LCDR1 | 19 | SSSNIGSND |
| (Chothia) | | |
| LCDR2 | 20 | KNY |
| (Chothia) | | |
| LCDR3 | 21 | WDQRQFDV |
| (Chothia) | | |
| LCDR1 (IMGT) | 22 | SSNIGSND |
| LCDR2 (IMGT) | 20 | KNY |
| LCDR3 (IMGT) | 18 | SAWDQRQFDVV |
| VL | 386 | QSVLTQPPSVSGAPGQRVTISCSGSSSNIGSNDVSWYQQ |
| | | LPGTAPKLLIYKNYNRPSGVPDRFSGSKSGTSASLAITG |
| | | LQAEDEADYYCSAWDQRQFDVVFGGGTKLTVL |
| DNA VL | 387 | CAGAGCGTGCTGACACAGCCTCCCTCCGTGTCTGGCGCC |
| | | CCTGGCCAGAGAGTGACCATCTCCTGCTCCGGCTCCTCC |
| | | TCCAACATCGGCTCCAACGACGTGTCCTGGTATCAGCAG |
| | | CTGCCCGGCACCGCCCCTAAGCTGCTGATCTACAAGAAC |
| | | TACAACCGGCCCTCCGGCGTGCCCGACCGGTTCTCTGGC |
| | | TCCAAGTCTGGCACCTCCGCCTCCCTGGCTATCACCGGC |
| | | CTGCAGGCTGAGGACGAGGCCGACTACTACTGCTCCGCC |
| | | TGGGACCAGCGGCAGTTCGACGTGGTGTTCGGCGGAGGC |
| | | ACCAAGCTGACCGTGCTG |
| Light Chain | 388 | QSVLTQPPSVSGAPGQRVTISCSGSSSNIGSNDVSWYQQ |
| | | LPGTAPKLLIYKNYNRPSGVPDRFSGSKSGTSASLAITG |
| | | LQAEDEADYYCSAWDQRQFDVVFGGGTKLTVLGQPKAAP |
| | | SVTLFPPSSEELQANKATLVCLISDFYPGAVTVAWKADS |
| | | SPVKAGVETTTPSKQSNNKYAASSYLSLTPEQWKSHRSY |
| | | SCQVTHEGSTVEKTVAPTECS |
| DNA Light | 389 | CAGAGCGTGCTGACACAGCCTCCCTCCGTGTCTGGCGCC |
| Chain | | CCTGGCCAGAGAGTGACCATCTCCTGCTCCGGCTCCTCC |
| | | TCCAACATCGGCTCCAACGACGTGTCCTGGTATCAGCAG |
| | | CTGCCCGGCACCGCCCCTAAGCTGCTGATCTACAAGAAC |
| | | TACAACCGGCCCTCCGGCGTGCCCGACCGGTTCTCTGGC |
| | | TCCAAGTCTGGCACCTCCGCCTCCCTGGCTATCACCGGC |
| | | CTGCAGGCTGAGGACGAGGCCGACTACTACTGCTCCGCC |
| | | TGGGACCAGCGGCAGTTCGACGTGGTGTTCGGCGGAGGC |
| | | ACCAAGCTGACCGTGCTGGGCCAGCCTAAGGCTGCCCCC |
| | | AGCGTGACCCTGTTCCCCCCCAGCAGCGAGGAGCTGCAG |
| | | GCCAACAAGGCCACCCTGGTGTGCCTGATCAGCGACTTC |
| | | TACCCAGGCGCCGTGACCGTGGCCTGGAAGGCCGACAGC |
| | | AGCCCCGTGAAGGCCGGCGTGGAGACCACCACCCCCAGC |
| | | AAGCAGAGCAACAACAAGTACGCCGCCAGCAGCTACCTG |
| | | AGCCTGACCCCCGAGCAGTGGAAGAGCCACAGGTCCTAC |
| | | AGCTGCCAGGTGACCCACGAGGGCAGCACCGTGGAAAAG |
| | | ACCGTGGCCCCAACCGAGTGCAGC |

### Binding/Reversal Agents

In one aspect, the present disclosure relates to a reversal binding agent which is an anti-idiotype antibody, such as a full length IgG, and fragments thereof (for example a Fab fragment) which specifically binds a target antibody that binds human Factor XI (“FXI”) and/or Factor XIa (“FXIa”) (“anti-FXI/FXIa antibody”), for example an anti-FXI/FXIa antibody described in Table 1, such as antibody NOV1401, or affinity matured variants thereof, such as antibody AM1, AM2, AM3, or AM4.

In a particular aspect, provided herein is a binding agent, as well as a pharmaceutical composition comprising such binding agent, which specifically binds a target antibody that binds human Factor XI (“FXI”) and/or Factor XIa (“FXIa”) (“anti-FXI/FXIa antibody”, such as antibody NOV1401) within the catalytic domain, wherein the binding agent inhibits or reverses an anticoagulant activity of the target antibody, wherein the binding agent binds to the target antibody with a dissociation constant (K_{D}) of 1 nM or less, and wherein the binding agent is capable of inhibiting the ability of the target antibody to delay activated partial thromboplastin time (aPTT) by at least 35%. In further specific aspects, the binding agent is capable of inhibiting the ability of the target antibody to delay activated partial thromboplastin time (aPTT) by at least 40%. In further specific aspects, the binding agent is capable of inhibiting the ability of the target antibody to delay activated partial thromboplastin time (aPTT) by at least 50%. In further specific aspects, the binding agent is capable of inhibiting the ability of the target antibody to delay activated partial thromboplastin time (aPTT) by at least 60%. In further specific aspects, the binding agent is capable of inhibiting the ability of the target antibody to delay activated partial thromboplastin time (aPTT) by at least 70%. Methods for determining aPTT and delay to aPTT have been described in the art, and are also described herein, e.g., Examples Section.

In specific aspects, provided herein are binding agents, as well as pharmaceutical compositions comprising such binding agents, which inhibit or reverses an anticoagulant activity of a target anti-FXI/FXIa antibody (e.g., NOV1401), wherein the binding agents are antigen-binding human antibody fragments such as human Fabs. In particular aspects, provided herein are binding agents, as well as pharmaceutical compositions comprising such binding agents, which inhibit or reverses an anticoagulant activity of a target anti-FXI/FXIa antibody (e.g., NOV1401), wherein the binding agents are human anti-idiotype Fabs. In particular aspects, provided herein are binding agents which inhibit or reverses an anticoagulant activity of a target anti-FXI/FXIa antibody (e.g., NOV1401), wherein the binding agents are human IgG1, IgG2, or IgG4 antibodies, or variants thereof.

In further specific aspects, provided herein is a binding agent (e.g., anti-idiotype antibody), as well as a pharmaceutical composition comprising such binding agent, which specifically binds a target anti-FXI/FXIa antibody, wherein the binding agent inhibits an anticoagulant activity of the target anti-FXI/FXIa antibody, and wherein the target anti-FXI/FXIa antibody comprises (i) a heavy chain variable region (VH) comprising the amino acid sequence of SEQ ID NO: 12 and a light chain variable region (VL) comprising the amino acid sequence of SEQ ID NO: 23; or (ii) a heavy chain comprising the amino acid sequence of SEQ ID NO: 14 and a light chain comprising the amino acid sequence of SEQ ID NO: 25.

In particular aspects, anti-FXI/FXIa antibody binding agents provided herein (e.g., IDT11 or IDT12) is capable of reducing, inhibiting, or reversing (e.g., partially reversing) one or more of the following anticoagulant effects mediated by an anti-FXI/FXIa antibody: (i) aPTT prolongation in aPTT assays and (ii) reduction in the amount of thrombin in a thrombin generation assay (TGA) in human plasma. Protocols and assays to measure these anticoagulant activities have been described, and exemplary assays are described herein, e.g., in the Examples Section.

In a specific aspect, an anti-FXI/FXIa antibody binding agent provided herein (e.g., IDT11 or IDT12) is capable reversing anticoagulant effects of a target FXI/FXIa antibody as characterized by reducing, inhibiting, or reversing aPTT prolongation by an anti-FXI/FXIa antibody (e.g., NOV1401) by at least 10%, at least 20%, at least 30%, at least 40%, at least 50%, at least 60%, at least 70%, at least 80%, or at least 90%, as determined by an aPTT assay, described in the art or herein.

In a specific aspect, an anti-FXI/FXIa antibody binding agent provided herein is capable reversing anticoagulant effects of a target FXI/FXIa antibody as characterized by reducing, inhibiting, or reversing reduction in the amount of thrombin in a thrombin generation assay (TGA) in human plasma by an anti-FXI/FXIa antibody (e.g., NOV1401) by at least 10%, at least 20%, at least 30%, at least 40%, at least 50%, at least 60%, at least 70%, at least 80%, or at least 90%.

In further specific aspects, provided herein is a binding agent (e.g., anti-idiotype antibody), as well as a pharmaceutical composition comprising such binding agent, which specifically binds a target anti-FXI/FXIa antibody, wherein the binding agent inhibits an anticoagulant activity of the target anti-FXI/FXIa antibody, wherein the target anti-FXI/FXIa antibody comprises (i) a heavy chain variable region (VH) comprising the amino acid sequence of SEQ ID NO: 12 and a light chain variable region (VL) comprising the amino acid sequence of SEQ ID NO: 23; or (ii) a heavy chain comprising the amino acid sequence of SEQ ID NO: 14 and a light chain comprising the amino acid sequence of SEQ ID NO: 25, and wherein the binding agent is an antibody or antigen-binding fragment thereof comprising (1) a VH comprising complementarity determining regions HCDR1, HCDR2, and HCDR3 selected from those set forth in Table 2, and (2) a VL comprising complementarity determining regions LCDR1, LCDR2, and LCDR3 selected from those set forth in Table 2. In a particular aspect, the binding agent (e.g., anti-idiotype antibody) comprises Combined HCDR1, HCDR2, and HCDR3 selected from those set forth in Table 2 and Combined LCDR1, LCDR2, and LCDR3 selected from those set forth in Table 2. In a particular aspect, the binding agent (e.g., anti-idiotype antibody) comprises Kabat HCDR1, HCDR2, and HCDR3 selected from those set forth in Table 2 and Kabat LCDR1, LCDR2, and LCDR3 selected from those set forth in Table 2. In a particular aspect, the binding agent (e.g., anti-idiotype antibody) comprises Chothia HCDR1, HCDR2, and HCDR3 selected from those set forth in Table 2 and Chothia LCDR1, LCDR2, and LCDR3 selected from those set forth in Table 2. In a particular aspect, the binding agent (e.g., anti-idiotype antibody) comprises IMGT HCDR1, HCDR2, and HCDR3 selected from those set forth in Table 2 and IMGT LCDR1, LCDR2, and LCDR3 selected from those set forth in Table 2.

TABLE 2
Examples of anti-FXI/FXIa Antibody Binding Agents (e.g., anti-idiotype antibody
and Fab fragments)

| | description | DNA or amino acid sequence |
|---|---|---|
| IDT1 | | |
| SEQ ID NO: 27 | HCDR1 | GFTFSDYAMS |
| (Combined) | | |
| SEQ ID NO: 28 | HCDR2 | VIDYSSSNTYYADSVKG |
| (Combined) | | |
| SEQ ID NO: 29 | HCDR3 | EGYSYRSIRFDY |
| (Combined) | | |
| SEQ ID NO: 30 | HCDR1 | DYAMS |
| (Kabat) | | |
| SEQ ID NO: 31 | HCDR2 | VIDYSSSNTYYADSVKG |
| (Kabat) | | |
| SEQ ID NO: 32 | HCDR3 | EGYSYRSIRFDY |
| (Kabat) | | |
| SEQ ID NO: 33 | HCDR1 | GFTFSDY |
| (Chothia) | | |
| SEQ ID NO: 34 | HCDR2 | DYSSSN |
| (Chothia) | | |
| SEQ ID NO: 35 | HCDR3 | EGYSYRSIRFDY |
| (Chothia) | | |
| SEQ ID NO: 36 | HCDR1 | GFTFSDYA |
| (IMGT) | | |
| SEQ ID NO: 37 | HCDR2 | IDYSSSNT |
| (IMGT) | | |
| SEQ ID NO: 38 | HCDR3 | AREGYSYRSIRFDY |
| (IMGT) | | |
| SEQ ID NO: 39 | VH | QVQLLESGGGLVQPGGSLRLSCAASGFTFSDYAMSWVRQAPGKG |
| | | LEWVSVIDYSSSNTYYADSVKGRFTISRDNSKNTLYLQMNSLRA |
| | | EDTAVYYCAREGYSYRSIRFDYWGQGTLVTVSS |
| SEQ ID NO: 40 | DNA VH | CAAGTGCAGCTGCTGGAATCTGGCGGCGGACTGGTGCAGCCTGG |
| | | CGGTAGTCTGAGACTGTCTTGCGCCGCCTCCGGCTTCACCTTCT |
| | | CCGACTACGCCATGTCCTGGGTCCGACAGGCCCCTGGCAAGGGC |
| | | CTGGAGTGGGTGTCCGTGATCGACTACTCCTCCTCCAACACCTA |
| | | CTACGCCGACTCCGTGAAGGGCCGGTTCACCATCTCCCGGGACA |
| | | ACTCCAAGAACACCCTGTACCTGCAGATGAACTCCCTGCGGGCC |
| | | GAGGACACCGCCGTGTACTACTGCGCCAGAGAGGGCTACTCCTA |
| | | CCGGTCCATCAGATTCGACTACTGGGGCCAGGGCACCCTGGTCA |
| | | CCGTGTCCTCT |
| SEQ ID NO: 41 | Heavy | QVQLLESGGGLVQPGGSLRLSCAASGFTFSDYAMSWVRQAPGKG |
| | Chain | LEWVSVIDYSSSNTYYADSVKGRFTISRDNSKNTLYLQMNSLRA |
| | | EDTAVYYCAREGYSYRSIRFDYWGQGTLVTVSSASTKGPSVFPL |
| | | APSSKSTSGGTAALGCLVKDYFPEPVTVSWNSGALTSGVHTFPA |
| | | VLQSSGLYSLSSVVTVPSSSLGTQTYICNVNHKPSNTKVDKRVE |
| | | PKSC |
| SEQ ID NO: 42 | DNA | CAAGTGCAGCTGCTGGAATCTGGCGGCGGACTGGTGCAGCCTGG |
| | Heavy | CGGTAGTCTGAGACTGTCTTGCGCCGCCTCCGGCTTCACCTTCT |
| | Chain | CCGACTACGCCATGTCCTGGGTCCGACAGGCCCCTGGCAAGGGC |
| | | CTGGAGTGGGTGTCCGTGATCGACTACTCCTCCTCCAACACCTA |
| | | CTACGCCGACTCCGTGAAGGGCCGGTTCACCATCTCCCGGGACA |
| | | ACTCCAAGAACACCCTGTACCTGCAGATGAACTCCCTGCGGGCC |
| | | GAGGACACCGCCGTGTACTACTGCGCCAGAGAGGGCTACTCCTA |
| | | CCGGTCCATCAGATTCGACTACTGGGGCCAGGGCACCCTGGTCA |
| | | CCGTGTCCTCTGCTAGCACCAAGGGCCCCTCCGTGTTCCCTCTG |
| | | GCCCCTTCCAGCAAGTCTACCTCTGGCGGCACCGCTGCTCTGGG |
| | | CTGCCTGGTGAAGGACTACTTCCCTGAGCCTGTGACAGTGTCCT |
| | | GGAACTCTGGCGCCCTGACCTCCGGCGTGCACACCTTCCCTGCC |
| | | GTGCTGCAGTCCTCCGGCCTGTACTCCCTGTCCTCCGTGGTGAC |
| | | AGTGCCTTCCTCCAGCCTGGGCACCCAGACCTATATCTGCAACG |
| | | TGAACCACAAGCCTTCCAACACCAAGGTGGACAAGCGGGTGGAG |
| | | CCTAAGTCATGC |
| SEQ ID NO: 43 | LCDR1 | RASQSISSNLN |
| (Combined) | | |
| SEQ ID NO: 44 | LCDR2 | AASNLQS |
| (Combined) | | |
| SEQ ID NO: 45 | LCDR3 | LQFDHTPFT |
| (Combined) | | |
| SEQ ID NO: 46 | LCDR1 | RASQSISSNLN |
| (Kabat) | | |
| SEQ ID NO: 47 | LCDR2 | AASNLQS |
| (Kabat) | | |
| SEQ ID NO: 48 | LCDR3 | LQFDHTPFT |
| (Kabat) | | |
| SEQ ID NO: 49 | LCDR1 | SQSISSN |
| (Chothia) | | |
| SEQ ID NO: 50 | LCDR2 | AAS |
| (Chothia) | | |
| SEQ ID NO: 51 | LCDR3 | FDHTPF |
| (Chothia) | | |
| SEQ ID NO: 52 | LCDR1 | QSISSN |
| (IMGT) | | |
| SEQ ID NO: 53 | LCDR2 | AAS |
| (IMGT) | | |
| SEQ ID NO: 54 | LCDR3 | LQFDHTPFT |
| (IMGT) | | |
| SEQ ID NO: 55 | VL | DIQMTQSPSSLSASVGDRVTITCRASQSISSNLNWYQQKPGKAP |
| | | KLLIYAASNLQSGVPSRFSGSGSGTDFTLTISSLQPEDFATYYC |
| | | LQFDHTPFTFGQGTKVEIK |
| SEQ ID NO: 56 | DNA VL | GACATCCAGATGACCCAGAGCCCCTCCAGCCTGTCCGCCTCCGT |
| | | GGGCGACAGAGTGACCATCACCTGTCGGGCCTCCCAGTCCATCT |
| | | CCTCCAACCTGAACTGGTATCAGCAGAAGCCCGGCAAGGCCCCT |
| | | AAGCTGCTGATCTACGCCGCCAGCAACCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | CTGCAGTTCGACCACACCCCTTTCACCTTCGGCCAGGGCACCAA |
| | | AGTGGAAATCAAG |
| SEQ ID NO: 57 | Light | DIQMTQSPSSLSASVGDRVTITCRASQSISSNLNWYQQKPGKAP |
| | Chain | KLLIYAASNLQSGVPSRFSGSGSGTDFTLTISSLQPEDFATYYC |
| | | LQFDHTPFTFGQGTKVEIKRTVAAPSVFIFPPSDEQLKSGTASV |
| | | VCLLNNFYPREAKVQWKVDNALQSGNSQESVTEQDSKDSTYSLS |
| | | STLTLSKADYEKHKVYACEVTHQGLSSPVTKSFNRGEC |
| SEQ ID NO: 58 | DNA | GACATCCAGATGACCCAGAGCCCCTCCAGCCTGTCCGCCTCCGT |
| | Light | GGGCGACAGAGTGACCATCACCTGTCGGGCCTCCCAGTCCATCT |
| | Chain | CCTCCAACCTGAACTGGTATCAGCAGAAGCCCGGCAAGGCCCCT |
| | | AAGCTGCTGATCTACGCCGCCAGCAACCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | CTGCAGTTCGACCACACCCCTTTCACCTTCGGCCAGGGCACCAA |
| | | AGTGGAAATCAAGCGTACGGTGGCCGCTCCCAGCGTGTTCATCT |
| | | TCCCCCCCAGCGACGAGCAGCTGAAGAGCGGCACCGCCAGCGTG |
| | | GTGTGCCTGCTGAACAACTTCTACCCCCGGGAGGCCAAGGTGCA |
| | | GTGGAAGGTGGACAACGCCCTGCAGAGCGGCAACAGCCAGGAGA |
| | | GCGTCACCGAGCAGGACAGCAAGGACTCCACCTACAGCCTGAGC |
| | | AGCACCCTGACCCTGAGCAAGGCCGACTACGAGAAGCATAAGGT |
| | | GTACGCCTGCGAGGTGACCCACCAGGGCCTGTCCAGCCCCGTGA |
| | | CCAAGAGCTTCAACAGGGGCGAGTGC |
| IDT2 | | |
| SEQ ID NO: 59 | HCDR1 | GFTFSSAAVH |
| (Combined) | | |
| SEQ ID NO: 60 | HCDR2 | RIKSKADGGTTDYAAPVKG |
| (Combined) | | |
| SEQ ID NO: 61 | HCDR3 | DSPSISSYSIPYFSGMDV |
| (Combined) | | |
| SEQ ID NO: 62 | HCDR1 | SAAVH |
| (Kabat) | | |
| SEQ ID NO: 63 | HCDR2 | RIKSKADGGTTDYAAPVKG |
| (Kabat) | | |
| SEQ ID NO: 64 | HCDR3 | DSPSISSYSIPYFSGMDV |
| (Kabat) | | |
| SEQ ID NO: 65 | HCDR1 | GFTFSSA |
| (Chothia) | | |
| SEQ ID NO: 66 | HCDR2 | KSKADGGT |
| (Chothia) | | |
| SEQ ID NO: 67 | HCDR3 | DSPSISSYSIPYFSGMDV |
| (Chothia) | | |
| SEQ ID NO: 68 | HCDR1 | GFTFSSAA |
| (IMGT) | | |
| SEQ ID NO: 69 | HCDR2 | IKSKADGGTT |
| (IMGT) | | |
| SEQ ID NO: 70 | HCDR3 | ARDSPSISSYSIPYFSGMDV |
| (IMGT) | | |
| SEQ ID NO: 71 | VH | QVQLVESGGGLVKPGGSLRLSCAASGFTFSSAAVHWVRQAPGKG |
| | | LEWVGRIKSKADGGITDYAAPVKGRFTISRDDSKNTLYLQMNSL |
| | | KTEDTAVYYCARDSPSISSYSIPYFSGMDVWGQGTLVTVSS |
| SEQ ID NO: 72 | DNA VH | CAAGTGCAGCTGGTGGAATCTGGCGGCGGACTGGTCAAGCCTGG |
| | | CGGTAGCCTGAGACTGTCTTGCGCCGCCTCCGGCTTCACCTTCT |
| | | CCTCTGCCGCTGTGCACTGGGTCCGACAGGCCCCTGGCAAGGGC |
| | | CTGGAGTGGGTCGGACGGATCAAGTCCAAGGCCGACGGCGGCAC |
| | | CACCGACTACGCTGCCCCTGTGAAGGGCCGGTTCACCATCTCCC |
| | | GGGACGACTCCAAGAACACCCTGTACCTGCAGATGAACTCCCTG |
| | | AAAACCGAGGACACCGCCGTGTACTACTGCGCCAGAGACTCCCC |
| | | ATCTATCTCCAGCTACTCCATCCCCTACTTCTCCGGCATGGACG |
| | | TGTGGGGCCAGGGCACCCTGGTCACCGTGTCCTCT |
| SEQ ID NO: 73 | Heavy | QVQLVESGGGLVKPGGSLRLSCAASGFTFSSAAVHWVRQAPGKG |
| | Chain | LEWVGRIKSKADGGTTDYAAPVKGRFTISRDDSKNTLYLQMNSL |
| | | KTEDTAVYYCARDSPSISSYSIPYFSGMDVWGQGTLVTVSSAST |
| | | KGPSVFPLAPSSKSTSGGTAALGCLVKDYFPEPVTVSWNSGALT |
| | | SGVHTFPAVLQSSGLYSLSSVVTVPSSSLGTQTYICNVNHKPSN |
| | | TKVDKRVEPKSC |
| SEQ ID NO: 74 | DNA | CAAGTGCAGCTGGTGGAATCTGGCGGCGGACTGGTCAAGCCTGG |
| | Heavy | CGGTAGCCTGAGACTGTCTTGCGCCGCCTCCGGCTTCACCTTCT |
| | Chain | CCTCTGCCGCTGTGCACTGGGTCCGACAGGCCCCTGGCAAGGGC |
| | | CTGGAGTGGGTCGGACGGATCAAGTCCAAGGCCGACGGCGGCAC |
| | | CACCGACTACGCTGCCCCTGTGAAGGGCCGGTTCACCATCTCCC |
| | | GGGACGACTCCAAGAACACCCTGTACCTGCAGATGAACTCCCTG |
| | | AAAACCGAGGACACCGCCGTGTACTACTGCGCCAGAGACTCCCC |
| | | ATCTATCTCCAGCTACTCCATCCCCTACTTCTCCGGCATGGACG |
| | | TGTGGGGCCAGGGCACCCTGGTCACCGTGTCCTCTGCTAGCACC |
| | | AAGGGCCCCTCCGTGTTCCCTCTGGCCCCTTCCAGCAAGTCTAC |
| | | CTCTGGCGGCACCGCTGCTCTGGGCTGCCTGGTGAAGGACTACT |
| | | TCCCTGAGCCTGTGACAGTGTCCTGGAACTCTGGCGCCCTGACC |
| | | TCCGGCGTGCACACCTTCCCTGCCGTGCTGCAGTCCTCCGGCCT |
| | | GTACTCCCTGTCCTCCGTGGTGACAGTGCCTTCCTCCAGCCTGG |
| | | GCACCCAGACCTATATCTGCAACGTGAACCACAAGCCTTCCAAC |
| | | ACCAAGGTGGACAAGCGGGTGGAGCCTAAGTCATGC |
| SEQ ID NO: 75 | LCDR1 | RASQGIRAWLN |
| (Combined) | | |
| SEQ ID NO: 76 | LCDR2 | AASSLQS |
| (Combined) | | |
| SEQ ID NO: 77 | LCDR3 | HQYITHPPT |
| (Combined) | | |
| SEQ ID NO: 78 | LCDR1 | RASQGIRAWLN |
| (Kabat) | | |
| SEQ ID NO: 79 | LCDR2 | AASSLQS |
| (Kabat) | | |
| SEQ ID NO: 80 | LCDR3 | HQYITHPPT |
| (Kabat) | | |
| SEQ ID NO: 81 | LCDR1 | SQGIRAW |
| (Chothia) | | |
| SEQ ID NO: 82 | LCDR2 | AAS |
| (Chothia) | | |
| SEQ ID NO: 83 | LCDR3 | YITHPP |
| (Chothia) | | |
| SEQ ID NO: 84 | LCDR1 | QGIRAW |
| (IMGT) | | |
| SEQ ID NO: 85 | LCDR2 | AAS |
| (IMGT) | | |
| SEQ ID NO: 86 | LCDR3 | HQYITHPPT |
| (IMGT) | | |
| SEQ ID NO: 87 | VL | DIQMTQSPSSLSASVGDRVTITCRASQGIRAWLNWYQQKPGKAP |
| | | KLLIYAASSLQSGVPSRFSGSGSGTDFTLTISSLQPEDFATYYC |
| | | HQYITHPPTFGQGTKVEIK |
| SEQ ID NO: 88 | DNA VL | GACATCCAGATGACCCAGAGCCCCTCCAGCCTGTCCGCCTCCGT |
| | | GGGCGACAGAGTGACCATCACCTGTCGGGCCTCTCAGGGCATCC |
| | | GGGCCTGGCTGAACTGGTATCAGCAGAAGCCCGGCAAGGCCCCT |
| | | AAGCTGCTGATCTACGCCGCCAGCTCCCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | CACCAGTACATCACCCACCCTCCCACCTTCGGCCAGGGCACCAA |
| | | AGTGGAAATCAAG |
| SEQ ID NO: 89 | Light | DIQMTQSPSSLSASVGDRVTITCRASQGIRAWLNWYQQKPGKAP |
| | Chain | KLLIYAASSLQSGVPSRFSGSGSGTDFTLTISSLQPEDFATYYC |
| | | HQYITHPPTFGQGTKVEIKRTVAAPSVFIFPPSDEQLKSGTASV |
| | | VCLLNNFYPREAKVQWKVDNALQSGNSQESVTEQDSKDSTYSLS |
| | | STLTLSKADYEKHKVYACEVTHQGLSSPVTKSFNRGEC |
| SEQ ID NO: 90 | DNA | GACATCCAGATGACCCAGAGCCCCTCCAGCCTGTCCGCCTCCGT |
| | Light | GGGCGACAGAGTGACCATCACCTGTCGGGCCTCTCAGGGCATCC |
| | Chain | GGGCCTGGCTGAACTGGTATCAGCAGAAGCCCGGCAAGGCCCCT |
| | | AAGCTGCTGATCTACGCCGCCAGCTCCCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | CACCAGTACATCACCCACCCTCCCACCTTCGGCCAGGGCACCAA |
| | | AGTGGAAATCAAGCGTACGGTGGCCGCTCCCAGCGTGTTCATCT |
| | | TCCCCCCCAGCGACGAGCAGCTGAAGAGCGGCACCGCCAGCGTG |
| | | GTGTGCCTGCTGAACAACTTCTACCCCCGGGAGGCCAAGGTGCA |
| | | GTGGAAGGTGGACAACGCCCTGCAGAGCGGCAACAGCCAGGAGA |
| | | GCGTCACCGAGCAGGACAGCAAGGACTCCACCTACAGCCTGAGC |
| | | AGCACCCTGACCCTGAGCAAGGCCGACTACGAGAAGCATAAGGT |
| | | GTACGCCTGCGAGGTGACCCACCAGGGCCTGTCCAGCCCCGTGA |
| | | CCAAGAGCTTCAACAGGGGCGAGTGC |
| IDT3 | | |
| SEQ ID NO: 91 | HCDR1 | GFTFQSAAVH |
| (Combined) | | |
| SEQ ID NO: 92 | HCDR2 | RIKSKADGGTTDYAAPVKG |
| (Combined) | | |
| SEQ ID NO: 93 | HCDR3 | DSPSISSYSIPYFSGMDV |
| (Combined) | | |
| SEQ ID NO: 94 | HCDR1 | SAAVH |
| (Kabat) | | |
| SEQ ID NO: 95 | HCDR2 | RIKSKADGGTTDYAAPVKG |
| (Kabat) | | |
| SEQ ID NO: 96 | HCDR3 | DSPSISSYSIPYFSGMDV |
| (Kabat) | | |
| SEQ ID NO: 97 | HCDR1 | GFTFQSA |
| (Chothia) | | |
| SEQ ID NO: 98 | HCDR2 | KSKADGGT |
| (Chothia) | | |
| SEQ ID NO: 99 | HCDR3 | DSPSISSYSIPYFSGMDV |
| (Chothia) | | |
| SEQ ID NO: 100 | HCDR1 | GFTFQSAA |
| (IMGT) | | |
| SEQ ID NO: 101 | HCDR2 | IKSKADGGTT |
| (IMGT) | | |
| SEQ ID NO: 102 | HCDR3 | ARDSPSISSYSIPYFSGMDV |
| (IMGT) | | |
| SEQ ID NO: 103 | VH | QVQLVESGGGLVKPGGSLRLSCAASGFTFQSAAVHWVRQAPGKG |
| | | LEWVGRIKSKADGGTTDYAAPVKGRFTISRDDSKNTLYLQMNSL |
| | | KTEDTAVYYCARDSPSISSYSIPYFSGMDVWGQGTLVTVSS |
| SEQ ID NO: 104 | DNA VH | CAAGTGCAGCTGGTGGAATCTGGCGGCGGACTGGTCAAGCCTGG |
| | | CGGTAGCCTGAGACTGTCTTGCGCCGCCTCCGGCTTCACCTTCC |
| | | AGTCTGCCGCTGTGCACTGGGTCCGACAGGCCCCTGGCAAGGGC |
| | | CTGGAGTGGGTCGGACGGATCAAGTCCAAGGCCGACGGCGGCAC |
| | | CACCGACTACGCTGCCCCTGTGAAGGGCCGGTTCACCATCTCCC |
| | | GGGACGACTCCAAGAACACCCTGTACCTGCAGATGAACTCCCTG |
| | | AAAACCGAGGACACCGCCGTGTACTACTGCGCCAGAGACTCCCC |
| | | ATCTATCTCCAGCTACTCCATCCCCTACTTCTCCGGCATGGACG |
| | | TGTGGGGCCAGGGCACCCTGGTCACCGTGTCCTCT |
| SEQ ID NO: 105 | Heavy | QVQLVESGGGLVKPGGSLRLSCAASGFTFQSAAVHWVRQAPGKG |
| | Chain | LEWVGRIKSKADGGTTDYAAPVKGRFTISRDDSKNTLYLQMNSL |
| | | KTEDTAVYYCARDSPSISSYSIPYFSGMDVWGQGTLVTVSSAST |
| | | KGPSVFPLAPSSKSTSGGTAALGCLVKDYFPEPVTVSWNSGALT |
| | | SGVHTFPAVLQSSGLYSLSSVVTVPSSSLGTQTYICNVNHKPSN |
| | | TKVDKRVEPKSC |
| SEQ ID NO: 106 | DNA | CAAGTGCAGCTGGTGGAATCTGGCGGCGGACTGGTCAAGCCTGG |
| | Heavy | CGGTAGCCTGAGACTGTCTTGCGCCGCCTCCGGCTTCACCTTCC |
| | Chain | AGTCTGCCGCTGTGCACTGGGTCCGACAGGCCCCTGGCAAGGGC |
| | | CTGGAGTGGGTCGGACGGATCAAGTCCAAGGCCGACGGCGGCAC |
| | | CACCGACTACGCTGCCCCTGTGAAGGGCCGGTTCACCATCTCCC |
| | | GGGACGACTCCAAGAACACCCTGTACCTGCAGATGAACTCCCTG |
| | | AAAACCGAGGACACCGCCGTGTACTACTGCGCCAGAGACTCCCC |
| | | ATCTATCTCCAGCTACTCCATCCCCTACTTCTCCGGCATGGACG |
| | | TGTGGGGCCAGGGCACCCTGGTCACCGTGTCCTCTGCTAGCACC |
| | | AAGGGCCCCTCCGTGTTCCCTCTGGCCCCTTCCAGCAAGTCTAC |
| | | CTCTGGCGGCACCGCTGCTCTGGGCTGCCTGGTGAAGGACTACT |
| | | TCCCTGAGCCTGTGACAGTGTCCTGGAACTCTGGCGCCCTGACC |
| | | TCCGGCGTGCACACCTTCCCTGCCGTGCTGCAGTCCTCCGGCCT |
| | | GTACTCCCTGTCCTCCGTGGTGACAGTGCCTTCCTCCAGCCTGG |
| | | GCACCCAGACCTATATCTGCAACGTGAACCACAAGCCTTCCAAC |
| | | ACCAAGGTGGACAAGCGGGTGGAGCCTAAGTCATGC |
| SEQ ID NO: 107 | LCDR1 | RASQGIRAWLN |
| (Combined) | | |
| SEQ ID NO: 108 | LCDR2 | AASSLQS |
| (Combined) | | |
| SEQ ID NO: 109 | LCDR3 | HQYITHPPT |
| (Combined) | | |
| SEQ ID NO: 110 | LCDR1 | RASQGIRAWLN |
| (Kabat) | | |
| SEQ ID NO: 111 | LCDR2 | AASSLQS |
| (Kabat) | | |
| SEQ ID NO: 112 | LCDR3 | HQYITHPPT |
| (Kabat) | | |
| SEQ ID NO: 113 | LCDR1 | SQGIRAW |
| (Chothia) | | |
| SEQ ID NO: 114 | LCDR2 | AAS |
| (Chothia) | | |
| SEQ ID NO: 115 | LCDR3 | YITHPP |
| (Chothia) | | |
| SEQ ID NO: 116 | LCDR1 | QGIRAW |
| (IMGT) | | |
| SEQ ID NO: 117 | LCDR2 | AAS |
| (IMGT) | | |
| SEQ ID NO: 118 | LCDR3 | HQYITHPPT |
| (IMGT) | | |
| SEQ ID NO: 119 | VL | DIQMTQSPSSLSASVGDRVTITCRASQGIRAWLNWYQQKPGKAP |
| | | KLLIYAASSLQSGVPSRFSGSGSGTDFTLTISSLQPEDFATYYC |
| | | HQYITHPPTFGQGTKVEIK |
| SEQ ID NO: 120 | DNA VL | GACATCCAGATGACCCAGAGCCCCTCCAGCCTGTCCGCCTCCGT |
| | | GGGCGACAGAGTGACCATCACCTGTCGGGCCTCTCAGGGCATCC |
| | | GGGCCTGGCTGAACTGGTATCAGCAGAAGCCCGGCAAGGCCCCT |
| | | AAGCTGCTGATCTACGCCGCCAGCTCCCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | CACCAGTACATCACCCACCCTCCCACCTTCGGCCAGGGCACCAA |
| | | AGTGGAAATCAAG |
| SEQ ID NO: 121 | Light | DIQMTQSPSSLSASVGDRVTITCRASQGIRAWLNWYQQKPGKAP |
| | Chain | KLLIYAASSLQSGVPSRFSGSGSGTDFTLTISSLQPEDFATYYC |
| | | HQYITHPPTFGQGTKVEIKRTVAAPSVFIFPPSDEQLKSGTASV |
| | | VCLLNNFYPREAKVQWKVDNALQSGNSQESVTEQDSKDSTYSLS |
| | | STLTLSKADYEKHKVYACEVTHQGLSSPVTKSFNRGEC |
| SEQ ID NO: 122 | DNA | GACATCCAGATGACCCAGAGCCCCTCCAGCCTGTCCGCCTCCGT |
| | Light | GGGCGACAGAGTGACCATCACCTGTCGGGCCTCTCAGGGCATCC |
| | Chain | GGGCCTGGCTGAACTGGTATCAGCAGAAGCCCGGCAAGGCCCCT |
| | | AAGCTGCTGATCTACGCCGCCAGCTCCCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | CACCAGTACATCACCCACCCTCCCACCTTCGGCCAGGGCACCAA |
| | | AGTGGAAATCAAGCGTACGGTGGCCGCTCCCAGCGTGTTCATCT |
| | | TCCCCCCCAGCGACGAGCAGCTGAAGAGCGGCACCGCCAGCGTG |
| | | GTGTGCCTGCTGAACAACTTCTACCCCCGGGAGGCCAAGGTGCA |
| | | GTGGAAGGTGGACAACGCCCTGCAGAGCGGCAACAGCCAGGAGA |
| | | GCGTCACCGAGCAGGACAGCAAGGACTCCACCTACAGCCTGAGC |
| | | AGCACCCTGACCCTGAGCAAGGCCGACTACGAGAAGCATAAGGT |
| | | GTACGCCTGCGAGGTGACCCACCAGGGCCTGTCCAGCCCCGTGA |
| | | CCAAGAGCTTCAACAGGGGCGAGTGC |
| IDT4 | | |
| SEQ ID NO: 123 | HCDR1 | GFTFSSAAVH |
| (Combined) | | |
| SEQ ID NO: 124 | HCDR2 | RIKSKASGGTTDYAAPVKG |
| (Combined) | | |
| SEQ ID NO: 125 | HCDR3 | DSPSISSYSIPYFSGMDV |
| (Combined) | | |
| SEQ ID NO: 126 | HCDR1 | SAAVH |
| (Kabat) | | |
| SEQ ID NO: 127 | HCDR2 | RIKSKASGGTTDYAAPVKG |
| (Kabat) | | |
| SEQ ID NO: 128 | HCDR3 | DSPSISSYSIPYFSGMDV |
| (Kabat) | | |
| SEQ ID NO: 129 | HCDR1 | GFTFSSA |
| (Chothia) | | |
| SEQ ID NO: 130 | HCDR2 | KSKASGGT |
| (Chothia) | | |
| SEQ ID NO: 131 | HCDR3 | DSPSISSYSIPYFSGMDV |
| (Chothia) | | |
| SEQ ID NO: 132 | HCDR1 | GFTFSSAA |
| (IMGT) | | |
| SEQ ID NO: 133 | HCDR2 | IKSKASGGTT |
| (IMGT) | | |
| SEQ ID NO: 134 | HCDR3 | ARDSPSISSYSIPYFSGMDV |
| (IMGT) | | |
| SEQ ID NO: 135 | VH | QVQLVESGGGLVKPGGSLRLSCAASGFTFSSAAVHWVRQAPGKG |
| | | LEWVGRIKSKASGGTTDYAAPVKGRFTISRDDSKNTLYLQMNSL |
| | | KTEDTAVYYCARDSPSISSYSIPYFSGMDVWGQGTLVTVSS |
| SEQ ID NO: 136 | DNA VH | CAAGTGCAGCTGGTGGAATCTGGCGGCGGACTGGTCAAGCCTGG |
| | | CGGTAGCCTGAGACTGTCTTGCGCCGCCTCCGGCTTCACCTTCT |
| | | CCTCTGCCGCTGTGCACTGGGTCCGACAGGCCCCTGGCAAGGGC |
| | | CTGGAGTGGGTCGGACGGATCAAGTCCAAGGCCTCCGGCGGCAC |
| | | CACCGACTACGCTGCCCCTGTGAAGGGCCGGTTCACCATCTCCC |
| | | GGGACGACTCCAAGAACACCCTGTACCTGCAGATGAACTCCCTG |
| | | AAAACCGAGGACACCGCCGTGTACTACTGCGCCAGAGACTCCCC |
| | | ATCTATCTCCAGCTACTCCATCCCCTACTTCTCCGGCATGGACG |
| | | TGTGGGGCCAGGGCACCCTGGTCACCGTGTCCTCT |
| SEQ ID NO: 137 | Heavy | QVQLVESGGGLVKPGGSLRLSCAASGFTFSSAAVHWVRQAPGKG |
| | Chain | LEWVGRIKSKASGGTTDYAAPVKGRFTISRDDSKNTLYLQMNSL |
| | | KTEDTAVYYCARDSPSISSYSIPYFSGMDVWGQGTLVTVSSAST |
| | | KGPSVFPLAPSSKSTSGGTAALGCLVKDYFPEPVTVSWNSGALT |
| | | SGVHTFPAVLQSSGLYSLSSVVTVPSSSLGTQTYICNVNHKPSN |
| | | TKVDKRVEPKSC |
| SEQ ID NO: 138 | DNA | CAAGTGCAGCTGGTGGAATCTGGCGGCGGACTGGTCAAGCCTGG |
| | Heavy | CGGTAGCCTGAGACTGTCTTGCGCCGCCTCCGGCTTCACCTTCT |
| | Chain | CCTCTGCCGCTGTGCACTGGGTCCGACAGGCCCCTGGCAAGGGC |
| | | CTGGAGTGGGTCGGACGGATCAAGTCCAAGGCCTCCGGCGGCAC |
| | | CACCGACTACGCTGCCCCTGTGAAGGGCCGGTTCACCATCTCCC |
| | | GGGACGACTCCAAGAACACCCTGTACCTGCAGATGAACTCCCTG |
| | | AAAACCGAGGACACCGCCGTGTACTACTGCGCCAGAGACTCCCC |
| | | ATCTATCTCCAGCTACTCCATCCCCTACTTCTCCGGCATGGACG |
| | | TGTGGGGCCAGGGCACCCTGGTCACCGTGTCCTCTGCTAGCACC |
| | | AAGGGCCCCTCCGTGTTCCCTCTGGCCCCTTCCAGCAAGTCTAC |
| | | CTCTGGCGGCACCGCTGCTCTGGGCTGCCTGGTGAAGGACTACT |
| | | TCCCTGAGCCTGTGACAGTGTCCTGGAACTCTGGCGCCCTGACC |
| | | TCCGGCGTGCACACCTTCCCTGCCGTGCTGCAGTCCTCCGGCCT |
| | | GTACTCCCTGTCCTCCGTGGTGACAGTGCCTTCCTCCAGCCTGG |
| | | GCACCCAGACCTATATCTGCAACGTGAACCACAAGCCTTCCAAC |
| | | ACCAAGGTGGACAAGCGGGTGGAGCCTAAGTCATGC |
| SEQ ID NO: 139 | LCDR1 | RASQGIRAWLN |
| (Combined) | | |
| SEQ ID NO: 140 | LCDR2 | AASSLQS |
| (Combined) | | |
| SEQ ID NO: 141 | LCDR3 | HQYITHPPT |
| (Combined) | | |
| SEQ ID NO: 142 | LCDR1 | RASQGIRAWLN |
| (Kabat) | | |
| SEQ ID NO: 143 | LCDR2 | AASSLQS |
| (Kabat) | | |
| SEQ ID NO: 144 | LCDR3 | HQYITHPPT |
| (Kabat) | | |
| SEQ ID NO: 145 | LCDR1 | SQGIRAW |
| (Chothia) | | |
| SEQ ID NO: 146 | LCDR2 | AAS |
| (Chothia) | | |
| SEQ ID NO: 147 | LCDR3 | YITHPP |
| (Chothia) | | |
| SEQ ID NO: 148 | LCDR1 | QGIRAW |
| (IMGT) | | |
| SEQ ID NO: 149 | LCDR2 | AAS |
| (IMGT) | | |
| SEQ ID NO: 150 | LCDR3 | HQYITHPPT |
| (IMGT) | | |
| SEQ ID NO: 151 | VL | DIQMTQSPSSLSASVGDRVTITCRASQGIRAWLNWYQQKPGKAP |
| | | KLLIYAASSLQSGVPSRFSGSGSGTDFTLTISSLQPEDFATYYC |
| | | HQYITHPPTFGQGTKVEIK |
| SEQ ID NO: 152 | DNA VL | GACATCCAGATGACCCAGAGCCCCTCCAGCCTGTCCGCCTCCGT |
| | | GGGCGACAGAGTGACCATCACCTGTCGGGCCTCTCAGGGCATCC |
| | | GGGCCTGGCTGAACTGGTATCAGCAGAAGCCCGGCAAGGCCCCT |
| | | AAGCTGCTGATCTACGCCGCCAGCTCCCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | CACCAGTACATCACCCACCCTCCCACCTTCGGCCAGGGCACCAA |
| | | AGTGGAAATCAAG |
| SEQ ID NO: 153 | Light | DIQMTQSPSSLSASVGDRVTITCRASQGIRAWLNWYQQKPGKAP |
| | Chain | KLLIYAASSLQSGVPSRFSGSGSGTDFTLTISSLQPEDFATYYC |
| | | HQYITHPPTFGQGTKVEIKRTVAAPSVFIFPPSDEQLKSGTASV |
| | | VCLLNNFYPREAKVQWKVDNALQSGNSQESVTEQDSKDSTYSLS |
| | | STLTLSKADYEKHKVYACEVTHQGLSSPVTKSFNRGEC |
| SEQ ID NO: 154 | DNA | GACATCCAGATGACCCAGAGCCCCTCCAGCCTGTCCGCCTCCGT |
| | Light | GGGCGACAGAGTGACCATCACCTGTCGGGCCTCTCAGGGCATCC |
| | Chain | GGGCCTGGCTGAACTGGTATCAGCAGAAGCCCGGCAAGGCCCCT |
| | | AAGCTGCTGATCTACGCCGCCAGCTCCCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | CACCAGTACATCACCCACCCTCCCACCTTCGGCCAGGGCACCAA |
| | | AGTGGAAATCAAGCGTACGGTGGCCGCTCCCAGCGTGTTCATCT |
| | | TCCCCCCCAGCGACGAGCAGCTGAAGAGCGGCACCGCCAGCGTG |
| | | GTGTGCCTGCTGAACAACTTCTACCCCCGGGAGGCCAAGGTGCA |
| | | GTGGAAGGTGGACAACGCCCTGCAGAGCGGCAACAGCCAGGAGA |
| | | GCGTCACCGAGCAGGACAGCAAGGACTCCACCTACAGCCTGAGC |
| | | AGCACCCTGACCCTGAGCAAGGCCGACTACGAGAAGCATAAGGT |
| | | GTACGCCTGCGAGGTGACCCACCAGGGCCTGTCCAGCCCCGTGA |
| | | CCAAGAGCTTCAACAGGGGCGAGTGC |
| IDT5 | | |
| SEQ ID NO: 155 | HCDR1 | GFTFSSAAVH |
| (Combined) | | |
| SEQ ID NO: 156 | HCDR2 | RIKSKADAGTTDYAAPVKG |
| (Combined) | | |
| SEQ ID NO: 157 | HCDR3 | DSPSISSYSIPYFSGMDV |
| (Combined) | | |
| SEQ ID NO: 158 | HCDR1 | SAAVH |
| (Kabat) | | |
| SEQ ID NO: 159 | HCDR2 | RIKSKADAGTTDYAAPVKG |
| (Kabat) | | |
| SEQ ID NO: 160 | HCDR3 | DSPSISSYSIPYFSGMDV |
| (Kabat) | | |
| SEQ ID NO: 161 | HCDR1 | GFTFSSA |
| (Chothia) | | |
| SEQ ID NO: 162 | HCDR2 | KSKADAGT |
| (Chothia) | | |
| SEQ ID NO: 163 | HCDR3 | DSPSISSYSIPYFSGMDV |
| (Chothia) | | |
| SEQ ID NO: 164 | HCDR1 | GFTFSSAA |
| (IMGT) | | |
| SEQ ID NO: 165 | HCDR2 | IKSKADAGTT |
| (IMGT) | | |
| SEQ ID NO: 166 | HCDR3 | ARDSPSISSYSIPYFSGMDV |
| (IMGT) | | |
| SEQ ID NO: 167 | VH | QVQLVESGGGLVKPGGSLRLSCAASGFTFSSAAVHWVRQAPGKG |
| | | LEWVGRIKSKADAGTIDYAAPVKGRFTISRDDSKNTLYLQMNSL |
| | | KTEDTAVYYCARDSPSISSYSIPYFSGMDVWGQGTLVTVSS |
| SEQ ID NO: 168 | DNA VH | CAAGTGCAGCTGGTGGAATCTGGCGGCGGACTGGTCAAGCCTGG |
| | | CGGTAGCCTGAGACTGTCTTGCGCCGCCTCCGGCTTCACCTTCT |
| | | CCTCTGCCGCTGTGCACTGGGTCCGACAGGCCCCTGGCAAGGGC |
| | | CTGGAGTGGGTCGGACGGATCAAGTCCAAGGCCGACGCCGGCAC |
| | | CACCGACTACGCTGCCCCTGTGAAGGGCCGGTTCACCATCTCCC |
| | | GGGACGACTCCAAGAACACCCTGTACCTGCAGATGAACTCCCTG |
| | | AAAACCGAGGACACCGCCGTGTACTACTGCGCCAGAGACTCCCC |
| | | ATCTATCTCCAGCTACTCCATCCCCTACTTCTCCGGCATGGACG |
| | | TGTGGGGCCAGGGCACCCTGGTCACCGTGTCCTCT |
| SEQ ID NO: 169 | Heavy | QVQLVESGGGLVKPGGSLRLSCAASGFTFSSAAVHWVRQAPGKG |
| | Chain | LEWVGRIKSKADAGTTDYAAPVKGRFTISRDDSKNTLYLQMNSL |
| | | KTEDTAVYYCARDSPSISSYSIPYFSGMDVWGQGTLVTVSSAST |
| | | KGPSVFPLAPSSKSTSGGTAALGCLVKDYFPEPVTVSWNSGALT |
| | | SGVHTFPAVLQSSGLYSLSSVVTVPSSSLGTQTYICNVNHKPSN |
| | | TKVDKRVEPKSC |
| SEQ ID NO: 170 | DNA | CAAGTGCAGCTGGTGGAATCTGGCGGCGGACTGGTCAAGCCTGG |
| | Heavy | CGGTAGCCTGAGACTGTCTTGCGCCGCCTCCGGCTTCACCTTCT |
| | Chain | CCTCTGCCGCTGTGCACTGGGTCCGACAGGCCCCTGGCAAGGGC |
| | | CTGGAGTGGGTCGGACGGATCAAGTCCAAGGCCGACGCCGGCAC |
| | | CACCGACTACGCTGCCCCTGTGAAGGGCCGGTTCACCATCTCCC |
| | | GGGACGACTCCAAGAACACCCTGTACCTGCAGATGAACTCCCTG |
| | | AAAACCGAGGACACCGCCGTGTACTACTGCGCCAGAGACTCCCC |
| | | ATCTATCTCCAGCTACTCCATCCCCTACTTCTCCGGCATGGACG |
| | | TGTGGGGCCAGGGCACCCTGGTCACCGTGTCCTCTGCTAGCACC |
| | | AAGGGCCCCTCCGTGTTCCCTCTGGCCCCTTCCAGCAAGTCTAC |
| | | CTCTGGCGGCACCGCTGCTCTGGGCTGCCTGGTGAAGGACTACT |
| | | TCCCTGAGCCTGTGACAGTGTCCTGGAACTCTGGCGCCCTGACC |
| | | TCCGGCGTGCACACCTTCCCTGCCGTGCTGCAGTCCTCCGGCCT |
| | | GTACTCCCTGTCCTCCGTGGTGACAGTGCCTTCCTCCAGCCTGG |
| | | GCACCCAGACCTATATCTGCAACGTGAACCACAAGCCTTCCAAC |
| | | ACCAAGGTGGACAAGCGGGTGGAGCCTAAGTCATGC |
| SEQ ID NO: 171 | LCDR1 | RASQGIRAWLN |
| (Combined) | | |
| SEQ ID NO: 172 | LCDR2 | AASSLQS |
| (Combined) | | |
| SEQ ID NO: 173 | LCDR3 | HQYITHPPT |
| (Combined) | | |
| SEQ ID NO: 174 | LCDR1 | RASQGIRAWLN |
| (Kabat) | | |
| SEQ ID NO: 175 | LCDR2 | AASSLQS |
| (Kabat) | | |
| SEQ ID NO: 176 | LCDR3 | HQYITHPPT |
| (Kabat) | | |
| SEQ ID NO: 177 | LCDR1 | SQGIRAW |
| (Chothia) | | |
| SEQ ID NO: 178 | LCDR2 | AAS |
| (Chothia) | | |
| SEQ ID NO: 179 | LCDR3 | YITHPP |
| (Chothia) | | |
| SEQ ID NO: 180 | LCDR1 | QGIRAW |
| (IMGT) | | |
| SEQ ID NO: 181 | LCDR2 | AAS |
| (IMGT) | | |
| SEQ ID NO: 182 | LCDR3 | HQYITHPPT |
| (IMGT) | | |
| SEQ ID NO: 183 | VL | DIQMTQSPSSLSASVGDRVTITCRASQGIRAWLNWYQQKPGKAP |
| | | KLLIYAASSLQSGVPSRFSGSGSGTDFTLTISSLQPEDFATYYC |
| | | HQYITHPPTFGQGTKVEIK |
| SEQ ID NO: 184 | DNA VL | GACATCCAGATGACCCAGAGCCCCTCCAGCCTGTCCGCCTCCGT |
| | | GGGCGACAGAGTGACCATCACCTGTCGGGCCTCTCAGGGCATCC |
| | | GGGCCTGGCTGAACTGGTATCAGCAGAAGCCCGGCAAGGCCCCT |
| | | AAGCTGCTGATCTACGCCGCCAGCTCCCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | CACCAGTACATCACCCACCCTCCCACCTTCGGCCAGGGCACCAA |
| | | AGTGGAAATCAAG |
| SEQ ID NO: 185 | Light | DIQMTQSPSSLSASVGDRVTITCRASQGIRAWLNWYQQKPGKAP |
| | Chain | KLLIYAASSLQSGVPSRFSGSGSGTDFTLTISSLQPEDFATYYC |
| | | HQYITHPPTFGQGTKVEIKRTVAAPSVFIFPPSDEQLKSGTASV |
| | | VCLLNNFYPREAKVQWKVDNALQSGNSQESVTEQDSKDSTYSLS |
| | | STLTLSKADYEKHKVYACEVTHQGLSSPVTKSFNRGEC |
| SEQ ID NO: 186 | DNA | GACATCCAGATGACCCAGAGCCCCTCCAGCCTGTCCGCCTCCGT |
| | Light | GGGCGACAGAGTGACCATCACCTGTCGGGCCTCTCAGGGCATCC |
| | Chain | GGGCCTGGCTGAACTGGTATCAGCAGAAGCCCGGCAAGGCCCCT |
| | | AAGCTGCTGATCTACGCCGCCAGCTCCCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | CACCAGTACATCACCCACCCTCCCACCTTCGGCCAGGGCACCAA |
| | | AGTGGAAATCAAGCGTACGGTGGCCGCTCCCAGCGTGTTCATCT |
| | | TCCCCCCCAGCGACGAGCAGCTGAAGAGCGGCACCGCCAGCGTG |
| | | GTGTGCCTGCTGAACAACTTCTACCCCCGGGAGGCCAAGGTGCA |
| | | GTGGAAGGTGGACAACGCCCTGCAGAGCGGCAACAGCCAGGAGA |
| | | GCGTCACCGAGCAGGACAGCAAGGACTCCACCTACAGCCTGAGC |
| | | AGCACCCTGACCCTGAGCAAGGCCGACTACGAGAAGCATAAGGT |
| | | GTACGCCTGCGAGGTGACCCACCAGGGCCTGTCCAGCCCCGTGA |
| | | CCAAGAGCTTCAACAGGGGCGAGTGC |
| IDT6 | | |
| SEQ ID NO: 187 | HCDR1 | GYSFTNYWIG |
| (Combined) | | |
| SEQ ID NO: 188 | HCDR2 | IIFPGVSYTKYSPSFQG |
| (Combined) | | |
| SEQ ID NO: 189 | HCDR3 | GSDQTLRGSRAMDY |
| (Combined) | | |
| SEQ ID NO: 190 | HCDR1 | NYWIG |
| (Kabat) | | |
| SEQ ID NO: 191 | HCDR2 | IIFPGVSYTKYSPSFQG |
| (Kabat) | | |
| SEQ ID NO: 192 | HCDR3 | GSDQTLRGSRAMDY |
| (Kabat) | | |
| SEQ ID NO: 193 | HCDR1 | GYSFTNY |
| (Chothia) | | |
| SEQ ID NO: 194 | HCDR2 | FPGVSY |
| (Chothia) | | |
| SEQ ID NO: 195 | HCDR3 | GSDQTLRGSRAMDY |
| (Chothia) | | |
| SEQ ID NO: 196 | HCDR1 | GYSFTNYW |
| (IMGT) | | |
| SEQ ID NO: 197 | HCDR2 | IFPGVSYT |
| (IMGT) | | |
| SEQ ID NO: 198 | HCDR3 | ARGSDQTLRGSRAMDY |
| (IMGT) | | |
| SEQ ID NO: 199 | VH | QVQLVQSGAEVKKPGESLKISCKGSGYSFTNYWIGWVRQMPGKG |
| | | LEWMGIIFPGVSYTKYSPSFQGQVTISADKSISTAYLQWSSLKA |
| | | SDTAMYYCARGSDQTLRGSRAMDYWGQGTLVTVSS |
| SEQ ID NO: 200 | DNA VH | CAAGTGCAGCTGGTGCAGTCTGGCGCTGAAGTGAAGAAGCCCGG |
| | | CGAGTCCCTGAAGATCTCCTGCAAGGGCTCCGGCTACTCCTTCA |
| | | CCAACTACTGGATCGGCTGGGTCCGACAGATGCCCGGCAAGGGC |
| | | CTGGAGTGGATGGGCATCATCTTCCCCGGCGTGTCCTACACCAA |
| | | GTACAGCCCCAGCTTCCAGGGCCAAGTCACAATCTCCGCCGACA |
| | | AGTCCATCTCCACCGCCTACCTGCAGTGGTCCTCCCTGAAGGCC |
| | | TCCGACACCGCCATGTACTACTGCGCCAGAGGCTCCGACCAGAC |
| | | CCTGCGGGGCTCCAGAGCCATGGATTACTGGGGCCAGGGCACCC |
| | | TGGTCACCGTGTCCTCT |
| SEQ ID NO: 201 | Heavy | QVQLVQSGAEVKKPGESLKISCKGSGYSFTNYWIGWVRQMPGKG |
| | Chain | LEWMGIIFPGVSYTKYSPSFQGQVTISADKSISTAYLQWSSLKA |
| | | SDTAMYYCARGSDQTLRGSRAMDYWGQGTLVTVSSASTKGPSVF |
| | | PLAPSSKSTSGGTAALGCLVKDYFPEPVTVSWNSGALTSGVHTF |
| | | PAVLQSSGLYSLSSVVTVPSSSLGTQTYICNVNHKPSNTKVDKR |
| | | VEPKSC |
| SEQ ID NO: 202 | DNA | CAAGTGCAGCTGGTGCAGTCTGGCGCTGAAGTGAAGAAGCCCGG |
| | Heavy | CGAGTCCCTGAAGATCTCCTGCAAGGGCTCCGGCTACTCCTTCA |
| | Chain | CCAACTACTGGATCGGCTGGGTCCGACAGATGCCCGGCAAGGGC |
| | | CTGGAGTGGATGGGCATCATCTTCCCCGGCGTGTCCTACACCAA |
| | | GTACAGCCCCAGCTTCCAGGGCCAAGTCACAATCTCCGCCGACA |
| | | AGTCCATCTCCACCGCCTACCTGCAGTGGTCCTCCCTGAAGGCC |
| | | TCCGACACCGCCATGTACTACTGCGCCAGAGGCTCCGACCAGAC |
| | | CCTGCGGGGCTCCAGAGCCATGGATTACTGGGGCCAGGGCACCC |
| | | TGGTCACCGTGTCCTCTGCTAGCACCAAGGGCCCCTCCGTGTTC |
| | | CCTCTGGCCCCTTCCAGCAAGTCTACCTCTGGCGGCACCGCTGC |
| | | TCTGGGCTGCCTGGTGAAGGACTACTTCCCTGAGCCTGTGACAG |
| | | TGTCCTGGAACTCTGGCGCCCTGACCTCCGGCGTGCACACCTTC |
| | | CCTGCCGTGCTGCAGTCCTCCGGCCTGTACTCCCTGTCCTCCGT |
| | | GGTGACAGTGCCTTCCTCCAGCCTGGGCACCCAGACCTATATCT |
| | | GCAACGTGAACCACAAGCCTTCCAACACCAAGGTGGACAAGCGG |
| | | GTGGAGCCTAAGTCATGC |
| SEQ ID NO: 203 | LCDR1 | TGTSSDVGISNYVS |
| (Combined) | | |
| SEQ ID NO: 204 | LCDR2 | EVSNRPS |
| (Combined) | | |
| SEQ ID NO: 205 | LCDR3 | QSYTSLNYV |
| (Combined) | | |
| SEQ ID NO: 206 | LCDR1 | TGTSSDVGISNYVS |
| (Kabat) | | |
| SEQ ID NO: 207 | LCDR2 | EVSNRPS |
| (Kabat) | | |
| SEQ ID NO: 208 | LCDR3 | QSYTSLNYV |
| (Kabat) | | |
| SEQ ID NO: 209 | LCDR1 | TSSDVGISNY |
| (Chothia) | | |
| SEQ ID NO: 210 | LCDR2 | EVS |
| (Chothia) | | |
| SEQ ID NO: 211 | LCDR3 | YTSLNY |
| (Chothia) | | |
| SEQ ID NO: 212 | LCDR1 | SSDVGISNY |
| (IMGT) | | |
| SEQ ID NO: 213 | LCDR2 | EVS |
| (IMGT) | | |
| SEQ ID NO: 214 | LCDR3 | QSYTSLNYV |
| (IMGT) | | |
| SEQ ID NO: 215 | VL | QSALTQPASVSGSPGQSITISCTGTSSDVGISNYVSWYQQHPGK |
| | | APKLMIYEVSNRPSGVSNRFSGSKSGNTASLTISGLQAEDEADY |
| | | YCQSYTSLNYVFGGGTKLTVL |
| SEQ ID NO: 216 | DNA VL | CAGTCCGCCCTGACCCAGCCTGCCTCCGTGTCTGGCTCCCCTGG |
| | | CCAGTCCATCACCATCAGCTGCACCGGCACCTCCAGCGACGTGG |
| | | GCATCTCCAACTACGTGTCCTGGTATCAGCAGCACCCCGGCAAG |
| | | GCCCCTAAGCTGATGATCTACGAAGTGTCCAACCGGCCCTCCGG |
| | | CGTGTCCAACAGATTCTCCGGCTCCAAGTCCGGCAACACCGCCT |
| | | CCCTGACCATCAGCGGCCTGCAGGCTGAGGACGAGGCCGACTAC |
| | | TACTGCCAGTCCTACACCTCCCTGAACTACGTGTTCGGCGGAGG |
| | | CACCAAGCTGACCGTGCTG |
| SEQ ID NO: 217 | Light | QSALTQPASVSGSPGQSITISCTGTSSDVGISNYVSWYQQHPGK |
| | Chain | APKLMIYEVSNRPSGVSNRFSGSKSGNTASLTISGLQAEDEADY |
| | | YCQSYTSLNYVFGGGTKLTVLGQPKAAPSVTLFPPSSEELQANK |
| | | ATLVCLISDFYPGAVTVAWKADSSPVKAGVETTTPSKQSNNKYA |
| | | ASSYLSLTPEQWKSHRSYSCQVTHEGSTVEKTVAPTECS |
| SEQ ID NO: 218 | DNA | CAGTCCGCCCTGACCCAGCCTGCCTCCGTGTCTGGCTCCCCTGG |
| | Light | CCAGTCCATCACCATCAGCTGCACCGGCACCTCCAGCGACGTGG |
| | Chain | GCATCTCCAACTACGTGTCCTGGTATCAGCAGCACCCCGGCAAG |
| | | GCCCCTAAGCTGATGATCTACGAAGTGTCCAACCGGCCCTCCGG |
| | | CGTGTCCAACAGATTCTCCGGCTCCAAGTCCGGCAACACCGCCT |
| | | CCCTGACCATCAGCGGCCTGCAGGCTGAGGACGAGGCCGACTAC |
| | | TACTGCCAGTCCTACACCTCCCTGAACTACGTGTTCGGCGGAGG |
| | | CACCAAGCTGACCGTGCTGGGCCAGCCTAAGGCTGCCCCCAGCG |
| | | TGACCCTGTTCCCCCCCAGCAGCGAGGAGCTGCAGGCCAACAAG |
| | | GCCACCCTGGTGTGCCTGATCAGCGACTTCTACCCAGGCGCCGT |
| | | GACCGTGGCCTGGAAGGCCGACAGCAGCCCCGTGAAGGCCGGCG |
| | | TGGAGACCACCACCCCCAGCAAGCAGAGCAACAACAAGTACGCC |
| | | GCCAGCAGCTACCTGAGCCTGACCCCCGAGCAGTGGAAGAGCCA |
| | | CAGGTCCTACAGCTGCCAGGTGACCCACGAGGGCAGCACCGTGG |
| | | AAAAGACCGTGGCCCCAACCGAGTGCAGC |
| IDT7 | | |
| SEQ ID NO: 219 | HCDR1 | GFTFSSNAMH |
| (Combined) | | |
| SEQ ID NO: 220 | HCDR2 | RIKSKTDGGTTDYAAPVKG |
| (Combined) | | |
| SEQ ID NO: 221 | HCDR3 | DHYYYPFAY |
| (Combined) | | |
| SEQ ID NO: 222 | HCDR1 | SNAMH |
| (Kabat) | | |
| SEQ ID NO: 223 | HCDR2 | RIKSKTDGGTTDYAAPVKG |
| (Kabat) | | |
| SEQ ID NO: 224 | HCDR3 | DHYYYPFAY |
| (Kabat) | | |
| SEQ ID NO: 225 | HCDR1 | GFTFSSN |
| (Chothia) | | |
| SEQ ID NO: 226 | HCDR2 | KSKTDGGT |
| (Chothia) | | |
| SEQ ID NO: 227 | HCDR3 | DHYYYPFAY |
| (Chothia) | | |
| SEQ ID NO: 228 | HCDR1 | GFTFSSNA |
| (IMGT) | | |
| SEQ ID NO: 229 | HCDR2 | IKSKTDGGTT |
| (IMGT) | | |
| SEQ ID NO: 230 | HCDR3 | ARDHYYYPFAY |
| (IMGT) | | |
| SEQ ID NO: 231 | VH | QVQLVESGGGLVKPGGSLRLSCAASGFTFSSNAMHWVRQAPGKG |
| | | LEWVGRIKSKTDGGTTDYAAPVKGRFTISRDDSKNTLYLQMNSL |
| | | KTEDTAVYYCARDHYYYPFAYWGQGTLVTVSS |
| SEQ ID NO: 232 | DNA VH | CAAGTGCAGCTGGTGGAATCTGGCGGCGGACTGGTCAAGCCTGG |
| | | CGGTAGCCTGAGACTGTCTTGCGCCGCCTCCGGCTTCACCTTCT |
| | | CCTCCAACGCCATGCACTGGGTCCGACAGGCCCCTGGCAAGGGC |
| | | CTGGAGTGGGTCGGACGGATCAAGTCCAAGACCGACGGCGGCAC |
| | | CACCGACTACGCTGCCCCTGTGAAGGGCCGGTTCACCATCTCCC |
| | | GGGACGACTCCAAGAACACCCTGTACCTGCAGATGAACTCCCTG |
| | | AAAACCGAGGACACCGCCGTGTACTACTGCGCCAGGGACCACTA |
| | | CTACTACCCCTTCGCCTACTGGGGCCAGGGCACCCTGGTCACCG |
| | | TGTCCTCT |
| SEQ ID NO: 233 | Heavy | QVQLVESGGGLVKPGGSLRLSCAASGFTFSSNAMHWVRQAPGKG |
| | Chain | LEWVGRIKSKTDGGTTDYAAPVKGRFTISRDDSKNTLYLQMNSL |
| | | KTEDTAVYYCARDHYYYPFAYWGQGTLVTVSSASTKGPSVFPLA |
| | | PSSKSTSGGTAALGCLVKDYFPEPVTVSWNSGALTSGVHTFPAV |
| | | LQSSGLYSLSSVVTVPSSSLGTQTYICNVNHKPSNTKVDKRVEP |
| | | KSC |
| SEQ ID NO: 234 | DNA | CAAGTGCAGCTGGTGGAATCTGGCGGCGGACTGGTCAAGCCTGG |
| | Heavy | CGGTAGCCTGAGACTGTCTTGCGCCGCCTCCGGCTTCACCTTCT |
| | Chain | CCTCCAACGCCATGCACTGGGTCCGACAGGCCCCTGGCAAGGGC |
| | | CTGGAGTGGGTCGGACGGATCAAGTCCAAGACCGACGGCGGCAC |
| | | CACCGACTACGCTGCCCCTGTGAAGGGCCGGTTCACCATCTCCC |
| | | GGGACGACTCCAAGAACACCCTGTACCTGCAGATGAACTCCCTG |
| | | AAAACCGAGGACACCGCCGTGTACTACTGCGCCAGGGACCACTA |
| | | CTACTACCCCTTCGCCTACTGGGGCCAGGGCACCCTGGTCACCG |
| | | TGTCCTCTGCTAGCACCAAGGGCCCCTCCGTGTTCCCTCTGGCC |
| | | CCTTCCAGCAAGTCTACCTCTGGCGGCACCGCTGCTCTGGGCTG |
| | | CCTGGTGAAGGACTACTTCCCTGAGCCTGTGACAGTGTCCTGGA |
| | | ACTCTGGCGCCCTGACCTCCGGCGTGCACACCTTCCCTGCCGTG |
| | | CTGCAGTCCTCCGGCCTGTACTCCCTGTCCTCCGTGGTGACAGT |
| | | GCCTTCCTCCAGCCTGGGCACCCAGACCTATATCTGCAACGTGA |
| | | ACCACAAGCCTTCCAACACCAAGGTGGACAAGCGGGTGGAGCCT |
| | | AAGTCATGC |
| SEQ ID NO: 235 | LCDR1 | RASQSIRYNLA |
| (Combined) | | |
| SEQ ID NO: 236 | LCDR2 | AASSLQS |
| (Combined) | | |
| SEQ ID NO: 237 | LCDR3 | HQYIAKPIT |
| (Combined) | | |
| SEQ ID NO: 238 | LCDR1 | RASQSIRYNLA |
| (Kabat) | | |
| SEQ ID NO: 239 | LCDR2 | AASSLQS |
| (Kabat) | | |
| SEQ ID NO: 240 | LCDR3 | HQYIAKPIT |
| (Kabat) | | |
| SEQ ID NO: 241 | LCDR1 | SQSIRYN |
| (Chothia) | | |
| SEQ ID NO: 242 | LCDR2 | AAS |
| (Chothia) | | |
| SEQ ID NO: 243 | LCDR3 | YIAKPI |
| (Chothia) | | |
| SEQ ID NO: 244 | LCDR1 | QSIRYN |
| (IMGT) | | |
| SEQ ID NO: 245 | LCDR2 | AAS |
| (IMGT) | | |
| SEQ ID NO: 246 | LCDR3 | HQYIAKPIT |
| (IMGT) | | |
| SEQ ID NO: 247 | VL | DIQMTQSPSSLSASVGDRVTITCRASQSIRYNLAWYQQKPGKAP |
| | | KLLIYAASSLQSGVPSRFSGSGSGTDFTLTISSLQPEDFATYYC |
| | | HQYIAKPITFGQGTKVEIK |
| SEQ ID NO: 248 | DNA VL | GACATCCAGATGACCCAGAGCCCCTCCAGCCTGTCCGCCTCCGT |
| | | GGGCGACAGAGTGACCATCACCTGTCGGGCCTCCCAGTCCATCC |
| | | GGTACAACCTGGCCTGGTATCAGCAGAAGCCCGGCAAGGCCCCT |
| | | AAGCTGCTGATCTACGCCGCCAGCTCCCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | CACCAGTATATCGCCAAGCCCATCACCTTCGGCCAGGGCACCAA |
| | | AGTGGAAATCAAG |
| SEQ ID NO: 249 | Light | DIQMTQSPSSLSASVGDRVTITCRASQSIRYNLAWYQQKPGKAP |
| | Chain | KLLIYAASSLQSGVPSRFSGSGSGTDFTLTISSLQPEDFATYYC |
| | | HQYIAKPITFGQGTKVEIKRTVAAPSVFIFPPSDEQLKSGTASV |
| | | VCLLNNFYPREAKVQWKVDNALQSGNSQESVTEQDSKDSTYSLS |
| | | STLTLSKADYEKHKVYACEVTHQGLSSPVTKSFNRGEC |
| SEQ ID NO: 250 | DNA | GACATCCAGATGACCCAGAGCCCCTCCAGCCTGTCCGCCTCCGT |
| | Light | GGGCGACAGAGTGACCATCACCTGTCGGGCCTCCCAGTCCATCC |
| | Chain | GGTACAACCTGGCCTGGTATCAGCAGAAGCCCGGCAAGGCCCCT |
| | | AAGCTGCTGATCTACGCCGCCAGCTCCCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | CACCAGTATATCGCCAAGCCCATCACCTTCGGCCAGGGCACCAA |
| | | AGTGGAAATCAAGCGTACGGTGGCCGCTCCCAGCGTGTTCATCT |
| | | TCCCCCCCAGCGACGAGCAGCTGAAGAGCGGCACCGCCAGCGTG |
| | | GTGTGCCTGCTGAACAACTTCTACCCCCGGGAGGCCAAGGTGCA |
| | | GTGGAAGGTGGACAACGCCCTGCAGAGCGGCAACAGCCAGGAGA |
| | | GCGTCACCGAGCAGGACAGCAAGGACTCCACCTACAGCCTGAGC |
| | | AGCACCCTGACCCTGAGCAAGGCCGACTACGAGAAGCATAAGGT |
| | | GTACGCCTGCGAGGTGACCCACCAGGGCCTGTCCAGCCCCGTGA |
| | | CCAAGAGCTTCAACAGGGGCGAGTGC |
| IDT8 | | |
| SEQ ID NO: 251 | HCDR1 | GFTFSSNAMH |
| (Combined) | | |
| SEQ ID NO: 252 | HCDR2 | RIKSKTSGGTTDYAAPVKG |
| (Combined) | | |
| SEQ ID NO: 253 | HCDR3 | DHYYYPFAY |
| (Combined) | | |
| SEQ ID NO: 254 | HCDR1 | SNAMH |
| (Kabat) | | |
| SEQ ID NO: 255 | HCDR2 | RIKSKTSGGTTDYAAPVKG |
| (Kabat) | | |
| SEQ ID NO: 256 | HCDR3 | DHYYYPFAY |
| (Kabat) | | |
| SEQ ID NO: 257 | HCDR1 | GFTFSSN |
| (Chothia) | | |
| SEQ ID NO: 258 | HCDR2 | KSKTSGGT |
| (Chothia) | | |
| SEQ ID NO: 259 | HCDR3 | DHYYYPFAY |
| (Chothia) | | |
| SEQ ID NO: 260 | HCDR1 | GFTFSSNA |
| (IMGT) | | |
| SEQ ID NO: 261 | HCDR2 | IKSKTSGGTT |
| (IMGT) | | |
| SEQ ID NO: 262 | HCDR3 | ARDHYYYPFAY |
| (IMGT) | | |
| SEQ ID NO: 263 | VH | QVQLVESGGGLVKPGGSLRLSCAASGFTFSSNAMHWVRQAPGKG |
| | | LEWVGRIKSKTSGGTTDYAAPVKGRFTISRDDSKNTLYLQMNSL |
| | | KTEDTAVYYCARDHYYYPFAYWGQGTLVTVSS |
| SEQ ID NO: 264 | DNA VH | CAAGTGCAGCTGGTGGAATCTGGCGGCGGACTGGTCAAGCCTGG |
| | | CGGTAGCCTGAGACTGTCTTGCGCCGCCTCCGGCTTCACCTTCT |
| | | CCTCCAACGCCATGCACTGGGTCCGACAGGCCCCTGGCAAGGGC |
| | | CTGGAGTGGGTCGGACGGATCAAGTCCAAGACCTCCGGCGGCAC |
| | | CACCGACTACGCTGCCCCTGTGAAGGGCCGGTTCACCATCTCCC |
| | | GGGACGACTCCAAGAACACCCTGTACCTGCAGATGAACTCCCTG |
| | | AAAACCGAGGACACCGCCGTGTACTACTGCGCCAGGGACCACTA |
| | | CTACTACCCCTTCGCCTACTGGGGCCAGGGCACCCTGGTCACCG |
| | | TGTCCTCT |
| SEQ ID NO: 265 | Heavy | QVQLVESGGGLVKPGGSLRLSCAASGFTFSSNAMHWVRQAPGKG |
| | Chain | LEWVGRIKSKTSGGTTDYAAPVKGRFTISRDDSKNTLYLQMNSL |
| | | KTEDTAVYYCARDHYYYPFAYWGQGTLVTVSSASTKGPSVFPLA |
| | | PSSKSTSGGTAALGCLVKDYFPEPVTVSWNSGALTSGVHTFPAV |
| | | LQSSGLYSLSSVVTVPSSSLGTQTYICNVNHKPSNTKVDKRVEP |
| | | KSC |
| SEQ ID NO: 266 | DNA | CAAGTGCAGCTGGTGGAATCTGGCGGCGGACTGGTCAAGCCTGG |
| | Heavy | CGGTAGCCTGAGACTGTCTTGCGCCGCCTCCGGCTTCACCTTCT |
| | Chain | CCTCCAACGCCATGCACTGGGTCCGACAGGCCCCTGGCAAGGGC |
| | | CTGGAGTGGGTCGGACGGATCAAGTCCAAGACCTCCGGCGGCAC |
| | | CACCGACTACGCTGCCCCTGTGAAGGGCCGGTTCACCATCTCCC |
| | | GGGACGACTCCAAGAACACCCTGTACCTGCAGATGAACTCCCTG |
| | | AAAACCGAGGACACCGCCGTGTACTACTGCGCCAGGGACCACTA |
| | | CTACTACCCCTTCGCCTACTGGGGCCAGGGCACCCTGGTCACCG |
| | | TGTCCTCTGCTAGCACCAAGGGCCCCTCCGTGTTCCCTCTGGCC |
| | | CCTTCCAGCAAGTCTACCTCTGGCGGCACCGCTGCTCTGGGCTG |
| | | CCTGGTGAAGGACTACTTCCCTGAGCCTGTGACAGTGTCCTGGA |
| | | ACTCTGGCGCCCTGACCTCCGGCGTGCACACCTTCCCTGCCGTG |
| | | CTGCAGTCCTCCGGCCTGTACTCCCTGTCCTCCGTGGTGACAGT |
| | | GCCTTCCTCCAGCCTGGGCACCCAGACCTATATCTGCAACGTGA |
| | | ACCACAAGCCTTCCAACACCAAGGTGGACAAGCGGGTGGAGCCT |
| | | AAGTCATGC |
| SEQ ID NO: 267 | LCDR1 | RASQSIRYNLA |
| (Combined) | | |
| SEQ ID NO: 268 | LCDR2 | AASSLQS |
| (Combined) | | |
| SEQ ID NO: 269 | LCDR3 | HQYIAKPIT |
| (Combined) | | |
| SEQ ID NO: 270 | LCDR1 | RASQSIRYNLA |
| (Kabat) | | |
| SEQ ID NO: 271 | LCDR2 | AASSLQS |
| (Kabat) | | |
| SEQ ID NO: 272 | LCDR3 | HQYIAKPIT |
| (Kabat) | | |
| SEQ ID NO: 273 | LCDR1 | SQSIRYN |
| (Chothia) | | |
| SEQ ID NO: 274 | LCDR2 | AAS |
| (Chothia) | | |
| SEQ ID NO: 275 | LCDR3 | YIAKPI |
| (Chothia) | | |
| SEQ ID NO: 276 | LCDR1 | QSIRYN |
| (IMGT) | | |
| SEQ ID NO: 277 | LCDR2 | AAS |
| (IMGT) | | |
| SEQ ID NO: 278 | LCDR3 | HQYIAKPIT |
| (IMGT) | | |
| SEQ ID NO: 279 | VL | DIQMTQSPSSLSASVGDRVTITCRASQSIRYNLAWYQQKPGKAP |
| | | KLLIYAASSLQSGVPSRFSGSGSGTDFTLTISSLQPEDFATYYC |
| | | HQYIAKPITFGQGTKVEIK |
| SEQ ID NO: 280 | DNA VL | GACATCCAGATGACCCAGAGCCCCTCCAGCCTGTCCGCCTCCGT |
| | | GGGCGACAGAGTGACCATCACCTGTCGGGCCTCCCAGTCCATCC |
| | | GGTACAACCTGGCCTGGTATCAGCAGAAGCCCGGCAAGGCCCCT |
| | | AAGCTGCTGATCTACGCCGCCAGCTCCCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | CACCAGTATATCGCCAAGCCCATCACCTTCGGCCAGGGCACCAA |
| | | AGTGGAAATCAAG |
| SEQ ID NO: 281 | Light | DIQMTQSPSSLSASVGDRVTITCRASQSIRYNLAWYQQKPGKAP |
| | Chain | KLLIYAASSLQSGVPSRFSGSGSGTDFTLTISSLQPEDFATYYC |
| | | HQYIAKPITFGQGTKVEIKRTVAAPSVFIFPPSDEQLKSGTASV |
| | | VCLLNNFYPREAKVQWKVDNALQSGNSQESVTEQDSKDSTYSLS |
| | | STLTLSKADYEKHKVYACEVTHQGLSSPVTKSFNRGEC |
| SEQ ID NO: 282 | DNA | GACATCCAGATGACCCAGAGCCCCTCCAGCCTGTCCGCCTCCGT |
| | Light | GGGCGACAGAGTGACCATCACCTGTCGGGCCTCCCAGTCCATCC |
| | Chain | GGTACAACCTGGCCTGGTATCAGCAGAAGCCCGGCAAGGCCCCT |
| | | AAGCTGCTGATCTACGCCGCCAGCTCCCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | CACCAGTATATCGCCAAGCCCATCACCTTCGGCCAGGGCACCAA |
| | | AGTGGAAATCAAGCGTACGGTGGCCGCTCCCAGCGTGTTCATCT |
| | | TCCCCCCCAGCGACGAGCAGCTGAAGAGCGGCACCGCCAGCGTG |
| | | GTGTGCCTGCTGAACAACTTCTACCCCCGGGAGGCCAAGGTGCA |
| | | GTGGAAGGTGGACAACGCCCTGCAGAGCGGCAACAGCCAGGAGA |
| | | GCGTCACCGAGCAGGACAGCAAGGACTCCACCTACAGCCTGAGC |
| | | AGCACCCTGACCCTGAGCAAGGCCGACTACGAGAAGCATAAGGT |
| | | GTACGCCTGCGAGGTGACCCACCAGGGCCTGTCCAGCCCCGTGA |
| | | CCAAGAGCTTCAACAGGGGCGAGTGC |
| IDT9 | | |
| SEQ ID NO: 283 | HCDR1 | GYTFTNYYVH |
| (Combined) | | |
| SEQ ID NO: 284 | HCDR2 | WINPYNGNTNYAQKFQG |
| (Combined) | | |
| SEQ ID NO: 285 | HCDR3 | GASSIRMSYYLDV |
| (Combined) | | |
| SEQ ID NO: 286 | HCDR1 | NYYVH |
| (Kabat) | | |
| SEQ ID NO: 287 | HCDR2 | WINPYNGNTNYAQKFQG |
| (Kabat) | | |
| SEQ ID NO: 288 | HCDR3 | GASSIRMSYYLDV |
| (Kabat) | | |
| SEQ ID NO: 289 | HCDR1 | GYTFTNY |
| (Chothia) | | |
| SEQ ID NO: 290 | HCDR2 | NPYNGN |
| (Chothia) | | |
| SEQ ID NO: 291 | HCDR3 | GASSIRMSYYLDV |
| (Chothia) | | |
| SEQ ID NO: 292 | HCDR1 | GYTFTNYY |
| (IMGT) | | |
| SEQ ID NO: 293 | HCDR2 | INPYNGNT |
| (IMGT) | | |
| SEQ ID NO: 294 | HCDR3 | ARGASSIRMSYYLDV |
| (IMGT) | | |
| SEQ ID NO: 295 | VH | QVQLVQSGAEVKKPGASVKVSCKASGYTFTNYYVHWVRQAPGQG |
| | | LEWMGWINPYNGNTNYAQKFQGRVTMTRDTSISTAYMELSRLRS |
| | | EDTAVYYCARGASSIRMSYYLDVWGQGTLVTVSS |
| SEQ ID NO: 296 | DNA VH | CAAGTGCAGCTGGTGCAGTCTGGCGCTGAAGTGAAGAAACCTGG |
| | | CGCCTCCGTGAAAGTGTCCTGCAAGGCCTCCGGCTACACCTTCA |
| | | CCAACTACTACGTGCACTGGGTCCGACAGGCCCCAGGCCAGGGC |
| | | CTGGAGTGGATGGGCTGGATCAACCCCTACAACGGCAACACCAA |
| | | CTACGCCCAGAAATTCCAGGGCAGAGTGACCATGACCCGGGACA |
| | | CCTCCATCTCCACCGCCTACATGGAACTGTCCCGGCTGCGGAGC |
| | | GAGGACACCGCCGTGTACTACTGTGCCAGAGGCGCCTCCTCCAT |
| | | CCGGATGTCCTACTACCTGGACGTGTGGGGCCAGGGCACCCTGG |
| | | TCACCGTGTCCTCT |
| SEQ ID NO: 297 | Heavy | QVQLVQSGAEVKKPGASVKVSCKASGYTFTNYYVHWVRQAPGQG |
| | Chain | LEWMGWINPYNGNTNYAQKFQGRVTMTRDTSISTAYMELSRLRS |
| | | EDTAVYYCARGASSIRMSYYLDVWGQGTLVTVSSASTKGPSVFP |
| | | LAPSSKSTSGGTAALGCLVKDYFPEPVTVSWNSGALTSGVHTFP |
| | | AVLQSSGLYSLSSVVTVPSSSLGTQTYICNVNHKPSNTKVDKRV |
| | | EPKSC |
| SEQ ID NO: 298 | DNA | CAAGTGCAGCTGGTGCAGTCTGGCGCTGAAGTGAAGAAACCTGG |
| | Heavy | CGCCTCCGTGAAAGTGTCCTGCAAGGCCTCCGGCTACACCTTCA |
| | Chain | CCAACTACTACGTGCACTGGGTCCGACAGGCCCCAGGCCAGGGC |
| | | CTGGAGTGGATGGGCTGGATCAACCCCTACAACGGCAACACCAA |
| | | CTACGCCCAGAAATTCCAGGGCAGAGTGACCATGACCCGGGACA |
| | | CCTCCATCTCCACCGCCTACATGGAACTGTCCCGGCTGCGGAGC |
| | | GAGGACACCGCCGTGTACTACTGTGCCAGAGGCGCCTCCTCCAT |
| | | CCGGATGTCCTACTACCTGGACGTGTGGGGCCAGGGCACCCTGG |
| | | TCACCGTGTCCTCTGCTAGCACCAAGGGCCCCTCCGTGTTCCCT |
| | | CTGGCCCCTTCCAGCAAGTCTACCTCTGGCGGCACCGCTGCTCT |
| | | GGGCTGCCTGGTGAAGGACTACTTCCCTGAGCCTGTGACAGTGT |
| | | CCTGGAACTCTGGCGCCCTGACCTCCGGCGTGCACACCTTCCCT |
| | | GCCGTGCTGCAGTCCTCCGGCCTGTACTCCCTGTCCTCCGTGGT |
| | | GACAGTGCCTTCCTCCAGCCTGGGCACCCAGACCTATATCTGCA |
| | | ACGTGAACCACAAGCCTTCCAACACCAAGGTGGACAAGCGGGTG |
| | | GAGCCTAAGTCATGC |
| SEQ ID NO: 299 | LCDR1 | RASQSISNYLN |
| (Combined) | | |
| SEQ ID NO: 300 | LCDR2 | AASNLQS |
| (Combined) | | |
| SEQ ID NO: 301 | LCDR3 | FQYTHSPAT |
| (Combined) | | |
| SEQ ID NO: 302 | LCDR1 | RASQSISNYLN |
| (Kabat) | | |
| SEQ ID NO: 303 | LCDR2 | AASNLQS |
| (Kabat) | | |
| SEQ ID NO: 304 | LCDR3 | FQYTHSPAT |
| (Kabat) | | |
| SEQ ID NO: 305 | LCDR1 | SQSISNY |
| (Chothia) | | |
| SEQ ID NO: 306 | LCDR2 | AAS |
| (Chothia) | | |
| SEQ ID NO: 307 | LCDR3 | YTHSPA |
| (Chothia) | | |
| SEQ ID NO: 308 | LCDR1 | QSISNY |
| (IMGT) | | |
| SEQ ID NO: 309 | LCDR2 | AAS |
| (IMGT) | | |
| SEQ ID NO: 310 | LCDR3 | FQYTHSPAT |
| (IMGT) | | |
| SEQ ID NO: 311 | VL | DIQMTQSPSSLSASVGDRVTITCRASQSISNYLNWYQQKPGKAP |
| | | KLLIYAASNLQSGVPSRFSGSGSGTDFTLTISSLQPEDFATYYC |
| | | FQYTHSPATFGQGTKVEIK |
| SEQ ID NO: 312 | DNA VL | GACATCCAGATGACCCAGAGCCCCTCCAGCCTGTCCGCCTCCGT |
| | | GGGCGACAGAGTGACCATCACCTGTCGGGCCTCCCAGTCCATCT |
| | | CCAACTACCTGAACTGGTATCAGCAGAAGCCCGGCAAGGCCCCT |
| | | AAGCTGCTGATCTACGCCGCCTCCAACCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | TTCCAGTACACCCACAGCCCCGCCACCTTCGGCCAGGGCACCAA |
| | | AGTGGAAATCAAG |
| SEQ ID NO: 313 | Light | DIQMTQSPSSLSASVGDRVTITCRASQSISNYLNWYQQKPGKAP |
| | Chain | KLLIYAASNLQSGVPSRFSGSGSGTDFTLTISSLQPEDFATYYC |
| | | FQYTHSPATFGQGTKVEIKRTVAAPSVFIFPPSDEQLKSGTASV |
| | | VCLLNNFYPREAKVQWKVDNALQSGNSQESVTEQDSKDSTYSLS |
| | | STLTLSKADYEKHKVYACEVTHQGLSSPVTKSFNRGEC |
| SEQ ID NO: 314 | DNA | GACATCCAGATGACCCAGAGCCCCTCCAGCCTGTCCGCCTCCGT |
| | Light | GGGCGACAGAGTGACCATCACCTGTCGGGCCTCCCAGTCCATCT |
| | Chain | CCAACTACCTGAACTGGTATCAGCAGAAGCCCGGCAAGGCCCCT |
| | | AAGCTGCTGATCTACGCCGCCTCCAACCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | TTCCAGTACACCCACAGCCCCGCCACCTTCGGCCAGGGCACCAA |
| | | AGTGGAAATCAAGCGTACGGTGGCCGCTCCCAGCGTGTTCATCT |
| | | TCCCCCCCAGCGACGAGCAGCTGAAGAGCGGCACCGCCAGCGTG |
| | | GTGTGCCTGCTGAACAACTTCTACCCCCGGGAGGCCAAGGTGCA |
| | | GTGGAAGGTGGACAACGCCCTGCAGAGCGGCAACAGCCAGGAGA |
| | | GCGTCACCGAGCAGGACAGCAAGGACTCCACCTACAGCCTGAGC |
| | | AGCACCCTGACCCTGAGCAAGGCCGACTACGAGAAGCATAAGGT |
| | | GTACGCCTGCGAGGTGACCCACCAGGGCCTGTCCAGCCCCGTGA |
| | | CCAAGAGCTTCAACAGGGGCGAGTGC |
| IDT10 | | |
| SEQ ID NO: 315 | HCDR1 | GYTFTNYYVH |
| (Combined) | | |
| SEQ ID NO: 316 | HCDR2 | WINPYSGNTNYAQKFQG |
| (Combined) | | |
| SEQ ID NO: 317 | HCDR3 | GASSIRMSYYLDV |
| (Combined) | | |
| SEQ ID NO: 318 | HCDR1 | NYYVH |
| (Kabat) | | |
| SEQ ID NO: 319 | HCDR2 | WINPYSGNTNYAQKFQG |
| (Kabat) | | |
| SEQ ID NO: 320 | HCDR3 | GASSIRMSYYLDV |
| (Kabat) | | |
| SEQ ID NO: 321 | HCDR1 | GYTFTNY |
| (Chothia) | | |
| SEQ ID NO: 322 | HCDR2 | NPYSGN |
| (Chothia) | | |
| SEQ ID NO: 323 | HCDR3 | GASSIRMSYYLDV |
| (Chothia) | | |
| SEQ ID NO: 324 | HCDR1 | GYTFTNYY |
| (IMGT) | | |
| SEQ ID NO: 325 | HCDR2 | INPYSGNT |
| (IMGT) | | |
| SEQ ID NO: 326 | HCDR3 | ARGASSIRMSYYLDV |
| (IMGT) | | |
| SEQ ID NO: 327 | VH | QVQLVQSGAEVKKPGASVKVSCKASGYTFTNYYVHWVRQAPGQG |
| | | LEWMGWINPYSGNTNYAQKFQGRVTMTRDTSISTAYMELSRLRS |
| | | EDTAVYYCARGASSIRMSYYLDVWGQGTLVTVSS |
| SEQ ID NO: 328 | DNA VH | CAAGTGCAGCTGGTGCAGTCTGGCGCTGAAGTGAAGAAACCTGG |
| | | CGCCTCCGTGAAAGTGTCCTGCAAGGCCTCCGGCTACACCTTCA |
| | | CCAACTACTACGTGCACTGGGTCCGACAGGCCCCAGGCCAGGGC |
| | | CTGGAGTGGATGGGCTGGATCAACCCCTACTCCGGCAACACCAA |
| | | CTACGCCCAGAAATTCCAGGGCAGAGTGACCATGACCCGGGACA |
| | | CCTCCATCTCCACCGCCTACATGGAACTGTCCCGGCTGCGGAGC |
| | | GAGGACACCGCCGTGTACTACTGTGCCAGAGGCGCCTCCTCCAT |
| | | CCGGATGTCCTACTACCTGGACGTGTGGGGCCAGGGCACCCTGG |
| | | TCACCGTGTCCTCT |
| SEQ ID NO: 329 | Heavy | QVQLVQSGAEVKKPGASVKVSCKASGYTFTNYYVHWVRQAPGQG |
| | Chain | LEWMGWINPYSGNTNYAQKFQGRVTMTRDTSISTAYMELSRLRS |
| | | EDTAVYYCARGASSIRMSYYLDVWGQGTLVTVSSASTKGPSVFP |
| | | LAPSSKSTSGGTAALGCLVKDYFPEPVTVSWNSGALTSGVHTFP |
| | | AVLQSSGLYSLSSVVTVPSSSLGTQTYICNVNHKPSNTKVDKRV |
| | | EPKSC |
| SEQ ID NO: 330 | DNA | CAAGTGCAGCTGGTGCAGTCTGGCGCTGAAGTGAAGAAACCTGG |
| | Heavy | CGCCTCCGTGAAAGTGTCCTGCAAGGCCTCCGGCTACACCTTCA |
| | Chain | CCAACTACTACGTGCACTGGGTCCGACAGGCCCCAGGCCAGGGC |
| | | CTGGAGTGGATGGGCTGGATCAACCCCTACTCCGGCAACACCAA |
| | | CTACGCCCAGAAATTCCAGGGCAGAGTGACCATGACCCGGGACA |
| | | CCTCCATCTCCACCGCCTACATGGAACTGTCCCGGCTGCGGAGC |
| | | GAGGACACCGCCGTGTACTACTGTGCCAGAGGCGCCTCCTCCAT |
| | | CCGGATGTCCTACTACCTGGACGTGTGGGGCCAGGGCACCCTGG |
| | | TCACCGTGTCCTCTGCTAGCACCAAGGGCCCCTCCGTGTTCCCT |
| | | CTGGCCCCTTCCAGCAAGTCTACCTCTGGCGGCACCGCTGCTCT |
| | | GGGCTGCCTGGTGAAGGACTACTTCCCTGAGCCTGTGACAGTGT |
| | | CCTGGAACTCTGGCGCCCTGACCTCCGGCGTGCACACCTTCCCT |
| | | GCCGTGCTGCAGTCCTCCGGCCTGTACTCCCTGTCCTCCGTGGT |
| | | GACAGTGCCTTCCTCCAGCCTGGGCACCCAGACCTATATCTGCA |
| | | ACGTGAACCACAAGCCTTCCAACACCAAGGTGGACAAGCGGGTG |
| | | GAGCCTAAGTCATGC |
| SEQ ID NO: 331 | LCDR1 | RASQSISNYLN |
| (Combined) | | |
| SEQ ID NO: 332 | LCDR2 | AASNLQS |
| (Combined) | | |
| SEQ ID NO: 333 | LCDR3 | FQYTHSPAT |
| (Combined) | | |
| SEQ ID NO: 334 | LCDR1 | RASQSISNYLN |
| (Kabat) | | |
| SEQ ID NO: 335 | LCDR2 | AASNLQS |
| (Kabat) | | |
| SEQ ID NO: 336 | LCDR3 | FQYTHSPAT |
| (Kabat) | | |
| SEQ ID NO: 337 | LCDR1 | SQSISNY |
| (Chothia) | | |
| SEQ ID NO: 338 | LCDR2 | AAS |
| (Chothia) | | |
| SEQ ID NO: 339 | LCDR3 | YTHSPA |
| (Chothia) | | |
| SEQ ID NO: 340 | LCDR1 | QSISNY |
| (IMGT) | | |
| SEQ ID NO: 341 | LCDR2 | AAS |
| (IMGT) | | |
| SEQ ID NO: 342 | LCDR3 | FQYTHSPAT |
| (IMGT) | | |
| SEQ ID NO: 343 | VL | DIQMTQSPSSLSASVGDRVTITCRASQSISNYLNWYQQKPGKAP |
| | | KLLIYAASNLQSGVPSRFSGSGSGTDFTLTISSLQPEDFATYYC |
| | | FQYTHSPATFGQGTKVEIK |
| SEQ ID NO: 344 | DNA VL | GACATCCAGATGACCCAGAGCCCCTCCAGCCTGTCCGCCTCCGT |
| | | GGGCGACAGAGTGACCATCACCTGTCGGGCCTCCCAGTCCATCT |
| | | CCAACTACCTGAACTGGTATCAGCAGAAGCCCGGCAAGGCCCCT |
| | | AAGCTGCTGATCTACGCCGCCTCCAACCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | TTCCAGTACACCCACAGCCCCGCCACCTTCGGCCAGGGCACCAA |
| | | AGTGGAAATCAAG |
| SEQ ID NO: 345 | Light | DIQMTQSPSSLSASVGDRVTITCRASQSISNYLNWYQQKPGKAP |
| | Chain | KLLIYAASNLQSGVPSRFSGSGSGTDFTLTISSLQPEDFATYYC |
| | | FQYTHSPATFGQGTKVEIKRTVAAPSVFIFPPSDEQLKSGTASV |
| | | VCLLNNFYPREAKVQWKVDNALQSGNSQESVTEQDSKDSTYSLS |
| | | STLTLSKADYEKHKVYACEVTHQGLSSPVTKSFNRGEC |
| SEQ ID NO: 346 | DNA | GACATCCAGATGACCCAGAGCCCCTCCAGCCTGTCCGCCTCCGT |
| | Light | GGGCGACAGAGTGACCATCACCTGTCGGGCCTCCCAGTCCATCT |
| | Chain | CCAACTACCTGAACTGGTATCAGCAGAAGCCCGGCAAGGCCCCT |
| | | AAGCTGCTGATCTACGCCGCCTCCAACCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | TTCCAGTACACCCACAGCCCCGCCACCTTCGGCCAGGGCACCAA |
| | | AGTGGAAATCAAGCGTACGGTGGCCGCTCCCAGCGTGTTCATCT |
| | | TCCCCCCCAGCGACGAGCAGCTGAAGAGCGGCACCGCCAGCGTG |
| | | GTGTGCCTGCTGAACAACTTCTACCCCCGGGAGGCCAAGGTGCA |
| | | GTGGAAGGTGGACAACGCCCTGCAGAGCGGCAACAGCCAGGAGA |
| | | GCGTCACCGAGCAGGACAGCAAGGACTCCACCTACAGCCTGAGC |
| | | AGCACCCTGACCCTGAGCAAGGCCGACTACGAGAAGCATAAGGT |
| | | GTACGCCTGCGAGGTGACCCACCAGGGCCTGTCCAGCCCCGTGA |
| | | CCAAGAGCTTCAACAGGGGCGAGTGC |
| IDT11 | | |
| SEQ ID NO: 27 | HCDR1 | GFTFSDYAMS |
| (Combined) | | |
| SEQ ID NO: 28 | HCDR2 | VIDYSSSNTYYADSVKG |
| (Combined) | | |
| SEQ ID NO: 29 | HCDR3 | EGYSYRSIRFDY |
| (Combined) | | |
| SEQ ID NO: 30 | HCDR1 | DYAMS |
| (Kabat) | | |
| SEQ ID NO: 31 | HCDR2 | VIDYSSSNTYYADSVKG |
| (Kabat) | | |
| SEQ ID NO: 32 | HCDR3 | EGYSYRSIRFDY |
| (Kabat) | | |
| SEQ ID NO: 33 | HCDR1 | GFTFSDY |
| (Chothia) | | |
| SEQ ID NO: 34 | HCDR2 | DYSSSN |
| (Chothia) | | |
| SEQ ID NO: 35 | HCDR3 | EGYSYRSIRFDY |
| (Chothia) | | |
| SEQ ID NO: 36 | HCDR1 | GFTFSDYA |
| (IMGT) | | |
| SEQ ID NO: 37 | HCDR2 | IDYSSSNT |
| (IMGT) | | |
| SEQ ID NO: 38 | HCDR3 | AREGYSYRSIRFDY |
| (IMGT) | | |
| SEQ ID NO: 39 | VH | QVQLLESGGGLVQPGGSLRLSCAASGFTFSDYAMSWVRQAPGKG |
| | | LEWVSVIDYSSSNTYYADSVKGRFTISRDNSKNTLYLQMNSLRA |
| | | EDTAVYYCAREGYSYRSIRFDYWGQGTLVTVSS |
| SEQ ID NO: 40 | DNA VH | CAAGTGCAGCTGCTGGAATCTGGCGGCGGACTGGTGCAGCCTGG |
| | | CGGTAGTCTGAGACTGTCTTGCGCCGCCTCCGGCTTCACCTTCT |
| | | CCGACTACGCCATGTCCTGGGTCCGACAGGCCCCTGGCAAGGGC |
| | | CTGGAGTGGGTGTCCGTGATCGACTACTCCTCCTCCAACACCTA |
| | | CTACGCCGACTCCGTGAAGGGCCGGTTCACCATCTCCCGGGACA |
| | | ACTCCAAGAACACCCTGTACCTGCAGATGAACTCCCTGCGGGCC |
| | | GAGGACACCGCCGTGTACTACTGCGCCAGAGAGGGCTACTCCTA |
| | | CCGGTCCATCAGATTCGACTACTGGGGCCAGGGCACCCTGGTCA |
| | | CCGTGTCCTCT |
| SEQ ID NO: 347 | Heavy | QVQLLESGGGLVQPGGSLRLSCAASGFTFSDYAMSWVRQAPGKG |
| | Chain | LEWVSVIDYSSSNTYYADSVKGRFTISRDNSKNTLYLQMNSLRA |
| | | EDTAVYYCAREGYSYRSIRFDYWGQGTLVTVSSASTKGPSVFPL |
| | | APSSKSTSGGTAALGCLVKDYFPEPVTVSWNSGALTSGVHTFPA |
| | | VLQSSGLYSLSSVVTVPSSSLGTQTYICNVNHKPSNTKVDKRVE |
| | | PKSCDKTHTCPPCPAPELLGGPSVFLFPPKPKDTLMISRTPEVT |
| | | CVVVAVSHEDPEVKFNWYVDGVEVHNAKTKPREEQYNSTYRVVS |
| | | VLTVLHQDWLNGKEYKCKVSNKALAAPIEKTISKAKGQPREPQV |
| | | YTLPPSREEMTKNQVSLTCLVKGFYPSDIAVEWESNGQPENNYK |
| | | TTPPVLDSDGSFFLYSKLTVDKSRWQQGNVFSCSVMHEALHNHY |
| | | TQKSLSLSPGK |
| SEQ ID NO: 348 | DNA | CAAGTGCAGCTGCTGGAATCTGGCGGCGGACTGGTGCAGCCTGG |
| | Heavy | CGGTAGTCTGAGACTGTCTTGCGCCGCCTCCGGCTTCACCTTCT |
| | Chain | CCGACTACGCCATGTCCTGGGTCCGACAGGCCCCTGGCAAGGGC |
| | | CTGGAGTGGGTGTCCGTGATCGACTACTCCTCCTCCAACACCTA |
| | | CTACGCCGACTCCGTGAAGGGCCGGTTCACCATCTCCCGGGACA |
| | | ACTCCAAGAACACCCTGTACCTGCAGATGAACTCCCTGCGGGCC |
| | | GAGGACACCGCCGTGTACTACTGCGCCAGAGAGGGCTACTCCTA |
| | | CCGGTCCATCAGATTCGACTACTGGGGCCAGGGCACCCTGGTCA |
| | | CCGTGTCCTCTGCTAGCACCAAGGGCCCCTCCGTGTTCCCTCTG |
| | | GCCCCTTCCAGCAAGTCTACCTCCGGCGGCACAGCTGCTCTGGG |
| | | CTGCCTGGTCAAGGACTACTTCCCTGAGCCTGTGACAGTGTCCT |
| | | GGAACTCTGGCGCCCTGACCTCTGGCGTGCACACCTTCCCTGCC |
| | | GTGCTGCAGTCCTCCGGCCTGTACTCCCTGTCCTCCGTGGTCAC |
| | | AGTGCCTTCAAGCAGCCTGGGCACCCAGACCTATATCTGCAACG |
| | | TGAACCACAAGCCTTCCAACACCAAGGTGGACAAGCGGGTGGAG |
| | | CCTAAGTCCTGCGACAAGACCCACACCTGTCCTCCCTGCCCTGC |
| | | TCCTGAACTGCTGGGCGGCCCTTCTGTGTTCCTGTTCCCTCCAA |
| | | AGCCCAAGGACACCCTGATGATCTCCCGGACCCCTGAAGTGACC |
| | | TGCGTGGTGGTGGCCGTGTCCCACGAGGATCCTGAAGTGAAGTT |
| | | CAATTGGTACGTGGACGGCGTGGAGGTGCACAACGCCAAGACCA |
| | | AGCCTCGGGAGGAACAGTACAACTCCACCTACCGGGTGGTGTCC |
| | | GTGCTGACCGTGCTGCACCAGGACTGGCTGAACGGCAAAGAGTA |
| | | CAAGTGCAAAGTCTCCAACAAGGCCCTGGCCGCCCCTATCGAAA |
| | | AGACAATCTCCAAGGCCAAGGGCCAGCCTAGGGAACCCCAGGTG |
| | | TACACCCTGCCACCCAGCCGGGAGGAAATGACCAAGAACCAGGT |
| | | GTCCCTGACCTGTCTGGTCAAGGGCTTCTACCCTTCCGATATCG |
| | | CCGTGGAGTGGGAGTCTAACGGCCAGCCTGAGAACAACTACAAG |
| | | ACCACCCCTCCTGTGCTGGACTCCGACGGCTCCTTCTTCCTGTA |
| | | CTCCAAACTGACCGTGGACAAGTCCCGGTGGCAGCAGGGCAACG |
| | | TGTTCTCCTGCTCCGTGATGCACGAGGCCCTGCACAACCACTAC |
| | | ACCCAGAAGTCCCTGTCCCTGTCTCCCGGCAAG |
| SEQ ID NO: 43 | LCDR1 | RASQSISSNLN |
| (Combined) | | |
| SEQ ID NO: 44 | LCDR2 | AASNLQS |
| (Combined) | | |
| SEQ ID NO: 45 | LCDR3 | LQFDHTPFT |
| (Combined) | | |
| SEQ ID NO: 46 | LCDR1 | RASQSISSNLN |
| (Kabat) | | |
| SEQ ID NO: 47 | LCDR2 | AASNLQS |
| (Kabat) | | |
| SEQ ID NO: 48 | LCDR3 | LQFDHTPFT |
| (Kabat) | | |
| SEQ ID NO: 49 | LCDR1 | SQSISSN |
| (Chothia) | | |
| SEQ ID NO: 50 | LCDR2 | AAS |
| (Chothia) | | |
| SEQ ID NO: 51 | LCDR3 | FDHTPF |
| (Chothia) | | |
| SEQ ID NO: 52 | LCDR1 | QSISSN |
| (IMGT) | | |
| SEQ ID NO: 53 | LCDR2 | AAS |
| (IMGT) | | |
| SEQ ID NO: 54 | LCDR3 | LQFDHTPFT |
| (IMGT) | | |
| SEQ ID NO: 55 | VL | DIQMTQSPSSLSASVGDRVTITCRASQSISSNLNWYQQKPGKAP |
| | | KLLIYAASNLQSGVPSRFSGSGSGTDFTLTISSLQPEDFATYYC |
| | | LQFDHTPFTFGQGTKVEIK |
| SEQ ID NO: 56 | DNA VL | GACATCCAGATGACCCAGAGCCCCTCCAGCCTGTCCGCCTCCGT |
| | | GGGCGACAGAGTGACCATCACCTGTCGGGCCTCCCAGTCCATCT |
| | | CCTCCAACCTGAACTGGTATCAGCAGAAGCCCGGCAAGGCCCCT |
| | | AAGCTGCTGATCTACGCCGCCAGCAACCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | CTGCAGTTCGACCACACCCCTTTCACCTTCGGCCAGGGCACCAA |
| | | AGTGGAAATCAAG |
| SEQ ID NO: 57 | Light | DIQMTQSPSSLSASVGDRVTITCRASQSISSNLNWYQQKPGKAP |
| | Chain | KLLIYAASNLQSGVPSRFSGSGSGTDFTLTISSLQPEDFATYYC |
| | | LQFDHTPFTFGQGTKVEIKRTVAAPSVFIFPPSDEQLKSGTASV |
| | | VCLLNNFYPREAKVQWKVDNALQSGNSQESVTEQDSKDSTYSLS |
| | | STLTLSKADYEKHKVYACEVTHQGLSSPVTKSFNRGEC |
| SEQ ID NO: 58 | DNA | GACATCCAGATGACCCAGAGCCCCTCCAGCCTGTCCGCCTCCGT |
| | Light | GGGCGACAGAGTGACCATCACCTGTCGGGCCTCCCAGTCCATCT |
| | Chain | CCTCCAACCTGAACTGGTATCAGCAGAAGCCCGGCAAGGCCCCT |
| | | AAGCTGCTGATCTACGCCGCCAGCAACCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | CTGCAGTTCGACCACACCCCTTTCACCTTCGGCCAGGGCACCAA |
| | | AGTGGAAATCAAGCGTACGGTGGCCGCTCCCAGCGTGTTCATCT |
| | | TCCCCCCCAGCGACGAGCAGCTGAAGAGCGGCACCGCCAGCGTG |
| | | GTGTGCCTGCTGAACAACTTCTACCCCCGGGAGGCCAAGGTGCA |
| | | GTGGAAGGTGGACAACGCCCTGCAGAGCGGCAACAGCCAGGAGA |
| | | GCGTCACCGAGCAGGACAGCAAGGACTCCACCTACAGCCTGAGC |
| | | AGCACCCTGACCCTGAGCAAGGCCGACTACGAGAAGCATAAGGT |
| | | GTACGCCTGCGAGGTGACCCACCAGGGCCTGTCCAGCCCCGTGA |
| | | CCAAGAGCTTCAACAGGGGCGAGTGC |
| IDT12 | | |
| SEQ ID NO: 59 | HCDR1 | GFTFSSAAVH |
| (Combined) | | |
| SEQ ID NO: 60 | HCDR2 | RIKSKADGGTTDYAAPVKG |
| (Combined) | | |
| SEQ ID NO: 61 | HCDR3 | DSPSISSYSIPYFSGMDV |
| (Combined) | | |
| SEQ ID NO: 62 | HCDR1 | SAAVH |
| (Kabat) | | |
| SEQ ID NO: 63 | HCDR2 | RIKSKADGGTTDYAAPVKG |
| (Kabat) | | |
| SEQ ID NO: 64 | HCDR3 | DSPSISSYSIPYFSGMDV |
| (Kabat) | | |
| SEQ ID NO: 65 | HCDR1 | GFTFSSA |
| (Chothia) | | |
| SEQ ID NO: 66 | HCDR2 | KSKADGGT |
| (Chothia) | | |
| SEQ ID NO: 67 | HCDR3 | DSPSISSYSIPYFSGMDV |
| (Chothia) | | |
| SEQ ID NO: 68 | HCDR1 | GFTFSSAA |
| (IMGT) | | |
| SEQ ID NO: 69 | HCDR2 | IKSKADGGTT |
| (IMGT) | | |
| SEQ ID NO: 70 | HCDR3 | ARDSPSISSYSIPYFSGMDV |
| (IMGT) | | |
| SEQ ID NO: 71 | VH | QVQLVESGGGLVKPGGSLRLSCAASGFTFSSAAVHWVRQAPGKG |
| | | LEWVGRIKSKADGGTTDYAAPVKGRFTISRDDSKNTLYLQMNSL |
| | | KTEDTAVYYCARDSPSISSYSIPYFSGMDVWGQGTLVTVSS |
| SEQ ID NO: 72 | DNA VH | CAAGTGCAGCTGGTGGAATCTGGCGGCGGACTGGTCAAGCCTGG |
| | | CGGTAGCCTGAGACTGTCTTGCGCCGCCTCCGGCTTCACCTTCT |
| | | CCTCTGCCGCTGTGCACTGGGTCCGACAGGCCCCTGGCAAGGGC |
| | | CTGGAGTGGGTCGGACGGATCAAGTCCAAGGCCGACGGCGGCAC |
| | | CACCGACTACGCTGCCCCTGTGAAGGGCCGGTTCACCATCTCCC |
| | | GGGACGACTCCAAGAACACCCTGTACCTGCAGATGAACTCCCTG |
| | | AAAACCGAGGACACCGCCGTGTACTACTGCGCCAGAGACTCCCC |
| | | ATCTATCTCCAGCTACTCCATCCCCTACTTCTCCGGCATGGACG |
| | | TGTGGGGCCAGGGCACCCTGGTCACCGTGTCCTCT |
| SEQ ID NO: 349 | Heavy | QVQLVESGGGLVKPGGSLRLSCAASGFTFSSAAVHWVRQAPGKG |
| | Chain | LEWVGRIKSKADGGTTDYAAPVKGRFTISRDDSKNTLYLQMNSL |
| | | KTEDTAVYYCARDSPSISSYSIPYFSGMDVWGQGTLVTVSSAST |
| | | KGPSVFPLAPSSKSTSGGTAALGCLVKDYFPEPVTVSWNSGALT |
| | | SGVHTFPAVLQSSGLYSLSSVVTVPSSSLGTQTYICNVNHKPSN |
| | | TKVDKRVEPKSCDKTHTCPPCPAPELLGGPSVFLFPPKPKDTLM |
| | | ISRTPEVTCVVVAVSHEDPEVKFNWYVDGVEVHNAKTKPREEQY |
| | | NSTYRVVSVLTVLHQDWLNGKEYKCKVSNKALAAPIEKTISKAK |
| | | GQPREPQVYTLPPSREEMTKNQVSLTCLVKGFYPSDIAVEWESN |
| | | GQPENNYKTTPPVLDSDGSFFLYSKLTVDKSRWQQGNVFSCSVM |
| | | HEALHNHYTQKSLSLSPGK |
| SEQ ID NO: 350 | DNA | CAAGTGCAGCTGGTGGAATCTGGCGGCGGACTGGTCAAGCCTGG |
| | Heavy | CGGTAGCCTGAGACTGTCTTGCGCCGCCTCCGGCTTCACCTTCT |
| | Chain | CCTCTGCCGCTGTGCACTGGGTCCGACAGGCCCCTGGCAAGGGC |
| | | CTGGAGTGGGTCGGACGGATCAAGTCCAAGGCCGACGGCGGCAC |
| | | CACCGACTACGCTGCCCCTGTGAAGGGCCGGTTCACCATCTCCC |
| | | GGGACGACTCCAAGAACACCCTGTACCTGCAGATGAACTCCCTG |
| | | AAAACCGAGGACACCGCCGTGTACTACTGCGCCAGAGACTCCCC |
| | | ATCTATCTCCAGCTACTCCATCCCCTACTTCTCCGGCATGGACG |
| | | TGTGGGGCCAGGGCACCCTGGTCACCGTGTCCTCTGCTAGCACC |
| | | AAGGGCCCCTCCGTGTTCCCTCTGGCCCCTTCCAGCAAGTCTAC |
| | | CTCCGGCGGCACAGCTGCTCTGGGCTGCCTGGTCAAGGACTACT |
| | | TCCCTGAGCCTGTGACAGTGTCCTGGAACTCTGGCGCCCTGACC |
| | | TCTGGCGTGCACACCTTCCCTGCCGTGCTGCAGTCCTCCGGCCT |
| | | GTACTCCCTGTCCTCCGTGGTCACAGTGCCTTCAAGCAGCCTGG |
| | | GCACCCAGACCTATATCTGCAACGTGAACCACAAGCCTTCCAAC |
| | | ACCAAGGTGGACAAGCGGGTGGAGCCTAAGTCCTGCGACAAGAC |
| | | CCACACCTGTCCTCCCTGCCCTGCTCCTGAACTGCTGGGCGGCC |
| | | CTTCTGTGTTCCTGTTCCCTCCAAAGCCCAAGGACACCCTGATG |
| | | ATCTCCCGGACCCCTGAAGTGACCTGCGTGGTGGTGGCCGTGTC |
| | | CCACGAGGATCCTGAAGTGAAGTTCAATTGGTACGTGGACGGCG |
| | | TGGAGGTGCACAACGCCAAGACCAAGCCTCGGGAGGAACAGTAC |
| | | AACTCCACCTACCGGGTGGTGTCCGTGCTGACCGTGCTGCACCA |
| | | GGACTGGCTGAACGGCAAAGAGTACAAGTGCAAAGTCTCCAACA |
| | | AGGCCCTGGCCGCCCCTATCGAAAAGACAATCTCCAAGGCCAAG |
| | | GGCCAGCCTAGGGAACCCCAGGTGTACACCCTGCCACCCAGCCG |
| | | GGAGGAAATGACCAAGAACCAGGTGTCCCTGACCTGTCTGGTCA |
| | | AGGGCTTCTACCCTTCCGATATCGCCGTGGAGTGGGAGTCTAAC |
| | | GGCCAGCCTGAGAACAACTACAAGACCACCCCTCCTGTGCTGGA |
| | | CTCCGACGGCTCCTTCTTCCTGTACTCCAAACTGACCGTGGACA |
| | | AGTCCCGGTGGCAGCAGGGCAACGTGTTCTCCTGCTCCGTGATG |
| | | CACGAGGCCCTGCACAACCACTACACCCAGAAGTCCCTGTCCCT |
| | | GTCTCCCGGCAAG |
| SEQ ID NO: 75 | LCDR1 | RASQGIRAWLN |
| (Combined) | | |
| SEQ ID NO: 76 | LCDR2 | AASSLQS |
| (Combined) | | |
| SEQ ID NO: 77 | LCDR3 | HQYITHPPT |
| (Combined) | | |
| SEQ ID NO: 78 | LCDR1 | RASQGIRAWLN |
| (Kabat) | | |
| SEQ ID NO: 79 | LCDR2 | AASSLQS |
| (Kabat) | | |
| SEQ ID NO: 80 | LCDR3 | HQYITHPPT |
| (Kabat) | | |
| SEQ ID NO: 81 | LCDR1 | SQGIRAW |
| (Chothia) | | |
| SEQ ID NO: 82 | LCDR2 | AAS |
| (Chothia) | | |
| SEQ ID NO: 83 | LCDR3 | YITHPP |
| (Chothia) | | |
| SEQ ID NO: 84 | LCDR1 | QGIRAW |
| (IMGT) | | |
| SEQ ID NO: 85 | LCDR2 | AAS |
| (IMGT) | | |
| SEQ ID NO: 86 | LCDR3 | HQYITHPPT |
| (IMGT) | | |
| SEQ ID NO: 87 | VL | DIQMTQSPSSLSASVGDRVTITCRASQGIRAWLNWYQQKPGKAP |
| | | KLLIYAASSLQSGVPSRFSGSGSGTDFTLTISSLQPEDFATYYC |
| | | HQYITHPPTFGQGTKVEIK |
| SEQ ID NO: 88 | DNA VL | GACATCCAGATGACCCAGAGCCCCTCCAGCCTGTCCGCCTCCGT |
| | | GGGCGACAGAGTGACCATCACCTGTCGGGCCTCTCAGGGCATCC |
| | | GGGCCTGGCTGAACTGGTATCAGCAGAAGCCCGGCAAGGCCCCT |
| | | AAGCTGCTGATCTACGCCGCCAGCTCCCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | CACCAGTACATCACCCACCCTCCCACCTTCGGCCAGGGCACCAA |
| | | AGTGGAAATCAAG |
| SEQ ID NO: 89 | Light | DIQMTQSPSSLSASVGDRVTITCRASQGIRAWLNWYQQKPGKAP |
| | Chain | KLLIYAASSLQSGVPSRFSGSGSGTDFTLTISSLQPEDFATYYC |
| | | HQYITHPPTFGQGTKVEIKRTVAAPSVFIFPPSDEQLKSGTASV |
| | | VCLLNNFYPREAKVQWKVDNALQSGNSQESVTEQDSKDSTYSLS |
| | | STLTLSKADYEKHKVYACEVTHQGLSSPVTKSFNRGEC |
| SEQ ID NO: 90 | DNA | GACATCCAGATGACCCAGAGCCCCTCCAGCCTGTCCGCCTCCGT |
| | Light | GGGCGACAGAGTGACCATCACCTGTCGGGCCTCTCAGGGCATCC |
| | Chain | GGGCCTGGCTGAACTGGTATCAGCAGAAGCCCGGCAAGGCCCCT |
| | | AAGCTGCTGATCTACGCCGCCAGCTCCCTGCAGTCCGGCGTGCC |
| | | CTCCAGATTCTCCGGCTCTGGCTCCGGCACCGACTTCACCCTGA |
| | | CCATCTCCAGCCTGCAGCCCGAGGACTTCGCCACCTACTACTGC |
| | | CACCAGTACATCACCCACCCTCCCACCTTCGGCCAGGGCACCAA |
| | | AGTGGAAATCAAGCGTACGGTGGCCGCTCCCAGCGTGTTCATCT |
| | | TCCCCCCCAGCGACGAGCAGCTGAAGAGCGGCACCGCCAGCGTG |
| | | GTGTGCCTGCTGAACAACTTCTACCCCCGGGAGGCCAAGGTGCA |
| | | GTGGAAGGTGGACAACGCCCTGCAGAGCGGCAACAGCCAGGAGA |
| | | GCGTCACCGAGCAGGACAGCAAGGACTCCACCTACAGCCTGAGC |
| | | AGCACCCTGACCCTGAGCAAGGCCGACTACGAGAAGCATAAGGT |
| | | GTACGCCTGCGAGGTGACCCACCAGGGCCTGTCCAGCCCCGTGA |
| | | CCAAGAGCTTCAACAGGGGCGAGTGC |

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme), or Lefranc et al., (2003) Dev. Comp. Immunol., 27, 55-77 (“IMGT” numbering scheme). Other methods for delineating the CDR regions may alternatively be used. For example, the CDR definitions of both Kabat and Chothia may be combined (“Combined” system).

For example, under Kabat, the CDR amino acid residues of an antibody in the heavy chain variable domain (VH) are numbered 31-35 (HCDR1), 50-66 (HCDR2), and 99-111 (HCDR3); and the CDR amino acid residues in the light chain variable domain (VL) are numbered 22-35 (LCDR1), 51-57 (LCDR2), and 90-100 (LCDR3). Under Chothia the CDR amino acids in the VH are numbered 26-32 (HCDR1), 52-57 (HCDR2), and 99-111 (HCDR3); and the amino acid residues in VL are numbered 25-33 (LCDR1), 51-53 (LCDR2), and 92-99 (LCDR3). By combining the CDR definitions of both Kabat and Chothia, the “Combined” CDRs consist of amino acid residues 26-35 (HCDR1), 50-66 (HCDR2), and 99-108 (HCDR3) in human VH and amino acid residues 24-38 (LCDR1), 54-60 (LCDR2), and 93-101 (LCDR3) in human VL. As another example, under IMGT, the CDR amino acid residues in the heavy chain variable domain (VH) are numbered 26-33 (HCDR1), 51-58 (HCDR2), and 97-108 (HCDR3); and the CDR amino acid residues in the light chain variable domain (VL) are numbered 27-36 (LCDR1), 54-56 (LCDR2), and 93-101 (LCDR3). Table 2 provides exemplary Kabat, Chothia, Combined, and IMGT HCDR1, HCDR2, HCDR3, LCDR1, LCDR2, and LCDR3 for anti-FXI/FXIa antibody binding agents (e.g., antibodies), e.g., IDT1-IDT10.

Since each of the antibodies disclosed in Table 2, can bind to anti-FXI/FXIa antibody NOV1401, and antigen-binding specificity is provided primarily by the CDR1, 2 and 3 regions, the VH CDR1, 2 and 3 sequences and VL CDR1, 2 and 3 sequences can be “mixed and matched” (i.e., CDRs from different antibodies can be mixed and matched), although each antibody preferably contains a VH CDR1, 2 and 3 and a VL CDR1, 2 and 3 to create other FXI and/or FXIa binding molecules provided herein. Such “mixed and matched” anti-FXI/FXIa antibody binding agents can be tested using the binding assays known in the art and those described in the Examples (e.g., ELISAs, SET, BIACORE™ assays). When VH CDR sequences are mixed and matched, the CDR1, CDR2 and/or CDR3 sequence from a particular VH sequence should be replaced with a structurally similar CDR sequence(s). Likewise, when VL CDR sequences are mixed and matched, the CDR1, CDR2 and/or CDR3 sequence from a particular VL sequence should be replaced with a structurally similar CDR sequence(s). It will be readily apparent to the ordinarily skilled artisan that novel VH and VL sequences can be created by substituting one or more VH and/or VL CDR region sequences with structurally similar sequences from the CDR sequences shown herein for antibodies provided herein. In addition to the foregoing, in one aspect, binding agents provided herein may be antigen-binding fragments of antibodies and can comprise a VH CDR1, 2, and 3, or a VL CDR 1, 2, and 3, wherein the fragment binds to an anti-FXI/FXIa antibody, such as NOV1401, as a single variable domain.

In particular aspects, provided herein is a binding agent (e.g., anti-idiotype antibody and fragments thereof) which specifically binds a target anti-FXI/FXIa antibody, as well as a pharmaceutical composition comprising such binding agent, wherein the binding agent inhibits an anticoagulant activity of the target anti-FXI/FXIa antibody, wherein the target anti-FXI/FXIa antibody is antibody NOV1401 (e.g., comprising a VH comprising the amino acid sequence of SEQ ID NO: 12 and a VL comprising the amino acid sequence of SEQ ID NO: 23), and wherein the binding agent is an antibody (e.g., full length IgG) or antigen-binding fragment thereof comprising (1) a VH comprising complementarity determining regions HCDR1, HCDR2, and HCDR3, and (2) a VL comprising complementarity determining regions LCDR1, LCDR2, and LCDR3; wherein:
a. the HCDR1 comprises the amino acid sequence of SEQ ID NO: 27, 59, 91, 123, 155, 187, 219, 251, 283, or 315;
b. the HCDR2 comprises the amino acid sequence of SEQ ID NO: 28, 60, 92, 124, 156, 188, 220, 252, 284, or 316;
c. the HCDR3 comprises the amino acid sequence of SEQ ID NO: 29, 61, 93, 125, 157, 189, 221, 253, 285, or 317;
d. the LCDR1 comprises the amino acid sequence of SEQ ID NO: 43, 75, 107, 139, 171, 203, 235, 267, 299, or 331;
e. the LCDR2 comprises the amino acid sequence of SEQ ID NO: 44, 76, 108, 140, 172, 204, 236, 268, 300, or 332; and
f. the LCDR3 comprises the amino acid sequence of SEQ ID NO: 45, 77, 109, 141, 173, 205, 237, 269, 301, or 333.

In particular aspects, provided herein is a binding agent (e.g., anti-idiotype antibody and fragments thereof) which specifically binds a target anti-FXI/FXIa antibody, as well as a pharmaceutical composition comprising such binding agent, wherein the binding agent inhibits an anticoagulant activity of the target anti-FXI/FXIa antibody, wherein the target anti-FXI/FXIa antibody is antibody NOV1401 (comprising a VH comprising the amino acid sequence of SEQ ID NO: 12 and a VL comprising the amino acid sequence of SEQ ID NO: 23), and wherein the binding agent is an antibody or antigen-binding fragment thereof comprising (1) a VH comprising complementarity determining regions HCDR1, HCDR2, and HCDR3, and (2) a VL comprising complementarity determining regions LCDR1, LCDR2, and LCDR3; wherein:
a. the HCDR1 comprises the amino acid sequence of SEQ ID NO: 30, 62, 94, 126, 158, 190, 222, 254, 286, or 318;
b. the HCDR2 comprises the amino acid sequence of SEQ ID NO: 31, 63, 95, 127, 159, 191, 223, 255, 287, or 319;
c. the HCDR3 comprises the amino acid sequence of SEQ ID NO: 32, 64, 96, 128, 160, 192, 224, 256, 288, or 320;
d. the LCDR1 comprises the amino acid sequence of SEQ ID NO: 46, 78, 110, 142, 174, 206, 238, 270, 302, or 334;
e. the LCDR2 comprises the amino acid sequence of SEQ ID NO: 47, 79, 111, 143, 175, 207, 239, 271, 303, or 335; and
f. the LCDR3 comprises the amino acid sequence of SEQ ID NO: 48, 80, 112, 144, 176, 208, 240, 272, 304, or 336.

Since each of the binding agents (e.g., antibodies) disclosed in Table 2, can bind to anti-FXI/FXIa antibody NOV1401, the VH, VL, full length light chain, and full length heavy chain sequences (amino acid sequences and the nucleotide sequences encoding the amino acid sequences) can be “mixed and matched” to create other anti-FXI/FXIa antibody binding agents. Such “mixed and matched” anti-FXI/FXIa antibody binding agents can be tested using the binding assays known in the art (e.g., ELISAs, and other assays described in the Example section). When these chains are mixed and matched, a VH sequence from a particular VH/VL pairing should be replaced with a structurally similar VH sequence. Likewise a full length heavy chain sequence from a particular full length heavy chain/full length light chain pairing should be replaced with a structurally similar full length heavy chain sequence. Likewise, a VL sequence from a particular VH/VL pairing should be replaced with a structurally similar VL sequence. Likewise a full length light chain sequence from a particular full length heavy chain/full length light chain pairing should be replaced with a structurally similar full length light chain sequence.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a VH and a VL, wherein the VH comprises the amino acid sequence of SEQ ID NO: 39, 71, 103, 135, 167, 199, 231, 263, 295, or 327, and the VL comprises the amino acid sequence of SEQ ID NO: 55, 87, 119, 151, 183, 215, 247, 279, 311, or 343.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a VH and a VL, wherein the VH comprises 3 VH CDRs of the VH amino acid sequence of SEQ ID NO: 39, 71, 103, 135, 167, 199, 231, 263, 295, or 327, and the VL comprises the 3 VL CDRs of the VL amino acid sequence of SEQ ID NO: 55, 87, 119, 151, 183, 215, 247, 279, 311, or 343.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a VH and a VL, wherein the VH comprises the amino acid sequence of SEQ ID NO: 39 and the VL comprises the amino acid sequence of SEQ ID NO: 55.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a VH and a VL, and wherein the VH comprises the amino acid sequence of SEQ ID NO: 71 and the VL comprises the amino acid sequence of SEQ ID NO: 87.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a VH and a VL, and wherein the VH comprises the amino acid sequence of SEQ ID NO: 103 and the VL comprises the amino acid sequence of SEQ ID NO: 119.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a VH and a VL, and wherein the VH comprises the amino acid sequence of SEQ ID NO: 135 and the VL comprises the amino acid sequence of SEQ ID NO: 151.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a VH and a VL, and wherein the VH comprises the amino acid sequence of SEQ ID NO: 167 and the VL comprises the amino acid sequence of SEQ ID NO: 183.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a VH and a VL, and wherein the VH comprises the amino acid sequence of SEQ ID NO: 199 and the VL comprises the amino acid sequence of SEQ ID NO: 215.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a VH and a VL, and wherein the VH comprises the amino acid sequence of SEQ ID NO: 231 and the VL comprises the amino acid sequence of SEQ ID NO: 247.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a VH and a VL, and wherein the VH comprises the amino acid sequence of SEQ ID NO: 263 and the VL comprises the amino acid sequence of SEQ ID NO: 279.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a VH and a VL, and wherein the VH comprises the amino acid sequence of SEQ ID NO: 295 and the VL comprises the amino acid sequence of SEQ ID NO: 311.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a VH and a VL, and wherein the VH comprises the amino acid sequence of SEQ ID NO: 327 and the VL comprises the amino acid sequence of SEQ ID NO: 343.

In particular aspects, provided herein is a binding agent (e.g., anti-idiotype antibody and fragments thereof, such as Fab fragment) or a pharmaceutical composition comprising such binding agent which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, and wherein the binding agent is an antibody or antigen-binding fragment thereof comprising a heavy chain and a light chain, wherein the heavy chain comprises the amino acid sequence of SEQ ID NO: 347, and the light chain comprises the amino acid sequence of SEQ ID NO: 57.

In particular aspects, provided herein is a binding agent (e.g., anti-idiotype antibody and fragments thereof, such as Fab fragment) or a pharmaceutical composition comprising such binding agent which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, and wherein the binding agent is an antibody or antigen-binding fragment thereof comprising a heavy chain and a light chain, wherein the heavy chain comprises the amino acid sequence of SEQ ID NO: 349, and the light chain comprises the amino acid sequence of SEQ ID NO: 89.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a heavy chain and a light chain, wherein the heavy chain comprises the amino acid sequence of SEQ ID NO: 41, 73, 105, 137, 169, 201, 233, 265, 297, or 329, and the light chain comprises the amino acid sequence of SEQ ID NO: 57, 89, 121, 153, 185, 217, 249, 281, 313, or 345.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a heavy chain and a light chain, and wherein the heavy chain comprises the amino acid sequence of SEQ ID NO: 41 and the light chain comprises the amino acid sequence of SEQ ID NO: 57.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a heavy chain and a light chain, and wherein the heavy chain comprises the amino acid sequence of SEQ ID NO: 73 and the light chain comprises the amino acid sequence of SEQ ID NO: 89.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a heavy chain and a light chain, and wherein the heavy chain comprises the amino acid sequence of SEQ ID NO: 105 and the light chain comprises the amino acid sequence of SEQ ID NO: 121.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a heavy chain and a light chain, and wherein the heavy chain comprises the amino acid sequence of SEQ ID NO: 137 and the light chain comprises the amino acid sequence of SEQ ID NO: 153.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a heavy chain and a light chain, and wherein the heavy chain comprises the amino acid sequence of SEQ ID NO: 169 and the light chain comprises the amino acid sequence of SEQ ID NO: 185.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a heavy chain and a light chain, and wherein the heavy chain comprises the amino acid sequence of SEQ ID NO: 201 and the light chain comprises the amino acid sequence of SEQ ID NO: 217.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a heavy chain and a light chain, and wherein the heavy chain comprises the amino acid sequence of SEQ ID NO: 233 and the light chain comprises the amino acid sequence of SEQ ID NO: 249.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a heavy chain and a light chain, and wherein the heavy chain comprises the amino acid sequence of SEQ ID NO: 265 and the light chain comprises the amino acid sequence of SEQ ID NO: 281.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a heavy chain and a light chain, and wherein the heavy chain comprises the amino acid sequence of SEQ ID NO: 297 and the light chain comprises the amino acid sequence of SEQ ID NO: 313.

In particular aspects, provided herein is a pharmaceutical composition comprising a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, wherein the binding agent is an antibody or antigen-binding fragment thereof comprising a heavy chain and a light chain, and wherein the heavy chain comprises the amino acid sequence of SEQ ID NO: 329 and the light chain comprises the amino acid sequence of SEQ ID NO: 345.

In certain aspects, provided herein is a pharmaceutical composition comprising a binding agent (e.g., anti-idiotype antibody and fragments thereof, such as Fab fragment) which specifically binds a target anti-FXI/FXIa antibody, such as NOV1401 (e.g., comprising a VH comprising the amino acid sequence of SEQ ID NO: 12 and a VL comprising the amino acid sequence of SEQ ID NO: 23), wherein the binding agent inhibits an anticoagulant activity of the target anti-FXI/FXIa antibody, and wherein the binding agent is an antibody Fab fragment of antibody IDT1, IDT2, IDT3, IDT4, IDT5, IDT6, IDT7, IDT8, IDT9, or IDT10, for example, as set forth in Table 2.

In certain aspects, provided herein is a pharmaceutical composition comprising a binding agent (e.g., anti-idiotype antibody and fragments thereof, such as Fab fragment) which specifically binds a target anti-FXI/FXIa antibody, such as NOV1401 (e.g., comprising a VH comprising the amino acid sequence of SEQ ID NO: 12 and a VL comprising the amino acid sequence of SEQ ID NO: 23), and wherein the binding agent inhibits an anticoagulant activity of the target anti-FXI/FXIa antibody, wherein the binding agent is an antibody Fab fragment of antibody IDT1, IDT2, IDT3, IDT4, IDT5, IDT6, IDT7, IDT8, IDT9, or IDT10, for example, as set forth in Table 2, and is a recombinant, monoclonal human antibody.

In certain aspects, provided herein is a pharmaceutical composition comprising a binding agent (e.g., anti-idiotype antibody and fragments thereof, such as Fab fragment) which specifically binds a target anti-FXI/FXIa antibody, such as NOV1401 (e.g., comprising a VH comprising the amino acid sequence of SEQ ID NO: 12 and a VL comprising the amino acid sequence of SEQ ID NO: 23), and wherein the binding agent inhibits an anticoagulant activity of the target anti-FXI/FXIa antibody, wherein the binding agent is antibody IDT1, IDT2, IDT3, IDT4, IDT5, IDT6, IDT7, IDT8, IDT9, IDT10, IDT11, or IDT12, for example, as set forth in Table 2, and wherein the binding agent is in a liquid formulation comprising sucrose in the range of 150 mM to 300 mM (e.g., 220 mM sucrose) and histidine in the range of 5 mM to 35 mM (e.g., 20 mM histidine), and wherein the formulation has a pH in the range of 4.5 to 6.5 (e.g., pH of 5.5). In a specific aspect, the pharmaceutical composition comprises the binding agent (e.g., binding agent set forth in Table 2) at a concentration of 150 mg/mL.

As used herein, a human antibody comprises heavy or light chain variable regions or full length heavy or light chains that are “the product of” or “derived from” a particular germline sequence if the variable regions or full length chains of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody.

A human antibody that is “the product of” or “derived from” a particular human germline immunoglobulin sequence may contain amino acid differences as compared to the germline sequence, due to, for example, naturally occurring somatic mutations or intentional introduction of site-directed mutations. However, in specific aspects, in the VH or VL framework regions, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene.

In specific aspects, typically, a recombinant human antibody will display no more than 10 amino acid differences from the amino acid sequence encoded by the human germline immunoglobulin gene in the VH or VL framework regions. In certain cases, the human antibody may display no more than 5, or even no more than 4, 3, 2, or 1 amino acid difference from the amino acid sequence encoded by the germline immunoglobulin gene. Examples of human germline immunoglobulin genes include, but are not limited to the variable domain germline fragments described here, as well as DP47 and DPK9.

### Homologous Antibodies

In another aspect, the present disclosure provides a binding agent comprising amino acid sequences that are homologous to sequences described in Table 2, as well as a pharmaceutical composition comprising such binding agent, wherein the binding agent binds to an anti-FXI/FXIa antibody, and retains the desired functional properties (e.g., reversal of one or more anticoagulant effects) of those antibodies described in Table 2 such as any one of antibodies IDT1-IDT12. In specific aspects, such homologous antibodies retain the CDR amino acid sequences described in Table 2 (e.g., Kabat CDRs, Chothia CDRs, IMGT CDRs, or Combined CDRs). In specific aspects, such homologous antibodies are human full length IgGs.

In particular aspects, provided herein is a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23) as well as a pharmaceutical composition comprising the binding agent, wherein the binding agent inhibits an anticoagulant activity of the target anti-FXI/FXIa antibody, wherein the binding agent is an antibody or antigen-binding fragment thereof comprising a VH and a VL, and wherein the VH and VL comprise amino acid sequences that are at least 80%, at least 85%, at least 90%, at least 91%, at least 92%, at least 93%, at least 94%, at least 95%, at least 96%, at least 97%, at least 98% or at least 99% identical to the VH and VL sequences selected from Table 2. In a further specific aspect, the differences in amino acid sequence in the VL and/or VH of the binding agent is not within the complementarity determining regions.

In particular aspects, provided herein is a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), as well as a pharmaceutical composition comprising the binding agent, wherein the binding agent inhibits an anticoagulant activity of the target anti-FXI/FXIa antibody, wherein the binding agent is an antibody or antigen-binding fragment thereof comprising a VH and a VL, and wherein the VH comprises an amino acid sequence that is at least 90% or at least 95% identical to the amino acid sequence of SEQ ID NO: 39 and the VL comprises an amino acid sequence that is at least 90% or at least 95% identical to the amino acid sequence of SEQ ID NO: 55. In a further specific aspect, the differences in amino acid sequence in the VL and/or VH of the binding agent is not within the complementarity determining regions.

In particular aspects, provided herein is a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), as well as a pharmaceutical composition comprising the binding agent, wherein the binding agent inhibits an anticoagulant activity of the target anti-FXI/FXIa antibody, wherein the binding agent is an antibody or antigen-binding fragment thereof comprising a VH and a VL, and wherein the VH comprises an amino acid sequence that is at least 90% or at least 95% identical to the amino acid sequence of SEQ ID NO: 71 and the VL comprises the amino acid sequence of SEQ ID NO: 87. In a further specific aspect, the differences in amino acid sequence in the VL and/or VH of the binding agent is not within the complementarity determining regions.

In particular aspects, provided herein is a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), as well as a pharmaceutical composition comprising such binding agent, wherein the binding agent inhibits an anticoagulant activity of the target anti-FXI/FXIa antibody, wherein the binding agent is an antibody or antigen-binding fragment thereof comprising a VH and a VL, and wherein the VH comprises an amino acid sequence that is at least 90% or at least 95% identical to the amino acid sequence of SEQ ID NO: 103 and the VL comprises the amino acid sequence of SEQ ID NO: 119. In a further specific aspect, the differences in amino acid sequence in the VL and/or VH of the binding agent is not within the complementarity determining regions.

In particular aspects, provided herein is a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), as well as a pharmaceutical composition comprising such binding agent, wherein the binding agent inhibits an anticoagulant activity of the target anti-FXI/FXIa antibody, wherein the binding agent is an antibody or antigen-binding fragment thereof comprising a VH and a VL, and wherein the VH comprises an amino acid sequence that is at least 90% or at least 95% identical to the amino acid sequence of SEQ ID NO: 135 and the VL comprises an amino acid sequence that is at least 90% or at least 95% identical to the amino acid sequence of SEQ ID NO: 151. In a further specific aspect, the differences in amino acid sequence in the VL and/or VH of the binding agent is not within the complementarity determining regions.

In particular aspects, provided herein is a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), as well as a pharmaceutical composition comprising such binding agent, wherein the binding agent inhibits an anticoagulant activity of the target anti-FXI/FXIa antibody, wherein the binding agent is an antibody or antigen-binding fragment thereof comprising a VH and a VL, and wherein the VH comprises an amino acid sequence that is at least 90% or at least 95% identical to the amino acid sequence of SEQ ID NO: 167 and the VL comprises an amino acid sequence that is at least 90% or at least 95% identical to the amino acid sequence of SEQ ID NO: 183. In a further specific aspect, the differences in amino acid sequence in the VL and/or VH of the binding agent is not within the complementarity determining regions.

In particular aspects, provided herein is a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), as well as a pharmaceutical composition comprising such binding agent, wherein the binding agent inhibits an anticoagulant activity of the target anti-FXI/FXIa antibody, wherein the binding agent is an antibody or antigen-binding fragment thereof comprising a VH and a VL, and wherein the VH comprises an amino acid sequence that is at least 90% or at least 95% identical to the amino acid sequence of SEQ ID NO: 199 and the VL comprises an amino acid sequence that is at least 90% or at least 95% identical to the amino acid sequence of SEQ ID NO: 215. In a further specific aspect, the differences in amino acid sequence in the VL and/or VH of the binding agent is not within the complementarity determining regions.

In particular aspects, provided herein is a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), as well as a pharmaceutical composition comprising such binding agent, wherein the binding agent inhibits an anticoagulant activity of the target anti-FXI/FXIa antibody, wherein the binding agent is an antibody or antigen-binding fragment thereof comprising a VH and a VL, and wherein the VH comprises an amino acid sequence that is at least 90% or at least 95% identical to the amino acid sequence of SEQ ID NO: 231 and the VL comprises an amino acid sequence that is at least 90% or at least 95% identical to the amino acid sequence of SEQ ID NO: 247. In a further specific aspect, the differences in amino acid sequence in the VL and/or VH of the binding agent is not within the complementarity determining regions.

In particular aspects, provided herein is a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), as well as a pharmaceutical composition comprising such binding agent, wherein the binding agent inhibits an anticoagulant activity of the target anti-FXI/FXIa antibody, wherein the binding agent is an antibody or antigen-binding fragment thereof comprising a VH and a VL, and wherein the VH comprises an amino acid sequence that is at least 90% or at least 95% identical to the amino acid sequence of SEQ ID NO: 263 and the VL comprises an amino acid sequence that is at least 90% or at least 95% identical to the amino acid sequence of SEQ ID NO: 279. In a further specific aspect, the differences in amino acid sequence in the VL and/or VH of the binding agent is not within the complementarity determining regions.

In particular aspects, provided herein is a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), as well as a pharmaceutical composition comprising such binding agent, wherein the binding agent inhibits an anticoagulant activity of the target anti-FXI/FXIa antibody, wherein the binding agent is an antibody or antigen-binding fragment thereof comprising a VH and a VL, and wherein the VH comprises an amino acid sequence that is at least 90% or at least 95% identical to the amino acid sequence of SEQ ID NO: 295 and the VL comprises an amino acid sequence that is at least 90% or at least 95% identical to the amino acid sequence of SEQ ID NO: 311. In a further specific aspect, the differences in amino acid sequence in the VL and/or VH of the binding agent is not within the complementarity determining regions.

In particular aspects, provided herein is a binding agent (e.g., anti-idiotype antibody and fragments thereof, such as Fab fragment) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), as well as a pharmaceutical composition comprising such binding agent, wherein the binding agent inhibits an anticoagulant activity of the target anti-FXI/FXIa antibody, wherein the binding agent is an antibody or antigen-binding fragment thereof comprising a VH and a VL, and wherein the VH comprises an amino acid sequence that is at least 90% or at least 95% identical to the amino acid sequence of SEQ ID NO: 327 and the VL comprises an amino acid sequence that is at least 90% or at least 95% identical to the amino acid sequence of SEQ ID NO: 343. In a further specific aspect, the differences in amino acid sequence in the VL and/or VH of the binding agent is not within the complementarity determining regions.

In particular aspects, provided herein is a binding agent (e.g., anti-idiotype antibody) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), as well as a pharmaceutical composition comprising such binding agent, wherein the binding agent inhibits an anticoagulant activity of the target anti-FXI/FXIa antibody, wherein the binding agent is an antibody comprising a heavy chain and a light chain, wherein the heavy comprises an amino acid sequence that is at least 90% or at least 95% or at least 98% identical to the amino acid sequence of SEQ ID NO: 347 and the light chain comprises an amino acid sequence that is at least 90% or at least 95% or at least 98% identical to the amino acid sequence of SEQ ID NO: 57. In a further specific aspect, the differences in amino acid sequence in the heavy chain and/or light chain of the binding agent is not within the complementarity determining regions.

In particular aspects, provided herein is a binding agent (e.g., anti-idiotype antibody) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), as well as a pharmaceutical composition comprising such binding agent, wherein the binding agent inhibits an anticoagulant activity of the target anti-FXI/FXIa antibody, wherein the binding agent is an antibody comprising a heavy chain and a light chain, wherein the heavy comprises an amino acid sequence that is at least 90% or at least 95% or at least 98% identical to the amino acid sequence of SEQ ID NO: 349 and the light chain comprises an amino acid sequence that is at least 90% or at least 95% or at least 98% identical to the amino acid sequence of SEQ ID NO: 89. In a further specific aspect, the differences in amino acid sequence in the heavy chain and/or light chain of the binding agent is not within the complementarity determining regions.

As used herein, the percent identity between the two sequences is a function of the number of identical positions shared by the sequences (i.e., % identity equals number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

Additionally or alternatively, the protein sequences of the present invention can further be used as a “query sequence” to perform a search against public databases to, for example, identify related sequences. For example, such searches can be performed using the BLAST program (version 2.0) of Altschul, et al., 1990 J. Mol. Biol. 215:403-10.

The present disclosure also provides a binding agent (e.g., anti-idiotype antibody and fragments thereof, such as Fab fragment) which specifically binds a target anti-FXI/FXIa antibody (e.g., NOV1401), wherein the binding agent is an antibody or antigen-binding fragment thereof comprising (or alternatively, consisting of) a VH amino acid sequence listed in Table 2, wherein no more than about 1, 2, 3, 4, 5, 6, 7, 8, 9, 10, 11, 12, 13, 14, 15, 16, 17, 18, 19, or 20 amino acids in a framework sequence (for example, a sequence which is not a CDR) have been mutated (wherein a mutation is, as various non-limiting examples, an addition, substitution or deletion).

The present disclosure also provides a binding agent (e.g., anti-idiotype antibody and fragments thereof, such as Fab fragment) which specifically binds a target anti-FXI/FXIa antibody (e.g., NOV1401), wherein the binding agent is an antibody or antigen-binding fragment thereof comprising (or alternatively, consisting of) a VL amino acid sequence listed in Table 2, wherein no more than about 1, 2, 3, 4, 5, 6, 7, 8, 9, 10, 11, 12, 13, 14, 15, 16, 17, 18, 19, or 20 amino acids in a framework sequence (for example, a sequence which is not a CDR) have been mutated (wherein a mutation is, as various non-limiting examples, an addition, substitution or deletion).

Antibodies with Conservative Modifications

In certain aspects, the present disclosure relates to a binding agent, which is an antibody or antigen-binding fragment thereof (e.g., Fab fragment) that specifically binds to an anti-FXI/FXIa antibody such as NOV1401, as well as a pharmaceutical composition comprising such binding agent, wherein the binding agent comprises VH comprising CDR1, CDR2, and CDR3 sequences and a VL comprising CDR1, CDR2, and CDR3 sequences, wherein one or more of these CDR sequences have specified amino acid sequences based on the antibodies described herein, such as those described in Table 2, or conservative modifications thereof, and wherein the binding agents retain the desired functional properties (e.g., reversing one or more anticoagulant effects of an anti-FXI/FXIa antibody) of the binding agents described herein, e.g., binding agents IDT1, IDT2, IDT3, IDT4, IDT5, IDT6, IDT7, IDT8, IDT9, IDT10, IDT11, or IDT12.

In specific aspects, a binding agent described herein, which is an antibody (e.g., full length IgG) or antigen-binding fragment thereof (e.g., Fab fragment) that specifically binds to an anti-FXI/FXIa antibody such as NOV1401, comprises VH comprising CDR1, CDR2, and CDR3 sequences and a VL comprising CDR1, CDR2, and CDR3 sequences set forth in Table 2 with one, two, three, or more conservative modifications in one or more CDRs, and wherein the binding agents retain the desired functional properties (e.g., binding to anti-FXI/FXIa antibody and/or reversing one or more anticoagulant effects of an anti-FXI/FXIa antibody) of the binding agents described herein, e.g., binding agents IDT1, IDT2, IDT3, IDT4, IDT5, IDT6, IDT7, IDT8, IDT9, IDT10, IDT11, or IDT12.

In further specific aspects, provided herein is a binding agent (e.g., anti-idiotype antibody) which specifically binds a target anti-FXI/FXIa antibody such as NOV1401, as well as a pharmaceutical composition comprising such binding agent, wherein the binding agent inhibits an anticoagulant activity of the target anti-FXI/FXIa antibody, wherein the binding agent is an antibody or antigen-binding fragment thereof comprising (1) a VH comprising complementarity determining regions HCDR1, HCDR2, and HCDR3 selected from those set forth in Table 2 and conservative modifications thereof, and (2) a VL comprising complementarity determining regions LCDR1, LCDR2, and LCDR3 selected from those set forth in Table 2 and conservative modifications thereof. In a particular aspect, the binding agent (e.g., anti-idiotype antibody) comprises Combined HCDR1, HCDR2, and HCDR3 selected from those set forth in Table 2 and conservative modifications thereof, and Combined LCDR1, LCDR2, and LCDR3 selected from those set forth in Table 2 and conservative modifications thereof. In a particular aspect, the binding agent (e.g., anti-idiotype antibody) comprises Kabat HCDR1, HCDR2, and HCDR3 selected from those set forth in Table 2 and conservative modifications thereof, and Kabat LCDR1, LCDR2, and LCDR3 selected from those set forth in Table 2 and conservative modifications thereof. In a particular aspect, the binding agent (e.g., anti-idiotype antibody) comprises Chothia HCDR1, HCDR2, and HCDR3 selected from those set forth in Table 2 and conservative modifications thereof, and Chothia LCDR1, LCDR2, and LCDR3 selected from those set forth in Table 2 and conservative modifications thereof. In a particular aspect, the binding agent (e.g., anti-idiotype antibody) comprises IMGT HCDR1, HCDR2, and HCDR3 selected from those set forth in Table 2 and conservative modifications thereof, and IMGT LCDR1, LCDR2, and LCDR3 selected from those set forth in Table 2 and conservative modifications thereof. In a specific aspect, the binding agent is a full length IgG.

In particular aspects, provided herein is a binding agent (e.g., anti-idiotype antibody and fragments thereof, such as Fab fragment) which specifically binds a target anti-FXI/FXIa antibody such as NOV1401, as well as a pharmaceutical composition comprising such binding agent, wherein the binding agent inhibits an anticoagulant activity of the target anti-FXI/FXIa antibody, wherein the binding agent is an antibody or antigen-binding fragment thereof comprising (1) a VH comprising complementarity determining regions HCDR1, HCDR2, and HCDR3, and (2) a VL comprising complementarity determining regions LCDR1, LCDR2, and LCDR3; wherein:
a. the HCDR1 comprises the amino acid sequence of SEQ ID NO: 27, 59, 91, 123, 155, 187, 219, 251, 283, or 315, or conservative modifications thereof,
b. the HCDR2 comprises the amino acid sequence of SEQ ID NO: 28, 60, 92, 124, 156, 188, 220, 252, 284, or 316, or conservative modifications thereof;
c. the HCDR3 comprises the amino acid sequence of SEQ ID NO: 29, 61, 93, 125, 157, 189, 221, 253, 285, or 317, or conservative modifications thereof;
d. the LCDR1 comprises the amino acid sequence of SEQ ID NO: 43, 75, 107, 139, 171, 203, 235, 267, 299, or 331, or conservative modifications thereof;
e. the LCDR2 comprises the amino acid sequence of SEQ ID NO: 44, 76, 108, 140, 172, 204, 236, 268, 300, or 332, or conservative modifications thereof; and
f. the LCDR3 comprises the amino acid sequence of SEQ ID NO: 45, 77, 109, 141, 173, 205, 237, 269, 301, or 333, or conservative modifications thereof.

In particular aspects, provided herein is a binding agent (e.g., anti-idiotype antibody and fragments thereof, such as Fab fragment) which specifically binds a target anti-FXI/FXIa antibody such as NOV1401, as well as a pharmaceutical composition comprising such binding agent, wherein the binding agent inhibits an anticoagulant activity of the target anti-FXI/FXIa antibody, wherein the binding agent is an antibody or antigen-binding fragment thereof comprising (1) a VH comprising complementarity determining regions HCDR1, HCDR2, and HCDR3, and (2) a VL comprising complementarity determining regions LCDR1, LCDR2, and LCDR3; wherein:
a. the HCDR1 comprises the amino acid sequence of SEQ ID NO: 30, 62, 94, 126, 158, 190, 222, 254, 286, or 318, or conservative modifications thereof;
b. the HCDR2 comprises the amino acid sequence of SEQ ID NO: 31, 63, 95, 127, 159, 191, 223, 255, 287, or 319, or conservative modifications thereof;
c. the HCDR3 comprises the amino acid sequence of SEQ ID NO: 32, 64, 96, 128, 160, 192, 224, 256, 288, or 320, or conservative modifications thereof;
d. the LCDR1 comprises the amino acid sequence of SEQ ID NO: 46, 78, 110, 142, 174, 206, 238, 270, 302, or 334, or conservative modifications thereof;
e. the LCDR2 comprises the amino acid sequence of SEQ ID NO: 47, 79, 111, 143, 175, 207, 239, 271, 303, or 335, or conservative modifications thereof; and
f. the LCDR3 comprises the amino acid sequence of SEQ ID NO: 48, 80, 112, 144, 176, 208, 240, 272, 304, or 336, or conservative modifications thereof.

The present disclosure also provides a binding agent (e.g., anti-idiotype antibody and fragments thereof, such as Fab fragment) which specifically binds a target anti-FXI/FXIa antibody (e.g., NOV1401), as well as a pharmaceutical composition comprising such binding agent, wherein the binding agent is an antibody or antigen-binding fragment thereof comprising (or alternatively, consisting of) a VH amino acid sequence listed in Table 2, wherein no more than about 1, 2, 3, 4, 5, 6, 7, 8, 9, 10, 11, 12, 13, 14, 15, 16, 17, 18, 19, or 20 amino acids in a framework sequence (for example, a sequence which is not a CDR) have conservative modifications.

The present disclosure also provides a binding agent (e.g., anti-idiotype antibody and fragments thereof, such as Fab fragment) which specifically binds a target anti-FXI/FXIa antibody (e.g., NOV1401), wherein the binding agent is an antibody or antigen-binding fragment thereof comprising (or alternatively, consisting of) a VL amino acid sequence listed in Table 2, wherein no more than about 1, 2, 3, 4, 5, 6, 7, 8, 9, 10, 11, 12, 13, 14, 15, 16, 17, 18, 19, or 20 amino acids in a framework sequence (for example, a sequence which is not a CDR) have conservative modifications.

In particular aspects, provided herein is a binding agent (e.g., anti-idiotype antibody) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), as well as a pharmaceutical composition comprising such binding agent, wherein the binding agent inhibits an anticoagulant activity of the target anti-FXI/FXIa antibody, wherein the binding agent is an antibody comprising a heavy chain and a light chain, and wherein the heavy comprises the amino acid sequence of SEQ ID NO: 347 with one, two, three or four mutations, such as conservative amino acid mutations, that do not substantially affect activity, and/or the light chain comprises the amino acid sequence of SEQ ID NO: 57 with one, two, three or four mutations, such as conservative amino acid mutations, that do not substantially affect activity. In a further specific aspect, the mutation is not within the complementarity determining regions.

In particular aspects, provided herein is a binding agent (e.g., anti-idiotype antibody) which specifically binds a target anti-FXI/FXIa antibody (e.g., NOV1401 comprising a VH comprising the amino acid sequence of SEQ ID NO: 12 and a VL comprising the amino acid sequence of SEQ ID NO: 23), as well as a pharmaceutical composition comprising such binding agent, wherein the binding agent inhibits an anticoagulant activity of the target anti-FXI/FXIa antibody, wherein the binding agent is an antibody comprising a heavy chain and a light chain, and wherein the heavy comprises the amino acid sequence of SEQ ID NO: 349 with one, two, three or four mutations, such as conservative amino acid mutations, that do not substantially affect activity, and/or the light chain comprises the amino acid sequence of SEQ ID NO: 89 with one, two, three or four mutations, such as conservative amino acid mutations, that do not substantially affect activity. In a further specific aspect, the mutation is not within the complementarity determining regions.

### Engineered and Modified Antibodies

Binding agents (e.g., anti-FXI/FXIa antibody binding agent) provided herein which are antibodies, such as a full length IgG or a Fab fragment, can be prepared using an antibody having one or more of the VH and/or VL sequences shown herein as starting material to engineer a modified antibody, which modified antibody may have altered properties from the starting antibody. An antibody can be engineered by modifying one or more residues within one or both variable regions (i. e., VH and/or VL), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al., 1998 Nature 332:323-327; Jones, P. et al., 1986 Nature 321:522-525; Queen, C. et al., 1989 Proc. Natl. Acad., U.S.A. 86:10029-10033; U.S. Pat. No. 5,225,539 to Winter, and U.S. Pat. Nos. 5,530,101; 5,585,089; 5,693,762 and 6,180,370 to Queen et al.)

Framework sequences can be obtained from public DNA databases or published references that include germline antibody gene sequences. For example, germline DNA sequences for human heavy and light chain variable region genes can be found in the “VBase” human germline sequence database (available on the world wide web at mrc-cpe.cam.ac.uk/vbase), as well as in Kabat, E. A., et al., 1991 Sequences of Proteins of Immunological Interest, Fifth Edition, U.S. Department of Health and Human Services, NIH Publication No. 91-3242; Tomlinson, I. M., et al., 1992 J. Mol. Biol. 227:776-798; and Cox, J. P. L. et al., 1994 Eur. J Immunol. 24:827-836; the contents of each of which are expressly incorporated herein by reference.

An example of framework sequences for use in antibodies of the present disclosure are those that are structurally similar to the framework sequences used by selected antibodies described herein, e.g., consensus sequences and/or framework sequences used by monoclonal antibodies of the invention. The VH CDR1, 2 and 3 sequences, and the VL CDR1, 2 and 3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al). Frameworks that can be utilized as scaffolds on which to build the antibodies and antigen binding fragments described herein include, but are not limited to VH1A, VH1B, VH3, Vk1, V12, and Vk2. Additional frameworks are known in the art and may be found, for example, in the vBase data base on the world wide web at vbase.mrc-cpe.cam.ac.uk/index.php?&MMN_position=1:1.

Accordingly, in specific aspects, the present disclosure relates to binding agents, such as isolated antibodies which bind an anti-FXI/FXIa antibody such as NOV1401, as well as a pharmaceutical composition comprising such binding agents, comprising a heavy chain variable region comprising an amino acid sequence selected from the group consisting of SEQ ID NOs: 39, 71, 103, 135, 167, 199, 231, 263, 295, and 327, or an amino acid sequence having one, two, three, four or five amino acid substitutions, deletions or additions in the framework region of such sequences, and further comprising a light chain variable region comprising an amino acid sequence selected from the group consisting of SEQ ID NOs: 55, 87, 119, 151, 183, 215, 247, 279, 311, and 343, or an amino acid sequence having one, two, three, four or five amino acid substitutions, deletions or additions in the framework region of such sequences.

Another type of variable region modification is to mutate amino acid residues within the VH and/or VL CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples Section. Conservative modifications (as discussed above) can be introduced. The mutations may be amino acid substitutions, additions or deletions. Moreover, typically no more than one, two, three, four or five residues within a CDR region are altered.

Accordingly, in specific aspects, provided herein are binding agents that are affinity matured variants of antibody IDT1, IDT2, IDT3, IDT4, IDT5, IDT6, IDT7, IDT8, IDT9, IDT10, IDT11, or IDT12, as well as a pharmaceutical composition comprising such binding agents, wherein the affinity matured variant has higher affinity for the anti-FXI/FXIa antibody NOV1401 than the parental, and is capable of reversing one or more anticoagulant effects of NOV1401. In particular aspects, provided herein is a binding agent (e.g., anti-idiotype antibody and fragments thereof) which specifically binds a target anti-FXI/FXIa antibody, as well as a pharmaceutical composition comprising such binding agent, wherein the binding agent inhibits an anticoagulant activity of the target anti-FXI/FXIa antibody, wherein the target anti-FXI/FXIa antibody is antibody NOV1401 (comprising a VH comprising the amino acid sequence of SEQ ID NO: 12 and a VL comprising the amino acid sequence of SEQ ID NO: 23), and wherein the binding agent is an antibody or antigen-binding fragment thereof comprising (1) a VH comprising complementarity determining regions HCDR1, HCDR2, and HCDR3, and (2) a VL comprising complementarity determining regions LCDR1, LCDR2, and LCDR3; wherein:
a. the HCDR1 comprises the amino acid sequence of SEQ ID NO: 27, 59, 91, 123, 155, 187, 219, 251, 283, or 315, or an amino acid sequence thereof having one, two, three, four or five amino acid substitutions, deletions or additions;
b. the HCDR2 comprises the amino acid sequence of SEQ ID NO: 28, 60, 92, 124, 156, 188, 220, 252, 284, or 316, or an amino acid sequence thereof having one, two, three, four or five amino acid substitutions, deletions or additions;
c. the HCDR3 comprises the amino acid sequence of SEQ ID NO: 29, 61, 93, 125, 157, 189, 221, 253, 285, or 317, or an amino acid sequence thereof having one, two, three, four or five amino acid substitutions, deletions or additions;
d. the LCDR1 comprises the amino acid sequence of SEQ ID NO: 43, 75, 107, 139, 171, 203, 235, 267, 299, or 331, or an amino acid sequence thereof having one, two, three, four or five amino acid substitutions, deletions or additions;
e. the LCDR2 comprises the amino acid sequence of SEQ ID NO: 44, 76, 108, 140, 172, 204, 236, 268, 300, or 332, or an amino acid sequence thereof having one, two, three, four or five amino acid substitutions, deletions or additions; and
f. the LCDR3 comprises the amino acid sequence of SEQ ID NO: 45, 77, 109, 141, 173, 205, 237, 269, 301, or 333, or an amino acid sequence thereof having one, two, three, four or five amino acid substitutions, deletions or additions.

In particular aspects, provided herein is a binding agent (e.g., anti-idiotype antibody and fragments thereof) which specifically binds a target anti-FXI/FXIa antibody, as well as a pharmaceutical composition comprising such binding agent, wherein the binding agent inhibits an anticoagulant activity of the target anti-FXI/FXIa antibody, wherein the target anti-FXI/FXIa antibody is antibody NOV1401 (comprising a VH comprising the amino acid sequence of SEQ ID NO: 12 and a VL comprising the amino acid sequence of SEQ ID NO: 23), and wherein the binding agent is an antibody or antigen-binding fragment thereof comprising (1) a VH comprising complementarity determining regions HCDR1, HCDR2, and HCDR3, and (2) a VL comprising complementarity determining regions LCDR1, LCDR2, and LCDR3; wherein:
a. the HCDR1 comprises the amino acid sequence of SEQ ID NO: 30, 62, 94, 126, 158, 190, 222, 254, 286, or 318, or an amino acid sequence thereof having one, two, three, four or five amino acid substitutions, deletions or additions;
b. the HCDR2 comprises the amino acid sequence of SEQ ID NO: 31, 63, 95, 127, 159, 191, 223, 255, 287, or 319, or an amino acid sequence thereof having one, two, three, four or five amino acid substitutions, deletions or additions;
c. the HCDR3 comprises the amino acid sequence of SEQ ID NO: 32, 64, 96, 128, 160, 192, 224, 256, 288, or 320, or an amino acid sequence thereof having one, two, three, four or five amino acid substitutions, deletions or additions;
d. the LCDR1 comprises the amino acid sequence of SEQ ID NO: 46, 78, 110, 142, 174, 206, 238, 270, 302, or 334, or an amino acid sequence thereof having one, two, three, four or five amino acid substitutions, deletions or additions;
e. the LCDR2 comprises the amino acid sequence of SEQ ID NO: 47, 79, 111, 143, 175, 207, 239, 271, 303, or 335, or an amino acid sequence thereof having one, two, three, four or five amino acid substitutions, deletions or additions; and
f. the LCDR3 comprises the amino acid sequence of SEQ ID NO: 48, 80, 112, 144, 176, 208, 240, 272, 304, or 336, or an amino acid sequence thereof having one, two, three, four or five amino acid substitutions, deletions or additions.
   Grafting Antigen-Binding Domains into Alternative Frameworks or Scaffolds

With respect to anti-FXI/FXIa antibody binding agents provided herein which are antibodies, a wide variety of antibody/immunoglobulin frameworks or scaffolds can be employed so long as the resulting polypeptide includes at least one binding region which specifically binds to a target anti-FXI/FXIa antibody. Such frameworks or scaffolds include the 5 main idiotypes of human immunoglobulins, or fragments thereof, and include immunoglobulins of other animal species, preferably having humanized aspects. Single heavy-chain antibodies such as those identified in camelids are of particular interest in this regard.

In one aspect, the present disclosure pertains to generating non-immunoglobulin based antibodies using non-immunoglobulin scaffolds onto which CDRs such as those described in Table 2 can be grafted. Known or future non-immunoglobulin frameworks and scaffolds may be employed, as long as they comprise a binding region specific for the target anti-FXI/FXIa antibody such as NOV1401. Known non-immunoglobulin frameworks or scaffolds include, but are not limited to, fibronectin (Compound Therapeutics, Inc., Waltham, Mass.), ankyrin (Molecular Partners AG, Zurich, Switzerland), domain antibodies (Domantis, Ltd., Cambridge, Mass., and Ablynx nv, Zwijnaarde, Belgium), lipocalin (Pieris Proteolab AG, Freising, Germany), small modular immuno-pharmaceuticals (Trubion Pharmaceuticals Inc., Seattle, Wash.), maxybodies (Avidia, Inc., Mountain View, Calif.), Protein A (Affibody AG, Sweden), and affilin (gamma-crystallin or ubiquitin) (Scil Proteins GmbH, Halle, Germany).

The fibronectin scaffolds are based on fibronectin type III domain (e.g., the tenth module of the fibronectin type III (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). These fibronectin-based scaffolds are not an immunoglobulin, although the overall fold is closely related to that of the smallest functional antibody fragment, the variable region of the heavy chain, which comprises the entire antigen recognition unit in camel and llama IgG. Because of this structure, the non-immunoglobulin antibody mimics antigen binding properties that are similar in nature and affinity to those of antibodies. These scaffolds can be used in a loop randomization and shuffling strategy in vitro that is similar to the process of affinity maturation of antibodies in vivo. These fibronectin-based molecules can be used as scaffolds where the loop regions of the molecule can be replaced with CDRs of the invention using standard cloning techniques.

The ankyrin technology is based on using proteins with ankyrin derived repeat modules as scaffolds for bearing variable regions which can be used for binding to different targets. The ankyrin repeat module is a 33 amino acid polypeptide consisting of two anti-parallel α-helices and a β-turn. Binding of the variable regions is mostly optimized by using ribosome display.

Avimers are derived from natural A-domain containing protein such as LRP-1. These domains are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, U.S. Patent Application Publication Nos. 20040175756; 20050053973; 20050048512; and 20060008844.

Affibody affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa, compared to the molecular weight of antibodies, which is 150 kDa. In spite of its small size, the binding site of affibody molecules is similar to that of an antibody.

Anticalins are products developed by the company Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids. The protein architecture is reminiscent of immunoglobulins, with hypervariable loops on top of a rigid framework. However, in contrast with antibodies or their recombinant fragments, lipocalins are composed of a single polypeptide chain with 160 to 180 amino acid residues, being just marginally bigger than a single immunoglobulin domain. The set of four loops, which makes up the binding pocket, shows pronounced structural plasticity and tolerates a variety of side chains. The binding site can thus be reshaped in a proprietary process in order to recognize prescribed target molecules of different shape with high affinity and specificity. One protein of lipocalin family, the bilin-binding protein (BBP) of Pieris Brassicae has been used to develop anticalins by mutagenizing the set of four loops. One example of a patent application describing anticalins is in PCT Publication No. WO 199916873.

Affilin molecules are small non-immunoglobulin proteins which are designed for specific affinities towards proteins and small molecules. New affilin molecules can be very quickly selected from two libraries, each of which is based on a different human derived scaffold protein. Affilin molecules do not show any structural homology to immunoglobulin proteins. Currently, two affilin scaffolds are employed, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

Protein epitope mimetics (PEM) are medium-sized, cyclic, peptide-like molecules (MW 1-2 kDa) mimicking beta-hairpin secondary structures of proteins, the major secondary structure involved in protein-protein interactions.

In specific aspects, the present disclosure provides fully human antibodies that specifically bind to a target anti-FXI/FXIa antibody such as NOV1401. Compared to the chimeric or humanized antibodies, human antibodies have further reduced antigenicity when administered to human subjects.

### Fc Engineering

Antibodies of the present disclosure may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the present disclosure may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one aspect, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another aspect, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another aspect, the antibody is modified to increase its biological half-life. Various approaches are possible. For example, one or more of the mutations as described in U.S. Pat. No. 6,277,375 to Ward can be used. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another aspect, one or more amino acids selected from amino acid residues can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another aspect, one or more amino acid residues are altered to thereby alter the ability of the antibody to fix complement. This approach is described further in PCT Publication WO 94/29351 by Bodmer et al.

In a specific aspect, a binding agent described herein (e.g., binding agent described in Table 2, such as IDT11 or IDT12), for example, a binding agent which is an antibody that binds an anti-FXI/FXIa-antibody (such as antibody NOV1401) comprises a human IgG (e.g., IgG1) Fc region comprising amino acid substitutions, D265A and/or P329A, to reduce the likelihood for ADCC or CDC caused by any surface-associated FXI. These Alanine substitutions have been shown to reduce ADCC and CDC (see, e.g., Idosugie et al., J. Immunol. 164:4178-4184, 2000; Shields et al., J. Biol. Chem. 276:6591-6604, 2001).

In other aspects, a binding agent described herein comprises a human IgG (e.g., IgG1) Fc region with Fc silencing mutations such as leucine (L) to alanine (A) substitution at the position 234 and 235 (LALA) and/or the alanine (A) to asparagine (N) substitution at position 297 (N297A) (see, e.g., Leabman et al., MAbs. 5:896-903, 2013.).

In yet another aspect, the Fc region of an antibody described herein is modified to increase the ability of the antibody to mediate antibody dependent cellular cytotoxicity (ADCC) and/or to increase the affinity of the antibody for an Fcγ receptor by modifying one or more amino acids. This approach is described further in PCT Publication WO 00/42072 by Presta. Moreover, the binding sites on human IgG1 for FcγR1, FcγRII, FcγRIII and FcRn have been mapped and variants with improved binding have been described (see Shields, R. L. et al., 2001 J. Biol. Chen. 276:6591-6604).

In still another aspect, the glycosylation of an antibody is modified. For example, an aglycoslated antibody can be made (i.e., the antibody lacks glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the present disclosure to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lecl3 cells, with reduced ability to attach fucose to Asn(297)-linked carbohydrates, also resulting in hypofucosylation of antibodies expressed in that host cell (see also Shields, R. L. et al., 2002 J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180).

### Methods of Producing Antibodies

Provided herein are nucleic acid molecules (e.g., substantially purified nucleic acid molecules) which encode polypeptides of binding agents described herein such as IDT11 or IDT12 as set forth in Table 2, vectors (e.g., expression vectors) comprising the same, host cells comprising such vectors or nucleic acid molecules, and methods of producing binding agents described herein, e.g., antibodies or antigen-binding fragment thereof, which specifically binds an anti-FXI/FXIa antibody, e.g., NOV1401.

In specific aspects, provided herein is a vector (e.g., expression vector) comprising a polynucleotide described herein (e.g., Table 2), e.g., polynucleotide encoding a heavy chain of IDT11 or IDT12 and/or a light chain of IDT11 or IDT12.

In certain aspects, provided herein is a host cell comprising a vector described herein or a polynucleotide described herein e.g., polynucleotide encoding a heavy chain of IDT11 or IDT12 and/or a light chain of IDT11 or IDT12. In specific aspects, the host cell is a eukaryotic cell. In certain aspects, the host cell is a mammalian cell (e.g., non-human mammalian cell, such as CHO cells). In particular aspects, a host cell comprises (i) a vector or polynucleotide comprising nucleotide sequences encoding a VH or a heavy chain of IDT11 or IDT12, and (ii) a vector or polynucleotide comprising nucleotide sequences encoding a VL or a light chain of IDT11 or IDT12. In specific aspects, a first host cell comprises a vector or polynucleotide comprising nucleotide sequences encoding a VH or a heavy chain of IDT11 or IDT12, and a second host cell comprises a vector or polynucleotide comprising nucleotide sequences encoding a VL or a light chain of IDT11 or IDT12.

In particular aspects, provided herein is a method of producing a binding agent, e.g., an antibody or antigen-binding fragment that binds an anti-FXI/FXIa antibody, such as NOV1401, comprising the step of culturing a host cell described herein under conditions suitable for expression of the binding agent.

In certain aspects, the method of producing a binding agent provided herein (e.g., IDT11 or IDT12) or fragment thereof further comprises purifying the binding agent or fragment thereof.

### Nucleic Acids Encoding Binding Agents

The present disclosure provides polynucleotides comprising nucleotide sequences encoding binding agents described herein. In specific aspects, the present disclosure provides polynucleotides comprising nucleic acid sequences that encode the VH, VL, full length heavy chain, and/or full length light chain of antibodies described herein that specifically bind to a target anti-FXI/FXIa antibody, for example, antibodies IDT11 and IDT12. Such nucleic acid sequences can be optimized for expression in mammalian cells (for example, see Table 2).

In specific aspects where a binding agent is an antibody or antigen-binding fragment thereof, provided herein is a polynucleotide comprising nucleotide sequences encoding a heavy chain, a light chain, or a heavy chain and a light chain of an anti-FXI/FXIa antibody binding agent described herein, e.g., antibody IDT11 or IDT12. In one aspect, a polynucleotide provided herein comprises a nucleotide sequence encoding a heavy chain of an anti-FXI/FXIa antibody binding agent described herein, e.g., antibody IDT11 or IDT12. In one aspect, a polynucleotide provided herein comprises a nucleotide sequence encoding a light chain of an anti-FXI/FXIa antibody binding agent described herein, e.g., antibody IDT11 or IDT12. In one aspect, a polynucleotide provided herein comprises a nucleotide sequence encoding a heavy chain and a light chain of an anti-FXI/FXIa antibody binding agent described herein, e.g., antibody IDT11 or IDT12.

In particular aspects, provided herein is a polynucleotide comprising one or more nucleotide sequences set forth in Table 2, for example, a polynucleotide comprising the nucleotide sequence of SEQ ID NO: 42, 74, 106, 138, 170, 202, 234, 266, 298, 330, 348, or 350 encoding a heavy chain; and a comprising the nucleotide sequence of SEQ ID NO: 58, 90, 122, 154, 186, 218, 250, 282, 314, or 346 encoding a light chain.

In certain aspects, polynucleotides provided herein comprise nucleotide sequences that are substantially identical (e.g., at least 65%, 80%, 80%, 90%, 95%, 98%, or 99%) to the nucleotide sequences of those identified in Table 2, for example, SEQ ID NO: 348 or 350 encoding a heavy chain of IDT11 or IDT12; and SEQ ID NO: 58 or 90 encoding a light chain of IDT11 or IDT12. When expressed from appropriate expression vectors, polypeptides encoded by these polynucleotides are capable of binding to an anti-FXI/FXIa antibody, such as antibody NOV1401.

Because of the degeneracy of the code, a variety of nucleic acid sequences will encode each of the immunoglobulin amino acid sequences.

Polynucleotide sequences can be produced by de novo solid-phase DNA synthesis or by PCR mutagenesis of an existing sequence (e.g., sequences as described herein) encoding a binding agent, e.g., a binding agent which is an antibody or antigen-binding fragment there of (e.g., Fab fragment) that binds an anti-FXI/FXIa-antibody. Direct chemical synthesis of nucleic acids can be accomplished by methods known in the art, such as the phosphotriester method of Narang et al., 1979, Meth. Enzymol. 68:90; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, NY, 1992; PCR Protocols: A Guide to Methods and Applications, Innis et al. (Ed.), Academic Press, San Diego, Calif., 1990; Mattila et al., Nucleic Acids Res. 19:967, 1991; and Eckert et al., PCR Methods and Applications 1:17, 1991.

Also provided in the present disclosure are expression vectors and host cells for producing a binding agent described herein, e.g., a binding agent which is an antibody that binds an anti-FXI/FXIa-antibody. Various expression vectors can be employed to express the polynucleotides encoding the FXIa-binding antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat Genet 15:345, 1997). For example, nonviral vectors useful for expression of polynucleotides and polypeptides in mammalian (e.g., human) cells include pThioHis A, B & C, pcDNA3.1/His, pEBVHis A, B & C, (Invitrogen, San Diego, Calif.), MPSV vectors, and numerous other vectors known in the art for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding a binding agent described herein, e.g., a binding agent which is an antibody that binds an anti-FXI/FXIa-antibody, such as NOV1401. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression of a binding agent, e.g., a binding agent which is an antibody that binds an anti-FXI/FXIa-antibody, such as NOV1401. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted anti-FXI/FXIa-antibody binding agent sequences. In specific aspects, inserted anti-FXI/FXIa-antibody binding agent sequences are linked to a signal sequences before inclusion in the vector. Vectors to be used to receive sequences encoding anti-FXI/FXIa-antibody binding agent (e.g., antibody NOV1401 binding agent) light and heavy chain variable domains, and in certain aspects, also encode constant regions or parts thereof. Such vectors allow expression of the variable regions as fusion proteins with the constant regions thereby leading to production of intact antibodies or fragments thereof. Typically, such constant regions are human.

Host cells for harboring and expressing an anti-FXI/FXIa-antibody binding agent (e.g., antibody NOV1401 binding agent) can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express FXIa-binding polypeptides of the present disclosure. Insect cells in combination with baculovirus vectors can also be used.

In some specific embodiments, mammalian host cells are used to express and produce anti-FXI/FXIa-antibody binding agent (e.g., antibody NOV1401 binding agent) polypeptides of the present disclosure. These include any normal mortal or normal or abnormal immortal animal or human cell. For example, a number of suitable host cell lines capable of secreting intact immunoglobulins have been developed including the CHO cell lines, various Cos cell lines, HeLa cells, myeloma cell lines, and transformed B-cells. The use of mammalian tissue cell culture to express polypeptides is discussed generally in, e.g., Winnacker, FROM GENES TO CLONES, VCH Publishers, N.Y., N.Y., 1987. Expression vectors for mammalian host cells can include expression control sequences, such as an origin of replication, a promoter, and an enhancer (see, e.g., Queen, et al., Immunol. Rev. 89:49-68, 1986), and necessary processing information sites, such as ribosome binding sites, RNA splice sites, polyadenylation sites, and transcriptional terminator sequences.

These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts. (See generally Sambrook, et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express FXIa-binding antibody chains or binding fragments can be prepared using expression vectors of the present disclosure which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

Accordingly, in another aspect, the present disclosure provides a method for preparing an anti-FXI/FXIa antibody binding agent (e.g., antibody NOV1401 binding agent) optimized for expression in a mammalian cell consisting of: a full length heavy chain antibody sequence having a sequence selected from those provided in Table 2; and a full length light chain antibody sequence having a sequence selected from those provided in Table 2; altering at least one amino acid residue within the full length heavy chain antibody sequence and/or the full length light chain antibody sequence to create at least one altered antibody sequence; and expressing the altered antibody sequence as a protein. In one embodiment, the alteration of the heavy or light chain is in the framework region of the heavy or light chain.

The altered antibody sequence can also be prepared by screening antibody libraries having fixed CDR3 sequences or minimal essential binding determinants as described in US2005/0255552 and diversity on CDR1 and CDR2 sequences. The screening can be performed according to any screening technology appropriate for screening antibodies from antibody libraries, such as phage display technology.

Standard molecular biology techniques can be used to prepare and express the altered antibody sequence. The antibody encoded by the altered antibody sequence(s) is one that retains one, some or all of the functional properties of anti-FXI/FXIa-antibody binding agents (e.g., antibody NOV1401 binding agents) described herein, which functional properties include, but are not limited to, specifically binding an anti-FXI/FXIa antibody (e.g., antibody NOV1401), for example, and contacting the one or more CDR amino acid residues of the anti-FXI/FXIa; inhibiting binding of a target anti-FXI/FXIa antibody (e.g., antibody NOV1401) to human FXI and/or FXIa; inhibiting the ability of a target anti-FXI/FXIa antibody (e.g., antibody NOV1401) to block the activity of FXIa; and inhibiting or reversing one or more anticoagulant effects of a target anti-FXI/FXIa antibody (e.g., antibody NOV1401).

In certain embodiments of the methods of engineering antibodies of the present disclosure, mutations can be introduced randomly or selectively along all or part of an anti-FXI/FXIa antibody binding agent coding sequence and the resulting modified anti-FXI/FXIa antibody binding agents can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

In certain aspects of the present disclosure anti-FXI/FXIa antibody binding agents (e.g., antibody NOV1401 binding agent) have been engineered to remove sites of deamidation. Deamidation is known to cause structural and functional changes in a peptide or protein. Deamindation can result in decreased bioactivity, as well as alterations in pharmacokinetics and antigenicity of the protein pharmaceutical. (Anal Chem. 2005 Mar. 1; 77(5):1432-9).

In certain aspects of the present disclosure anti-FXI/FXIa antibody binding agents (e.g., antibody NOV1401 binding agent) described herein have been engineered to increase pI and improve their drug-like properties. The pI of a protein is a key determinant of the overall biophysical properties of a molecule. Antibodies and polypeptides that have low pIs have been known to be less soluble, less stable, and prone to aggregation. Further, the purification of antibodies and polypeptides with low pI is challenging and can be problematic especially during scale-up for clinical use. Increasing the pI of binding agents, such as antibodies, or Fabs, of the present disclosure improved their solubility, enabling the antibodies to be formulated at higher concentrations (>100 mg/ml). Formulation of the antibodies at high concentrations (e.g. >100 mg/ml) offers the advantage of being able to administer higher doses of the antibodies, which in turn may enable reduced dosing frequency, a significant advantage for treatment of chronic diseases including thrombotic and/or thromboembolic disorders. Higher pIs may also increase the FcRn-mediated recycling of the IgG version of the antibody thus enabling the drug to persist in the body for a longer duration, requiring fewer injections. Finally, the overall stability of the antibodies is significantly improved due to the higher pI resulting in longer shelf-life and bioactivity in vivo. In specific aspects, the pI of an anti-FXI/FXIa antibody binding agent is greater than or equal to 8.2.

The functional properties of the altered antibodies can be assessed using standard assays available in the art and/or described herein, such as those set forth in the Examples (e.g., ELISAs, aPTT assay, TGA assay).

### Prophylactic and Therapeutic Uses

The present disclosure relates to methods for reversing (e.g., partially reversing) or decreasing the anticoagulant effect of an anti-FXI/FXIa antibody (e.g., antibody NOV1401) in a patient being treated with the anti-FXI/FXIa antibody or antigen-binding fragment thereof, comprising administering an effective amount of a binding agent provided herein, e.g., a binding agent (e.g., antibody IDT11 or IDT12) which binds an anti-FXI/FXIa antibody and is capable of reversing one or more anticoagulant effects. In certain aspects, the present disclosure relates to methods for reversing (e.g., partially reversing) or decreasing the anticoagulant effect of an anti-FXI/FXIa antibody (e.g., antibody NOV1401) in a patient being treated with the anti-FXI/FXIa antibody or antigen-binding fragment thereof, comprising administering an effective amount of a pharmaceutical composition comprising a binding agent provided herein, e.g., a binding agent (e.g., antibody or antigen-binding fragment thereof as set forth in Table 2) which binds an anti-FXI/FXIa antibody and is capable of reversing one or more anticoagulant effects.

In specific aspects, reversal of the anticoagulant effects of an anti-FXI/FXIa antibody may be needed by a patient for emergency surgery/urgent procedures and in life-threatening or uncontrolled bleeding. In certain aspects, reversal (e.g., partial reversal) of the anticoagulant effects of an anti-FXI/FXIa antibody may be needed by a patient in the case of uncontrolled bleeding such as gastrointestinal (GI) bleeding, intracranial (IC) bleeding, or hemorrhagic stroke. In particular aspects, a patient is being treated with an anti-FXI/FXIa antibody to manage, treat, prevent, or reduce the risk of a thromboembolic disease or disorder, for example reducing the risk of stroke or thrombosis (e.g., systemic embolism) in patients with atrial fibrillation (e.g., non-valvular atrial fibrillation), chronic kidney disease, such as end stage renal failure (ESRD) undergoing hemodialysis. In further specific aspects, the patient has a demonstrated high risk of bleeding. In specific aspects, non-limiting examples of anti-FXI/FXIa antibody binding agents for use in these methods include antibodies (e.g., anti-idiotype antibodies) and antigen-binding fragments described herein, e.g., in Table 2, for example, antibodies IDT11 and IDT12.

In certain aspects, the present disclosure relates to methods for reducing clotting time in a subject administered an anti-FXI/FXIa antibody (e.g., antibody described in Table 1 such as antibody NOV1401), comprising administering an effective amount of a binding agent provided herein, e.g., a binding agent (e.g., anti-idiotype antibody or antigen-binding fragment thereof as set forth in Table 2) which binds the anti-FXI/FXIa antibody and is capable of inhibiting binding of the anti-FXI/FXIa antibody to human FXI/FXIa. In certain aspects, the present disclosure relates to methods for reducing clotting time in a subject administered an anti-FXI/FXIa antibody (e.g., antibody described in Table 1 such as antibody NOV1401), comprising administering an effective amount of a pharmaceutical compositions comprising a binding agent provided herein, e.g., a binding agent (e.g., anti-idiotype antibody or antigen-binding fragment thereof as set forth in Table 2) which binds the anti-FXI/FXIa antibody and is capable of inhibiting binding of the anti-FXI/FXIa antibody to human FXI/FXIa.

In specific aspects, the present disclosure relates to methods for managing bleeding or bleeding risk or for reducing bleeding or bleeding risk in a patient being treated with an anti-FXI/FXIa antibody (e.g., antibody described in Table 1 such as antibody NOV1401), comprising administering an effective amount of a binding agent provided herein, e.g., a binding agent (e.g., antibody or antigen-binding fragment thereof as described in Table 2) which binds an anti-FXI/FXIa antibody and is capable of reversing one or more anticoagulant effects, or administering an effective amount of a pharmaceutical composition comprising such binding agent provided herein. In specific aspects, reversal of the anticoagulant effects of an anti-FXI/FXIa antibody may be needed by a patient for emergency surgery/urgent procedures and in life-threatening or uncontrolled bleeding (e.g., GI bleeding, IC bleeding, or hemorrhagic stroke). In particular aspects, a patient is being treated with an anti-FXI/FXIa antibody to manage, treat, prevent, or reduce the risk of a thromboembolic disease or disorder, for example reducing the risk of stroke or thrombosis (e.g., systemic embolism) in patients with atrial fibrillation (e.g., non-valvular atrial fibrillation), chronic kidney disease, such as end stage renal failure (ESRD) undergoing hemodialysis. In further specific aspects, the patient has a demonstrated high risk of bleeding. In specific aspects, non-limiting examples of anti-FXI/FXIa antibody binding agents for use in these methods include antibodies (e.g., anti-idiotype antibodies and fragments thereof such as Fabs) and antigen-binding fragments described herein, e.g., in Table 2, for example, antibodies IDT11 and IDT12; antibodies comprising VH CDRs and VL CDRs of such antibodies; antibodies that bind the same epitope(s) within target antibody NOV1401 as such antibodies.

In a particular aspect, provided herein are methods of managing bleeding or bleeding risk in a patient treated or administered an anti-FXI antibody described herein (e.g., antibody described in Table 1 such as NOV1401 or an anti-FXI antibody comprising HCDRs and LCDRs of NOV1401), comprising the step of administering to the patient in need thereof, an anti-idiotype antibody, or antigen binding fragment thereof (e.g., Fab), of the anti-FXI antibody, wherein the anti-idiotype or antigen binding fragment thereof (e.g., Fab) specifically binds to the anti-FXI antibody and blocks the anti-FXI antibody from binding to FXI. In specific embodiments, an anti-idiotype antibody (e.g., IDT11 or IDT12) or antigen binding fragment thereof reverses the effects of an anti-FXI antibody described herein to mitigate bleeding risks, for example during urgent major surgery or trauma.

In specific aspects, an anti-idiotype antibody or antigen binding fragment thereof reverses (e.g., partially reverses) or inhibits an anti-FXI antibody's anti-coagulant effects. In particular aspects, the anti-idiotype antibody or antigen binding fragment thereof is administered to a patient in need thereof to temporarily reverse the anti-coagulant effect of an anti-FXI antibody described herein (e.g., antibody described in Table 1 such as NOV1401 or an anti-FXI antibody comprising HCDRs and LCDRs of NOV1401).

In a particular aspect, provided herein are methods of managing bleeding or bleeding risk in a patient treated or administered an anti-FXI antibody such as NOV1401 (e.g., SEQ ID NOs: 14 and 25), comprising the step of administering to the patient in need thereof, an anti-idiotype antibody (e.g., IDT11 or IDT12), or antigen binding fragment thereof, of the anti-FXI antibody such as NOV1401 (e.g., SEQ ID NOs: 14 and 25), wherein the anti-idiotype, or antigen binding fragment thereof (e.g., Fab), specifically binds to the antigen-binding region of an anti-FXI antibody such as NOV1401 (e.g., SEQ ID NOs: 14 and 25) and blocks the anti-FXI antibody from binding to FXI and/or FXIa. In a specific embodiment, the anti-idiotype antibody (e.g., IDT11 or IDT12), or antigen binding fragment thereof, of an anti-FXI antibody such as NOV1401 (e.g., SEQ ID NOs: 14 and 25) reverses or inhibits one or more of the anti-coagulant effects of the anti-FXI antibody (e.g., NOV1401). In certain embodiments, a temporary reversal or inhibition of one or more of the anti-coagulant effects of the anti-FXI antibody (e.g., NOV1401) is achieved. In specific embodiments, following the temporary reversal or inhibition of the anti-FXI antibody (e.g., NOV1401), the anti-FXI antibody (e.g., NOV1401) is again administered to the patient.

As used herein, the terms “effective amount” or “therapeutically effective amount” refer to an amount of a therapy (e.g., a binding agent provided herein such as an anti-idiotype antibody that binds an anti-FXI/FXIa antibody (e.g., NOV1401) or a pharmaceutical composition provided herein) which is sufficient to reduce and/or ameliorate the severity and/or duration of a given condition, disorder, or disease and/or a symptom related thereto. These terms also encompass an amount necessary for the reduction, slowing, or amelioration of the advancement or progression of a given condition, disorder, or disease, reduction, slowing, or amelioration of the recurrence, development or onset of a given condition, disorder or disease, and/or to improve or enhance the prophylactic or therapeutic effect(s) of another therapy (e.g., a therapy other than an anti-FXI/FXIa antibody binding agent provided herein). In some aspects, “effective amount” as used herein also refers to the amount of an antibody described herein to achieve a specified result, for example, reduction or reversal in one or more anticoagulant effects (e.g., aPTT prolongation, and reduction in the amount of thrombin in a thrombin generation assay (TGA) in human plasma) of a target anti-FXI/FXIa antibody; and reduction in, or blocking, binding of a target anti-FXI/FXIa antibody to FXI/FXIa.

In specific aspects, a patient, who may be in need of, or may benefit from, the methods described herein (e.g., methods for reversing anticoagulant effects with anti-FXI/FXIa antibody binding agents), has been treated with an anti-FXI/FXIa antibody (e.g., antibody described in Table 1 such as antibody NOV1401) to manage, treat, prevent, or reduce the risk of a thromboembolic disease or disorder, e.g., thrombic stroke, atrial fibrillation, stroke prevention in atrial fibrillation (SPAF), deep vein thrombosis, venous thromboembolism, pulmonary embolism, acute coronary syndromes (ACS), ischemic stroke, acute limb ischemia, chronic thromboembolic pulmonary hypertension, or systemic embolism. In further specific aspects, the patient has a demonstrated high risk of bleeding.

In other aspects, a patient, who may be in need of, or may benefit from, the methods described herein (e.g., methods for reversing anticoagulant effects with anti-FXI/FXIa antibody binding agents), has been treated with an anti-FXI/FXIa antibody (e.g., antibody described in Table 1 such as antibody NOV1401) for treatment of acute VTE, primary and extended secondary prevention of VTE, prevention of major adverse thromboembolic events in patient undergoing dialysis (with or without AF), prevention of major cardiovascular and cerebral events (MACCE) in patients with CAD undergoing PCI and receiving single or dual antiplatelet therapy, post-acute coronary syndromes (ACS) patients, heparin induced thrombocytopenia (HIT), prevention of thromboembolic events in heart failure patients and secondary stroke prevention.

In specific aspects, one of the following groups of subjects is being treated with an anti-FXI/FXIa antibody (e.g., antibody described in Table 1 such as antibody NOV1401) and may be in need of, or benefit from, the methods described herein (e.g., methods for reversing anticoagulant effects with anti-FXI/FXIa antibody binding agents):
Subjects with indications for chronic anticoagulation therapy (e.g., AF, left ventricular thrombus, prior cardioembolic stroke)
subjects at intermediate-to-high risk for major bleeding;
subjects undergoing elective or primary percutaneous coronary intervention (PCI) with stenting which may be require to receive dual antiplatelet therapy (aspirin and P2Y12 receptor antagonists) to prevent stent thrombosis.

In specific aspects, a subject, who may be in need of, or benefit from, the methods described herein (e.g., methods for reversing anticoagulant effects with anti-FXI/FXIa antibody binding agents), has been treated with an anti-FXI/FXIa antibody (e.g., antibody described in Table 1 such as antibody NOV1401) to manage, treat, prevent, or reduce the risk of one of the following conditions:
thromboembolism in subjects with suspected or confirmed cardiac arrhythmia such as paroxysmal, persistent or permanent atrial fibrillation or atrial flutter;
stroke prevention in atrial fibrillation (SPAF), a subpopulation of which is AF patients undergoing percutaneous coronary interventions (PCI);
acute venous thromboembolic events (VTE) treatment and extended secondary VTE prevention in patients at high risk for bleeding;
cerebral and cardiovascular events in secondary prevention after transient ischemic attack (TIA) or non-disabling stroke and prevention of thromboembolic events in heart failure with sinus rhythm;
clot formation in left atrium and thromboembolism in subjects undergoing cardioversion for cardiac arrhythmia;
thrombosis before, during and after ablation procedure for cardiac arrhythmia;
venous thrombosis, this includes but not exclusively, treatment and secondary prevention of deep or superficial veins thrombosis in the lower members or upper member, thrombosis in the abdominal and thoracic veins, sinus thrombosis and thrombosis of jugular veins;
thrombosis on any artificial surface in the veins like catheter or pacemaker wires;
pulmonary embolism in patients with or without venous thrombosis;
Chronic Thromboembolic Pulmonary Hypertension (CTEPH);
arterial thrombosis on ruptured atherosclerotic plaque, thrombosis on intra-arterial prosthesis or catheter and thrombosis in apparently normal arteries, this includes but not exclusively acute coronary syndromes, ST elevation myocardial infarction, non ST elevation myocardial infarction, unstable angina, stent thrombosis, thrombosis of any artificial surface in the arterial system and thrombosis of pulmonary arteries in subjects with or without pulmonary hypertension;
thrombosis and thromboembolism in patients undergoing percutaneous coronary interventions (PCI);
cardioembolic and cryptogenic strokes;
thrombosis in patients with invasive and non-invasive cancer malignancies;
thrombosis over an indwelling catheter;
thrombosis and thromboembolism in severely ill patients;
cardiac thrombosis and thromboembolism, this includes but not exclusively cardiac thrombosis after myocardial infarction, cardiac thrombosis related to condition such as cardiac aneurysm, myocardial fibrosis, cardiac enlargement and insufficiency, myocarditis and artificial surface in the heart;
thromboembolism in patients with valvular heart disease with or without atrial fibrillation;
thromboembolism over valvular mechanic or biologic prostheses;
injuries or trauma in patients who had native or artificial cardiac patches, arterial or venous conduit tubes after heart repair of simple or complex cardiac malformations;
venous thrombosis and thromboembolism after knee replacement surgery, hip replacement surgery, and orthopedic surgery, thoracic or abdominal surgery;
arterial or venous thrombosis after neurosurgery including intracranial and spinal cord interventions;
congenital or acquired thrombophilia including but not exclusively factor V Leiden, prothrombin mutation, antithrombin III, protein C and protein S deficiencies, factor XIII mutation, familial dysfibrinogenemia, congenital deficiency of plasminogen, increased levels of factor XI, sickle cell disease, antiphospholipid syndrome, autoimmune disease, chronic bowel disease, nephrotic syndrome, hemolytic uremia, myeloproliferative disease, disseminated intra vascular coagulation, paroxysmal nocturnal hemoglobinuria and heparin induced thrombopenia;
thrombosis and thromboembolism in chronic kidney disease;
thrombosis and thromboembolism in end stage renal disease (ESRD);
thrombosis and thromboembolism in patients with chronic kidney disease or ESRD undergoing hemodialysis; and
thrombosis and thromboembolism in patients undergoing hemodialysis and/or extra-corporal membrane oxygenation.

In a specific aspect, an anti-FXI/FXIa antibody binding agent (e.g., IDT11 or IDT12) or a pharmaceutical composition comprising such binding agent is for use in methods of reducing bleeding or bleeding risk, or managing bleeding or bleeding risk, in a patient being treated or administered an anti-FXI/FXIa antibody (e.g., antibody described in Table 1 such as antibody NOV1401) to reduce the risk of stroke and/or systemic embolism, wherein the patient has non-valvular atrial fibrillation.

In a specific aspect, an anti-FXI/FXIa antibody binding agent (e.g., IDT11 or IDT12) or a pharmaceutical composition comprising such binding agent is for use in methods of reducing bleeding or bleeding risk, or managing bleeding or bleeding risk, in a patient being treated or administered an anti-FXI/FXIa antibody (e.g., antibody described in Table 1 such as antibody NOV1401) to reduce the risk of stroke and/or systemic embolism, wherein the patient has non-valvular atrial fibrillation with a demonstrated high risk of bleeding.

In a specific aspect, an anti-FXI/FXIa antibody binding agent (e.g., IDT11 or IDT12) or a pharmaceutical composition comprising such binding agent is for use in methods of reducing bleeding or bleeding risk, or managing bleeding or bleeding risk, in a patient being treated or administered an anti-FXI/FXIa antibody (e.g., antibody described in Table 1 such as antibody NOV1401) to reduce the risk of stroke and/or systemic embolism, wherein the patient has ESRD and is undergoing dialysis.

In a specific aspect, an anti-FXI/FXIa antibody binding agent (e.g., IDT11 or IDT12) or a pharmaceutical composition comprising such binding agent is for use in methods of reducing bleeding or bleeding risk, or managing bleeding or bleeding risk, in a patient being treated or administered an anti-FXI/FXIa antibody (e.g., antibody described in Table 1 such as antibody NOV1401) to reduce the risk of stroke and/or systemic embolism, wherein the patient has non-valvular atrial fibrillation and ESRD and is undergoing dialysis.

In specific aspects, a subject, who may be in need of, or benefit from, the methods described herein (e.g., methods for reversing anticoagulant effects with anti-FXI/FXIa antibody binding agents), has been treated with an anti-FXI/FXIa antibody (e.g., antibody described in Table 1 such as antibody NOV1401) in combination with other agents for the prevention, treatment, or improvement of thromboembolic disorders. For example, statin therapies may be used in combination with the FXIa antibodies and antigen binding fragments of the present disclosure for the treatment of patients with thrombotic and/or thromboembolic disorders. Such subjects undergoing combination therapy may be in need of, or benefit from, the methods described herein (e.g., methods for reversing anticoagulant effects with anti-FXI/FXIa antibody binding agents).

In a specific aspect, provided herein are methods of reducing bleeding or bleeding risk, or managing bleeding or bleeding risk, in a patient being treated or administered an anti-FXI/FXIa antibody (e.g., antibody described in Table 1 such as antibody NOV1401), said method comprises administering a binding agent which specifically binds to the anti-FXI/FXIa antibody (e.g., antibody NOV1401), and reverses an anticoagulant effect of the anti-FXI/FXIa antibody. In particular aspects, the bleeding or bleeding risk is associated with trauma, surgery, or post-delivery. In another particular aspect, the bleeding or bleeding risk is associated with emergency surgery or urgent procedures. In other particular aspects, the bleeding is life-threatening or uncontrolled, such as GI bleed or IC bleed. In specific aspects, the binding agent is an antibody, such as an anti-idiotype antibody (e.g., IDT11 or IDT12) which specifically binds an anti-FXI/FXIa antibody (e.g., NOV1401). In additional specific aspects, the binding agent is an anti-idiotype antibody which specifically binds to one or more epitopes within the variable regions of an anti-FXI/FXIa antibody (e.g., NOV1401). In more specific aspects, the binding agent is a full length IgG anti-idiotype antibody which specifically binds to an anti-FXI/FXIa antibody (e.g., NOV1401). In particular aspects, the binding agent is an anti-idiotype antibody or antigen-binding fragment thereof comprising amino acid sequences selected from Table 2. In particular aspects, the binding agent is an anti-idiotype antibody or antigen-binding fragment thereof, such as antibody IDT11 or IDT12, as set forth in Table 2. In particular aspects, the binding agent is an anti-idiotype antibody or antigen-binding fragment thereof, such as IDT11, as set forth in Table 2. In particular aspects, the binding agent is an anti-idiotype antibody or antigen-binding fragment thereof, such as IDT12, as set forth in Table 2.

In specific aspects, bleeding is typically associated with, but not limited to, trauma, surgery, menstruation or post-delivery. Therefore, under these circumstances, a subject, who has been treated with an anti-FXI/FXIa antibody (e.g., antibody described in Table 1 such as NOV1401), may be in need of quick and effective therapy, such as an anti-FXI/FXIa antibody binding agent described herein, to reduce bleeding or to reduce bleeding risk. In specific aspects, prolonged bleeding may occur after a major trauma or after surgery, such as surgery involving organs with high fibrinolytic area such as buccal, nasal, genital or urinary mucosa. Tooth extraction, tonsillectomy and ablation of the uterus or prostate are more non-limiting examples of surgeries that entail a high risk of bleeding. In specific aspects, concomitant use of antiplatelets, other anticoagulants and fibrinolytic agents can increase the risk of bleeding.

In certain aspects, a temporary reversal or inhibition of one or more of the anticoagulant effects of an anti-FXI antibody (e.g., antibody described in Table 1 such as antibody NOV1401) is desired. In a particular aspect, provided herein are methods of reducing or managing bleeding or bleeding risk in a patient treated or administered an anti-FXI/FXIa antibody such as antibody NOV1401, comprising the step of administering to the patient in need thereof, a pharmaceutical composition comprising a binding agent described herein, such as antibody IDT1, IDT2, IDT3, IDT4, IDT5, IDT6, IDT7, IDT8, IDT9, or IDT10, once or twice, over a period of time (e.g., 1 hour to 24 hours or to 48 hours), followed by administering the anti-FXI/FXIa antibody, wherein a temporary reversal or inhibition of one or more of the anticoagulant effects of the anti-FXI antibody is achieved. In a particular aspect, provided herein are methods of reducing or managing bleeding or bleeding risk in a patient treated or administered an anti-FXI/FXIa antibody such as antibody NOV1401, comprising the step of administering to the patient in need thereof, IDT11 or IDT12 or a pharmaceutical composition comprising IDT11 or IDT12, once or twice or more, over a period of time (e.g., 1 hour to 24 hours or to 48 hours), followed by administering the anti-FXI/FXIa antibody, wherein a temporary reversal or inhibition of one or more of the anticoagulant effects of the anti-FXI antibody is achieved.

In certain aspects, an anti-FXI/FXIa antibody binding agent described herein (e.g., IDT11 or IDT12) can be administered in combination with another anticoagulant reversal therapy. Non-limiting examples of conventional strategies for reversing anticoagulant effects include (i) fluid replacement using colloids, crystalloids, human plasma or plasma proteins such as albumin; or (ii) transfusion with packed red blood or whole blood. Examples of therapies for reversal of the effects of anticoagulants, for example, in cases of severe emergency, include, but are not limited to, prohemostasis blood components such as fresh frozen plasma (FFP), prothrombin complex concentrates (PCC) and activated PCC [(APCC); e.g. factor VIII inhibitor bypass activity (FEIBA)] and recombinant activated factor VII (rFVIIa).

In specific aspects, the present disclosure relates to methods for reversing the anticoagulant effect of an anti-FXI/FXIa antibody (e.g., antibody described in Table 1 such as antibody NOV1401) in a patient being treated with the anti-FXI/FXIa antibody or antigen-binding fragment thereof, comprising (i) administering to the patient an effective amount of a binding agent provided herein, e.g., a binding agent (e.g., IDT11 or IDT12) which binds an anti-FXI/FXIa antibody and is capable of reversing one or more anticoagulant effects; and (ii) administering to the patient another anticoagulant reversal therapy, such as fresh frozen plasma (FFP), prothrombin complex concentrates (PCC), activated PCC or recombinant activated factor VII (rFVIIa). In specific aspects, the present disclosure relates to methods for reversing the anticoagulant effect of an anti-FXI/FXIa antibody (e.g., antibody NOV1401) in a patient being treated with the anti-FXI/FXIa antibody or antigen-binding fragment thereof, comprising (i) administering to the patient an effective amount of a binding agent provided herein, e.g., a binding agent (e.g., antibody or antigen-binding fragment thereof, such as a Fab fragment) which binds an anti-FXI/FXIa antibody and is capable of reversing one or more anticoagulant effects; and (ii) administering to the patient fresh frozen plasma (FFP). In specific aspects, such method achieves homeostasis.

In certain aspects, provided herein is a method of managing bleeding in a patient being treated with an anti-FXI antibody provided herein (e.g., an antibody described in Table 1, such as, an anti-FXI antibody comprising VL CDRs and VHCDRs of NOV1401), said method comprises temporarily reversing of the anticoagulant effect for a sufficient time to manage the bleeding. In specific embodiments, the step of reversing of the anticoagulant effect comprises (i) fluid replacement using colloids, crystalloids, human plasma or plasma proteins such as albumin; or (ii) transfusion with packed red blood or whole blood. In specific aspects, therapeutic agents for reversal of the effect of anticoagulants, for example, in cases of severe emergency, include, but are not limited to, prohemostasis blood components such as fresh frozen plasma (FFP), prothrombin complex concentrates (PCC) and activated PCC (APCC) (e.g. factor VIII inhibitor bypass activity (FEIBA)), and recombinant activated factor VII (rFVIIa). In one particular aspect, a regimen may comprise administration of rFVIIa at a dose of 30 μg/kg followed by administration of rFVIIa at a dose of 15-30 μg/kg every 2-4 hours for 24-48 hours in addition to tranexamic acid 1 g every 6 hours for 5 to 7 days may have potential to recover hemostasis and stop bleeding in subjects treated with an anti-FXI antibody (e.g., NOV1401 or an antibody comprising VL CDRs and VH CDRs of NOV1401) who are undergoing major surgery and in patients with an active non-accessible bleeding site. For instance, Riddell et al reported experience in 4 patients with severe FXI deficiency without inhibitor undergoing surgery (Riddell et al., 2011, Thromb. Haemost., 106: 521-527); patients were administered rFVIIa 30 g/kg and tranexamic acid 1 g i.v. at induction of anesthesia. Subsequent bolus doses of rFVIIa 15-30 μg/kg were administered at 2 to 4 hourly intervals as guided by rotational thromboelastometry (ROTEM) results. In specific examples, patients were treated with rFVIIa at above mentioned doses for 24-48 hours. In particular examples, tranexamic acid 1 g every six-hourly was continued for five days. In this small series, rFVIIa at doses as low as 15-30 μg/kg in combination with tranexamic acid was safe and effective in correcting the hemostatic defect in severe FXI deficiency in this study. In another study comprising 4 patients with severe FXI deficiency with inhibitor (autologous neutralizing FXI antibodies usually acquired after transfusion or administration of blood products to patients with severe FXI deficiency) who experienced 5 surgeries, the authors (Livnat et al., 2009, Thromb. Haemost.; 102: 487-492) applied the following protocol: 1 g of tranexamic acid orally two hours before surgery, then patients received immediately prior to the interventions another 1 g tranexamic acid i.v. Recombinant FVIIa at doses ranging from 15 to 30 μg/kg was infused at the completion of surgery. Subsequently, oral tranexamic acid 1 g was given every 6 hour for at least 7 days. Fibrin glue was sprayed at the bed of the extirpated gallbladder in one patient. This protocol secured normal hemostasis in patients with severe FXI deficiency with inhibitor. In one aspect, fibrin glue can be used to restore local hemostasis during dental surgery in patients with FXI deficiency (Bolton-Maggs (2000) Haemophilia; 6 (S1):100-9). In a certain embodiment with respect to methods to manage bleeding in patients being treated with an anti-FXI antibody provided herein (e.g., antibody described in Table 1 such as NOV1401), a regimen consisting of tranexamic acid, for example, 1 g every 6 hours for 5 to 7 days associated with the use of fibrin glue could be used to establish local hemostasis in subjects undergoing minor surgery and in subjects with accessible bleeding site, including oral and nasal bleeding events.

In certain aspects, provided herein is a method of managing bleeding or bleeding risk in a patient being treated with an anti-FXI/FXIa antibody provided herein (e.g., an antibody described in Table 1, such as, NOV1401 or an anti-FXI/FXIa antibody comprising VL CDRs and VHCDRs of NOV1401), said method comprising administering to the patient an anticoagulant reversal therapy capable of reversing (e.g., partially reversing) the anticoagulant effects of the anti-FXI/FXIa antibody. In specific aspects, the anticoagulant reversal therapy capable of reversing the anticoagulant effect of the anti-FXI/FXIa antibody is rFVIIa (recombinant Factor VIIa), emicizumab (ACE910), tranexamic acid, Fresh Frozen Plasma (FFP), Hemoeleven, Prothrombin Complex Concentrate (PCC), Activated PCC, or FEIBA (a FVIII inhibitor complex). In specific aspects, the anticoagulant reversal therapy is administered alone, or in combination with a binding agent provided herein (e.g., binding agent described in Table 2, such as IDT11 or IDT12) or a pharmaceutical composition comprising such binding agent.

In specific aspects, the present disclosure relates to methods for reversing (e.g., partially reversing) the anticoagulant effect of an anti-FXI/FXIa antibody (e.g., an anti-FXI/FXIa antibody described in Table 1 such as antibody NOV1401 or an anti-FXI/FXIa antibody comprising VH CDRs and VL CDRs of NOV1401) in a patient being treated with the anti-FXI/FXIa antibody or antigen-binding fragment thereof, comprising administering to the patient an anticoagulant reversal therapy, such as rFVIIa (recombinant Factor VIIa), emicizumab (ACE910), tranexamic acid, Fresh Frozen Plasma (FFP), Hemoeleven, Prothrombin Complex Concentrate (PCC), Activated PCC, or FEIBA (a FVIII inhibitor complex).

In specific aspects, the present disclosure relates to methods for reversing the anticoagulant effect of an anti-FXI/FXIa antibody (e.g., an anti-FXI/FXIa antibody described in Table 1 such as antibody NOV1401 or an anti-FXI/FXIa antibody comprising VH CDRs and VL CDRs of NOV1401) in a patient being treated with the anti-FXI/FXIa antibody or antigen-binding fragment thereof, comprising (i) administering to the patient an effective amount of a binding agent provided herein, e.g., a binding agent (e.g., antibody or antigen-binding fragment thereof as set forth in Table 2 such as IDT11 or IDT12) which binds an anti-FXI/FXIa antibody and is capable of reversing one or more anticoagulant effects or a pharmaceutical composition comprising such binding agent; and (ii) administering to the patient another anticoagulant reversal therapy, such as rFVIIa (recombinant Factor VIIa), emicizumab (ACE910), tranexamic acid, Fresh Frozen Plasma (FFP), Hemoeleven, Prothrombin Complex Concentrate (PCC), Activated PCC, or FEIBA (a FVIII inhibitor complex).

In specific aspects, the risk of thromboembolic events including stroke, systemic embolism, coronary or peripheral artery thrombosis, venous thrombosis and pulmonary embolism increases with presence of predisposing factors such as thrombophilia, vessel wall damage and stasis. Evaluation of medical history, familiar antecedents and associated comorbidities can help to stratify patients according to their thromboembolic risks. In patients with atrial fibrillation, several scoring systems e.g., CHADS2 and CHA2DS2-VASc have been developed to assess stroke risk. Each was developed based on data from randomized trials, and clinical and epidemiologic cohort studies, and translated a weighted, multivariate formula of stroke risk factors to a simplified, easy-to-use mnemonic device, algorithm, calculator, or online tool. The CHADS2 risk score was used stratification tool to predict thromboembolic risk in atrial fibrillation patients (Lip (2011) Am J Med; 124(2): 111-4; Camm et al (2012) Eur Heart J; 33: 2719-2747); however, accumulated evidence shows that CHA2DS2-VASc is at least as good as or possibly better than, scores such as CHADS2 in identifying patients who develop stroke and thromboembolism and definitively better at identifying ‘truly low-risk’ patients with atrial fibrillation. The CHA2DS2-VASc score is presently recommended by Guidelines (Camm et al (2012) Eur Heart J 33, 2719-2747; January et al, AHA/ACC/HRS Atrial Fibrillation Guideline; J Am Coll Cardiol 2014; 64:2246-80) to guide the decision with regard to patients who should benefit of anticoagulant therapy and also to identify low risk patients in whom anticoagulation therapy is not warranted.

Bleeding risk assessment tools specific to the atrial fibrillation patients e.g., HAS-BLED, ATRIA, HEMORR2HAGES; ORBIT and ABC risk score were developed to predict the bleeding risk in patients with atrial fibrillation. Unfortunately, as the bleeding risk is tightly correlated with the stroke risk, those risk score were of rather limited value to guide therapeutic decisions to use vitamin K antagonists such as warfarin or NOACS. However, bleeding risk scores may become of considerable help to identify patients who can benefit of a new therapy with a reduced bleeding risk e.g. anti-FXI/FXIa antibody (e.g., antibody NOV1401).

In certain aspects, subjects with a bleeding risk, for example a demonstrated high risk of bleeding, may be identified by previous medical history of bleeding, for example, bleeding during or after surgery or bleeding when treated with an anticoagulant (e.g. Warfarin). In addition, subjects with a bleeding risk, for example a demonstrated high risk of bleeding, may be identified by in vitro/ex vivo assays known in the art, for example, assays with a subject's plasma measuring aPTT and other biomarkers of the extrinsic coagulation pathways, such as prothrombin time (PT) and thrombin time (TT).

In particular aspects, subjects with moderate to high risk for stroke and systemic embolism have a CHA2DS2VASc risk score ≥2. In further particular aspects, subjects with a HAS BLED risk score ≥3 is characterized as having a high risk of bleeding (see Gallego, et al., (2012) Carc Arrhythm Electrophysiol.; 5:312-318, and Friberg et al., (2012) Circulation.; 125:2298-2307). In particular aspects, subjects being treated by the methods provided herein has a CHA2DS2VASc risk score ≥2.

In specific aspects, a subject being treated by the methods provided herein is a human subject at least 18 years old. In another aspect, a subject being treated by the methods provided herein is a human subject at least 50 years old. In another aspect, a subject being treated by the methods provided herein is a human subject at least 55 years old. In another aspect, a subject being treated by the methods provided herein is a human subject at least 60 years old. In another aspect, a subject being treated by the methods provided herein a human subject is at least 65 years old.

In particular aspects, a subject being treated by the methods provided herein (e.g., methods for treating VTE or for secondary prevention of VTE) is between the age of 2 and 18 years old. In particular aspects, a subject being treated by the methods provided herein (e.g., methods for treating VTE or for secondary prevention of VTE) is between the age of 12 and 18 years old. In particular aspects, a subject being treated by the methods provided herein (e.g., methods for treating VTE or for secondary prevention of VTE) is a child at least 2 years old and under 18 years old. In particular aspects, a subject being treated by the methods provided herein (e.g., methods for treating VTE or for secondary prevention of VTE) is a child at least 12 years old and under 18 years old.

In specific aspects, a subject (e.g., human subject) being treated by the methods provided herein has a body mass index (BMI) that is greater than or equal to 18 kg/m². In another aspect, a subject being treated by the methods provided herein has a BMI that is greater than or equal to 30 kg/m². In another aspect, a subject being treated by the methods provided herein has a BMI that is greater than or equal to 35 kg/m². In another aspect, a subject being treated by the methods provided herein has a BMI that is greater than or equal to 40 kg/m².

In certain aspects, methods for reversing the anticoagulant effects of an anti-FXI/FXIa antibody (e.g., antibody described in Table 1 such as antibody NOV1401) with an anti-FXI/FXIa antibody binding agent described herein (e.g., IDT11 or IDT12) or a pharmaceutical composition comprising such anti-FXI/FXIa antibody binding agent, results in (i) reduction or reversal in aPTT prolongation as determined with aPTT assays with human plasma; (ii) reduction in the amount of thrombin in a thrombin generation assay (TGA) amount of thrombin in a thrombin generation assay (TGA) in human plasma; and/or (iii) reduction or reversal of bleeding or bleeding risk. In specific aspects, reversal of the anticoagulant effects is less than 100%, but is sufficient to achieve a clinically beneficial outcome, e.g., reduction or stop in bleeding.

In certain aspects, methods for reversing the anticoagulant effect of an anti-FXI/FXIa antibody (e.g., antibody described in Table 1 such as antibody NOV1401) with an anti-FXI/FXIa antibody binding agent described herein (e.g., IDT11 or IDT12) or a pharmaceutical composition comprising such anti-FXI/FXIa antibody binding agent, results in reduction or reversal in aPTT prolongation as determined with aPTT assays with human plasma, by at least 10%, at least 20%, at least 30%, at least 40%, at least 50%, at least 60%, at least 70%, at least 80%, or at least 90%. In certain aspects, methods for reversing the anticoagulant effect of an anti-FXI/FXIa antibody (e.g., antibody described in Table 1 such as antibody NOV1401) with an anti-FXI/FXIa antibody binding agent described herein (e.g., IDT11 or IDT12) or a pharmaceutical composition comprising such anti-FXI/FXIa antibody binding agent, results in reduction or reversal in aPTT prolongation as determined with aPTT assays with human plasma, by at least 40%, at least 50%, at least 60%, or at least 70%.

In certain aspects, methods for reversing the anticoagulant effect of an anti-FXI/FXIa antibody (e.g., antibody described in Table 1 such as antibody NOV1401) with an anti-FXI/FXIa antibody binding agent described herein (e.g., antibody as set forth in Table 2 such as IDT11 or IDT12) or a pharmaceutical composition comprising such anti-FXI/FXIa antibody binding agent, results in an increase in the serum level of free FXI/FXIa relative to levels prior to administration of the anti-FXI/FXIa antibody binding agent. In certain aspects, methods for reversing the anticoagulant effect of an anti-FXI/FXIa antibody (e.g., antibody described in Table 1 such as antibody NOV1401) with an anti-FXI/FXIa antibody binding agent described herein (e.g., antibody as set forth in Table 2 such as IDT11 or IDT12) or a pharmaceutical composition comprising such anti-FXI/FXIa antibody binding agent, results in an increase in the serum level of free FXI/FXIa relative to levels prior to administration of the anti-FXI/FXIa antibody binding agent, by at least 10%, at least 20%, at least 30%, at least 40%, at least 50%, at least 60%, at least 70%, at least 80%, or at least 90%. In certain aspects, methods for reversing the anticoagulant effect of an anti-FXI/FXIa antibody (e.g., antibody described in Table 1 such as antibody NOV1401) with an anti-FXI/FXIa antibody binding agent described herein (e.g., antibody as set forth in Table 2 such as IDT11 or IDT12) or a pharmaceutical composition comprising such anti-FXI/FXIa antibody binding agent, results in an increase in the serum level of free FXI/FXIa relative to levels prior to administration of the anti-FXI/FXIa antibody binding agent, by at least 40%, at least 50%, at least 60%, or at least 70%. The serum level of free FXI/FXIa can be determined by any methods previously described, e.g., by ELISA.

### Pharmaceutical Compositions

The present disclosure provides pharmaceutical compositions comprising anti-FXI/FXIa antibody-binding agents described herein (e.g., antibody described in Table 2 and Fab fragments thereof) formulated together with a pharmaceutically acceptable carrier. The compositions can additionally contain one or more other therapeutic agents that are suitable for treating or preventing, for example, thromboembolic disorders (e.g., thrombotic disorders). Pharmaceutically acceptable carriers enhance or stabilize the composition, or can be used to facilitate preparation of the composition. Pharmaceutically acceptable carriers include solvents, dispersion media, coatings, antibacterial and antifungal agents, isotonic and absorption delaying agents, and the like that are physiologically compatible.

A pharmaceutical composition of the present disclosure can be administered by a variety of methods known in the art. The route and/or mode of administration vary depending upon the desired results. It is preferred that administration be intravenous, intramuscular, intraperitoneal, or subcutaneous, or administered proximal to the site of the target. The pharmaceutically acceptable carrier should be suitable for intravenous, intramuscular, subcutaneous, parenteral, spinal or epidermal administration (e.g., by injection or infusion). Depending on the route of administration, the active compound, i.e., antibody, bispecific and multispecific molecule, may be coated in a material to protect the compound from the action of acids and other natural conditions that may inactivate the compound.

In specific aspects, a composition should be sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

In particular aspects, provided herein is a pharmaceutical composition comprising an anti-FXI/FXIa antibody binding agent provided herein (e.g., antibody as set forth in Table 2), wherein the binding agent inhibits an anticoagulant activity of the target anti-FXI/FXIa antibody, and wherein the binding agent is in a liquid formulation comprising histidine and/or sugars such as sucrose.

In specific aspects, provided herein is a pharmaceutical composition comprising an anti-FXI/FXIa antibody binding agent provided herein (e.g., antibody as set forth in Table 2), wherein the binding agent inhibits an anticoagulant activity of the target anti-FXI/FXIa antibody, and wherein the binding agent is in a liquid formulation comprising sucrose. In particular aspects, the sucrose concentration in the liquid formulation is in the range of 150 mM to 300 mM or 200 mM to 250 mM. In particular aspects, the liquid formulation comprises at least 200, 210, 220, 230, 240, or 250 mM sucrose. In particular aspects, the sucrose concentration in the liquid formulation is 220 mM.

In specific aspects, provided herein is a pharmaceutical composition comprising an anti-FXI/FXIa antibody binding agent provided herein (e.g., antibody Fab or IgG as set forth in Table 2), wherein the binding agent inhibits an anticoagulant activity of the target anti-FXI/FXIa antibody, and wherein the binding agent is in a liquid formulation comprising histidine. In specific aspects, the histidine concentration in the liquid formulation is in the range of 5 mM to 35 mM or 10 mM to 30 mM. In specific aspects, the histidine concentration in the liquid formulation is in the range of 15 mM to 25 mM. In specific aspects, the liquid formulation comprises at least 10 mM histidine, or at least 15 mM histidine, or at least 20 mM histidine. In specific aspects, the liquid formulation comprises at least 20 mM histidine.

In specific aspects, provided herein is a pharmaceutical composition comprising an anti-FXI/FXIa antibody binding agent provided herein (e.g., antibody as set forth in Table 2), wherein the binding agent inhibits an anticoagulant activity of the target anti-FXI/FXIa antibody, and wherein the binding agent is in a liquid formulation comprising histidine and sucrose. In particular aspects, the sucrose concentration in the liquid formulation is in the range of 150 mM to 300 mM or 200 mM to 250 mM; and the histidine concentration in the liquid formulation is in the range of 5 mM to 35 mM or 10 mM to 30 mM or 15 mM to 25 mM. In particular aspects, the liquid formulation comprises at least 200, 210, 220, 230, 240, or 250 mM sucrose; and at least 10 mM histidine, or at least 15 mM histidine, or at least 20 mM histidine. In particular aspects, a liquid formulation comprises 220 mM sucrose and 20 mM histidine.

In particular aspects, provided herein is a pharmaceutical composition comprising an anti-FXI/FXIa antibody binding agent provided herein (e.g., antibody as set forth in Table 2), wherein the binding agent inhibits an anticoagulant activity of the target anti-FXI/FXIa antibody, and wherein the binding agent is in a liquid formulation with a pH in the range of 4 to 6.5, or 4.5 to 7, or 4.5 to 6. In certain aspects, a liquid formulation has a pH in the range of 5 to 6. In particular aspects, a liquid formulation has a pH of at least 4.0. In particular aspects, a liquid formulation has a pH of at least 4.5. In particular aspects, a liquid formulation has a pH of at least 5.0. In particular aspects, a liquid formulation has a pH of at least 5.5. In particular aspects, a liquid formulation has a pH of at least 6.6. In particular aspects, a liquid formulation has a pH of 5. In particular aspects, a liquid formulation has a pH of 5.5. In particular aspects, a liquid formulation has a pH of 6.0.

In specific aspects, provided herein is a pharmaceutical composition comprising an anti-FXI/FXIa antibody binding agent provided herein (e.g., antibody as set forth in Table 2), wherein the binding agent inhibits an anticoagulant activity of the target anti-FXI/FXIa antibody, and wherein the binding agent is in a liquid formulation at a concentration of approximately 50 mg/mL to 200 mg/mL or approximately 100 mg/mL to 200 mg/mL. In particular aspects, the binding agent is in a liquid formulation at a concentration of at least 50 mg/mL, at least 100 mg/mL, at least 110 mg/mL, at least 120 mg/mL, at least 130 mg/mL, at least 140 mg/mL, or at least 150 mg/mL. In particular aspects, the binding agent is in a liquid formulation at a concentration of 150 mg/mL. In particular aspects, the binding agent is in a liquid formulation at a concentration of 140 mg/mL. In particular aspects, the binding agent is in a liquid formulation at a concentration of 130 mg/mL.

In specific aspects, provided herein is a pharmaceutical composition comprising an anti-FXI/FXIa antibody binding agent provided herein (e.g., antibody as set forth in Table 2), wherein the binding agent inhibits an anticoagulant activity of the target anti-FXI/FXIa antibody, and wherein the binding agent is in a liquid formulation comprising Polysorbate 20, for example, 0.01% to 0.08% Polysorbate 20. In particular aspects, the liquid formulation comprises 0.02% to 0.06% Polysorbate 20. In particular aspects, the liquid formulation comprises approximately 0.03% Polysorbate 20, 0.04% Polysorbate 20, or 0.05% Polysorbate 20. In particular aspects, the liquid formulation comprises approximately 0.04% Polysorbate 20.

In specific aspects, provided herein is a pharmaceutical composition comprising an anti-FXI/FXIa antibody binding agent provided herein (e.g., antibody as set forth in Table 2), wherein the binding agent inhibits an anticoagulant activity of the target anti-FXI/FXIa antibody, wherein the binding agent is in a liquid formulation at a concentration of 150 mg/mL, and wherein the liquid formulation comprises 220 mM sucrose and 20 mM histidine at pH 5.5. In specific aspects, a pharmaceutical composition provided herein is for subcutaneous administration. In certain aspects, a pharmaceutical composition provided herein is for intravenous administration.

In specific aspects, provided herein is a pharmaceutical composition comprising an anti-FXI/FXIa antibody binding agent provided herein (e.g., antibody as set forth in Table 2), wherein the binding agent inhibits an anticoagulant activity of the target anti-FXI/FXIa antibody, wherein the binding agent is in a liquid formulation at a concentration of 150 mg/mL, and wherein the liquid formulation comprises 220 mM sucrose, 20 mM histidine, and 0.04% Polysorbate 20, at pH 5.5. In specific aspects, a pharmaceutical composition provided herein is for subcutaneous administration. In certain aspects, a pharmaceutical composition provided herein is for intravenous administration.

Pharmaceutical compositions of the present disclosure can be prepared in accordance with methods well known and routinely practiced in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the FXIa-binding antibody is employed in the pharmaceutical compositions of the present disclosure. The FXIa-binding antibodies are formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the present disclosure can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present disclosure employed, thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A physician can start doses of the antibodies of the present disclosure employed in the pharmaceutical composition at levels lower than that required to achieve the desired therapeutic effect and gradually increase the dosage until the desired effect is achieved. In general, effective doses of the compositions of the present disclosure, for the treatment of a thrombotic and/or thromboembolic disorders described herein vary depending upon many different factors, including means of administration, target site, physiological state of the patient, other medications administered, and whether treatment is prophylactic or therapeutic. Treatment dosages need to be titrated to optimize safety and efficacy. For systemic administration with an antibody, in certain aspects, the dosage may range from about 0.01 to 15 mg/kg of the host body weight. For administration with an antibody, the dosage may range from 0.1 mg to 500 mg.

In a certain aspect, an anti-FXI/FXIa antibody described herein is administered, for example by i.v. or s.c. route, at a dose in the range of 5 mg to 600 mg.

In a certain aspect, an anti-FXI/FXIa antibody described herein is administered, for example by i.v. or s.c. route, at a dose of approximately 5 mg, 10 mg, 15 mg, 20 mg, 30 mg, 40 mg, 50 mg, 60 mg, 90 mg, 100 mg, 120 mg, 150 mg, 180 mg, 200 mg, 210 mg, 240 mg, 250 mg, 270 mg, 300 mg, 330 mg, 350 mg, 360 mg, 390 mg, 400 mg, 420 mg, 450 mg, 480 mg, 500 mg, 510 mg, 540 mg, 550 mg, 570 mg, or 600 mg.

In particular aspects, an antibody is usually administered on multiple occasions. Intervals can also be irregular as indicated by measuring blood levels of antibody in the patient. In addition alternative dosing intervals can be determined by a physician and administered monthly or as necessary to be efficacious. In some methods of systemic administration, dosage is adjusted to achieve a plasma antibody concentration of 1-1000 μg/ml and in some methods 25-500 μg/ml. Dosage and frequency vary depending on the half-life of the antibody in the patient. In general, humanized antibodies show longer half-life than that of chimeric antibodies and nonhuman antibodies. The dosage and frequency of administration can vary depending on whether the treatment is prophylactic or therapeutic. In certain aspects for prophylactic applications, a relatively low dosage is administered at relatively infrequent intervals over a long period of time. Some patients continue to receive treatment for the rest of their lives. In certain aspects for therapeutic applications, a relatively high dosage at relatively short intervals is sometimes required until progression of the bleeding or bleeding risk is reduced or terminated, and in certain cases until the patient shows partial or complete amelioration of bleeding or risk of bleeding.

In specific aspects, an anti-FXI/FXIa binding agent described herein (e.g., antibody as set forth in Table 2 such as IDT11 or IDT12) is administered for a temporary duration or period of time when reversal of anticoagulant effects of an anti-FXI/FXIa antibody (e.g., antibody described in Table 1 such as NOV1401) is desired. In specific aspects, an anti-FXI/FXIa binding agent described herein (e.g., antibody as set forth in Table 2 such as IDT11 or IDT12) is administered once, or a few times, for a temporary duration or period of time (e.g., 1 hour to 24 hours or to 48 hours but generally not exceeding 7 days) when reversal of anticoagulant effects of an anti-FXI/FXIa antibody (e.g., antibody described in Table 1 such as NOV1401) is desired to achieve homeostasis.

### EXAMPLES

The following examples are provided to further illustrate the present disclosure but not to limit its scope. Other variants of the present disclosure will be readily apparent to one of ordinary skill in the art and are encompassed by the appended claims.

### Example 1

### Human Fab Phage Library Panning

### Phage Display Panning

Antibodies against NOV1401 were generated by the selection of clones that bound to NOV1401 using as a source of antibody a commercially available phage display library, the Morphosys HuCAL PLATINUM® library. The phagemid library is based on the HuCAL® concept (Knappik et al., 2000, J Mol Biol 296: 57-86) and employs the CysDisplay™ technology for displaying the Fab on the phage surface (WO01/05950). For the isolation of anti-NOV1401 antibodies a solid phase panning strategy was employed with direct coating of NOV1401 to a Maxisorp™ (Nunc) 96 well plate followed by three rounds of panning with increasing washing stringency.

### Subcloning and Microexpression of Selected Fab Fragments

To facilitate rapid expression of soluble Fab, the Fab encoding inserts of the selected HuCAL PLATINUM® phage were subcloned from pMORPH®30 display vector into pMORPH®x11 expression vector pMORPH®x11_FH.

For initial screening and characterization an overnight cultures of individual Fab-expressing E. coli clones were lysed using 2×BBS solution (400 mM Boric acid, 300 mM Sodium chloride, 5 mM EDTA) supplemented with 2.5 mg/mL lysozyme. Fab containing E. coli lysates were used for ELISA screening.

### ELISA Screening

Using ELISA screening, single Fab clones were identified from panning output for binding to NOV1401. Fabs were tested using Fab containing crude E. coli lysates.

For identification of NOV1401 binding Fab fragments Maxisorp™ (Nunc) 384 well plates were directly coated with 5 ug/ml NOV1401. After blocking of plates with Superblock®, Fab-containing E. coli lysates were added. Binding of Fabs was detected by F(ab)2 specific goat anti-human IgG conjugated to alkaline phosphatase (diluted 1:5000) using Attophos fluorescence substrate (Roche, catalogue #11681982001). Fluorescence emission at 535 nm was recorded with excitation at 430 nm.

### Engineering to Remove Potential Deamidation Sites

In order to remove potential liabilities in long term storage potential deamidation sites (e.g., Asn-Gly or Asn-Ser) were removed by replacing asparagine to serine or glutamine. Genes including the altered amino acids were generated via gene synthesis.

### Expression and Purification of HuCAL® Fab Fragments

Expression of Fab fragments was performed in E. coli TG1 F-cells. Cultures were incubated at 37° C. until the OD600 reached a value of 0.5. Fab expression was induced by addition of IPTG to a final concentration of 0.75 mM and cultures were further incubated o/n at 30° C. and 180 rpm. Cells were harvested and disrupted. His6-tagged Fab fragments were isolated via IMAC and gel filtration and protein concentrations were determined by UV-spectrophotometry at 280 nm.

Mammalian expression vectors for Fab fragments were generated. Full length IgG formats of Fabs (e.g., IDT1-IDT10 as described in Table 2) were also generated. Exemplary full length IgG formats for IDT1 and IDT2 were generated. In particular, IDT11 is an exemplary full length IgG format for IDT1, and IDT12 is an exemplary full length IgG format for IDT2.

IDT3 is a variant of IDT2 with a mutation in the VH at position 30, in particular, S to Q mutation at position 30 of the VH (SEQ ID NO: 71) or heavy chain (SEQ ID NO: 73). Accordingly, an exemplary full length IgG format for IDT3 (IDT3-IgG) comprises (i) a heavy chain comprising the amino acid sequence of SEQ ID NO: 349 with a mutation at position 30, such as S to Q mutation, and (ii) a light chain comprising the amino acid sequence of SEQ ID NO: 89.

In specific aspects, exemplary full length IgG formats of any one of the Fabs described in Table 2, e.g., IDT1-IDT10, can be generated by cloning the following Fc region to the C-terminus of the Fab heavy chain:

(SEQ ID NO: 351)
DKTHTCPPCPAPELLGGPSVFLFPPKPKDTLMISRTPEVTCVVVAVSHED
PEVKFNWYVDGVEVHNAKTKPREEQYNSTYRVVSVLTVLHQDWLNGKEYK
CKVSNKALAAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGK.

In certain aspects, other exemplary full length IgG formats for IDT1 can have at least 90%, at least 95%, at least 96%, at least 97%, at least 98%, or at least 99% sequence identity as IDT11 with comparable activity. In certain aspects, other exemplary full length IgG formats for IDT2 can have at least 90%, at least 95%, at least 96%, at least 97%, at least 98%, or at least 99% sequence identity as IDT12 with comparable activity.

### Example 2

### Binding Data

### Surface Plasmon Resonance (SPR) Binding Analysis of Anti-NOV1401 Fab Binding to NOV1401

SPR binding experiments were performed on a ProteOn XPR36 instrument (Bio-Rad Laboratories, Inc.) in PBS/T buffer (50 mM phosphate, 150 mM NaCl, pH 7.4, 0.005% v/v Tween-20) at 25° C. NOV1401 (‘Ligand’) was immobilized onto an activated ProteOn GLC sensor chip (Bio-Rad Laboratories, Inc.) using standard amine coupling procedures as described by the manufacturer. Briefly, NOV1401 was injected at a concentration of 10 μg/ml in 20 mM sodium acetate, pH 5.0 and at a flow rate of 30 μl/min for 10 min. Unreacted groups were blocked by injecting 1 M ethanolamine.

For kinetic studies anti-NOV1401 Fabs (‘Analytes’) were diluted in PBS/T buffer to generate a dilution series with concentrations ranging from 0.125-4 nM. Fabs were injected onto surfaces with immobilized NOV1401 at a flow rate of 100 μL/min and sensorgrams were recorded for association and dissociation times of 220 s and 1800 s, respectively. Blank surfaces were used for background corrections. There was no need to regenerate surfaces since the ProteOn protein interaction array system allows to run up to six binding experiments on an identical surface in parallel.

Data processing and analysis including kon, koff, and K_{D} determination were performed with the ProteOn Manager™ software version 3.1.0.6. Sensorgrams were fitted by applying a Langmuir 1:1 binding model (Rmax set at global) and dissociation constants were calculated from kon and koff Table 3 shows the dissociation constants for 10 anti-NOV1401 Fab determined by SPR.

TABLE 3
Summary of SPR binding data

| | Average K_{D} | StDEV | |
|---|---|---|---|
| Anti-NOV1401 | [nM] | [nM] | n |
| IDT1 | 0.44 | 0.02 | 2 |
| IDT2 | 0.23 | NA | 1 |
| IDT3 | 0.24 | 0.02 | 2 |
| IDT4 | 0.31 | 0.02 | 2 |
| IDT5 | 0.35 | 0.04 | 2 |
| IDT6 | 10.08 | 0.13 | 2 |
| IDT7 | 0.33 | 0.01 | 2 |
| IDT8 | 2.15 | 0.15 | 2 |
| IDT9 | 1.92 | 0.00 | 2 |
| IDT10 | 5.97 | 0.03 | 2 |

### Solution Equilibrium Titration (SET) Binding Analysis of Anti-NOV1401 Fab and IgG Binding to NOV1401

14 serial (2×) dilutions of NOV1401 were prepared in sample buffer (PBS pH 7.4 containing 0.5% (w/v) BSA and 0.02% Tween 20) and a constant concentration of the anti-NOV1401 Fab or IgG was added to each NOV1401 concentration ranging from 40 pM to 240 pM. Optimal constant anti-NOV1401 Fab or IgG concentrations and optimal starting concentrations for NOV1401 dilution series were determined in pilot experiments. A starting concentration of 10 nM NOV1401 was used for weaker binders (K_{D}˜InM or higher) and a starting concentration of 2 nM was used for stronger binders (K_{D}<0.2 nM). For anti-NOV1401 IgGs (K_{D}<0.01 nM) starting concentrations of 0.5 or 0.25 nM were used.

30 l/well of each dilution mix was distributed in duplicates to a 384-well polypropylene Eppendorf microplate (MTP). Sample buffer served as negative control and a sample containing no antigen as positive control (Bmax). The plate was sealed and incubated overnight at RT on a plate shaker. A streptavidin (SA) plate from Pierce® (pre-blocked Streptavidin High Binding Capacity 384-Well Plate, #15505) was coated by adding 30 μl/well of 0.5 μg/ml biotinylated NOV1401 diluted in PBS, sealed and incubated for 2 h at RT on a MTP shaker.

After incubation and three times washing with PBST (PBS containing 0.05% Tween 20) 30 μl/well of the NOV1401/anti-NOV1401 Fab or IgG preparation was transferred from the polypropylene MTP to the NOV1401-coated SA plate and incubated for 30 min at RT on a MTP shaker. After three additional wash steps, 30 μl of 0.5 μg/ml detection antibody (Goat anti-human Kappa LC-HRP, BETHYL # A80-115P) diluted in sample buffer was added to each well and incubated for 1 h at RT with shaking. After washing the plate again three times, 30 μl of detection reagent (LumiGLO Peroxidase Chemiluminescent Substrate, KPL #54-61-01) was added to each well. Electrochemiluminescence (ECL) signals were generated and detected immediately with a luminescence imager (SpectraMax M5, Molecular Devices, LLC).

Average ECL-signals were calculated from duplicate measurements within each assay. Data were baseline adjusted by subtracting the lowest value from all data points and plotted against the corresponding antigen concentration. K_{D} values were determined by fitting the plot with the following non-linear curve fitting model for 1:1 binding according to Haenel et al 2005:

$$y = {B}_{ma x} - ( \frac{{B}_{ma x}}{2 [ Fab ]} ( [ Fab ] + x + {K}_{D} - \sqrt{{( [ Fab ] + x + {K}_{D} )}^{2} - 4 x [ Fab ]} ) )$$

where y is the blank-subtracted ECL signal, [Fab] is the applied Fab concentration, x is the applied total soluble antigen (here NOV1401), Bmax is the blank-subtracted ECL signal for x=0, and K_{D} is the dissociation constant.

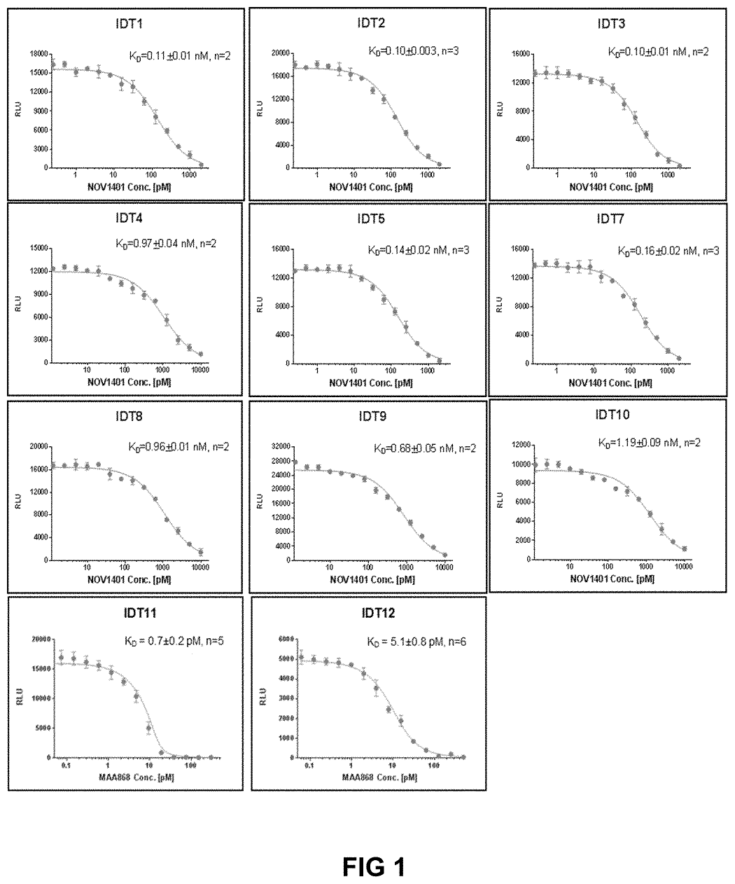

The SET results for nine anti-NOV1401 Fabs and two IgGs are summarized in Table 4 and representative binding response curves are shown in FIG. 1. SET data could not be fit for IDT6 and have not been included.

TABLE 4
Summary of SET results for anti-NOV1401 antibodies

| Antidote | Average K_{D} (nM) | Stdev | n |
|---|---|---|---|
| IDT1 | 0.11 | 0.01 | 2 |
| IDT2 | 0.10 | 0.003 | 3 |
| IDT3 | 0.10 | 0.01 | 2 |
| IDT4 | 0.97 | 0.04 | 2 |
| IDT5 | 0.14 | 0.02 | 3 |
| IDT7 | 0.96 | 0.01 | 2 |
| IDT8 | 0.16 | 0.02 | 3 |
| IDT9 | 0.68 | 0.05 | 2 |
| IDT10 | 1.19 | 0.09 | 2 |
| IDT11 | 0.0007 | 0.0002 | 5 |
| IDT12 | 0.0051 | 0.0008 | 6 |

### Example 3

### SPR Binding Competition

SPR experiments were performed in principle as described in Example 2 with the following changes. Human plasma-derived FXIa was used as ligand and immobilized on an activated ProteOn GLC sensor chip (Bio-Rad Laboratories, Inc.) using standard amine coupling procedures as described and by injecting FXIa at a concentration of 10 μg/ml in 20 mM sodium acetate, pH 5.0 and at a flow rate of 30 μl/min for 10 min.

For binding competition studies NOV1401 and three mixture of NOV1401 with anti-NOV1401 Fab at molar ratios of 1:1, 1:2, and 1:10 were prepared in PBS/T buffer and injected in simultaneously onto surfaces with immobilized FXI at a flow rate of 100 μL/min. Sensorgrams were recorded for association and dissociation times of 220 s and 1800 s, respectively. Blank surfaces were used for background corrections.

NOV1401/anti-NOV1401 Fab mixtures yielded significantly lower binding responses to immobilized FXIa than NOV1401 alone with a 1/10 mixture (NOV1401/anti-NOV1401 Fab) showing no binding to FXIa. As the response units (RUs) in SPR are directly proportional to the mass bound to the chip, increasing concentrations of anti-NOV1401 Fab seems to prevent NOV1401 from binding to FXIa indicating that anti-NOV1401 can bind to NOV1401 and block NOV1401 from binding to FXIa.

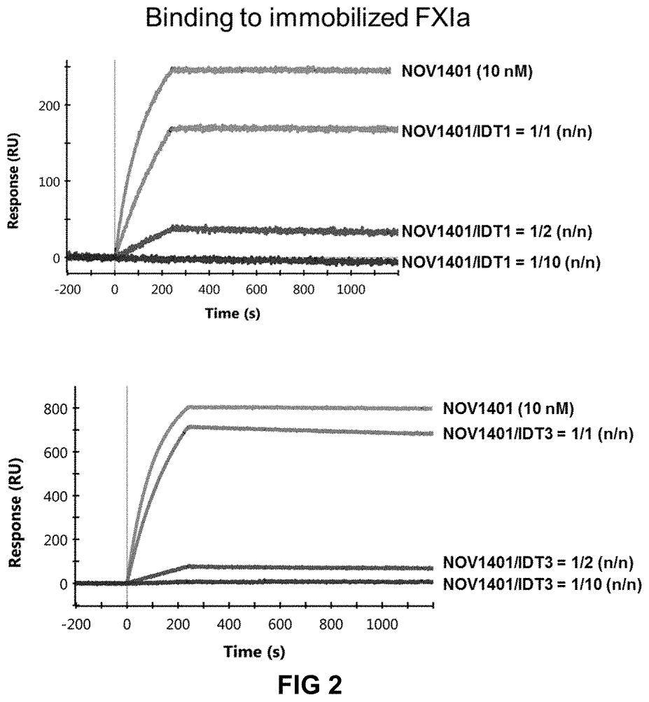

FIG. 2 shows two representative examples for anti-NOV1401 Fabs (IDT1, IDT3). Anti-NOV1401 Fabs clearly reduce NOV1401 binding to its antigen FXIa, therefore compete with FXIa for binding to NOV1401. Added to a NOV1401 solution at 10× molar excess (5× molar excess per NOV1401 binding sites), anti-NOV1401 Fab completely prevents NOV1401 from binding to FXIa, indicating that anti-NOV1401 Fabs are capable of neutralizing free NOV1401 in solution.

### Example 4

### Reversal of the Anticoagulant Activity of NOV1401 in Human Plasma

Effects of anti-NOV1401 antibodies on the anticoagulant activity of NOV1401 was tested by using the activated partial thromboplastin time (aPTT) assay and the thrombin generation assay (TGA).

aPTT Assay:

Lyophilized normal human plasma ‘Coagulation Control N’ (Cat #5020050) was purchased from Technoclone GmbH (Vienna, Austria). It was pooled from citrated plasma of selected healthy donors. The clotting time obtained with this normal plasma reflects normal concentrations of the coagulation factors involved in clotting. The lyophilized plasma was stored at 4° C. Prior to its use, the plasma was re-suspended in 1 mL of distilled water by carefully rotating the vial and then keeping it for 10 minutes at room temperature.

The intrinsic pathway triggering Dapttin TC (Cat #5035090) was purchased from Technoclone GmbH (Vienna, Austria), containing phospholipid, sulfatide, and silicate. The lyophilized trigger was reconstituted in distilled water with the volume indicated on the vial.

Calcium Chloride (Fluka, Cat #21115) was prepared in distillated water at a stock concentration of 25 mM. Phosphate Buffered Saline (PBS, Life Technologies, Cat #10010-023) was used as antibody dilution buffer.

The measurements of the clotting time were performed in a ball coagulometer model MC10 (Merlin medical, Germany), which is a semi-automated mechanical clot detection system. The system utilizes a special cuvette in which a stainless steel ball is distributed (Merlin medical, Cat # Z05100).

The cuvette is placed into the measuring well of the ball coagulometer. After the sample, plasma, and trigger are added to the cuvette, the measuring well rotates slowly causing the cuvette to rotate along its longitudinal axis. Because the cuvette is positioned at a slight angle, gravity and inertia always position the ball at the lowest point of the cuvette. Exactly opposite the ball-position is a magnetic sensor. After an appropriate incubation period, a timer is started with the addition of the calcium chloride solution. As coagulation takes place, fibrin strands form in the reaction mixture. The fibrin strands pull the ball away from its inertia position that triggers an impulse in the magnetic sensor. This impulse electronically stops the timer.

Serial dilutions of NOV1401 were prepared in PBS. The reconstituted human blood plasma, trigger reagent (Dapttin), calcium chloride were warmed up in a water bath at 37° C. for 10 minutes.

The assay was performed exclusively in specialized cuvettes containing a stainless steel ball. The pipetting scheme is outlined in Table 5.

TABLE 5
Pipetting scheme for measuring NOV1401 activity in aPTT assay

| Assay | | aPTT assay |
|---|---|---|
| step | Solution | Volume [μL] |
| 1 | antibody dilution or diluent | 50 |
| 2 | human blood plasma | 50 |
| 3 | Dapttin | 50 |
| 4 | | Incubate 3 minutes at 37° C. |
| | | under rotation |
| 5 | 25 mM Calcium Chloride | 50 |

| | |
|---|---|
| 6 | Immediately start the timer |
| 7 | The timer stops when the clot is formed |

The samples were measured in duplicates at a temperature of 37° C. in the Merlin ball coagulometer described above.

The clot formation was timed for each concentration of NOV1401 and plotted versus the corresponding antibody concentrations. The resulting dose-response curve was fitted using the non-linear regression program GraphPad Prism (GraphPad Software Inc., La Jolla, Calif., USA). From fitting the dose response curve the NOV1401 concentration for doubling of the initial clotting time (sample containing plasma without antibody), also described as ‘2×aPTT’, was determined.

### Anti-NOV1401 Fabs Block the Anticoagulant Activity of NOV1401:

To determine if anti-NOV1401 Fabs can block NOV1401's ability to prolong clotting times in the aPTT assay, several NOV1401/anti-NOV1401 Fab mixtures in PBS were generated where the NOV1401 concentration was kept constant at a value required for 2×aPTT which was determined in a separate experiment as described above. Anti-NOV1401 Fab was added at equimolar amount (1/1) or at molar excess, typically 1/3 or 1/5, and 1/10 (n/n). The pipetting scheme is shown in Table 6.

TABLE 6
Pipetting scheme for measuring the effect of anti-
NOV1401 Fab on NOV1401 activity in the aPTT assay.

| Assay | | aPTT assay |
|---|---|---|
| step | Solution | Volume [μL] |
| 1 | NOV1401 or diluent | 25 |
| 2 | anti-NOV1401 Fab or | 25 |
| | control Ig | |
| 3 | | Incubate 10 minutes |
| 4 | Human blood plasma | 50 |
| 5 | Dapttin | 50 |
| 6 | | Incubate 3 minutes at 37° C. |
| | | under rotation |
| 7 | 25 mM Calcium Chloride | 50 |

| | |
|---|---|
| 8 | Immediately start the timer |
| 9 | The timer stops when the clot is formed |

The samples were measured in duplicates at a temperature of 37° C. in the Merlin ball coagulometer described above.

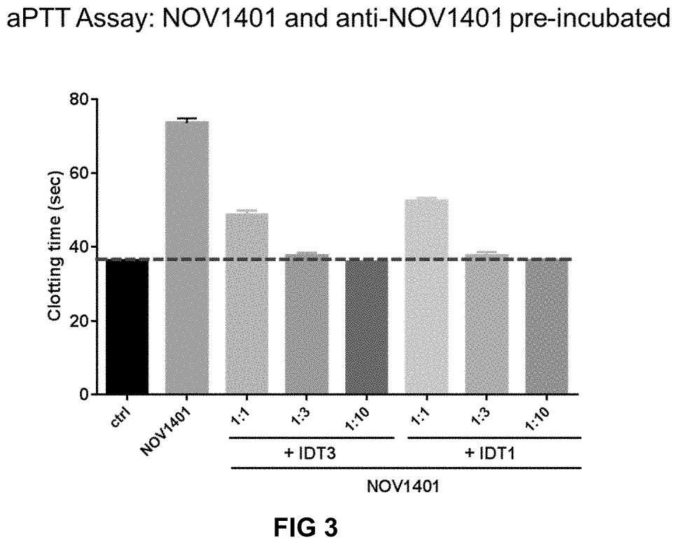

The results for two anti-NOV1401 Fabs—IDT1 and IDT3—are shown in FIG. 3. At a constant NOV1401 concentration of 0.096 μM increasing amounts of NOV1401 Fab block the effect of NOV1401 on coagulation as measured in the aPTT assay. A three times molar excess of IDT1 or IDT3 (1.5× molar excess per binding site) was sufficient to completely inhibit the effect of NOV1401 on aPTT.

These data confirm and extend the results from SPR competition experiments as they suggest that anti-NOV1401 Fabs block the function of NOV1401 when pre-mixed with NOV1401. Together these results suggest that anti-NOV1401 Fabs are capable to prevent free NOV1401 from binding to FXI and blocking the effects of FXI.

### Anti-NOV1401 Fabs and IgGs Partially Reverse the Anticoagulant Activity of NOV1401:

To determine if anti-NOV1401 Fabs and IgGs can reverse NOV1401's ability to prolong clotting times in the aPTT, NOV1401 was preincubated with FXI-containing human plasma for 5 min before anti-NOV1401 Fab or IgG was added. As in the blocking experiment the concentration of NOV1401 was kept constant a value required for 2×aPTT determined separately in a dose response experiment as described above.

Anti-NOV1401 Fab or IgG was added at equimolar amount (1/1) or at molar excess, typically 1/3 and 1/10 (n/n). The pipetting scheme is shown in Table 7.

TABLE 7
Pipetting scheme for measuring reversal of the effects
of NOV1401 on aPTT by anti-NOV1401 Fabs and IgGs.

| Assay | | aPTT assay |
|---|---|---|
| step | Solution | Volume [μL] |
| 1 | Human blood plasma | 50 |
| 2 | NOV1401 or diluent | 25 |
| 3 | | Incubate 5 minutes |
| 4 | anti-NOV1401 | 25 |
| | antibody (Fab, IgG) or | |
| | control Ig | |
| 5 | | Incubate 10 minutes |
| 6 | Dapttin | 50 |
| 7 | | Incubate 3 minutes at 37° C. |
| | | under rotation |
| 8 | 25 mM Calcium | 50 |
| | Chloride | |

| | |
|---|---|
| 9 | Immediately start the timer |
| 10 | The timer stops when the clot is formed |

The samples were measured in duplicates at a temperature of 37° C. in the Merlin ball coagulometer described above. The reversal percentage of NOV1401 clotting time was determined for each anti-NOV1401 antibody using the following equation:

Percentage reversal=(NOV1401 clotting time−antidote clotting time)/(NOV1401clotting time−initial clotting time)*100.

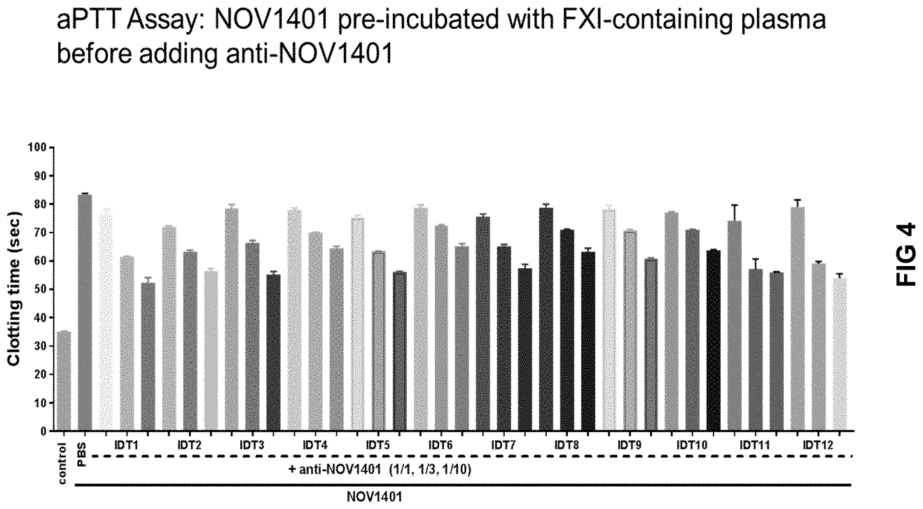

The results for 10 anti-NOV1401 Fabs, IDT1-IDT10, and two anti-NOV1401 IgGs, IDT11 and IDT12, are shown in FIG. 4. At a constant NOV1401 concentration of 0.096 M increasing amounts of anti-NOV1401 Fab added after NOV1401 was incubated with FXI containing human plasma partially reverse the effect of NOV1401 on coagulation as measured in the aPTT assay. While all anti-NOV1401 show some reversal, the degree of reversal at a given concentration varies and a maximum reversal of 55-65% was observed at 10× molar excess. The reversal percentages for all anti-NOV1401 Fabs and IgGs are summarized in Table 8. These results suggest that anti-NOV1401 Fabs and IgGs are capable of reversing, at least partially reversing, the anticoagulant effects of NOV1401 as measured in the aPTT assay.

In these aPTT assays, anti-NOV1401 IgGs IDT11 and IDT12 reached similar maximum reversal effects compared to IDT3 (approximately 50-60% maximal reversal observed). However, values close to the maximum effect were already reached at ˜3× molar excess, as well as at 10× molar excess. This data indicates that a full length IgG format (e.g., full length IgG format for any one of Fabs IDT1-IDT10 described in Table 2) may be effective at lower concentrations.

TABLE 8
Summary of aPTT reversal data for anti-NOV1401 antibodies

| anti-NOV1401 | NOV1401/anti NOV1401 ratio (n/n) | | |
|---|---|---|---|
| Fab | 1/10 | 1/3 | 1/1 |
| IDT1 | 64% | 45% | 12% |
| IDT2 | 55% | 42% | 21% |
| IDT3 | 58% | 35% | 9% |
| IDT4 | 39% | 27% | 10% |
| IDT5 | 56% | 42% | 17% |
| IDT6 | 38% | 22% | 9% |
| IDT7 | 54% | 37% | 16% |
| IDT8 | 42% | 25% | 8% |
| IDT9 | 46% | 25% | 10% |
| IDT10 | 40% | 25% | 12% |
| IDT11 | 57% | 55% | 19% |
| IDT12 | 61% | 51% | 9% |

### Thrombin Generation Assay:

To confirm the reversal of NOV1401 anticoagulant activity observed in the aPTT assay in another functional assay, the TGA was employed to measure thrombin generated through the thrombin feedback loop, which depends on the activity of FXIa.

For the TGA lyophilized normal human plasma (Coagulation control N) was purchased from Technoclone GmbH (Cat #5020040) and reconstituted in distilled water in a volume suggested by the manufacturer.

The substrate solution was prepared using the fluorogenic substrate Z-Gly-Gly-Arg-AMC from Technoclone GmbH (Cat #5006230). Aliquots of the lyophilized substrate were kept at 4° C. The substrate was dissolved freshly in the volume of distilled water indicated on the vial 20 minutes prior its use in the assay. The reconstituted substrate solution contains the fluorogenic peptide at a concentration of 1 mM and CaCl2 at a concentration of 15 mM.

The trigger reagent ‘platelet poor plasma (PPP)-reagent low’ was purchased from Thrombinoscope (Cat # TS31.00) and reconstituted in distilled water as indicated on the vial. ‘PPP-reagent low’ contains a mixture of phospholipids and tissue factor at very low concentration. The reagent was 8-fold diluted in 80 mM Tris/HCl at pH7.4, 0.05% (w/v) CHAPS immediately before use.

The samples were aliquoted and measured in 96 well black/clear bottom plates purchased from Costar (product no 3603). For automation transfer samples were placed in V-bottom 96 well plate (Costar, 3894) and transferred using a CyBio automation system (Analytik Jena US, Woburn, Mass., USA).

The reconstituted human blood plasma, trigger reagent ‘PPP-reagent low’ and substrate were pre-warmed for 10 minutes in a water bath at 37° C. Serial 1:3 antibody dilutions in PBS were prepared in a 96 well plate starting with a NOV1401 concentration of 5 μM (5× the highest final concentration of 1 μM) for a total of 8 dilutions. 222 μl of trigger reagent was mixed with 1108 μl of substrate solution to generate the 10+50 trigger reagent substrate mix. 80 μl per well was added into a V-bottom 96 well plate for later transfer using an automation system. The plate was kept at 37° C. The reagents were added according to the scheme given in Table 9.

TABLE 9
Pipetting scheme for TGA with NOV1401

| Assay | | |
|---|---|---|
| step | Solution | Volume [ul] |
| 1 | Antibody solutions (8 dilutions) | 20 |
| 2 | Plasma stock solution | 20 |

5 minutes incubation at 37° C. in a thermomixer at 300 rpm.

| | | |
|---|---|---|
| 3 | Trigger reagent/substrate mixture | 10 + 50 |

Trigger/substrate mixtures were transferred using automation. After adding the mixtures, excitation and emission at 360 nm at 460 nm, respectively, were recorded immediately using a Synergy Neo instrument (BioTek Instrument Inc., Winooski, Vt., USA). The samples were measured in duplicates at a temperature of 37° C. in the plate reader for 90 minutes at intervals of 55 seconds.

To generate peak thrombin concentration values data were processed using the TGA evaluation software file provided by Technoclone. To generate plots for peak thrombin concentration vs antibody concentration data were fit using GraphPad software. These data were fit to a non-linear regression model in the GraphPad PrismS software (GraphPad Software Inc., La Jolla, Calif., USA). The IC50 value was determined using the built-in four-parameter dose-response curve equation (variable slope): y=Bottom+(Top−Bottom)/(1+10\hat{ }((Log IC50−x)*Hillslope)) where y is the maximal concentration of thrombin formed at the inhibitor concentration, x, and top and bottom represent the concentration of thrombin without inhibitor and at the highest concentration of inhibitor, respectively.

NOV1401 dose-dependently reduces thrombin in the TGA and the IC₅₀ value determined by this method was used as the concentration of NOV1401 in reversal experiments with anti-NOV1401 Fabs and IgGs.

### Anti-NOV1401 Fabs and IgGs Partially Reverses the Effect of NOV1401 on Thrombin Generation in the TGA:

To determine if anti-NOV1401 antibodies (Fabs and IgGs) can reverse NOV1401's ability to reduce thrombin generation in the TGA, NOV1401 was preincubated with FXI-containing human plasma for 5 min before anti-NOV1401 Fab or IgG was added. The concentration of NOV1401 was kept constant at the IC₅₀ value determined separately in a dose response experiment as described above. Anti-NOV1401 Fab or IgG was added at equimolar amount (1/1) or at molar excess, typically 1/3 and 1/10 (n/n). The pipetting scheme is shown in Table 10.

TABLE 10
Pipetting scheme for measuring the reversal of the effects
of NOV1401 on TGA by anti-NOV1401 Fab or IgG.

| Assay | | |
|---|---|---|
| step | Solution | Volume [uL] |
| 1 | NOV1401 solution or PBS | 10 |
| 2 | anti-NOV1401 antibody (Fab or IgG) or | 10 |
| | control IgG | |

10 minutes incubation at 37° C.

| | | |
|---|---|---|
| 3 | Plasma stock solution | 20 |

5 minutes incubation at 37° C. in a thermomixer at 300 rpm.

| | | |
|---|---|---|
| 4 | Trigger reagent/substrate mixture | 10 |
| | | 50 |

The maximum concentrations of thrombin generated for each assay conditions are plotted and the percentage reversal was determined using the following equation:

y=(A−B)/(C−B)*100.

where y is the percentage reversal, A the thrombin concentration for assay conditions with anti-NOV1401, B is the thrombin concentration for assay conditions without anti-NOV1401, C is the initial thrombin concentration.

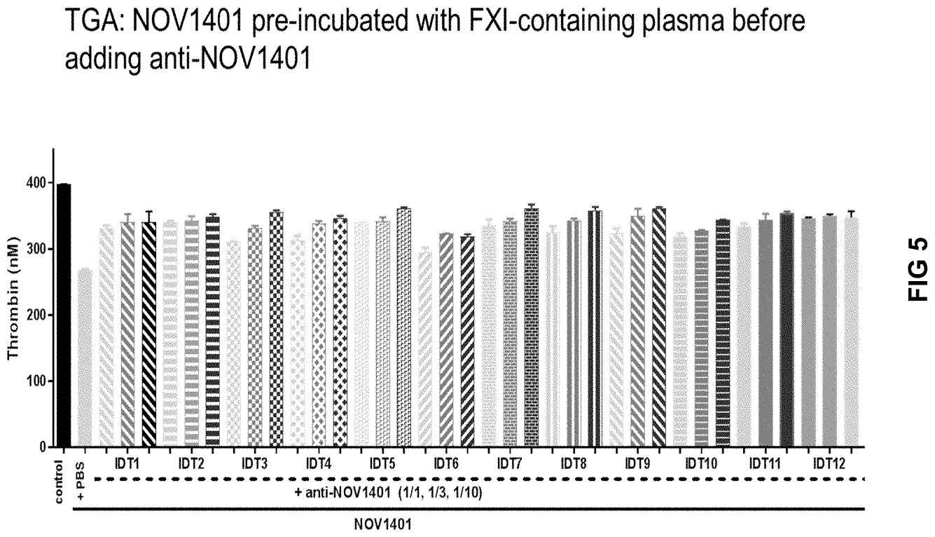

The results for 10 anti-NOV1401 Fabs, IDT1-IDT10, and 2 anti-NOV1401 IgGs, IDT11 and IDT12, are shown in FIG. 5. At a constant NOV1401 concentration of 0.05 μM increasing amounts of anti-NOV1401 Fab or IgG added after NOV1401 was incubated with FXI-containing human plasma led to an increase in thrombin concentration, hence reversing (e.g., at least partially reversing) the effect of NOV1401 on thrombin generation in the TGA. While all anti-NOV1401 show some reversal, the degree of reversal at a given concentration varies and a maximum reversal of 37-72% was observed at 10× molar excess. The reversal percentages for all anti-NOV1401 Fabs and IgGs are summarized in Table 11. These results suggest that anti-NOV1401 Fabs and IgGs can reverse (e.g., at least partially reverse) the reduction of thrombin by NOV1401 in the TGA.

TABLE 11
Summary of TGA reversal data for anti-NOV1401 antibodies

| anti-NOV1401 | NOV1401/anti NOV1401 ratio (n/n) | | |
|---|---|---|---|
| Fab | 1/10 | 1/3 | 1/1 |
| IDT1 | 55% | 55% | 49% |
| IDT2 | 61% | 57% | 55% |
| IDT3 | 67% | 47% | 31% |
| IDT4 | 60% | 53% | 33% |
| IDT5 | 72% | 56% | 55% |
| IDT6 | 37% | 41% | 18% |
| IDT7 | 71% | 66% | 50% |
| IDT8 | 69% | 56% | 41% |
| IDT9 | 72% | 61% | 41% |
| IDT10 | 57% | 45% | 37% |
| IDT11 | 67% | 59% | 50% |
| IDT12 | 62% | 64% | 60% |

### Anti-NOV1401 Fabs Acutely Reverses the Anticoagulant Effects of NOV1401 in Cynomolgus Monkeys:

To test if an anti-NOV1401 Fab can reverse NOV1401's ability to prolong clotting times in vivo we administered a single 3 mg/kg subcutaneous dose of NOV1401 to cynomolgus monkeys on study day one followed by two i.v. doses of IDT3 on study day 4 and 5, respectively. A dose of 3 mg/kg s.c. was chosen for NOV1401 since it has been demonstrated that this dose leads to sustained aPTT prolongation in cynomolgus monkeys. Based on our in vitro experiments with human plasma, anti-NOV1401 Fab was administered in molar excess, for example, IDT3 was administered i.v. at 10 mg/kg followed by 30 mg/kg in one animal and at 30 mg/kg followed by 90 mg/kg in a second animal. Additional animals (N=2) were also administered NOV1401 only (one dose of 3 mg/kg s.c. on study day one), or IDT3 only (two i.v. doses of 30 mg/kg and 90 mg/kg on study day 4 and 5, respectively).

For ex-vivo aPTT analysis, blood samples were collected into sodium citrate coagulation tubes on study day 3, and 30 min, 2 hours, 8 hours and 12 hours post IDT3 dose on study days 4 and 5. Additional samples were collected on study days 6, 7, 8, and 9. All blood samples were centrifuged; plasma samples were obtained and frozen at approximately −70° C. or below.

In animals treated with NOV1401 alone, a single subcutaneous dose of 3 mg/kg prolonged aPTT by 1.7 to 1.8× throughout the end of the study demonstrating that NOV1401 has potent anticoagulant effects in cynomolgus monkeys and confirming earlier studies.

In animals that were dosed at 10 mg/kg i.v. with IDT3 three days after NOV1401, aPTT was normalized immediately and had reached baseline levels at the earliest time point of 30 min after dosing (FIG. 6). After 8-12 hours, the aPTT prolongating effect of NOV1401 had returned close to maximum levels, but was reduced again to baseline after a second dose of 30 mg/kg was administered. Very similar effects were observed when IDT3 was dosed at 30 mg/kg and 90 mg/kg. The higher doses seem to extend the reversal effect. No effect on aPTT was observed in animals that were only administered IDT3 at 30 mg/kg and 90 mg/kg (FIG. 6).

These data suggest that anti-NOV1401 Fabs such as IDT3 are able to acutely reverse the effects of NOV1401 on aPTT in vivo and anti-NOV1401 Fabs provided herein such as IDT3 can serve as an effective reversal agent for anti-FXI/FXIa antibody NOV1401, for example in cases when quick neutralization of anti-FXI/FXIa antibody NOV1401 be needed. These data also indicate that the acute reversal observed in vivo in this monkey study correlates with the partial reversal observed in the in vitro experiments with human plasma, such as the aPTT assays described herein.

### Example 5

### Developability and Formulation Assessments

The heavy and light chains of anti-NOV1401 Fabs and IgGs were cloned into an expression vector system suitable for secretion in mammalian cells.

Production process and formulation studies for anti-NOV1401 Fabs and IgGs were carried out to assess characteristics such as formation of high molecular weight species (HMW) as an indication of aggregation in solution, solubility, and clipping.

Anti-NOV1401 Fabs and IgGs, in particular IDT1-IDT12, were recombinantly expressed and purified from CHO cells. For formulation studies, anti-NOV1401 Fabs and IgGs, in particular IDT1-IDT12, were assessed in a liquid formulation with 220 mM sucrose, 20 mM histidine, 0.04% Polysorbate 20 at pH 5.5, at a concentration of 150 mg/mL, and were found to be well behaved, such as low aggregation tendencies, low clipping, and high solubility. For example, parameters such as formation of HMW, solubility, and clipping were found to be within acceptable ranges for typical Fabs and IgGs in liquid formulations. Similar results were achieved when tested at slightly different pH. These results indicate that the tested liquid formulations and certain variants thereof are suitable for anti-NOV1401 Fabs and IgGs.

### INCORPORATION BY REFERENCE

All references cited herein, including patents, patent applications, papers, publications, text books, and the like, and the references cited therein, to the extent that they are not already, are hereby incorporated herein by reference in their entirety.

1. A binding agent which specifically binds a target antibody that binds human FXI and/or FXIa within the catalytic domain, wherein the binding agent inhibits an anticoagulant activity of the target antibody,
wherein the target antibody comprises (i) a heavy chain variable region (VH) comprising the amino acid sequence of SEQ ID NO: 12 and a light chain variable region (VL) comprising the amino acid sequence of SEQ ID NO: 23; or (ii) a heavy chain comprising the amino acid sequence of SEQ ID NO: 14 and a light chain comprising the amino acid sequence of SEQ ID NO: 25; and
wherein the binding agent is an antibody or antigen-binding fragment thereof comprising (a) a heavy chain comprising the amino acid sequence of SEQ ID NO: 347 and a light chain comprising the amino acid sequence of SEQ ID NO: 57, or (b) a heavy chain comprising the amino acid sequence of SEQ ID NO: 349 and a light chain comprising the amino acid sequence of SEQ ID NO: 89.

2. A binding agent which specifically binds a target antibody that binds human FXI and/or FXIa within the catalytic domain, wherein the binding agent reverses an anticoagulant activity of the target antibody, and wherein the binding agent is anti-idiotype antibody IDT11 or IDT12 as set forth in Table 2.

3. A binding agent which specifically binds a target antibody that binds human FXI and/or FXIa within the catalytic domain, wherein the binding agent reverses an anticoagulant activity of the target antibody, and wherein the binding agent is an anti-idiotype antibody comprising a heavy chain and a light chain, wherein
(i) the heavy chain comprises an amino acid sequence that is at 90% identical to SEQ ID NO: 347 and the light chain comprises an amino acid sequence that is at least 90% identical to SEQ ID NO: 57;
(ii) the heavy chain comprises an amino acid sequence that is at least 90% identical to SEQ ID NO: 349 and the light chain comprises an amino acid sequence that is at least 90% identical to SEQ ID NO: 89;
(iii) the heavy chain comprises an amino acid sequence that is at least 95% identical to SEQ ID NO: 347 and the light chain comprises an amino acid sequence that is at least 95% identical to SEQ ID NO: 57;
(iv) the heavy chain comprises an amino acid sequence that is at 95% identical to SEQ ID NO: 349 and the light chain comprises an amino acid sequence that is at least 95% identical to SEQ ID NO: 89;
(v) the heavy chain comprises an amino acid sequence that is at least 98% identical to SEQ ID NO: 347 and the light chain comprises an amino acid sequence that is at least 98% identical to SEQ ID NO: 57;
(vi) the heavy chain comprises an amino acid sequence that is at 98% identical to SEQ ID NO: 349 and the light chain comprises an amino acid sequence that is at least 98% identical to SEQ ID NO: 89;
(vii) the heavy chain comprises the amino acid sequence of SEQ ID NO: 347 with one, two, three, or four amino acid mutations that does not substantially affect activity and the light chain comprises the amino acid sequence of SEQ ID NO: 57 with one, two, three, or four amino acid mutations that does not substantially affect activity;
(viii) the heavy chain comprises the amino acid sequence of SEQ ID NO: 349 with one, two, three, or four amino acid mutations that does not substantially affect activity and the light chain comprises the amino acid sequence of SEQ ID NO: 89 with one, two, three, or four amino acid mutations that does not substantially affect activity; or
(ix) the heavy chain comprises the amino acid sequence of SEQ ID NO: 349 with a mutation, such as a conservative mutation, at position 30, such as S to Q mutation, of SEQ ID NO: 349 that does not substantially affect activity and the light chain comprises the amino acid sequence of SEQ ID NO: 89.

4. A polynucleotide comprising nucleotide sequences encoding the binding agent of any one of the preceding claims.

5. A vector comprising the polynucleotide of claim 4.

6. A host cell comprising the polynucleotide of claim 4.

7. A host cell comprising the vector of claim 4.

8. A method of producing a binding agent, said method comprises culturing the host cell of claim 6 or 7 under suitable conditions for expression of the binding agent or a portion thereof, wherein the method optionally comprises purifying the binding agent.

9. A pharmaceutical composition comprising the binding agent of any one of the preceding claims.

10. A pharmaceutical composition for use as a medicament for reversing the anticoagulant effect of an anti-FXI/FXIa antibody in a patient being treated with the anti-Factor XI/Factor XIa antibody, wherein the pharmaceutical composition comprises an effective amount of the binding agent of any one of the preceding claims.

11. A method for reversing the anticoagulant effect of an anti-FXI/FXIa antibody in a patient being treated with the anti-FXI/FXIa antibody or antigen-binding fragment thereof, comprising administering an effective amount of the binding agent of any one of the preceding claims to a patient in need thereof.

12. The method of claim 11, wherein the anti-FXI/FXIa antibody or antigen-binding fragment thereof comprises (i) a VH comprising the amino acid sequence of SEQ ID NO: 12 and a VL comprising the amino acid sequence of SEQ ID NO: 23; or (ii) a heavy chain comprising the amino acid sequence of SEQ ID NO: 14 and a light chain comprising the amino acid sequence of SEQ ID NO: 25.

13. The method of claim 11, wherein the anti-FXI/FXIa antibody or antigen-binding fragment thereof comprises (i) a VH comprising complementarity determining regions HCDR1, HCDR2 and HCDR3 of a VH comprising the amino acid sequence of SEQ ID NO: 12 and (ii) a VL comprising complementarity determining regions LCDR1, LCDR2, and LCDR3 of a VL comprising the amino acid sequence of SEQ ID NO: 23.

14. The method of any one of claims 11-13, wherein the method further comprises applying one of the following to the patient: (i) fluid replacement using colloids, crystalloids, human plasma or plasma proteins such as albumin; (ii) transfusion with packed red blood or whole blood; or (iii) administration of fresh frozen plasma (FFP), prothrombin complex concentrates (PCC), activated PCC (APCC), such as, factor VIII inhibitor, and/or recombinant, activated factor VII.

15. The method of any one of claims 11-14, wherein the patient has or is at risk of developing thrombosis.

16. The method of any one of claims 11-15, wherein the patient has
a. atrial fibrillation;
b. suspected or confirmed cardiac arrhythmia such as paroxysmal, persistent or permanent atrial fibrillation or atrial flutter;
c. Chronic Thromboembolic Pulmonary Hypertension (CTEPH);
d. valvular heart disease with or without atrial fibrillation;
e. pulmonary hypertension;
f. congenital or acquired thrombophilia including but not exclusively factor V Leiden, prothrombin mutation, antithrombin III, protein C and protein S deficiencies, factor XIII mutation, familial dysfibrinogenemia, congenital deficiency of plasminogen, increased levels of factor XI, sickle cell disease, antiphospholipid syndrome, autoimmune disease, chronic bowel disease, nephrotic syndrome, hemolytic uremia, myeloproliferative disease, disseminated intra vascular coagulation, paroxysmal nocturnal hemoglobinuria and heparin induced thrombopenia; or
g. chronic kidney disease.

17. The method of any one of claims 11-16, wherein the patient has non-valvular atrial fibrillation.

18. The method of any one of claims 11-17, wherein the patient has a demonstrated high risk of bleeding.

19. The method of any one of claims 11-18, wherein the patient has chronic kidney disease.

20. The method of claim 19, wherein patient has end stage renal disease (ESRD).

21. The method of claim 20, wherein the patient has ESRD and is undergoing dialysis.

22. The method of claim 21, wherein the patient has non-valvular atrial fibrillation.

23. The method of any one of claims 11-22, wherein the patient is being administered the anti-FXI/FXIa antibody or antigen-binding fragment thereof to reduce the risk of stroke and/or systemic embolism.

24. The method of any one of claims 11-23, wherein reversal of the anticoagulant effect of the anti-FXI/FXIa antibody or antigen-binding fragment thereof is needed for emergency surgery/urgent procedures and in life-threatening or uncontrolled bleeding.


SEQUENCE LISTING (394 sequences)

SEQ ID NO: 1 - protein, 625 aa, Homo sapiens [sequence omitted]

SEQ ID NO: 2 - DNA, 3278 bp, Homo sapiens
aggcacacaggcaaaatcaagttctacatctgtccctgtgtatgtcacttgtttgaatacgaaataaaattaaaaaaataaattcagtgtattgagaaagcaagcaattctctcaaggtatatttctgacatactaagattttaacgactttcacaaatatgctgtactgagagagaatgttacataacattgagaactagtacaagtaaatattaaagtgaagtgaccatttcctacacaagctcattcagaggaggatgaagaccattttggaggaagaaaagcacccttattaagaattgcagcaagtaagccaacaaggtcttttcaggatgattttcttatatcaagtggtacatttcattttatttacttcagtttctggtgaatgtgtgactcagttgttgaaggacacctgctttgaaggaggggacattactacggtcttcacaccaagcgccaagtactgccaggtagtctgcacttaccacccaagatgtttactcttcactttcacggcggaatcaccatctgaggatcccacccgatggtttacttgtgtcctgaaagacagtgttacagaaacactgccaagagtgaataggacagcagcgatttctgggtattctttcaagcaatgctcacaccaaataagcgcttgcaacaaagacatttatgtggacctagacatgaagggcataaactataacagctcagttgccaagagtgctcaagaatgccaagaaagatgcacggatgacgtccactgccactttttcacgtacgccacaaggcagtttcccagcctggagcatcgtaacatttgtctactgaagcacacccaaacagggacaccaaccagaataacgaagctcgataaagtggtgtctggattttcactgaaatcctgtgcactttctaatctggcttgtattagggacattttccctaatacggtgtttgcagacagcaacatcgacagtgtcatggctcccgatgcttttgtctgtggccgaatctgcactcatcatcccggttgcttgttttttaccttcttttcccaggaatggcccaaagaatctcaaagaaatctttgtctccttaaaacatctgagagtggattgcccagtacacgcattaaaaagagcaaagctctttctggtttcagtctacaaagctgcaggcacagcatcccagtgttctgccattcttcattttaccatgacactgatttcttgggagaagaactggatattgttgctgcaaaaagtcacgaggcctgccagaaactgtgcaccaatgccgtccgctgccagttttttacctataccccagcccaagcatcctgcaacgaagggaagggcaagtgttacttaaagctttcttcaaacggatctccaactaaaatacttcacgggagaggaggcatctctggatacacattaaggttgtgtaaaatggataatgagtgtaccaccaaaatcaagcccaggatcgttggaggaactgcgtctgttcgtggtgagtggccgtggcaggtgaccctgcacacaacctcacccactcagagacacctgtgtggaggctccatcattggaaaccagtggatattaacagccgctcactgtttctatggggtagagtcacctaagattttgcgtgtctacagtggcattttaaatcaatctgaaataaaagaggacacatctttctttggggttcaagaaataataatccatgatcagtataaaatggcagaaagcgggtatgatattgccttgttgaaactggaaaccacagtgaattacacagattctcaacgacccatatgcctgccttccaaaggagatagaaatgtaatatacactgattgctgggtgactggatgggggtacagaaaactaagagacaaaatacaaaatactctccagaaagccaagatacccttagtgaccaacgaagagtgccagaagagatacagaggacataaaataacccataagatgatctgtgccggctacagggaaggagggaaggacgcttgcaagggagattcgggaggccctctgtcctgcaaacacaatgaggtctggcatctggtaggcatcacgagctggggcgaaggctgtgctcaaagggagcggccaggtgtttacaccaacgtggtcgagtacgtggactggattctggagaaaactcaagcagtgtgaatgggttcccaggggccattggagtccctgaaggacccaggatttgctgggagagggtgttgagttcactgtgccagcatgcttcctccacagtaacacgctgaaggggcttggtgtttgtaagaaaatgctagaagaaaacaaactgtcacaagttgttatgtccaaaactcccgttctatgatcgttgtagtttgtttgagcattcagtctctttgtttttgatcacgcttctatggagtccaagaattaccataaggcaatatttctgaagattactatataggcagatatagcagaaaataaccaagtagtggcagtggggatcaggcagaagaactggtaaaagaagccaccataaatagatttgttcgatgaaagatgaaaactggaagaaaggagaacaaagacagtcttcaccattttgcaggaatctacactctgcctatgtgaacacatttcttttgtaaagaaagaaattgattgcatttaatggcagattttcagaatagtcaggaattcttgtcatttccattttaaaatatatattaaaaaaaatcagttcgagtagacacgagctaagagtgaatgtgaagataacagaatttctgtgtggaagaggattacaagcagcaatttacctggaagtgataccttaggggcaatcttgaagatacactttcctgaaaaatgatttgtgatggattgtatatttatttaaaatatcttgggaggggaggctgatggagatagggagcatgctcaaacctccctaagacaagctgctgctgtgactatgggctcccaaagagctagatcgtatatttatttgacaaaaatcaccatagactgcatccatactacagagaaaaaacaattagggcgcaaatggatagttacagtaaagtcttcagcaagcagctgcctgtattctaagcactgggattttctgtttcgtgcaaatatttatctcattattgttgtgatctagttcaataacctagaatttgaattgtcaccacatagctttcaatctgtgccaacaactatacaattcatcaagtgtg

SEQ ID NO: 3 - protein, 10 aa, Artificial Sequence
  source: /note=“Description of Artificial Sequence: Synthetic peptide”
GFTFSTAAMS

SEQ ID NO: 4 - protein, 17 aa, Artificial Sequence
  source: /note=“Description of Artificial Sequence: Synthetic peptide”
GISGSGSSTYYADSVKG

SEQ ID NO: 5 - protein, 13 aa, Artificial Sequence
  source: /note=“Description of Artificial Sequence: Synthetic peptide”
ELSYLYSGYYFDY

SEQ ID NO: 6 - protein, 5 aa, Artificial Sequence
  source: /note=“Description of Artificial Sequence: Synthetic peptide”
TAAMS

SEQ ID NO: 7 - protein, 7 aa, Artificial Sequence
  source: /note=“Description of Artificial Sequence: Synthetic peptide”
GFTFSTA

SEQ ID NO: 8 - protein, 6 aa, Artificial Sequence
  source: /note=“Description of Artificial Sequence: Synthetic peptide”
SGSGSS

SEQ ID NO: 9 - protein, 8 aa, Artificial Sequence
  source: /note=“Description of Artificial Sequence: Synthetic peptide”
GFTFSTAA

SEQ ID NO: 10 - protein, 8 aa, Artificial Sequence
  source: /note=“Description of Artificial Sequence: Synthetic peptide”
ISGSGSST

SEQ ID NO: 11 - protein, 15 aa, Artificial Sequence
  source: /note=“Description of Artificial Sequence: Synthetic peptide”
ARELSYLYSGYYFDY

SEQ ID NO: 12 - protein, 122 aa, Artificial Sequence
  source: /note=“Description of Artificial Sequence: Synthetic polypeptide”
QVQLLESGGGLVQPGGSLRLSCAASGFTFSTAAMSWVRQAPGKGLEWVSGISGSGSSTYYADSVKGRFTISRDNSKNTLYLQMNSLRAEDTAVYYCARELSYLYSGYYFDYWGQGTLVTVSS

SEQ ID NO: 13 - DNA, 366 bp, Artificial Sequence
  source: /note=“Description of Artificial Sequence: Synthetic polynucleotide”
caggtgcagctgctggaatcaggcggcggactggtgcagcctggcggtagcctgagactgagctgcgctgctagtggcttcacctttagcaccgccgctatgagctgggttcgacaggccccagggaaaggcctcgagtgggtctcagggattagcggtagcggctctagcacctactacgccgatagcgtgaagggccggttcactatctctagggataactctaagaacaccctgtacctgcagatgaatagcctgagagccgaggacaccgccgtctactactgcgctagagagctgagctacctgtatagcggctactacttcgactactggggtcaaggcaccctggtcaccgtgtctagc

SEQ ID NO: 14 - protein, 452 aa, Artificial Sequence
  source: /note=“Description of Artificial Sequence: Synthetic polypeptide”
QVQLLESGGGLVQPGGSLRLSCAASGFTFSTAAMSWVRQAPGKGLEWVSGISGSGSSTYYADSVKGRFTISRDNSKNTLYLQMNSLRAEDTAVYYCARELSYLYSGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1356 bp, Artificial Sequence
  source: /note=“Description of Artificial Sequence: Synthetic polynucleotide”
caggtgcagctgctggaatcaggcggcggactggtgcagcctggcggtagcctgagactgagctgcgctgctagtggcttcacctttagcaccgccgctatgagctgggttcgacaggccccagggaaaggcctcgagtgggtctcagggattagcggtagcggctctagcacctactacgccgatagcgtgaagggccggttcactatctctagggataactctaagaacaccctgtacctgcagatgaatagcctgagagccgaggacaccgccgtctactactgcgctagagagctgagctacctgtatagcggctactacttcgactactggggtcaaggcaccctggtcaccgtgtctagcgctagcactaagggcccctccgtgttccctctggccccttccagcaagtctacctccggcggcacagctgctctgggctgcctggtcaaggactacttccctgagcctgtgacagtgtcctggaactctggcgccctgacctctggcgtgcacaccttccctgccgtgctgcagtcctccggcctgtactccctgtcctccgtggtcacagtgccttcaagcagcctgggcacccagacctatatctgcaacgtgaaccacaagccttccaacaccaaggtggacaagcgggtggagcctaagtcctgcgacaagacccacacctgtcctccctgccctgctcctgaactgctgggcggcccttctgtgttcctgttccctccaaagcccaaggacaccctgatgatctcccggacccctgaagtgacctgcgtggtggtggccgtgtcccacgaggatcctgaagtgaagttcaattggtacgtggacggcgtggaggtgcacaacgccaagaccaagcctcgggaggaacagtacaactccacctaccgggtggtgtccgtgctgaccgtgctgcaccaggactggctgaacggcaaagagtacaagtgcaaagtctccaacaaggccctggccgcccctatcgaaaagacaatctccaaggccaagggccagcctagggaaccccaggtgtacaccctgccacccagccgggaggaaatgaccaagaaccaggtgtccctgacctgtctggtcaagggcttctacccttccgatatcgccgtggagtgggagtctaacggccagcctgagaacaactacaagaccacccctcctgtgctggactccgacggctccttcttcctgtactccaaactgaccgtggacaagtcccggtggcagcagggcaacgtgttctcctgctccgtgatgcacgaggccctgcacaaccactacacccagaagtccctgtccctgtctcccggcaag

SEQ ID NO: 16 - protein, 13 aa, Artificial Sequence
  source: /note=“Description of Artificial Sequence: Synthetic peptide”
SGSSSNIGSNDVS

SEQ ID NO: 17 - protein, 7 aa, Artificial Sequence
  source: /note=“Description of Artificial Sequence: Synthetic peptide”
KNYNRPS

SEQ ID NO: 18 - protein, 11 aa, Artificial Sequence
  source: /note=“Description of Artificial Sequence: Synthetic peptide”
SAWDQRQFDVV

SEQ ID NO: 19 - protein, 9 aa, Artificial Sequence
  source: /note=“Description of Artificial Sequence: Synthetic peptide”
SSSNIGSND

SEQ ID NO: 20 - protein, 3 aa, Artificial Sequence
  source: /note=“Description of Artificial Sequence: Synthetic peptide”
KNY

SEQ ID NO: 21 - protein, 8 aa, Artificial Sequence
  source: /note=“Description of Artificial Sequence: Synthetic peptide”
WDQRQFDV

SEQ ID NO: 22 - protein, 8 aa, Artificial Sequence
  source: /note=“Description of Artificial Sequence: Synthetic peptide”
SSNIGSND

SEQ ID NO: 23 - protein, 110 aa, Artificial Sequence
  source: /note=“Description of Artificial Sequence: Synthetic polypeptide”
QSVLTQPPSASGTPGQRVTISCSGSSSNIGSNDVSWYQQLPGTAPKLLIYKNYNRPSGVPDRFSGSKSGTSASLAISGLQSEDEADYYCSAWDQRQFDVVFGGGTKLTVL

SEQ ID NO: 24 - DNA, 330 bp, Artificial Sequence
  source: /note=“Description of Artificial Sequence: Synthetic polynucleotide”
cagtcagtcctgactcagccccctagcgctagtggcacccctggtcaaagagtgactattagctgtagcggctctagctctaatatcggctctaacgacgtcagctggtatcagcagctgcccggcaccgcccctaagctgctgatctataagaactataataggcctagcggcgtgcccgataggtttagcggatctaaatcagggacttctgctagtctggctattagcggcctgcagtcagaggacgaggccgactactactgtagcgcctgggatcagcgtcagttcgacgtggtgttcggcggaggcactaagctgaccgtgctg

SEQ ID NO: 25 - protein, 216 aa, Artificial Sequence
  source: /note=“Description of Artificial Sequence: Synthetic polypeptide”
QSVLTQPPSASGTPGQRVTISCSGSSSNIGSNDVSWYQQLPGTAPKLLIYKNYNRPSGVPDRFSGSKSGTSASLAISGLQSEDEADYYCSAWDQRQFDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 26 - DNA, 648 bp, Artificial Sequence
  source: /note=“Description of Artificial Sequence: Synthetic polynucleotide”
cagtcagtcctgactcagccccctagcgctagtggcacccctggtcaaagagtgactattagctgtagcggctctagctctaatatcggctctaacgacgtcagctggtatcagcagctgcccggcaccgcccctaagctgctgatctataagaactataataggcctagcggcgtgcccgataggtttagcggatctaaatcagggacttctgctagtctggctattagcggcctgcagtcagaggacgaggccgactactactgtagcgcctgggatcagcgtcagttcgacgtggtgttcggcggaggcactaagctgaccgtgctgggtcaacctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 27 - protein, 10 aa, Artificial Sequence
  source: /note=“Description of Artificial Sequence: Synthetic peptide”
GFTFSDYAMS

SEQ ID NO: 28 - protein, 17 aa, Artificial Sequence
  source: /note=“Description of Artificial Sequence: Synthetic peptide”
VIDYSSSNTYYADSVKG

SEQ ID NO: 29 - protein, 12 aa, Artificial Sequence
  source: /note=“Description of Artificial Sequence: Synthetic peptide”
EGYSYRSIRFDY

SEQ ID NO: 30 - protein, 5 aa, Artificial Sequence
  source: /note=“Description of Artificial Sequence: Synthetic peptide”
DYAMS

SEQ ID NO: 31 - protein, 17 aa, Artificial Sequence
  source: /note=“Description of Artificial Sequence: Synthetic peptide”
VIDYSSSNTYYADSVKG

SEQ ID NO: 32 - protein, 12 aa, Artificial Sequence
  source: /note=“Description of Artificial Sequence: Synthetic peptide”
EGYSYRSIRFDY

SEQ ID NO: 33 - protein, 7 aa, Artificial Sequence
  source: /note=“Description of Artificial Sequence: Synthetic peptide”
GFTFSDY

SEQ ID NO: 34 - protein, 6 aa, Artificial Sequence
  source: /note=“Description of Artificial Sequence: Synthetic peptide”
DYSSSN

SEQ ID NO: 35 - protein, 12 aa, Artificial Sequence
  source: /note=“Description of Artificial Sequence: Synthetic peptide”
EGYSYRSIRFDY

SEQ ID NO: 36 - protein, 8 aa, Artificial Sequence
  source: /note=“Description of Artificial Sequence: Synthetic peptide”
GFTFSDYA

SEQ ID NO: 37 - protein, 8 aa, Artificial Sequence
  source: /note=“Description of Artificial Sequence: Synthetic peptide”
IDYSSSNT

SEQ ID NO: 38 - protein, 14 aa, Artificial Sequence
  source: /note=“Description of Artificial Sequence: Synthetic peptide”
AREGYSYRSIRFDY

SEQ ID NO: 39 - protein, 121 aa, Artificial Sequence
  source: /note=“Description of Artificial Sequence: Synthetic polypeptide”
QVQLLESGGGLVQPGGSLRLSCAASGFTFSDYAMSWVRQAPGKGLEWVSVIDYSSSNTYYADSVKGRFTISRDNSKNTLYLQMNSLRAEDTAVYYCAREGYSYRSIRFDYWGQGTLVTVSS

SEQ ID NO: 40 - DNA, 363 bp, Artificial Sequence
  source: /note=“Description of Artificial Sequence: Synthetic polynucleotide”
caagtgcagctgctggaatctggcggcggactggtgcagcctggcggtagtctgagactgtcttgcgccgcctccggcttcaccttctccgactacgccatgtcctgggtccgacaggcccctggcaagggcctggagtgggtgtccgtgatcgactactcctcctccaacacctactacgccgactccgtgaagggccggttcaccatctcccgggacaactccaagaacaccctgtacctgcagatgaactccctgcgggccgaggacaccgccgtgtactactgcgccagagagggctactcctaccggtccatcagattcgactactggggccagggcaccctggtcaccgtgtcctct

SEQ ID NO: 41 - protein, 224 aa, Artificial Sequence
  source: /note=“Description of Artificial Sequence: Synthetic polypeptide”
QVQLLESGGGLVQPGGSLRLSCAASGFTFSDYAMSWVRQAPGKGLEWVSVIDYSSSNTYYADSVKGRFTISRDNSKNTLYLQMNSLRAEDTAVYYCAREGYSYRSIRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 42 - DNA, 672 bp, Artificial Sequence
  source: /note=“Description of Artificial Sequence: Synthetic polynucleotide”
caagtgcagctgctggaatctggcggcggactggtgcagcctggcggtagtctgagactgtcttgcgccgcctccggcttcaccttctccgactacgccatgtcctgggtccgacaggcccctggcaagggcctggagtgggtgtccgtgatcgactactcctcctccaacacctactacgccgactccgtgaagggccggttcaccatctcccgggacaactccaagaacaccctgtacctgcagatgaactccctgcgggccgaggacaccgccgtgtactactgcgccagagagggctactcctaccggtccatcagattcgactactggggccagggcaccctggtcaccgtgtcctctgctagcaccaagggcccctccgtgttccctctggccccttccagcaagtctacctctggcggcaccgctgctctgggctgcctggtgaaggactacttccctgagcctgtgacagtgtcctggaactctggcgccctgacctccggcgtgcacaccttccctgccgtgctgcagtcctccggcctgtactccctgtcctccgtggtgacagtgccttcctccagcctgggcacccagacctatatctgcaacgtgaaccacaagccttccaacaccaaggtggacaagcgggtggagcctaagtcatgc

SEQ ID NO: 43 - protein, 11 aa, Artificial Sequence
  source: /note=“Description of Artificial Sequence: Synthetic peptide”
RASQSISSNLN

SEQ ID NO: 44 - protein, 7 aa, Artificial Sequence
  source: /note=“Description of Artificial Sequence: Synthetic peptide”
AASNLQS

SEQ ID NO: 45 - protein, 9 aa, Artificial Sequence
  source: /note=“Description of Artificial Sequence: Synthetic peptide”
LQFDHTPFT

SEQ ID NO: 46 - protein, 11 aa, Artificial Sequence
  source: /note=“Description of Artificial Sequence: Synthetic peptide”
RASQSISSNLN

SEQ ID NO: 47 - protein, 7 aa, Artificial Sequence
  source: /note=“Description of Artificial Sequence: Synthetic peptide”
AASNLQS

SEQ ID NO: 48 - protein, 9 aa, Artificial Sequence
  source: /note=“Description of Artificial Sequence: Synthetic peptide”
LQFDHTPFT

SEQ ID NO: 49 - protein, 7 aa, Artificial Sequence
  source: /note=“Description of Artificial Sequence: Synthetic peptide”
SQSISSN

SEQ ID NO: 50 - protein, 3 aa, Artificial Sequence
  source: /note=“Description of Artificial Sequence: Synthetic peptide”
AAS

SEQ ID NO: 51 - protein, 6 aa, Artificial Sequence
  source: /note=“Description of Artificial Sequence: Synthetic peptide”
FDHTPF

SEQ ID NO: 52 - protein, 6 aa, Artificial Sequence
  source: /note=“Description of Artificial Sequence: Synthetic peptide”
QSISSN

SEQ ID NO: 53 - protein, 3 aa, Artificial Sequence
  source: /note=“Description of Artificial Sequence: Synthetic peptide”
AAS

SEQ ID NO: 54 - protein, 9 aa, Artificial Sequence
  source: /note=“Description of Artificial Sequence: Synthetic peptide”
LQFDHTPFT

SEQ ID NO: 55 - protein, 107 aa, Artificial Sequence
  source: /note=“Description of Artificial Sequence: Synthetic polypeptide”
DIQMTQSPSSLSASVGDRVTITCRASQSISSNLNWYQQKPGKAPKLLIYAASNLQSGVPSRFSGSGSGTDFTLTISSLQPEDFATYYCLQFDHTPFTFGQGTKVEIK

SEQ ID NO: 56 - DNA, 321 bp, Artificial Sequence
  source: /note=“Description of Artificial Sequence: Synthetic polynucleotide”
gacatccagatgacccagagcccctccagcctgtccgcctccgtgggcgacagagtgaccatcacctgtcgggcctcccagtccatctcctccaacctgaactggtatcagcagaagcccggcaaggcccctaagctgctgatctacgccgccagcaacctgcagtccggcgtgccctccagattctccggctctggctccggcaccgacttcaccctgaccatctccagcctgcagcccgaggacttcgccacctactactgcctgcagttcgaccacacccctttcaccttcggccagggcaccaaagtggaaatcaag

SEQ ID NO: 57 - protein, 214 aa, Artificial Sequence
  source: /note=“Description of Artificial Sequence: Synthetic polypeptide”
DIQMTQSPSSLSASVGDRVTITCRASQSISSNLNWYQQKPGKAPKLLIYAASNLQSGVPSRFSGSGSGTDFTLTISSLQPEDFATYYCLQFDHTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - DNA, 642 bp, Artificial Sequence
  source: /note=“Description of Artificial Sequence: Synthetic polynucleotide”
gacatccagatgacccagagcccctccagcctgtccgcctccgtgggcgacagagtgaccatcacctgtcgggcctcccagtccatctcctccaacctgaactggtatcagcagaagcccggcaaggcccctaagctgctgatctacgccgccagcaacctgcagtccggcgtgccctccagattctccggctctggctccggcaccgacttcaccctgaccatctccagcctgcagcccgaggacttcgccacctactactgcctgcagttcgaccacacccctttcaccttcggccagggcaccaaagtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 59 - protein, 10 aa, Artificial Sequence
  source: /note=“Description of Artificial Sequence: Synthetic peptide”
GFTFSSAAVH

SEQ ID NO: 60 - protein, 19 aa, Artificial Sequence
  source: /note=“Description of Artificial Sequence: Synthetic peptide”
RIKSKADGGTTDYAAPVKG

SEQ ID NO: 61 - protein, 18 aa, Artificial Sequence
  source: /note=“Description of Artificial Sequence: Synthetic peptide”
DSPSISSYSIPYFSGMDV

SEQ ID NO: 62 - protein, 5 aa, Artificial Sequence
  source: /note=“Description of Artificial Sequence: Synthetic peptide”
SAAVH

SEQ ID NO: 63 - protein, 19 aa, Artificial Sequence
  source: /note=“Description of Artificial Sequence: Synthetic peptide”
RIKSKADGGTTDYAAPVKG

SEQ ID NO: 64 - protein, 18 aa, Artificial Sequence
  source: /note=“Description of Artificial Sequence: Synthetic peptide”
DSPSISSYSIPYFSGMDV

SEQ ID NO: 65 - protein, 7 aa, Artificial Sequence
  source: /note=“Description of Artificial Sequence: Synthetic peptide”
GFTFSSA

SEQ ID NO: 66 - protein, 8 aa, Artificial Sequence
  source: /note=“Description of Artificial Sequence: Synthetic peptide”
KSKADGGT

SEQ ID NO: 67 - protein, 18 aa, Artificial Sequence
  source: /note=“Description of Artificial Sequence: Synthetic peptide”
DSPSISSYSIPYFSGMDV

SEQ ID NO: 68 - protein, 8 aa, Artificial Sequence
  source: /note=“Description of Artificial Sequence: Synthetic peptide”
GFTFSSAA

SEQ ID NO: 69 - protein, 10 aa, Artificial Sequence
  source: /note=“Description of Artificial Sequence: Synthetic peptide”
IKSKADGGTT

SEQ ID NO: 70 - protein, 20 aa, Artificial Sequence
  source: /note=“Description of Artificial Sequence: Synthetic peptide”
ARDSPSISSYSIPYFSGMDV

SEQ ID NO: 71 - protein, 129 aa, Artificial Sequence
  source: /note=“Description of Artificial Sequence: Synthetic polypeptide”
QVQLVESGGGLVKPGGSLRLSCAASGFTFSSAAVHWVRQAPGKGLEWVGRIKSKADGGTTDYAAPVKGRFTISRDDSKNTLYLQMNSLKTEDTAVYYCARDSPSISSYSIPYFSGMDVWGQGTLVTVSS

SEQ ID NO: 72 - DNA, 387 bp, Artificial Sequence
  source: /note=“Description of Artificial Sequence: Synthetic polynucleotide”
caagtgcagctggtggaatctggcggcggactggtcaagcctggcggtagcctgagactgtcttgcgccgcctccggcttcaccttctcctctgccgctgtgcactgggtccgacaggcccctggcaagggcctggagtgggtcggacggatcaagtccaaggccgacggcggcaccaccgactacgctgcccctgtgaagggccggttcaccatctcccgggacgactccaagaacaccctgtacctgcagatgaactccctgaaaaccgaggacaccgccgtgtactactgcgccagagactccccatctatctccagctactccatcccctacttctccggcatggacgtgtggggccagggcaccctggtcaccgtgtcctct

SEQ ID NO: 73 - protein, 232 aa, Artificial Sequence
  source: /note=“Description of Artificial Sequence: Synthetic polypeptide”
QVQLVESGGGLVKPGGSLRLSCAASGFTFSSAAVHWVRQAPGKGLEWVGRIKSKADGGTTDYAAPVKGRFTISRDDSKNTLYLQMNSLKTEDTAVYYCARDSPSISSYSIPYFSGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 74 - DNA, 696 bp, Artificial Sequence
  source: /note=“Description of Artificial Sequence: Synthetic polynucleotide”
caagtgcagctggtggaatctggcggcggactggtcaagcctggcggtagcctgagactgtcttgcgccgcctccggcttcaccttctcctctgccgctgtgcactgggtccgacaggcccctggcaagggcctggagtgggtcggacggatcaagtccaaggccgacggcggcaccaccgactacgctgcccctgtgaagggccggttcaccatctcccgggacgactccaagaacaccctgtacctgcagatgaactccctgaaaaccgaggacaccgccgtgtactactgcgccagagactccccatctatctccagctactccatcccctacttctccggcatggacgtgtggggccagggcaccctggtcaccgtgtcctctgctagcaccaagggcccctccgtgttccctctggccccttccagcaagtctacctctggcggcaccgctgctctgggctgcctggtgaaggactacttccctgagcctgtgacagtgtcctggaactctggcgccctgacctccggcgtgcacaccttccctgccgtgctgcagtcctccggcctgtactccctgtcctccgtggtgacagtgccttcctccagcctgggcacccagacctatatctgcaacgtgaaccacaagccttccaacaccaaggtggacaagcgggtggagcctaagtcatgc

SEQ ID NO: 75 - protein, 11 aa, Artificial Sequence
  source: /note=“Description of Artificial Sequence: Synthetic peptide”
RASQGIRAWLN

SEQ ID NO: 76 - protein, 7 aa, Artificial Sequence
  source: /note=“Description of Artificial Sequence: Synthetic peptide”
AASSLQS

SEQ ID NO: 77 - protein, 9 aa, Artificial Sequence
  source: /note=“Description of Artificial Sequence: Synthetic peptide”
HQYITHPPT

SEQ ID NO: 78 - protein, 11 aa, Artificial Sequence
  source: /note=“Description of Artificial Sequence: Synthetic peptide”
RASQGIRAWLN

SEQ ID NO: 79 - protein, 7 aa, Artificial Sequence
  source: /note=“Description of Artificial Sequence: Synthetic peptide”
AASSLQS

SEQ ID NO: 80 - protein, 9 aa, Artificial Sequence
  source: /note=“Description of Artificial Sequence: Synthetic peptide”
HQYITHPPT

SEQ ID NO: 81 - protein, 7 aa, Artificial Sequence
  source: /note=“Description of Artificial Sequence: Synthetic peptide”
SQGIRAW

SEQ ID NO: 82 - protein, 3 aa, Artificial Sequence
  source: /note=“Description of Artificial Sequence: Synthetic peptide”
AAS

SEQ ID NO: 83 - protein, 6 aa, Artificial Sequence
  source: /note=“Description of Artificial Sequence: Synthetic peptide”
YITHPP

SEQ ID NO: 84 - protein, 6 aa, Artificial Sequence
  source: /note=“Description of Artificial Sequence: Synthetic peptide”
QGIRAW

SEQ ID NO: 85 - protein, 3 aa, Artificial Sequence
  source: /note=“Description of Artificial Sequence: Synthetic peptide”
AAS

SEQ ID NO: 86 - protein, 9 aa, Artificial Sequence
  source: /note=“Description of Artificial Sequence: Synthetic peptide”
HQYITHPPT

SEQ ID NO: 87 - protein, 107 aa, Artificial Sequence
  source: /note=“Description of Artificial Sequence: Synthetic polypeptide”
DIQMTQSPSSLSASVGDRVTITCRASQGIRAWLNWYQQKPGKAPKLLIYAASSLQSGVPSRFSGSGSGTDFTLTISSLQPEDFATYYCHQYITHPPTFGQGTKVEIK

SEQ ID NO: 88 - DNA, 321 bp, Artificial Sequence
  source: /note=“Description of Artificial Sequence: Synthetic polynucleotide”
gacatccagatgacccagagcccctccagcctgtccgcctccgtgggcgacagagtgaccatcacctgtcgggcctctcagggcatccgggcctggctgaactggtatcagcagaagcccggcaaggcccctaagctgctgatctacgccgccagctccctgcagtccggcgtgccctccagattctccggctctggctccggcaccgacttcaccctgaccatctccagcctgcagcccgaggacttcgccacctactactgccaccagtacatcacccaccctcccaccttcggccagggcaccaaagtggaaatcaag

SEQ ID NO: 89 - protein, 214 aa, Artificial Sequence
  source: /note=“Description of Artificial Sequence: Synthetic polypeptide”
DIQMTQSPSSLSASVGDRVTITCRASQGIRAWLNWYQQKPGKAPKLLIYAASSLQSGVPSRFSGSGSGTDFTLTISSLQPEDFATYYCHQYITHPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - DNA, 642 bp, Artificial Sequence
  source: /note=“Description of Artificial Sequence: Synthetic polynucleotide”
gacatccagatgacccagagcccctccagcctgtccgcctccgtgggcgacagagtgaccatcacctgtcgggcctctcagggcatccgggcctggctgaactggtatcagcagaagcccggcaaggcccctaagctgctgatctacgccgccagctccctgcagtccggcgtgccctccagattctccggctctggctccggcaccgacttcaccctgaccatctccagcctgcagcccgaggacttcgccacctactactgccaccagtacatcacccaccctcccaccttcggccagggcaccaaagtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 91 - protein, 10 aa, Artificial Sequence
  source: /note=“Description of Artificial Sequence: Synthetic peptide”
GFTFQSAAVH

SEQ ID NO: 92 - protein, 19 aa, Artificial Sequence
  source: /note=“Description of Artificial Sequence: Synthetic peptide”
RIKSKADGGTTDYAAPVKG

SEQ ID NO: 93 - protein, 18 aa, Artificial Sequence
  source: /note=“Description of Artificial Sequence: Synthetic peptide”
DSPSISSYSIPYFSGMDV

SEQ ID NO: 94 - protein, 5 aa, Artificial Sequence
  source: /note=“Description of Artificial Sequence: Synthetic peptide”
SAAVH

SEQ ID NO: 95 - protein, 19 aa, Artificial Sequence
  source: /note=“Description of Artificial Sequence: Synthetic peptide”
RIKSKADGGTTDYAAPVKG

SEQ ID NO: 96 - protein, 18 aa, Artificial Sequence
  source: /note=“Description of Artificial Sequence: Synthetic peptide”
DSPSISSYSIPYFSGMDV

SEQ ID NO: 97 - protein, 7 aa, Artificial Sequence
  source: /note=“Description of Artificial Sequence: Synthetic peptide”
GFTFQSA

SEQ ID NO: 98 - protein, 8 aa, Artificial Sequence
  source: /note=“Description of Artificial Sequence: Synthetic peptide”
KSKADGGT

SEQ ID NO: 99 - protein, 18 aa, Artificial Sequence
  source: /note=“Description of Artificial Sequence: Synthetic peptide”
DSPSISSYSIPYFSGMDV

SEQ ID NO: 100 - protein, 8 aa, Artificial Sequence
  source: /note=“Description of Artificial Sequence: Synthetic peptide”
GFTFQSAA

SEQ ID NO: 101 - protein, 10 aa, Artificial Sequence
  source: /note=“Description of Artificial Sequence: Synthetic peptide”
IKSKADGGTT

SEQ ID NO: 102 - protein, 20 aa, Artificial Sequence
  source: /note=“Description of Artificial Sequence: Synthetic peptide”
ARDSPSISSYSIPYFSGMDV

SEQ ID NO: 103 - protein, 129 aa, Artificial Sequence
  source: /note=“Description of Artificial Sequence: Synthetic polypeptide”
QVQLVESGGGLVKPGGSLRLSCAASGFTFQSAAVHWVRQAPGKGLEWVGRIKSKADGGTTDYAAPVKGRFTISRDDSKNTLYLQMNSLKTEDTAVYYCARDSPSISSYSIPYFSGMDVWGQGTLVTVSS

SEQ ID NO: 104 - DNA, 387 bp, Artificial Sequence
  source: /note=“Description of Artificial Sequence: Synthetic polynucleotide”
caagtgcagctggtggaatctggcggcggactggtcaagcctggcggtagcctgagactgtcttgcgccgcctccggcttcaccttccagtctgccgctgtgcactgggtccgacaggcccctggcaagggcctggagtgggtcggacggatcaagtccaaggccgacggcggcaccaccgactacgctgcccctgtgaagggccggttcaccatctcccgggacgactccaagaacaccctgtacctgcagatgaactccctgaaaaccgaggacaccgccgtgtactactgcgccagagactccccatctatctccagctactccatcccctacttctccggcatggacgtgtggggccagggcaccctggtcaccgtgtcctct

SEQ ID NO: 105 - protein, 232 aa, Artificial Sequence
  source: /note=“Description of Artificial Sequence: Synthetic polypeptide”
QVQLVESGGGLVKPGGSLRLSCAASGFTFQSAAVHWVRQAPGKGLEWVGRIKSKADGGTTDYAAPVKGRFTISRDDSKNTLYLQMNSLKTEDTAVYYCARDSPSISSYSIPYFSGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 106 - DNA, 696 bp, Artificial Sequence
  source: /note=“Description of Artificial Sequence: Synthetic polynucleotide”
caagtgcagctggtggaatctggcggcggactggtcaagcctggcggtagcctgagactgtcttgcgccgcctccggcttcaccttccagtctgccgctgtgcactgggtccgacaggcccctggcaagggcctggagtgggtcggacggatcaagtccaaggccgacggcggcaccaccgactacgctgcccctgtgaagggccggttcaccatctcccgggacgactccaagaacaccctgtacctgcagatgaactccctgaaaaccgaggacaccgccgtgtactactgcgccagagactccccatctatctccagctactccatcccctacttctccggcatggacgtgtggggccagggcaccctggtcaccgtgtcctctgctagcaccaagggcccctccgtgttccctctggccccttccagcaagtctacctctggcggcaccgctgctctgggctgcctggtgaaggactacttccctgagcctgtgacagtgtcctggaactctggcgccctgacctccggcgtgcacaccttccctgccgtgctgcagtcctccggcctgtactccctgtcctccgtggtgacagtgccttcctccagcctgggcacccagacctatatctgcaacgtgaaccacaagccttccaacaccaaggtggacaagcgggtggagcctaagtcatgc

SEQ ID NO: 107 - protein, 11 aa, Artificial Sequence
  source: /note=“Description of Artificial Sequence: Synthetic peptide”
RASQGIRAWLN

SEQ ID NO: 108 - protein, 7 aa, Artificial Sequence
  source: /note=“Description of Artificial Sequence: Synthetic peptide”
AASSLQS

SEQ ID NO: 109 - protein, 9 aa, Artificial Sequence
  source: /note=“Description of Artificial Sequence: Synthetic peptide”
HQYITHPPT

SEQ ID NO: 110 - protein, 11 aa, Artificial Sequence
  source: /note=“Description of Artificial Sequence: Synthetic peptide”
RASQGIRAWLN

SEQ ID NO: 111 - protein, 7 aa, Artificial Sequence
  source: /note=“Description of Artificial Sequence: Synthetic peptide”
AASSLQS

SEQ ID NO: 112 - protein, 9 aa, Artificial Sequence
  source: /note=“Description of Artificial Sequence: Synthetic peptide”
HQYITHPPT

SEQ ID NO: 113 - protein, 7 aa, Artificial Sequence
  source: /note=“Description of Artificial Sequence: Synthetic peptide”
SQGIRAW

SEQ ID NO: 114 - protein, 3 aa, Artificial Sequence
  source: /note=“Description of Artificial Sequence: Synthetic peptide”
AAS

SEQ ID NO: 115 - protein, 6 aa, Artificial Sequence
  source: /note=“Description of Artificial Sequence: Synthetic peptide”
YITHPP

SEQ ID NO: 116 - protein, 6 aa, Artificial Sequence
  source: /note=“Description of Artificial Sequence: Synthetic peptide”
QGIRAW

SEQ ID NO: 117 - protein, 3 aa, Artificial Sequence
  source: /note=“Description of Artificial Sequence: Synthetic peptide”
AAS

SEQ ID NO: 118 - protein, 9 aa, Artificial Sequence
  source: /note=“Description of Artificial Sequence: Synthetic peptide”
HQYITHPPT

SEQ ID NO: 119 - protein, 107 aa, Artificial Sequence; identical to SEQ ID NO: 87
  source: /note=“Description of Artificial Sequence: Synthetic polypeptide”

SEQ ID NO: 120 - DNA, 321 bp, Artificial Sequence; identical to SEQ ID NO: 88
  source: /note=“Description of Artificial Sequence: Synthetic polynucleotide”

SEQ ID NO: 121 - protein, 214 aa, Artificial Sequence; identical to SEQ ID NO: 89
  source: /note=“Description of Artificial Sequence: Synthetic polypeptide”

SEQ ID NO: 122 - DNA, 642 bp, Artificial Sequence; identical to SEQ ID NO: 90
  source: /note=“Description of Artificial Sequence: Synthetic polynucleotide”

SEQ ID NO: 123 - protein, 10 aa, Artificial Sequence
  source: /note=“Description of Artificial Sequence: Synthetic peptide”
GFTFSSAAVH

SEQ ID NO: 124 - protein, 19 aa, Artificial Sequence
  source: /note=“Description of Artificial Sequence: Synthetic peptide”
RIKSKASGGTTDYAAPVKG

SEQ ID NO: 125 - protein, 18 aa, Artificial Sequence
  source: /note=“Description of Artificial Sequence: Synthetic peptide”
DSPSISSYSIPYFSGMDV

SEQ ID NO: 126 - protein, 5 aa, Artificial Sequence
  source: /note=“Description of Artificial Sequence: Synthetic peptide”
SAAVH

SEQ ID NO: 127 - protein, 19 aa, Artificial Sequence
  source: /note=“Description of Artificial Sequence: Synthetic peptide”
RIKSKASGGTTDYAAPVKG

SEQ ID NO: 128 - protein, 18 aa, Artificial Sequence
  source: /note=“Description of Artificial Sequence: Synthetic peptide”
DSPSISSYSIPYFSGMDV

SEQ ID NO: 129 - protein, 7 aa, Artificial Sequence
  source: /note=“Description of Artificial Sequence: Synthetic peptide”
GFTFSSA

SEQ ID NO: 130 - protein, 8 aa, Artificial Sequence
  source: /note=“Description of Artificial Sequence: Synthetic peptide”
KSKASGGT

SEQ ID NO: 131 - protein, 18 aa, Artificial Sequence
  source: /note=“Description of Artificial Sequence: Synthetic peptide”
DSPSISSYSIPYFSGMDV

SEQ ID NO: 132 - protein, 8 aa, Artificial Sequence
  source: /note=“Description of Artificial Sequence: Synthetic peptide”
GFTFSSAA

SEQ ID NO: 133 - protein, 10 aa, Artificial Sequence
  source: /note=“Description of Artificial Sequence: Synthetic peptide”
IKSKASGGTT

SEQ ID NO: 134 - protein, 20 aa, Artificial Sequence
  source: /note=“Description of Artificial Sequence: Synthetic peptide”
ARDSPSISSYSIPYFSGMDV

SEQ ID NO: 135 - protein, 129 aa, Artificial Sequence
  source: /note=“Description of Artificial Sequence: Synthetic polypeptide”
QVQLVESGGGLVKPGGSLRLSCAASGFTFSSAAVHWVRQAPGKGLEWVGRIKSKASGGTTDYAAPVKGRFTISRDDSKNTLYLQMNSLKTEDTAVYYCARDSPSISSYSIPYFSGMDVWGQGTLVTVSS

SEQ ID NO: 136 - DNA, 387 bp, Artificial Sequence
  source: /note=“Description of Artificial Sequence: Synthetic polynucleotide”
caagtgcagctggtggaatctggcggcggactggtcaagcctggcggtagcctgagactgtcttgcgccgcctccggcttcaccttctcctctgccgctgtgcactgggtccgacaggcccctggcaagggcctggagtgggtcggacggatcaagtccaaggcctccggcggcaccaccgactacgctgcccctgtgaagggccggttcaccatctcccgggacgactccaagaacaccctgtacctgcagatgaactccctgaaaaccgaggacaccgccgtgtactactgcgccagagactccccatctatctccagctactccatcccctacttctccggcatggacgtgtggggccagggcaccctggtcaccgtgtcctct

SEQ ID NO: 137 - protein, 232 aa, Artificial Sequence
  source: /note=“Description of Artificial Sequence: Synthetic polypeptide”
QVQLVESGGGLVKPGGSLRLSCAASGFTFSSAAVHWVRQAPGKGLEWVGRIKSKASGGTTDYAAPVKGRFTISRDDSKNTLYLQMNSLKTEDTAVYYCARDSPSISSYSIPYFSGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 138 - DNA, 696 bp, Artificial Sequence
  source: /note=“Description of Artificial Sequence: Synthetic polynucleotide”
caagtgcagctggtggaatctggcggcggactggtcaagcctggcggtagcctgagactgtcttgcgccgcctccggcttcaccttctcctctgccgctgtgcactgggtccgacaggcccctggcaagggcctggagtgggtcggacggatcaagtccaaggcctccggcggcaccaccgactacgctgcccctgtgaagggccggttcaccatctcccgggacgactccaagaacaccctgtacctgcagatgaactccctgaaaaccgaggacaccgccgtgtactactgcgccagagactccccatctatctccagctactccatcccctacttctccggcatggacgtgtggggccagggcaccctggtcaccgtgtcctctgctagcaccaagggcccctccgtgttccctctggccccttccagcaagtctacctctggcggcaccgctgctctgggctgcctggtgaaggactacttccctgagcctgtgacagtgtcctggaactctggcgccctgacctccggcgtgcacaccttccctgccgtgctgcagtcctccggcctgtactccctgtcctccgtggtgacagtgccttcctccagcctgggcacccagacctatatctgcaacgtgaaccacaagccttccaacaccaaggtggacaagcgggtggagcctaagtcatgc

SEQ ID NO: 139 - protein, 11 aa, Artificial Sequence
  source: /note=“Description of Artificial Sequence: Synthetic peptide”
RASQGIRAWLN

SEQ ID NO: 140 - protein, 7 aa, Artificial Sequence
  source: /note=“Description of Artificial Sequence: Synthetic peptide”
AASSLQS

SEQ ID NO: 141 - protein, 9 aa, Artificial Sequence
  source: /note=“Description of Artificial Sequence: Synthetic peptide”
HQYITHPPT

SEQ ID NO: 142 - protein, 11 aa, Artificial Sequence
  source: /note=“Description of Artificial Sequence: Synthetic peptide”
RASQGIRAWLN

SEQ ID NO: 143 - protein, 7 aa, Artificial Sequence
  source: /note=“Description of Artificial Sequence: Synthetic peptide”
AASSLQS

SEQ ID NO: 144 - protein, 9 aa, Artificial Sequence
  source: /note=“Description of Artificial Sequence: Synthetic peptide”
HQYITHPPT

SEQ ID NO: 145 - protein, 7 aa, Artificial Sequence
  source: /note=“Description of Artificial Sequence: Synthetic peptide”
SQGIRAW

SEQ ID NO: 146 - protein, 3 aa, Artificial Sequence
  source: /note=“Description of Artificial Sequence: Synthetic peptide”
AAS

SEQ ID NO: 147 - protein, 6 aa, Artificial Sequence
  source: /note=“Description of Artificial Sequence: Synthetic peptide”
YITHPP

SEQ ID NO: 148 - protein, 6 aa, Artificial Sequence
  source: /note=“Description of Artificial Sequence: Synthetic peptide”
QGIRAW

SEQ ID NO: 149 - protein, 3 aa, Artificial Sequence
  source: /note=“Description of Artificial Sequence: Synthetic peptide”
AAS

SEQ ID NO: 150 - protein, 9 aa, Artificial Sequence
  source: /note=“Description of Artificial Sequence: Synthetic peptide”
HQYITHPPT

SEQ ID NO: 151 - protein, 107 aa, Artificial Sequence; identical to SEQ ID NO: 87
  source: /note=“Description of Artificial Sequence: Synthetic polypeptide”

SEQ ID NO: 152 - DNA, 321 bp, Artificial Sequence; identical to SEQ ID NO: 88
  source: /note=“Description of Artificial Sequence: Synthetic polynucleotide”

SEQ ID NO: 153 - protein, 214 aa, Artificial Sequence; identical to SEQ ID NO: 89
  source: /note=“Description of Artificial Sequence: Synthetic polypeptide”

SEQ ID NO: 154 - DNA, 642 bp, Artificial Sequence; identical to SEQ ID NO: 90
  source: /note=“Description of Artificial Sequence: Synthetic polynucleotide”

SEQ ID NO: 155 - protein, 10 aa, Artificial Sequence
  source: /note=“Description of Artificial Sequence: Synthetic peptide”
GFTFSSAAVH

SEQ ID NO: 156 - protein, 19 aa, Artificial Sequence
  source: /note=“Description of Artificial Sequence: Synthetic peptide”
RIKSKADAGTTDYAAPVKG

SEQ ID NO: 157 - protein, 18 aa, Artificial Sequence
  source: /note=“Description of Artificial Sequence: Synthetic peptide”
DSPSISSYSIPYFSGMDV

SEQ ID NO: 158 - protein, 5 aa, Artificial Sequence
  source: /note=“Description of Artificial Sequence: Synthetic peptide”
SAAVH

SEQ ID NO: 159 - protein, 19 aa, Artificial Sequence
  source: /note=“Description of Artificial Sequence: Synthetic peptide”
RIKSKADAGTTDYAAPVKG

SEQ ID NO: 160 - protein, 18 aa, Artificial Sequence
  source: /note=“Description of Artificial Sequence: Synthetic peptide”
DSPSISSYSIPYFSGMDV

SEQ ID NO: 161 - protein, 7 aa, Artificial Sequence
  source: /note=“Description of Artificial Sequence: Synthetic peptide”
GFTFSSA

SEQ ID NO: 162 - protein, 8 aa, Artificial Sequence
  source: /note=“Description of Artificial Sequence: Synthetic peptide”
KSKADAGT

SEQ ID NO: 163 - protein, 18 aa, Artificial Sequence
  source: /note=“Description of Artificial Sequence: Synthetic peptide”
DSPSISSYSIPYFSGMDV

SEQ ID NO: 164 - protein, 8 aa, Artificial Sequence
  source: /note=“Description of Artificial Sequence: Synthetic peptide”
GFTFSSAA

SEQ ID NO: 165 - protein, 10 aa, Artificial Sequence
  source: /note=“Description of Artificial Sequence: Synthetic peptide”
IKSKADAGTT

SEQ ID NO: 166 - protein, 20 aa, Artificial Sequence
  source: /note=“Description of Artificial Sequence: Synthetic peptide”
ARDSPSISSYSIPYFSGMDV

SEQ ID NO: 167 - protein, 129 aa, Artificial Sequence
  source: /note=“Description of Artificial Sequence: Synthetic polypeptide”
QVQLVESGGGLVKPGGSLRLSCAASGFTFSSAAVHWVRQAPGKGLEWVGRIKSKADAGTTDYAAPVKGRFTISRDDSKNTLYLQMNSLKTEDTAVYYCARDSPSISSYSIPYFSGMDVWGQGTLVTVSS

SEQ ID NO: 168 - DNA, 387 bp, Artificial Sequence
  source: /note=“Description of Artificial Sequence: Synthetic polynucleotide”
caagtgcagctggtggaatctggcggcggactggtcaagcctggcggtagcctgagactgtcttgcgccgcctccggcttcaccttctcctctgccgctgtgcactgggtccgacaggcccctggcaagggcctggagtgggtcggacggatcaagtccaaggccgacgccggcaccaccgactacgctgcccctgtgaagggccggttcaccatctcccgggacgactccaagaacaccctgtacctgcagatgaactccctgaaaaccgaggacaccgccgtgtactactgcgccagagactccccatctatctccagctactccatcccctacttctccggcatggacgtgtggggccagggcaccctggtcaccgtgtcctct

SEQ ID NO: 169 - protein, 232 aa, Artificial Sequence
  source: /note=“Description of Artificial Sequence: Synthetic polypeptide”
QVQLVESGGGLVKPGGSLRLSCAASGFTFSSAAVHWVRQAPGKGLEWVGRIKSKADAGTTDYAAPVKGRFTISRDDSKNTLYLQMNSLKTEDTAVYYCARDSPSISSYSIPYFSGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 170 - DNA, 696 bp, Artificial Sequence
  source: /note=“Description of Artificial Sequence: Synthetic polynucleotide”
caagtgcagctggtggaatctggcggcggactggtcaagcctggcggtagcctgagactgtcttgcgccgcctccggcttcaccttctcctctgccgctgtgcactgggtccgacaggcccctggcaagggcctggagtgggtcggacggatcaagtccaaggccgacgccggcaccaccgactacgctgcccctgtgaagggccggttcaccatctcccgggacgactccaagaacaccctgtacctgcagatgaactccctgaaaaccgaggacaccgccgtgtactactgcgccagagactccccatctatctccagctactccatcccctacttctccggcatggacgtgtggggccagggcaccctggtcaccgtgtcctctgctagcaccaagggcccctccgtgttccctctggccccttccagcaagtctacctctggcggcaccgctgctctgggctgcctggtgaaggactacttccctgagcctgtgacagtgtcctggaactctggcgccctgacctccggcgtgcacaccttccctgccgtgctgcagtcctccggcctgtactccctgtcctccgtggtgacagtgccttcctccagcctgggcacccagacctatatctgcaacgtgaaccacaagccttccaacaccaaggtggacaagcgggtggagcctaagtcatgc

SEQ ID NO: 171 - protein, 11 aa, Artificial Sequence
  source: /note=“Description of Artificial Sequence: Synthetic peptide”
RASQGIRAWLN

SEQ ID NO: 172 - protein, 7 aa, Artificial Sequence
  source: /note=“Description of Artificial Sequence: Synthetic peptide”
AASSLQS

SEQ ID NO: 173 - protein, 9 aa, Artificial Sequence
  source: /note=“Description of Artificial Sequence: Synthetic peptide”
HQYITHPPT

SEQ ID NO: 174 - protein, 11 aa, Artificial Sequence
  source: /note=“Description of Artificial Sequence: Synthetic peptide”
RASQGIRAWLN

SEQ ID NO: 175 - protein, 7 aa, Artificial Sequence
  source: /note=“Description of Artificial Sequence: Synthetic peptide”
AASSLQS

SEQ ID NO: 176 - protein, 9 aa, Artificial Sequence
  source: /note=“Description of Artificial Sequence: Synthetic peptide”
HQYITHPPT

SEQ ID NO: 177 - protein, 7 aa, Artificial Sequence
  source: /note=“Description of Artificial Sequence: Synthetic peptide”
SQGIRAW

SEQ ID NO: 178 - protein, 3 aa, Artificial Sequence
  source: /note=“Description of Artificial Sequence: Synthetic peptide”
AAS

SEQ ID NO: 179 - protein, 6 aa, Artificial Sequence
  source: /note=“Description of Artificial Sequence: Synthetic peptide”
YITHPP

SEQ ID NO: 180 - protein, 6 aa, Artificial Sequence
  source: /note=“Description of Artificial Sequence: Synthetic peptide”
QGIRAW

SEQ ID NO: 181 - protein, 3 aa, Artificial Sequence
  source: /note=“Description of Artificial Sequence: Synthetic peptide”
AAS

SEQ ID NO: 182 - protein, 9 aa, Artificial Sequence
  source: /note=“Description of Artificial Sequence: Synthetic peptide”
HQYITHPPT

SEQ ID NO: 183 - protein, 107 aa, Artificial Sequence; identical to SEQ ID NO: 87
  source: /note=“Description of Artificial Sequence: Synthetic polypeptide”

SEQ ID NO: 184 - DNA, 321 bp, Artificial Sequence; identical to SEQ ID NO: 88
  source: /note=“Description of Artificial Sequence: Synthetic polynucleotide”

SEQ ID NO: 185 - protein, 214 aa, Artificial Sequence; identical to SEQ ID NO: 89
  source: /note=“Description of Artificial Sequence: Synthetic polypeptide”

SEQ ID NO: 186 - DNA, 642 bp, Artificial Sequence; identical to SEQ ID NO: 90
  source: /note=“Description of Artificial Sequence: Synthetic polynucleotide”

SEQ ID NO: 187 - protein, 10 aa, Artificial Sequence
  source: /note=“Description of Artificial Sequence: Synthetic peptide”
GYSFTNYWIG

SEQ ID NO: 188 - protein, 17 aa, Artificial Sequence
  source: /note=“Description of Artificial Sequence: Synthetic peptide”
IIFPGVSYTKYSPSFQG

SEQ ID NO: 189 - protein, 14 aa, Artificial Sequence
  source: /note=“Description of Artificial Sequence: Synthetic peptide”
GSDQTLRGSRAMDY

SEQ ID NO: 190 - protein, 5 aa, Artificial Sequence
  source: /note=“Description of Artificial Sequence: Synthetic peptide”
NYWIG

SEQ ID NO: 191 - protein, 17 aa, Artificial Sequence
  source: /note=“Description of Artificial Sequence: Synthetic peptide”
IIFPGVSYTKYSPSFQG

SEQ ID NO: 192 - protein, 14 aa, Artificial Sequence
  source: /note=“Description of Artificial Sequence: Synthetic peptide”
GSDQTLRGSRAMDY

SEQ ID NO: 193 - protein, 7 aa, Artificial Sequence
  source: /note=“Description of Artificial Sequence: Synthetic peptide”
GYSFTNY

SEQ ID NO: 194 - protein, 6 aa, Artificial Sequence
  source: /note=“Description of Artificial Sequence: Synthetic peptide”
FPGVSY

SEQ ID NO: 195 - protein, 14 aa, Artificial Sequence
  source: /note=“Description of Artificial Sequence: Synthetic peptide”
GSDQTLRGSRAMDY

SEQ ID NO: 196 - protein, 8 aa, Artificial Sequence
  source: /note=“Description of Artificial Sequence: Synthetic peptide”
GYSFTNYW

SEQ ID NO: 197 - protein, 8 aa, Artificial Sequence
  source: /note=“Description of Artificial Sequence: Synthetic peptide”
IFPGVSYT

SEQ ID NO: 198 - protein, 16 aa, Artificial Sequence
  source: /note=“Description of Artificial Sequence: Synthetic peptide”
ARGSDQTLRGSRAMDY

SEQ ID NO: 199 - protein, 123 aa, Artificial Sequence
  source: /note=“Description of Artificial Sequence: Synthetic polypeptide”
QVQLVQSGAEVKKPGESLKISCKGSGYSFTNYWIGWVRQMPGKGLEWMGIIFPGVSYTKYSPSFQGQVTISADKSISTAYLQWSSLKASDTAMYYCARGSDQTLRGSRAMDYWGQGTLVTVSS

SEQ ID NO: 200 - DNA, 369 bp, Artificial Sequence
  source: /note=“Description of Artificial Sequence: Synthetic polynucleotide”
caagtgcagctggtgcagtctggcgctgaagtgaagaagcccggcgagtccctgaagatctcctgcaagggctccggctactccttcaccaactactggatcggctgggtccgacagatgcccggcaagggcctggagtggatgggcatcatcttccccggcgtgtcctacaccaagtacagccccagcttccagggccaagtcacaatctccgccgacaagtccatctccaccgcctacctgcagtggtcctccctgaaggcctccgacaccgccatgtactactgcgccagaggctccgaccagaccctgcggggctccagagccatggattactggggccagggcaccctggtcaccgtgtcctct

SEQ ID NO: 201 - protein, 226 aa, Artificial Sequence
  source: /note=“Description of Artificial Sequence: Synthetic polypeptide”
QVQLVQSGAEVKKPGESLKISCKGSGYSFTNYWIGWVRQMPGKGLEWMGIIFPGVSYTKYSPSFQGQVTISADKSISTAYLQWSSLKASDTAMYYCARGSDQTLRGSRAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 202 - DNA, 678 bp, Artificial Sequence
  source: /note=“Description of Artificial Sequence: Synthetic polynucleotide”
caagtgcagctggtgcagtctggcgctgaagtgaagaagcccggcgagtccctgaagatctcctgcaagggctccggctactccttcaccaactactggatcggctgggtccgacagatgcccggcaagggcctggagtggatgggcatcatcttccccggcgtgtcctacaccaagtacagccccagcttccagggccaagtcacaatctccgccgacaagtccatctccaccgcctacctgcagtggtcctccctgaaggcctccgacaccgccatgtactactgcgccagaggctccgaccagaccctgcggggctccagagccatggattactggggccagggcaccctggtcaccgtgtcctctgctagcaccaagggcccctccgtgttccctctggccccttccagcaagtctacctctggcggcaccgctgctctgggctgcctggtgaaggactacttccctgagcctgtgacagtgtcctggaactctggcgccctgacctccggcgtgcacaccttccctgccgtgctgcagtcctccggcctgtactccctgtcctccgtggtgacagtgccttcctccagcctgggcacccagacctatatctgcaacgtgaaccacaagccttccaacaccaaggtggacaagcgggtggagcctaagtcatgc

SEQ ID NO: 203 - protein, 14 aa, Artificial Sequence
  source: /note=“Description of Artificial Sequence: Synthetic peptide”
TGTSSDVGISNYVS

SEQ ID NO: 204 - protein, 7 aa, Artificial Sequence
  source: /note=“Description of Artificial Sequence: Synthetic peptide”
EVSNRPS

SEQ ID NO: 205 - protein, 9 aa, Artificial Sequence
  source: /note=“Description of Artificial Sequence: Synthetic peptide”
QSYTSLNYV

SEQ ID NO: 206 - protein, 14 aa, Artificial Sequence
  source: /note=“Description of Artificial Sequence: Synthetic peptide”
TGTSSDVGISNYVS

SEQ ID NO: 207 - protein, 7 aa, Artificial Sequence
  source: /note=“Description of Artificial Sequence: Synthetic peptide”
EVSNRPS

SEQ ID NO: 208 - protein, 9 aa, Artificial Sequence
  source: /note=“Description of Artificial Sequence: Synthetic peptide”
QSYTSLNYV

SEQ ID NO: 209 - protein, 10 aa, Artificial Sequence
  source: /note=“Description of Artificial Sequence: Synthetic peptide”
TSSDVGISNY

SEQ ID NO: 210 - protein, 3 aa, Artificial Sequence
  source: /note=“Description of Artificial Sequence: Synthetic peptide”
EVS

SEQ ID NO: 211 - protein, 6 aa, Artificial Sequence
  source: /note=“Description of Artificial Sequence: Synthetic peptide”
YTSLNY

SEQ ID NO: 212 - protein, 9 aa, Artificial Sequence
  source: /note=“Description of Artificial Sequence: Synthetic peptide”
SSDVGISNY

SEQ ID NO: 213 - protein, 3 aa, Artificial Sequence
  source: /note=“Description of Artificial Sequence: Synthetic peptide”
EVS

SEQ ID NO: 214 - protein, 9 aa, Artificial Sequence
  source: /note=“Description of Artificial Sequence: Synthetic peptide”
QSYTSLNYV

SEQ ID NO: 215 - protein, 109 aa, Artificial Sequence
  source: /note=“Description of Artificial Sequence: Synthetic polypeptide”
QSALTQPASVSGSPGQSITISCTGTSSDVGISNYVSWYQQHPGKAPKLMIYEVSNRPSGVSNRFSGSKSGNTASLTISGLQAEDEADYYCQSYTSLNYVFGGGTKLTVL

SEQ ID NO: 216 - DNA, 327 bp, Artificial Sequence
  source: /note=“Description of Artificial Sequence: Synthetic polynucleotide”
cagtccgccctgacccagcctgcctccgtgtctggctcccctggccagtccatcaccatcagctgcaccggcacctccagcgacgtgggcatctccaactacgtgtcctggtatcagcagcaccccggcaaggcccctaagctgatgatctacgaagtgtccaaccggccctccggcgtgtccaacagattctccggctccaagtccggcaacaccgcctccctgaccatcagcggcctgcaggctgaggacgaggccgactactactgccagtcctacacctccctgaactacgtgttcggcggaggcaccaagctgaccgtgctg

SEQ ID NO: 217 - protein, 215 aa, Artificial Sequence
  source: /note=“Description of Artificial Sequence: Synthetic polypeptide”
QSALTQPASVSGSPGQSITISCTGTSSDVGISNYVSWYQQHPGKAPKLMIYEVSNRPSGVSNRFSGSKSGNTASLTISGLQAEDEADYYCQSYTSLNYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 218 - DNA, 645 bp, Artificial Sequence
  source: /note=“Description of Artificial Sequence: Synthetic polynucleotide”
cagtccgccctgacccagcctgcctccgtgtctggctcccctggccagtccatcaccatcagctgcaccggcacctccagcgacgtgggcatctccaactacgtgtcctggtatcagcagcaccccggcaaggcccctaagctgatgatctacgaagtgtccaaccggccctccggcgtgtccaacagattctccggctccaagtccggcaacaccgcctccctgaccatcagcggcctgcaggctgaggacgaggccgactactactgccagtcctacacctccctgaactacgtgttcggcggaggcaccaagctgaccgtgctgggccagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 219 - protein, 10 aa, Artificial Sequence
  source: /note=“Description of Artificial Sequence: Synthetic peptide”
GFTFSSNAMH

SEQ ID NO: 220 - protein, 19 aa, Artificial Sequence
  source: /note=“Description of Artificial Sequence: Synthetic peptide”
RIKSKTDGGTTDYAAPVKG

SEQ ID NO: 221 - protein, 9 aa, Artificial Sequence
  source: /note=“Description of Artificial Sequence: Synthetic peptide”
DHYYYPFAY

SEQ ID NO: 222 - protein, 5 aa, Artificial Sequence
  source: /note=“Description of Artificial Sequence: Synthetic peptide”
SNAMH

SEQ ID NO: 223 - protein, 19 aa, Artificial Sequence
  source: /note=“Description of Artificial Sequence: Synthetic peptide”
RIKSKTDGGTTDYAAPVKG

SEQ ID NO: 224 - protein, 9 aa, Artificial Sequence
  source: /note=“Description of Artificial Sequence: Synthetic peptide”
DHYYYPFAY

SEQ ID NO: 225 - protein, 7 aa, Artificial Sequence
  source: /note=“Description of Artificial Sequence: Synthetic peptide”
GFTFSSN

SEQ ID NO: 226 - protein, 8 aa, Artificial Sequence
  source: /note=“Description of Artificial Sequence: Synthetic peptide”
KSKTDGGT

SEQ ID NO: 227 - protein, 9 aa, Artificial Sequence
  source: /note=“Description of Artificial Sequence: Synthetic peptide”
DHYYYPFAY

SEQ ID NO: 228 - protein, 8 aa, Artificial Sequence
  source: /note=“Description of Artificial Sequence: Synthetic peptide”
GFTFSSNA

SEQ ID NO: 229 - protein, 10 aa, Artificial Sequence
  source: /note=“Description of Artificial Sequence: Synthetic peptide”
IKSKTDGGTT

SEQ ID NO: 230 - protein, 11 aa, Artificial Sequence
  source: /note=“Description of Artificial Sequence: Synthetic peptide”
ARDHYYYPFAY

SEQ ID NO: 231 - protein, 120 aa, Artificial Sequence
  source: /note=“Description of Artificial Sequence: Synthetic polypeptide”
QVQLVESGGGLVKPGGSLRLSCAASGFTFSSNAMHWVRQAPGKGLEWVGRIKSKTDGGTTDYAAPVKGRFTISRDDSKNTLYLQMNSLKTEDTAVYYCARDHYYYPFAYWGQGTLVTVSS

SEQ ID NO: 232 - DNA, 360 bp, Artificial Sequence
  source: /note=“Description of Artificial Sequence: Synthetic polynucleotide”
caagtgcagctggtggaatctggcggcggactggtcaagcctggcggtagcctgagactgtcttgcgccgcctccggcttcaccttctcctccaacgccatgcactgggtccgacaggcccctggcaagggcctggagtgggtcggacggatcaagtccaagaccgacggcggcaccaccgactacgctgcccctgtgaagggccggttcaccatctcccgggacgactccaagaacaccctgtacctgcagatgaactccctgaaaaccgaggacaccgccgtgtactactgcgccagggaccactactactaccccttcgcctactggggccagggcaccctggtcaccgtgtcctct

SEQ ID NO: 233 - protein, 223 aa, Artificial Sequence
  source: /note=“Description of Artificial Sequence: Synthetic polypeptide”
QVQLVESGGGLVKPGGSLRLSCAASGFTFSSNAMHWVRQAPGKGLEWVGRIKSKTDGGTTDYAAPVKGRFTISRDDSKNTLYLQMNSLKTEDTAVYYCARDHYYYPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 234 - DNA, 669 bp, Artificial Sequence
  source: /note=“Description of Artificial Sequence: Synthetic polynucleotide”
caagtgcagctggtggaatctggcggcggactggtcaagcctggcggtagcctgagactgtcttgcgccgcctccggcttcaccttctcctccaacgccatgcactgggtccgacaggcccctggcaagggcctggagtgggtcggacggatcaagtccaagaccgacggcggcaccaccgactacgctgcccctgtgaagggccggttcaccatctcccgggacgactccaagaacaccctgtacctgcagatgaactccctgaaaaccgaggacaccgccgtgtactactgcgccagggaccactactactaccccttcgcctactggggccagggcaccctggtcaccgtgtcctctgctagcaccaagggcccctccgtgttccctctggccccttccagcaagtctacctctggcggcaccgctgctctgggctgcctggtgaaggactacttccctgagcctgtgacagtgtcctggaactctggcgccctgacctccggcgtgcacaccttccctgccgtgctgcagtcctccggcctgtactccctgtcctccgtggtgacagtgccttcctccagcctgggcacccagacctatatctgcaacgtgaaccacaagccttccaacaccaaggtggacaagcgggtggagcctaagtcatgc

SEQ ID NO: 235 - protein, 11 aa, Artificial Sequence
  source: /note=“Description of Artificial Sequence: Synthetic peptide”
RASQSIRYNLA

SEQ ID NO: 236 - protein, 7 aa, Artificial Sequence
  source: /note=“Description of Artificial Sequence: Synthetic peptide”
AASSLQS

SEQ ID NO: 237 - protein, 9 aa, Artificial Sequence
  source: /note=“Description of Artificial Sequence: Synthetic peptide”
HQYIAKPIT

SEQ ID NO: 238 - protein, 11 aa, Artificial Sequence
  source: /note=“Description of Artificial Sequence: Synthetic peptide”
RASQSIRYNLA

SEQ ID NO: 239 - protein, 7 aa, Artificial Sequence
  source: /note=“Description of Artificial Sequence: Synthetic peptide”
AASSLQS

SEQ ID NO: 240 - protein, 9 aa, Artificial Sequence
  source: /note=“Description of Artificial Sequence: Synthetic peptide”
HQYIAKPIT

SEQ ID NO: 241 - protein, 7 aa, Artificial Sequence
  source: /note=“Description of Artificial Sequence: Synthetic peptide”
SQSIRYN

SEQ ID NO: 242 - protein, 3 aa, Artificial Sequence
  source: /note=“Description of Artificial Sequence: Synthetic peptide”
AAS

SEQ ID NO: 243 - protein, 6 aa, Artificial Sequence
  source: /note=“Description of Artificial Sequence: Synthetic peptide”
YIAKPI

SEQ ID NO: 244 - protein, 6 aa, Artificial Sequence
  source: /note=“Description of Artificial Sequence: Synthetic peptide”
QSIRYN

SEQ ID NO: 245 - protein, 3 aa, Artificial Sequence
  source: /note=“Description of Artificial Sequence: Synthetic peptide”
AAS

SEQ ID NO: 246 - protein, 9 aa, Artificial Sequence
  source: /note=“Description of Artificial Sequence: Synthetic peptide”
HQYIAKPIT

SEQ ID NO: 247 - protein, 107 aa, Artificial Sequence
  source: /note=“Description of Artificial Sequence: Synthetic polypeptide”
DIQMTQSPSSLSASVGDRVTITCRASQSIRYNLAWYQQKPGKAPKLLIYAASSLQSGVPSRFSGSGSGTDFTLTISSLQPEDFATYYCHQYIAKPITFGQGTKVEIK

SEQ ID NO: 248 - DNA, 321 bp, Artificial Sequence
  source: /note=“Description of Artificial Sequence: Synthetic polynucleotide”
gacatccagatgacccagagcccctccagcctgtccgcctccgtgggcgacagagtgaccatcacctgtcgggcctcccagtccatccggtacaacctggcctggtatcagcagaagcccggcaaggcccctaagctgctgatctacgccgccagctccctgcagtccggcgtgccctccagattctccggctctggctccggcaccgacttcaccctgaccatctccagcctgcagcccgaggacttcgccacctactactgccaccagtatatcgccaagcccatcaccttcggccagggcaccaaagtggaaatcaag

SEQ ID NO: 249 - protein, 214 aa, Artificial Sequence
  source: /note=“Description of Artificial Sequence: Synthetic polypeptide”
DIQMTQSPSSLSASVGDRVTITCRASQSIRYNLAWYQQKPGKAPKLLIYAASSLQSGVPSRFSGSGSGTDFTLTISSLQPEDFATYYCHQYIAKPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 250 - DNA, 642 bp, Artificial Sequence
  source: /note=“Description of Artificial Sequence: Synthetic polynucleotide”
gacatccagatgacccagagcccctccagcctgtccgcctccgtgggcgacagagtgaccatcacctgtcgggcctcccagtccatccggtacaacctggcctggtatcagcagaagcccggcaaggcccctaagctgctgatctacgccgccagctccctgcagtccggcgtgccctccagattctccggctctggctccggcaccgacttcaccctgaccatctccagcctgcagcccgaggacttcgccacctactactgccaccagtatatcgccaagcccatcaccttcggccagggcaccaaagtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 251 - protein, 10 aa, Artificial Sequence
  source: /note=“Description of Artificial Sequence: Synthetic peptide”
GFTFSSNAMH

SEQ ID NO: 252 - protein, 19 aa, Artificial Sequence
  source: /note=“Description of Artificial Sequence: Synthetic peptide”
RIKSKTSGGTTDYAAPVKG

SEQ ID NO: 253 - protein, 9 aa, Artificial Sequence
  source: /note=“Description of Artificial Sequence: Synthetic peptide”
DHYYYPFAY

SEQ ID NO: 254 - protein, 5 aa, Artificial Sequence
  source: /note=“Description of Artificial Sequence: Synthetic peptide”
SNAMH

SEQ ID NO: 255 - protein, 19 aa, Artificial Sequence
  source: /note=“Description of Artificial Sequence: Synthetic peptide”
RIKSKTSGGTTDYAAPVKG

SEQ ID NO: 256 - protein, 9 aa, Artificial Sequence
  source: /note=“Description of Artificial Sequence: Synthetic peptide”
DHYYYPFAY

SEQ ID NO: 257 - protein, 7 aa, Artificial Sequence
  source: /note=“Description of Artificial Sequence: Synthetic peptide”
GFTFSSN

SEQ ID NO: 258 - protein, 8 aa, Artificial Sequence
  source: /note=“Description of Artificial Sequence: Synthetic peptide”
KSKTSGGT

SEQ ID NO: 259 - protein, 9 aa, Artificial Sequence
  source: /note=“Description of Artificial Sequence: Synthetic peptide”
DHYYYPFAY

SEQ ID NO: 260 - protein, 8 aa, Artificial Sequence
  source: /note=“Description of Artificial Sequence: Synthetic peptide”
GFTFSSNA

SEQ ID NO: 261 - protein, 10 aa, Artificial Sequence
  source: /note=“Description of Artificial Sequence: Synthetic peptide”
IKSKTSGGTT

SEQ ID NO: 262 - protein, 11 aa, Artificial Sequence
  source: /note=“Description of Artificial Sequence: Synthetic peptide”
ARDHYYYPFAY

SEQ ID NO: 263 - protein, 120 aa, Artificial Sequence
  source: /note=“Description of Artificial Sequence: Synthetic polypeptide”
QVQLVESGGGLVKPGGSLRLSCAASGFTFSSNAMHWVRQAPGKGLEWVGRIKSKTSGGTTDYAAPVKGRFTISRDDSKNTLYLQMNSLKTEDTAVYYCARDHYYYPFAYWGQGTLVTVSS

SEQ ID NO: 264 - DNA, 360 bp, Artificial Sequence
  source: /note=“Description of Artificial Sequence: Synthetic polynucleotide”
caagtgcagctggtggaatctggcggcggactggtcaagcctggcggtagcctgagactgtcttgcgccgcctccggcttcaccttctcctccaacgccatgcactgggtccgacaggcccctggcaagggcctggagtgggtcggacggatcaagtccaagacctccggcggcaccaccgactacgctgcccctgtgaagggccggttcaccatctcccgggacgactccaagaacaccctgtacctgcagatgaactccctgaaaaccgaggacaccgccgtgtactactgcgccagggaccactactactaccccttcgcctactggggccagggcaccctggtcaccgtgtcctct

SEQ ID NO: 265 - protein, 223 aa, Artificial Sequence
  source: /note=“Description of Artificial Sequence: Synthetic polypeptide”
QVQLVESGGGLVKPGGSLRLSCAASGFTFSSNAMHWVRQAPGKGLEWVGRIKSKTSGGTTDYAAPVKGRFTISRDDSKNTLYLQMNSLKTEDTAVYYCARDHYYYPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 266 - DNA, 669 bp, Artificial Sequence
  source: /note=“Description of Artificial Sequence: Synthetic polynucleotide”
caagtgcagctggtggaatctggcggcggactggtcaagcctggcggtagcctgagactgtcttgcgccgcctccggcttcaccttctcctccaacgccatgcactgggtccgacaggcccctggcaagggcctggagtgggtcggacggatcaagtccaagacctccggcggcaccaccgactacgctgcccctgtgaagggccggttcaccatctcccgggacgactccaagaacaccctgtacctgcagatgaactccctgaaaaccgaggacaccgccgtgtactactgcgccagggaccactactactaccccttcgcctactggggccagggcaccctggtcaccgtgtcctctgctagcaccaagggcccctccgtgttccctctggccccttccagcaagtctacctctggcggcaccgctgctctgggctgcctggtgaaggactacttccctgagcctgtgacagtgtcctggaactctggcgccctgacctccggcgtgcacaccttccctgccgtgctgcagtcctccggcctgtactccctgtcctccgtggtgacagtgccttcctccagcctgggcacccagacctatatctgcaacgtgaaccacaagccttccaacaccaaggtggacaagcgggtggagcctaagtcatgc

SEQ ID NO: 267 - protein, 11 aa, Artificial Sequence
  source: /note=“Description of Artificial Sequence: Synthetic peptide”
RASQSIRYNLA

SEQ ID NO: 268 - protein, 7 aa, Artificial Sequence
  source: /note=“Description of Artificial Sequence: Synthetic peptide”
AASSLQS

SEQ ID NO: 269 - protein, 9 aa, Artificial Sequence
  source: /note=“Description of Artificial Sequence: Synthetic peptide”
HQYIAKPIT

SEQ ID NO: 270 - protein, 11 aa, Artificial Sequence
  source: /note=“Description of Artificial Sequence: Synthetic peptide”
RASQSIRYNLA

SEQ ID NO: 271 - protein, 7 aa, Artificial Sequence
  source: /note=“Description of Artificial Sequence: Synthetic peptide”
AASSLQS

SEQ ID NO: 272 - protein, 9 aa, Artificial Sequence
  source: /note=“Description of Artificial Sequence: Synthetic peptide”
HQYIAKPIT

SEQ ID NO: 273 - protein, 7 aa, Artificial Sequence
  source: /note=“Description of Artificial Sequence: Synthetic peptide”
SQSIRYN

SEQ ID NO: 274 - protein, 3 aa, Artificial Sequence
  source: /note=“Description of Artificial Sequence: Synthetic peptide”
AAS

SEQ ID NO: 275 - protein, 6 aa, Artificial Sequence
  source: /note=“Description of Artificial Sequence: Synthetic peptide”
YIAKPI

SEQ ID NO: 276 - protein, 6 aa, Artificial Sequence
  source: /note=“Description of Artificial Sequence: Synthetic peptide”
QSIRYN

SEQ ID NO: 277 - protein, 3 aa, Artificial Sequence
  source: /note=“Description of Artificial Sequence: Synthetic peptide”
AAS

SEQ ID NO: 278 - protein, 9 aa, Artificial Sequence
  source: /note=“Description of Artificial Sequence: Synthetic peptide”
HQYIAKPIT

SEQ ID NO: 279 - protein, 107 aa, Artificial Sequence; identical to SEQ ID NO: 247
  source: /note=“Description of Artificial Sequence: Synthetic polypeptide”

SEQ ID NO: 280 - DNA, 321 bp, Artificial Sequence; identical to SEQ ID NO: 248
  source: /note=“Description of Artificial Sequence: Synthetic polynucleotide”

SEQ ID NO: 281 - protein, 214 aa, Artificial Sequence; identical to SEQ ID NO: 249
  source: /note=“Description of Artificial Sequence: Synthetic polypeptide”

SEQ ID NO: 282 - DNA, 642 bp, Artificial Sequence; identical to SEQ ID NO: 250
  source: /note=“Description of Artificial Sequence: Synthetic polynucleotide”

SEQ ID NO: 283 - protein, 10 aa, Artificial Sequence
  source: /note=“Description of Artificial Sequence: Synthetic peptide”
GYTFTNYYVH

SEQ ID NO: 284 - protein, 17 aa, Artificial Sequence
  source: /note=“Description of Artificial Sequence: Synthetic peptide”
WINPYNGNTNYAQKFQG

SEQ ID NO: 285 - protein, 13 aa, Artificial Sequence
  source: /note=“Description of Artificial Sequence: Synthetic peptide”
GASSIRMSYYLDV

SEQ ID NO: 286 - protein, 5 aa, Artificial Sequence
  source: /note=“Description of Artificial Sequence: Synthetic peptide”
NYYVH

SEQ ID NO: 287 - protein, 17 aa, Artificial Sequence
  source: /note=“Description of Artificial Sequence: Synthetic peptide”
WINPYNGNTNYAQKFQG

SEQ ID NO: 288 - protein, 13 aa, Artificial Sequence
  source: /note=“Description of Artificial Sequence: Synthetic peptide”
GASSIRMSYYLDV

SEQ ID NO: 289 - protein, 7 aa, Artificial Sequence
  source: /note=“Description of Artificial Sequence: Synthetic peptide”
GYTFTNY

SEQ ID NO: 290 - protein, 6 aa, Artificial Sequence
  source: /note=“Description of Artificial Sequence: Synthetic peptide”
NPYNGN

SEQ ID NO: 291 - protein, 13 aa, Artificial Sequence
  source: /note=“Description of Artificial Sequence: Synthetic peptide”
GASSIRMSYYLDV

SEQ ID NO: 292 - protein, 8 aa, Artificial Sequence
  source: /note=“Description of Artificial Sequence: Synthetic peptide”
GYTFTNYY

SEQ ID NO: 293 - protein, 8 aa, Artificial Sequence
  source: /note=“Description of Artificial Sequence: Synthetic peptide”
INPYNGNT

SEQ ID NO: 294 - protein, 15 aa, Artificial Sequence
  source: /note=“Description of Artificial Sequence: Synthetic peptide”
ARGASSIRMSYYLDV

SEQ ID NO: 295 - protein, 122 aa, Artificial Sequence
  source: /note=“Description of Artificial Sequence: Synthetic polypeptide”
QVQLVQSGAEVKKPGASVKVSCKASGYTFTNYYVHWVRQAPGQGLEWMGWINPYNGNTNYAQKFQGRVTMTRDTSISTAYMELSRLRSEDTAVYYCARGASSIRMSYYLDVWGQGTLVTVSS

SEQ ID NO: 296 - DNA, 366 bp, Artificial Sequence
  source: /note=“Description of Artificial Sequence: Synthetic polynucleotide”
caagtgcagctggtgcagtctggcgctgaagtgaagaaacctggcgcctccgtgaaagtgtcctgcaaggcctccggctacaccttcaccaactactacgtgcactgggtccgacaggccccaggccagggcctggagtggatgggctggatcaacccctacaacggcaacaccaactacgcccagaaattccagggcagagtgaccatgacccgggacacctccatctccaccgcctacatggaactgtcccggctgcggagcgaggacaccgccgtgtactactgtgccagaggcgcctcctccatccggatgtcctactacctggacgtgtggggccagggcaccctggtcaccgtgtcctct

SEQ ID NO: 297 - protein, 225 aa, Artificial Sequence
  source: /note=“Description of Artificial Sequence: Synthetic polypeptide”
QVQLVQSGAEVKKPGASVKVSCKASGYTFTNYYVHWVRQAPGQGLEWMGWINPYNGNTNYAQKFQGRVTMTRDTSISTAYMELSRLRSEDTAVYYCARGASSIRMSYYLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 298 - DNA, 675 bp, Artificial Sequence
  source: /note=“Description of Artificial Sequence: Synthetic polynucleotide”
caagtgcagctggtgcagtctggcgctgaagtgaagaaacctggcgcctccgtgaaagtgtcctgcaaggcctccggctacaccttcaccaactactacgtgcactgggtccgacaggccccaggccagggcctggagtggatgggctggatcaacccctacaacggcaacaccaactacgcccagaaattccagggcagagtgaccatgacccgggacacctccatctccaccgcctacatggaactgtcccggctgcggagcgaggacaccgccgtgtactactgtgccagaggcgcctcctccatccggatgtcctactacctggacgtgtggggccagggcaccctggtcaccgtgtcctctgctagcaccaagggcccctccgtgttccctctggccccttccagcaagtctacctctggcggcaccgctgctctgggctgcctggtgaaggactacttccctgagcctgtgacagtgtcctggaactctggcgccctgacctccggcgtgcacaccttccctgccgtgctgcagtcctccggcctgtactccctgtcctccgtggtgacagtgccttcctccagcctgggcacccagacctatatctgcaacgtgaaccacaagccttccaacaccaaggtggacaagcgggtggagcctaagtcatgc

SEQ ID NO: 299 - protein, 11 aa, Artificial Sequence
  source: /note=“Description of Artificial Sequence: Synthetic peptide”
RASQSISNYLN

SEQ ID NO: 300 - protein, 7 aa, Artificial Sequence
  source: /note=“Description of Artificial Sequence: Synthetic peptide”
AASNLQS

SEQ ID NO: 301 - protein, 9 aa, Artificial Sequence
  source: /note=“Description of Artificial Sequence: Synthetic peptide”
FQYTHSPAT

SEQ ID NO: 302 - protein, 11 aa, Artificial Sequence
  source: /note=“Description of Artificial Sequence: Synthetic peptide”
RASQSISNYLN

SEQ ID NO: 303 - protein, 7 aa, Artificial Sequence
  source: /note=“Description of Artificial Sequence: Synthetic peptide”
AASNLQS

SEQ ID NO: 304 - protein, 9 aa, Artificial Sequence
  source: /note=“Description of Artificial Sequence: Synthetic peptide”
FQYTHSPAT

SEQ ID NO: 305 - protein, 7 aa, Artificial Sequence
  source: /note=“Description of Artificial Sequence: Synthetic peptide”
SQSISNY

SEQ ID NO: 306 - protein, 3 aa, Artificial Sequence
  source: /note=“Description of Artificial Sequence: Synthetic peptide”
AAS

SEQ ID NO: 307 - protein, 6 aa, Artificial Sequence
  source: /note=“Description of Artificial Sequence: Synthetic peptide”
YTHSPA

SEQ ID NO: 308 - protein, 6 aa, Artificial Sequence
  source: /note=“Description of Artificial Sequence: Synthetic peptide”
QSISNY

SEQ ID NO: 309 - protein, 3 aa, Artificial Sequence
  source: /note=“Description of Artificial Sequence: Synthetic peptide”
AAS

SEQ ID NO: 310 - protein, 9 aa, Artificial Sequence
  source: /note=“Description of Artificial Sequence: Synthetic peptide”
FQYTHSPAT

SEQ ID NO: 311 - protein, 107 aa, Artificial Sequence
  source: /note=“Description of Artificial Sequence: Synthetic polypeptide”
DIQMTQSPSSLSASVGDRVTITCRASQSISNYLNWYQQKPGKAPKLLIYAASNLQSGVPSRFSGSGSGTDFTLTISSLQPEDFATYYCFQYTHSPATFGQGTKVEIK

SEQ ID NO: 312 - DNA, 321 bp, Artificial Sequence
  source: /note=“Description of Artificial Sequence: Synthetic polynucleotide”
gacatccagatgacccagagcccctccagcctgtccgcctccgtgggcgacagagtgaccatcacctgtcgggcctcccagtccatctccaactacctgaactggtatcagcagaagcccggcaaggcccctaagctgctgatctacgccgcctccaacctgcagtccggcgtgccctccagattctccggctctggctccggcaccgacttcaccctgaccatctccagcctgcagcccgaggacttcgccacctactactgcttccagtacacccacagccccgccaccttcggccagggcaccaaagtggaaatcaag

SEQ ID NO: 313 - protein, 214 aa, Artificial Sequence
  source: /note=“Description of Artificial Sequence: Synthetic polypeptide”
DIQMTQSPSSLSASVGDRVTITCRASQSISNYLNWYQQKPGKAPKLLIYAASNLQSGVPSRFSGSGSGTDFTLTISSLQPEDFATYYCFQYTHSPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 314 - DNA, 642 bp, Artificial Sequence
  source: /note=“Description of Artificial Sequence: Synthetic polynucleotide”
gacatccagatgacccagagcccctccagcctgtccgcctccgtgggcgacagagtgaccatcacctgtcgggcctcccagtccatctccaactacctgaactggtatcagcagaagcccggcaaggcccctaagctgctgatctacgccgcctccaacctgcagtccggcgtgccctccagattctccggctctggctccggcaccgacttcaccctgaccatctccagcctgcagcccgaggacttcgccacctactactgcttccagtacacccacagccccgccaccttcggccagggcaccaaagtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 315 - protein, 10 aa, Artificial Sequence
  source: /note=“Description of Artificial Sequence: Synthetic peptide”
GYTFTNYYVH

SEQ ID NO: 316 - protein, 17 aa, Artificial Sequence
  source: /note=“Description of Artificial Sequence: Synthetic peptide”
WINPYSGNTNYAQKFQG

SEQ ID NO: 317 - protein, 13 aa, Artificial Sequence
  source: /note=“Description of Artificial Sequence: Synthetic peptide”
GASSIRMSYYLDV

SEQ ID NO: 318 - protein, 5 aa, Artificial Sequence
  source: /note=“Description of Artificial Sequence: Synthetic peptide”
NYYVH

SEQ ID NO: 319 - protein, 17 aa, Artificial Sequence
  source: /note=“Description of Artificial Sequence: Synthetic peptide”
WINPYSGNTNYAQKFQG

SEQ ID NO: 320 - protein, 13 aa, Artificial Sequence
  source: /note=“Description of Artificial Sequence: Synthetic peptide”
GASSIRMSYYLDV

SEQ ID NO: 321 - protein, 7 aa, Artificial Sequence
  source: /note=“Description of Artificial Sequence: Synthetic peptide”
GYTFTNY

SEQ ID NO: 322 - protein, 6 aa, Artificial Sequence
  source: /note=“Description of Artificial Sequence: Synthetic peptide”
NPYSGN

SEQ ID NO: 323 - protein, 13 aa, Artificial Sequence
  source: /note=“Description of Artificial Sequence: Synthetic peptide”
GASSIRMSYYLDV

SEQ ID NO: 324 - protein, 8 aa, Artificial Sequence
  source: /note=“Description of Artificial Sequence: Synthetic peptide”
GYTFTNYY

SEQ ID NO: 325 - protein, 8 aa, Artificial Sequence
  source: /note=“Description of Artificial Sequence: Synthetic peptide”
INPYSGNT

SEQ ID NO: 326 - protein, 15 aa, Artificial Sequence
  source: /note=“Description of Artificial Sequence: Synthetic peptide”
ARGASSIRMSYYLDV

SEQ ID NO: 327 - protein, 122 aa, Artificial Sequence
  source: /note=“Description of Artificial Sequence: Synthetic polypeptide”
QVQLVQSGAEVKKPGASVKVSCKASGYTFTNYYVHWVRQAPGQGLEWMGWINPYSGNTNYAQKFQGRVTMTRDTSISTAYMELSRLRSEDTAVYYCARGASSIRMSYYLDVWGQGTLVTVSS

SEQ ID NO: 328 - DNA, 366 bp, Artificial Sequence
  source: /note=“Description of Artificial Sequence: Synthetic polynucleotide”
caagtgcagctggtgcagtctggcgctgaagtgaagaaacctggcgcctccgtgaaagtgtcctgcaaggcctccggctacaccttcaccaactactacgtgcactgggtccgacaggccccaggccagggcctggagtggatgggctggatcaacccctactccggcaacaccaactacgcccagaaattccagggcagagtgaccatgacccgggacacctccatctccaccgcctacatggaactgtcccggctgcggagcgaggacaccgccgtgtactactgtgccagaggcgcctcctccatccggatgtcctactacctggacgtgtggggccagggcaccctggtcaccgtgtcctct

SEQ ID NO: 329 - protein, 225 aa, Artificial Sequence
  source: /note=“Description of Artificial Sequence: Synthetic polypeptide”
QVQLVQSGAEVKKPGASVKVSCKASGYTFTNYYVHWVRQAPGQGLEWMGWINPYSGNTNYAQKFQGRVTMTRDTSISTAYMELSRLRSEDTAVYYCARGASSIRMSYYLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 330 - DNA, 675 bp, Artificial Sequence
  source: /note=“Description of Artificial Sequence: Synthetic polynucleotide”
caagtgcagctggtgcagtctggcgctgaagtgaagaaacctggcgcctccgtgaaagtgtcctgcaaggcctccggctacaccttcaccaactactacgtgcactgggtccgacaggccccaggccagggcctggagtggatgggctggatcaacccctactccggcaacaccaactacgcccagaaattccagggcagagtgaccatgacccgggacacctccatctccaccgcctacatggaactgtcccggctgcggagcgaggacaccgccgtgtactactgtgccagaggcgcctcctccatccggatgtcctactacctggacgtgtggggccagggcaccctggtcaccgtgtcctctgctagcaccaagggcccctccgtgttccctctggccccttccagcaagtctacctctggcggcaccgctgctctgggctgcctggtgaaggactacttccctgagcctgtgacagtgtcctggaactctggcgccctgacctccggcgtgcacaccttccctgccgtgctgcagtcctccggcctgtactccctgtcctccgtggtgacagtgccttcctccagcctgggcacccagacctatatctgcaacgtgaaccacaagccttccaacaccaaggtggacaagcgggtggagcctaagtcatgc

SEQ ID NO: 331 - protein, 11 aa, Artificial Sequence
  source: /note=“Description of Artificial Sequence: Synthetic peptide”
RASQSISNYLN

SEQ ID NO: 332 - protein, 7 aa, Artificial Sequence
  source: /note=“Description of Artificial Sequence: Synthetic peptide”
AASNLQS

SEQ ID NO: 333 - protein, 9 aa, Artificial Sequence
  source: /note=“Description of Artificial Sequence: Synthetic peptide”
FQYTHSPAT

SEQ ID NO: 334 - protein, 11 aa, Artificial Sequence
  source: /note=“Description of Artificial Sequence: Synthetic peptide”
RASQSISNYLN

SEQ ID NO: 335 - protein, 7 aa, Artificial Sequence
  source: /note=“Description of Artificial Sequence: Synthetic peptide”
AASNLQS

SEQ ID NO: 336 - protein, 9 aa, Artificial Sequence
  source: /note=“Description of Artificial Sequence: Synthetic peptide”
FQYTHSPAT

SEQ ID NO: 337 - protein, 7 aa, Artificial Sequence
  source: /note=“Description of Artificial Sequence: Synthetic peptide”
SQSISNY

SEQ ID NO: 338 - protein, 3 aa, Artificial Sequence
  source: /note=“Description of Artificial Sequence: Synthetic peptide”
AAS

SEQ ID NO: 339 - protein, 6 aa, Artificial Sequence
  source: /note=“Description of Artificial Sequence: Synthetic peptide”
YTHSPA

SEQ ID NO: 340 - protein, 6 aa, Artificial Sequence
  source: /note=“Description of Artificial Sequence: Synthetic peptide”
QSISNY

SEQ ID NO: 341 - protein, 3 aa, Artificial Sequence
  source: /note=“Description of Artificial Sequence: Synthetic peptide”
AAS

SEQ ID NO: 342 - protein, 9 aa, Artificial Sequence
  source: /note=“Description of Artificial Sequence: Synthetic peptide”
FQYTHSPAT

SEQ ID NO: 343 - protein, 107 aa, Artificial Sequence; identical to SEQ ID NO: 311
  source: /note=“Description of Artificial Sequence: Synthetic polypeptide”

SEQ ID NO: 344 - DNA, 321 bp, Artificial Sequence; identical to SEQ ID NO: 312
  source: /note=“Description of Artificial Sequence: Synthetic polynucleotide”

SEQ ID NO: 345 - protein, 214 aa, Artificial Sequence; identical to SEQ ID NO: 313
  source: /note=“Description of Artificial Sequence: Synthetic polypeptide”

SEQ ID NO: 346 - DNA, 642 bp, Artificial Sequence; identical to SEQ ID NO: 314
  source: /note=“Description of Artificial Sequence: Synthetic polynucleotide”

SEQ ID NO: 347 - protein, 451 aa, Artificial Sequence
  source: /note=“Description of Artificial Sequence: Synthetic polypeptide”
QVQLLESGGGLVQPGGSLRLSCAASGFTFSDYAMSWVRQAPGKGLEWVSVIDYSSSNTYYADSVKGRFTISRDNSKNTLYLQMNSLRAEDTAVYYCAREGYSYRSIRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 348 - DNA, 1353 bp, Artificial Sequence
  source: /note=“Description of Artificial Sequence: Synthetic polynucleotide”
caagtgcagctgctggaatctggcggcggactggtgcagcctggcggtagtctgagactgtcttgcgccgcctccggcttcaccttctccgactacgccatgtcctgggtccgacaggcccctggcaagggcctggagtgggtgtccgtgatcgactactcctcctccaacacctactacgccgactccgtgaagggccggttcaccatctcccgggacaactccaagaacaccctgtacctgcagatgaactccctgcgggccgaggacaccgccgtgtactactgcgccagagagggctactcctaccggtccatcagattcgactactggggccagggcaccctggtcaccgtgtcctctgctagcaccaagggcccctccgtgttccctctggccccttccagcaagtctacctccggcggcacagctgctctgggctgcctggtcaaggactacttccctgagcctgtgacagtgtcctggaactctggcgccctgacctctggcgtgcacaccttccctgccgtgctgcagtcctccggcctgtactccctgtcctccgtggtcacagtgccttcaagcagcctgggcacccagacctatatctgcaacgtgaaccacaagccttccaacaccaaggtggacaagcgggtggagcctaagtcctgcgacaagacccacacctgtcctccctgccctgctcctgaactgctgggcggcccttctgtgttcctgttccctccaaagcccaaggacaccctgatgatctcccggacccctgaagtgacctgcgtggtggtggccgtgtcccacgaggatcctgaagtgaagttcaattggtacgtggacggcgtggaggtgcacaacgccaagaccaagcctcgggaggaacagtacaactccacctaccgggtggtgtccgtgctgaccgtgctgcaccaggactggctgaacggcaaagagtacaagtgcaaagtctccaacaaggccctggccgcccctatcgaaaagacaatctccaaggccaagggccagcctagggaaccccaggtgtacaccctgccacccagccgggaggaaatgaccaagaaccaggtgtccctgacctgtctggtcaagggcttctacccttccgatatcgccgtggagtgggagtctaacggccagcctgagaacaactacaagaccacccctcctgtgctggactccgacggctccttcttcctgtactccaaactgaccgtggacaagtcccggtggcagcagggcaacgtgttctcctgctccgtgatgcacgaggccctgcacaaccactacacccagaagtccctgtccctgtctcccggcaag

SEQ ID NO: 349 - protein, 459 aa, Artificial Sequence
  source: /note=“Description of Artificial Sequence: Synthetic polypeptide”
QVQLVESGGGLVKPGGSLRLSCAASGFTFSSAAVHWVRQAPGKGLEWVGRIKSKADGGTTDYAAPVKGRFTISRDDSKNTLYLQMNSLKTEDTAVYYCARDSPSISSYSIPYFSGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 350 - DNA, 1377 bp, Artificial Sequence
  source: /note=“Description of Artificial Sequence: Synthetic polynucleotide”
caagtgcagctggtggaatctggcggcggactggtcaagcctggcggtagcctgagactgtcttgcgccgcctccggcttcaccttctcctctgccgctgtgcactgggtccgacaggcccctggcaagggcctggagtgggtcggacggatcaagtccaaggccgacggcggcaccaccgactacgctgcccctgtgaagggccggttcaccatctcccgggacgactccaagaacaccctgtacctgcagatgaactccctgaaaaccgaggacaccgccgtgtactactgcgccagagactccccatctatctccagctactccatcccctacttctccggcatggacgtgtggggccagggcaccctggtcaccgtgtcctctgctagcaccaagggcccctccgtgttccctctggccccttccagcaagtctacctccggcggcacagctgctctgggctgcctggtcaaggactacttccctgagcctgtgacagtgtcctggaactctggcgccctgacctctggcgtgcacaccttccctgccgtgctgcagtcctccggcctgtactccctgtcctccgtggtcacagtgccttcaagcagcctgggcacccagacctatatctgcaacgtgaaccacaagccttccaacaccaaggtggacaagcgggtggagcctaagtcctgcgacaagacccacacctgtcctccctgccctgctcctgaactgctgggcggcccttctgtgttcctgttccctccaaagcccaaggacaccctgatgatctcccggacccctgaagtgacctgcgtggtggtggccgtgtcccacgaggatcctgaagtgaagttcaattggtacgtggacggcgtggaggtgcacaacgccaagaccaagcctcgggaggaacagtacaactccacctaccgggtggtgtccgtgctgaccgtgctgcaccaggactggctgaacggcaaagagtacaagtgcaaagtctccaacaaggccctggccgcccctatcgaaaagacaatctccaaggccaagggccagcctagggaaccccaggtgtacaccctgccacccagccgggaggaaatgaccaagaaccaggtgtccctgacctgtctggtcaagggcttctacccttccgatatcgccgtggagtgggagtctaacggccagcctgagaacaactacaagaccacccctcctgtgctggactccgacggctccttcttcctgtactccaaactgaccgtggacaagtcccggtggcagcagggcaacgtgttctcctgctccgtgatgcacgaggccctgcacaaccactacacccagaagtccctgtccctgtctcccggcaag

SEQ ID NO: 351 - protein, 227 aa, Artificial Sequence
  source: /note=“Description of Artificial Sequence: Synthetic polypeptide”
DKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 352-359 - skipped (8 entries)

SEQ ID NO: 360 - protein, 452 aa, Artificial Sequence
  source: /note=“Description of Artificial Sequence: Synthetic polypeptide”
QVQLLESGGGLVQPGGSLRLSCAASGFTFSTAAMSWVRQAPGKGLEWVSGISGSGSSTYYADSVKGRFTISRDNSKNTLYLQMNSLRAEDTAVYYCARELSYLYSGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 361 - DNA, 1356 bp, Artificial Sequence
  source: /note=“Description of Artificial Sequence: Synthetic polynucleotide”
caggtgcaattgctggaaagcggcggtggcctggtgcagccgggtggcagcctgcgtctgagctgcgcggcgtccggattcaccttttctactgctgctatgtcttgggtgcgccaggccccgggcaaaggtctcgagtgggtttccggtatctctggttctggttcttctacctactatgcggatagcgtgaaaggccgctttaccatcagccgcgataattcgaaaaacaccctgtatctgcaaatgaacagcctgcgtgcggaagatacggccgtgtattattgcgcgcgtgaactgtcttacctgtactctggttactacttcgattactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 362 - protein, 110 aa, Artificial Sequence
  source: /note=“Description of Artificial Sequence: Synthetic polypeptide”
DIVLTQPPSVSGAPGQRVTISCSGSSSNIGSNDVSWYQQLPGTAPKLLIYKNYNRPSGVPDRFSGSKSGTSASLAITGLQAEDEADYYCSAWDQRQFDVVFGGGTKLTVL

SEQ ID NO: 363 - DNA, 330 bp, Artificial Sequence
  source: /note=“Description of Artificial Sequence: Synthetic polynucleotide”
gatatcgtgctgacccagccgccgagcgtgagcggtgcaccgggccagcgcgtgaccattagctgtagcggcagcagcagcaacattggttctaacgacgtgtcttggtaccagcagctgccgggcacggcgccgaaactgctgatctacaaaaactacaaccgcccgagcggcgtgccggatcgctttagcggatccaaaagcggcaccagcgccagcctggcgattaccggcctgcaagcagaagacgaagcggattattactgctctgcttgggaccagcgtcagttcgacgttgtgtttggcggcggcacgaagttaaccgtccta

SEQ ID NO: 364 - protein, 216 aa, Artificial Sequence
  source: /note=“Description of Artificial Sequence: Synthetic polypeptide”
DIVLTQPPSVSGAPGQRVTISCSGSSSNIGSNDVSWYQQLPGTAPKLLIYKNYNRPSGVPDRFSGSKSGTSASLAITGLQAEDEADYYCSAWDQRQFDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 365 - DNA, 648 bp, Artificial Sequence
  source: /note=“Description of Artificial Sequence: Synthetic polynucleotide”
gatatcgtgctgacccagccgccgagcgtgagcggtgcaccgggccagcgcgtgaccattagctgtagcggcagcagcagcaacattggttctaacgacgtgtcttggtaccagcagctgccgggcacggcgccgaaactgctgatctacaaaaactacaaccgcccgagcggcgtgccggatcgctttagcggatccaaaagcggcaccagcgccagcctggcgattaccggcctgcaagcagaagacgaagcggattattactgctctgcttgggaccagcgtcagttcgacgttgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 366 - protein, 17 aa, Artificial Sequence
  source: /note=“Description of Artificial Sequence: Synthetic peptide”
TIDSWGDDTDYADSVKG

SEQ ID NO: 367 - protein, 6 aa, Artificial Sequence
  source: /note=“Description of Artificial Sequence: Synthetic peptide”
DSWGDD

SEQ ID NO: 368 - protein, 8 aa, Artificial Sequence
  source: /note=“Description of Artificial Sequence: Synthetic peptide”
IDSWGDDT

SEQ ID NO: 369 - protein, 122 aa, Artificial Sequence
  source: /note=“Description of Artificial Sequence: Synthetic polypeptide”
QVQLLESGGGLVQPGGSLRLSCAASGFTFSTAAMSWVRQAPGKGLEWVSTIDSWGDDTDYADSVKGRFTISRDNSKNTLYLQMNSLRAEDTAVYYCARELSYLYSGYYFDYWGQGTLVTVSS

SEQ ID NO: 370 - DNA, 366 bp, Artificial Sequence
  source: /note=“Description of Artificial Sequence: Synthetic polynucleotide”
caggtgcaattgctggaaagcggcggtggcctggtgcagccgggtggcagcctgcgtctgagctgcgcggcgtccggattcaccttttctactgctgctatgtcttgggtgcgccaggccccgggcaaaggtctcgagtgggtttccactatcgactcttggggcgacgacactgactatgcggatagcgtgaaaggccgctttaccatcagccgcgataattcgaaaaacaccctgtatctgcaaatgaacagcctgcgtgcggaagatacggccgtgtattattgcgcgcgtgaactgtcttacctgtactctggttactacttcgattactggggccaaggcaccctggtgactgttagctca

SEQ ID NO: 371 - protein, 452 aa, Artificial Sequence
  source: /note=“Description of Artificial Sequence: Synthetic polypeptide”
QVQLLESGGGLVQPGGSLRLSCAASGFTFSTAAMSWVRQAPGKGLEWVSTIDSWGDDTDYADSVKGRFTISRDNSKNTLYLQMNSLRAEDTAVYYCARELSYLYSGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 372 - protein, 17 aa, Artificial Sequence
  source: /note=“Description of Artificial Sequence: Synthetic peptide”
SIEYYDTDTHYADSVKG

SEQ ID NO: 373 - protein, 6 aa, Artificial Sequence
  source: /note=“Description of Artificial Sequence: Synthetic peptide”
EYYDTD

SEQ ID NO: 374 - protein, 8 aa, Artificial Sequence
  source: /note=“Description of Artificial Sequence: Synthetic peptide”
IEYYDTDT

SEQ ID NO: 375 - protein, 122 aa, Artificial Sequence
  source: /note=“Description of Artificial Sequence: Synthetic polypeptide”
QVQLLESGGGLVQPGGSLRLSCAASGFTFSTAAMSWVRQAPGKGLEWVSSIEYYDTDTHYADSVKGRFTISRDNSKNTLYLQMNSLRAEDTAVYYCARELSYLYSGYYFDYWGQGTLVTVSS

SEQ ID NO: 376 - DNA, 366 bp, Artificial Sequence
  source: /note=“Description of Artificial Sequence: Synthetic polynucleotide”
caggtgcaattgctggaaagcggcggtggcctggtgcagccgggtggcagcctgcgtctgagctgcgcggcgtccggattcaccttttctactgctgctatgtcttgggtgcgccaggccccgggcaaaggtctcgagtgggtttcctctatcgaatactacgacactgacactcattatgcggatagcgtgaaaggccgctttaccatcagccgcgataattcgaaaaacaccctgtatctgcaaatgaacagcctgcgtgcggaagatacggccgtgtattattgcgcgcgtgaactgtcttacctgtactctggttactacttcgattactggggccaaggcaccctggtgactgttagctca

SEQ ID NO: 377 - protein, 452 aa, Artificial Sequence
  source: /note=“Description of Artificial Sequence: Synthetic polypeptide”
QVQLLESGGGLVQPGGSLRLSCAASGFTFSTAAMSWVRQAPGKGLEWVSSIEYYDTDTHYADSVKGRFTISRDNSKNTLYLQMNSLRAEDTAVYYCARELSYLYSGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 378 - DNA, 1356 bp, Artificial Sequence
  source: /note=“Description of Artificial Sequence: Synthetic polynucleotide”
caggtgcaattgctggaaagcggcggtggcctggtgcagccgggtggcagcctgcgtctgagctgcgcggcgtccggattcaccttttctactgctgctatgtcttgggtgcgccaggccccgggcaaaggtctcgagtgggtttcctctatcgaatactacgacactgacactcattatgcggatagcgtgaaaggccgctttaccatcagccgcgataattcgaaaaacaccctgtatctgcaaatgaacagcctgcgtgcggaagatacggccgtgtattattgcgcgcgtgaactgtcttacctgtactctggttactacttcgattactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 379 - protein, 17 aa, Artificial Sequence
  source: /note=“Description of Artificial Sequence: Synthetic peptide”
TIEYSSQETYYADSVKG

SEQ ID NO: 380 - protein, 6 aa, Artificial Sequence
  source: /note=“Description of Artificial Sequence: Synthetic peptide”
EYSSQE

SEQ ID NO: 381 - protein, 8 aa, Artificial Sequence
  source: /note=“Description of Artificial Sequence: Synthetic peptide”
IEYSSQET

SEQ ID NO: 382 - protein, 122 aa, Artificial Sequence
  source: /note=“Description of Artificial Sequence: Synthetic polypeptide”
QVQLLESGGGLVQPGGSLRLSCAASGFTFSTAAMSWVRQAPGKGLEWVSTIEYSSQETYYADSVKGRFTISRDNSKNTLYLQMNSLRAEDTAVYYCARELSYLYSGYYFDYWGQGTLVTVSS

SEQ ID NO: 383 - DNA, 366 bp, Artificial Sequence
  source: /note=“Description of Artificial Sequence: Synthetic polynucleotide”
caggtgcaattgctggaaagcggcggtggcctggtgcagccgggtggcagcctgcgtctgagctgcgcggcgtccggattcaccttttctactgctgctatgtcttgggtgcgccaggccccgggcaaaggtctcgagtgggtttccactatcgaatactctagccaggaaacttactatgcggatagcgtgaaaggccgctttaccatcagccgcgataattcgaaaaacaccctgtatctgcaaatgaacagcctgcgtgcggaagatacggccgtgtattattgcgcgcgtgaactgtcttacctgtactctggttactacttcgattactggggccaaggcaccctggtgactgttagctca

SEQ ID NO: 384 - protein, 452 aa, Artificial Sequence
  source: /note=“Description of Artificial Sequence: Synthetic polypeptide”
QVQLLESGGGLVQPGGSLRLSCAASGFTFSTAAMSWVRQAPGKGLEWVSTIEYSSQETYYADSVKGRFTISRDNSKNTLYLQMNSLRAEDTAVYYCARELSYLYSGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 385 - DNA, 1356 bp, Artificial Sequence
  source: /note=“Description of Artificial Sequence: Synthetic polynucleotide”
caggtgcaattgctggaaagcggcggtggcctggtgcagccgggtggcagcctgcgtctgagctgcgcggcgtccggattcaccttttctactgctgctatgtcttgggtgcgccaggccccgggcaaaggtctcgagtgggtttccactatcgaatactctagccaggaaacttactatgcggatagcgtgaaaggccgctttaccatcagccgcgataattcgaaaaacaccctgtatctgcaaatgaacagcctgcgtgcggaagatacggccgtgtattattgcgcgcgtgaactgtcttacctgtactctggttactacttcgattactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 386 - protein, 110 aa, Artificial Sequence
  source: /note=“Description of Artificial Sequence: Synthetic polypeptide”
QSVLTQPPSVSGAPGQRVTISCSGSSSNIGSNDVSWYQQLPGTAPKLLIYKNYNRPSGVPDRFSGSKSGTSASLAITGLQAEDEADYYCSAWDQRQFDVVFGGGTKLTVL

SEQ ID NO: 387 - DNA, 330 bp, Artificial Sequence
  source: /note=“Description of Artificial Sequence: Synthetic polynucleotide”
cagagcgtgctgacacagcctccctccgtgtctggcgcccctggccagagagtgaccatctcctgctccggctcctcctccaacatcggctccaacgacgtgtcctggtatcagcagctgcccggcaccgcccctaagctgctgatctacaagaactacaaccggccctccggcgtgcccgaccggttctctggctccaagtctggcacctccgcctccctggctatcaccggcctgcaggctgaggacgaggccgactactactgctccgcctgggaccagcggcagttcgacgtggtgttcggcggaggcaccaagctgaccgtgctg

SEQ ID NO: 388 - protein, 216 aa, Artificial Sequence
  source: /note=“Description of Artificial Sequence: Synthetic polypeptide”
QSVLTQPPSVSGAPGQRVTISCSGSSSNIGSNDVSWYQQLPGTAPKLLIYKNYNRPSGVPDRFSGSKSGTSASLAITGLQAEDEADYYCSAWDQRQFDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 389 - DNA, 648 bp, Artificial Sequence
  source: /note=“Description of Artificial Sequence: Synthetic polynucleotide”
cagagcgtgctgacacagcctccctccgtgtctggcgcccctggccagagagtgaccatctcctgctccggctcctcctccaacatcggctccaacgacgtgtcctggtatcagcagctgcccggcaccgcccctaagctgctgatctacaagaactacaaccggccctccggcgtgcccgaccggttctctggctccaagtctggcacctccgcctccctggctatcaccggcctgcaggctgaggacgaggccgactactactgctccgcctgggaccagcggcagttcgacgtggtgttcggcggaggcaccaagctgaccgtgctgggccagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 390 - DNA, 366 bp, Artificial Sequence
  source: /note=“Description of Artificial Sequence: Synthetic polynucleotide”
caggtgcaattgctggaaagcggcggtggcctggtgcagccgggtggcagcctgcgtctgagctgcgcggcgtccggattcaccttttctactgctgctatgtcttgggtgcgccaggccccgggcaaaggtctcgagtgggtttccggtatctctggttctggttcttctacctactatgcggatagcgtgaaaggccgctttaccatcagccgcgataattcgaaaaacaccctgtatctgcaaatgaacagcctgcgtgcggaagatacggccgtgtattattgcgcgcgtgaactgtcttacctgtactctggttactacttcgattactggggccaaggcaccctggtgactgttagctca

SEQ ID NO: 391 - DNA, 1356 bp, Artificial Sequence
  source: /note=“Description of Artificial Sequence: Synthetic polynucleotide”
caggtgcaattgctggaaagcggcggtggcctggtgcagccgggtggcagcctgcgtctgagctgcgcggcgtccggattcaccttttctactgctgctatgtcttgggtgcgccaggccccgggcaaaggtctcgagtgggtttccactatcgactcttggggcgacgacactgactatgcggatagcgtgaaaggccgctttaccatcagccgcgataattcgaaaaacaccctgtatctgcaaatgaacagcctgcgtgcggaagatacggccgtgtattattgcgcgcgtgaactgtcttacctgtactctggttactacttcgattactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 392 - DNA, 366 bp, Artificial Sequence
  source: /note=“Description of Artificial Sequence: Synthetic polynucleotide”
caagtgcagctgcttgaatctggcggcggactggtgcagcctggcggctccctgagactgtcttgcgccgcctccggcttcaccttctccaccgccgctatgtcctgggtccgacaggctcccggcaagggcctggaatgggtgtccaccattgagtactccagccaggaaacctactacgccgactccgtgaagggccggttcaccatctcccgggacaactccaagaacaccctgtacctgcagatgaactccctgcgggccgaggacaccgccgtgtactactgcgccagagagctgtcctacctgtactccggctactacttcgactactggggccagggcaccctggtcaccgtgtcctct

SEQ ID NO: 393 - DNA, 1356 bp, Artificial Sequence
  source: /note=“Description of Artificial Sequence: Synthetic polynucleotide”
caagtgcagctgcttgaatctggcggcggactggtgcagcctggcggctccctgagactgtcttgcgccgcctccggcttcaccttctccaccgccgctatgtcctgggtccgacaggctcccggcaagggcctggaatgggtgtccaccattgagtactccagccaggaaacctactacgccgactccgtgaagggccggttcaccatctcccgggacaactccaagaacaccctgtacctgcagatgaactccctgcgggccgaggacaccgccgtgtactactgcgccagagagctgtcctacctgtactccggctactacttcgactactggggccagggcaccctggtcaccgtgtcctctgctagcaccaagggcccctccgtgttccctctggccccttccagcaagtctacctccggcggcacagctgctctgggctgcctggtcaaggactacttccctgagcctgtgacagtgtcctggaactctggcgccctgacctctggcgtgcacaccttccctgccgtgctgcagtcctccggcctgtactccctgtcctccgtggtcacagtgccttcaagcagcctgggcacccagacctatatctgcaacgtgaaccacaagccttccaacaccaaggtggacaagcgggtggagcctaagtcctgcgacaagacccacacctgtcctccctgccctgctcctgaagctgctggcggcccttctgtgttcctgttccctccaaagcccaaggacaccctgatgatctcccggacccctgaagtgacctgcgtggtggtggacgtgtcccacgaggatcctgaagtgaagttcaattggtacgtggacggcgtggaggtgcacaacgccaagaccaagcctcgggaggaacagtacaactccacctaccgggtggtgtccgtgctgaccgtgctgcaccaggactggctgaacggcaaagagtacaagtgcaaagtctccaacaaggccctgcctgcccctatcgaaaagacaatctccaaggccaagggccagcctagggaaccccaggtgtacaccctgccacccagccgggaggaaatgaccaagaaccaggtgtccctgacctgtctggtcaagggcttctacccttccgatatcgccgtggagtgggagtctaacggccagcctgagaacaactacaagaccacccctcctgtgctggactccgacggctccttcttcctgtactccaaactgaccgtggacaagtcccggtggcagcagggcaacgtgttctcctgctccgtgatgcacgaggccctgcacaaccactacacccagaagtccctgtccctgtctcccggcaag

SEQ ID NO: 394 - protein, 6 aa, Artificial Sequence
  source: /note=“Description of Artificial Sequence: Synthetic 6xHis tag”
HHHHHH